SEQUENCE LISTING (184 sequences)

SEQ ID NO: 1 - protein, 1455 aa, synthetic construct
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPGKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHMAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF

SEQ ID NO: 2 - RNA, 4365 nt, synthetic construct
  source 1..4365: mol_type = other RNA
atgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttc

SEQ ID NO: 3 - RNA, 4365 nt, synthetic construct
  source 1..4365: mol_type = other RNA
atgagcgacagctgggtgcctaatagcgccagcggacaggatcctggtggaaggagaagggcgtgggcagagctactggctggacgggtgaagcgagagaagtacaaccccgagcgagcacagaagctgaaggagtcagccgtgagactgctgcggagccaccaagacctgaacgccttactgctggaagtggagggacccctgtgcaagaagctgagcctgagcaaggtgatcgactgcgacagcagcgaggcctacgccaaccacagcagcagcttcatcggcagcgcattgcaggaccaagcttctaggctaggcgtccccgttggtatcctgagcgccggtatggtagcaagcagcgtgggccagatttgtaccgcacctgccgagactagccaccccgtgcttctgaccgtggagcagcggaagaagctgagcagcctgctggagttcgcccagtacctgcttgcgcacagcatgttcagccggctgagcttctgccaggagctgtggaagatccagagcagcctgctgctggaagccgtgtggcatctgcacgtgcagggcatcgtgagcctgcaggagctgctggaaagccacccagacatgcacgccgtagggagttggctgttccggaacctgtgctgcctgtgcgagcagatggaggccagctgtcaacacgctgacgtagctcgcgcaatgctgagcgacttcgtgcagatgttcgtgctgcggggcttccagaagaacagcgacctcagacggaccgtggaacccgagaagatgccacaggtgacagtggacgtgctgcagcggatgctgattttcgcgttagacgcactagccgcaggcgttcaggaggagagcagcacccacaagatagtgcggtgctggttcggcgtgttctctggccacacactggggagcgtgatcagcaccgatcccctgaagcggttcttcagccacaccctgacccagatcctgacccatagccccgtgctgaaggcaagcgacgccgtgcagatgcagcgtgagtggagcttcgcacggacccatccgctactgaccagcctataccggcgcctgttcgtgatgctatccgccgaggagttggtcggacacctgcaggaggtgctggagacccaggaggtgcactggcagagagtgctgagcttcgtgagcgccctggtggtgtgctttcccgaggcacagcagctgctggaagactgggtagctcggcttatggcccaggccttcgagagctgccagctggacagcatggtgaccgcctttctcgtggtgaggcaagcagccctagaaggccctagcgcctttctgagctacgccgactggttcaaagccagcttcggctccactcggggttaccacggctgcagcaagaaggccctggtgttcctgttcaccttcctgagcgagctggtgcccttcgagtcaccccggtacctgcaggtgcacatcctgcatccaccactggtccccgggaaataccggagcctgctgaccgactacatcagcctggccaagactcggctggcagacctgaaggtgagcatcgagaacatgggcctgtacgaggatctgagcagcgctggcgacatcacagaaccgcacagccaggccttgcaggacgtggagaaggccatcatggtgttcgagcacaccggcaacatccccgtgaccgtgatggaggccagcattttccgccggccatactacgttagccacttcctgcccgcactgctgactccacgcgtgctgccaaaggtccccgatagcagagtggctttcatcgagagcctgaaacgggccgacaagatcccacctagcctgtacagcacctactgccaagcttgtagcgctgccgaggagaaaccggaagacgctgcactgggtgttagggccgagcccaatagcgcagaggaacccttgggccaactgactgctgcacttggtgagcttcgggcaagcatgaccgacccctctcaacgggacgtgatcagcgcccaagtggccgtgatcagcgagcggttacgtgccgtgctgggccacaacgaggacgacagcagcgtggagatcagcaagatccagctgagcatcaatacgcctcgactggaacctcgggagcacatggccgtggacctgctgctgaccagcttctgccagaacttgatggccgctagcagtgtggctccacccgaacggcaaggaccttgggctgcgctgttcgtcaggaccatgtgtggccgggttcttcccgctgtgttgacccggctttgccagctgctgcggcatcaagggccatctttgtccgcacctcacgttctgggtcttgctgctctggccgtgcatctgggcgagagcagatcggcactgccggaagtggacgtgggacctcctgcacctggtgcaggtcttcccgtgccagccttattcgacagcctgcttacctgccgtactcgggacagcctgttcttctgcctgaagttctgcaccgccgccatcagctacagcctgtgcaagttcagcagccagagccgggacactctgtgtagctgtctgagccctggcctcatcaagaagttccagttcctgatgttcagattatttagcgaggctcggcaacctctgagcgaggaggacgttgccagcctgagttggcgtcccctacaccttcccagcgccgattggcagagggctgctctgtccctgtggactcaccgcactttccgggaggtgctgaaggaggaggacgtgcacctgacctaccaggactggctgcacctggagctcgagatacagcccgaagctgacgccctgtcagacaccgagcggcaggacttccaccagtgggccatccacgagcacttcctgcctgagagcagcgcctcaggagggtgtgacggggacctgcaagcagcctgcaccatcctggtgaacgccctgatggacttccaccagagcagccggagctacgaccacagcgagaacagcgatctagtcttcggcggcagaacaggtaacgaggacatcatcagccggctgcaggagatggtggccgacctggaactgcagcaggacctgattgtccccttgggtcacacccctagccaggagcacttcctgttcgagattttcaggaggcggctgcaagcactgaccagcggctggtctgtcgctgcttcccttcagcgtcagcgggagctgctgatgtacaagcggattctgctgcggctacccagcagtgtgctgtgcggctctagctttcaagccgagcagccaatcaccgctcgctgcgagcagttcttccacctggtgaacagcgagatgcggaatttctgttcccacggaggtgctctgacccaggacatcaccgcccatttcttccggggcctgctgaacgcttgtctccgatcgagagaccccagcctgatggtggacttcatcctggccaagtgccagaccaagtgtcccctgatcctgacctctgccctggtgtggtggccttcactggagcccgtgttgctgtgtcgctggcggagacattgtcaatcaccactgccacgggagctgcagaagctgcaagagggtcggcaattcgcctcggactttctgtctcctgaagctgctagccctgcccctaatcctgactggttgtctgcggccgcactacactttgccatccagcaggtgcgggaggagaacatccggaagcagctgaagaaactggactgcgagcgggaagagctgctggtgttcctgttcttcttcagcctgatgggcctgctgagctcacacctgaccagcaacagcactaccgacctgcccaaagccttccacgtgtgcgctgccatcctggagtgcctggagaagcggaagatcagctggctggccctgttccagctgacagagtctgatcttcggctggggcggttactcctgagagtagcacccgatcagcataccaggctgctccccttcgcattctacagcctgctgagctacttccacgaagacgccgccatacgcgaggaagctttcctgcacgtggccgtggacatgtacctgaagctggtgcagctgttcgttgctggcgacaccagcaccgtgtcacctcctgcaggcaggagcctggagctgaaaggtcagggcaaccccgtggagctgatcaccaaggcccggctgttcctgcttcagctgataccccggtgccccaagaagagctttagccacgtggccgaactgctggcagaccggggtgattgcgacccagaagtgtcagccgcccttcaaagccggcaacaagctgcccctgacgccgacttgagccaggagccgcacctgttc

SEQ ID NO: 4 - RNA, 4563 nt, synthetic construct
  source 1..4563: mol_type = other RNA
ggaaattattattatttctagctacaatttatcattgtattattttagctattcatcattatttacttggtgatcaacaatgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttctaaagctccccgggggcctcggtggcctagcttcttgccccttgggcctccccccagcccctcctccccttcctgcacccgtacccccgtggtctttgaataaagtctgagtgggcggc

SEQ ID NO: 5 - RNA, 4559 nt, synthetic construct
  source 1..4559: mol_type = other RNA
ggaaatcgcaaaatttgctcttcgcgttagatttcttttagttttctcgcaactagcaagctttttgttctcgccatgagcgacagctgggtgcctaatagcgccagcggacaggatcctggtggaaggagaagggcgtgggcagagctactggctggacgggtgaagcgagagaagtacaaccccgagcgagcacagaagctgaaggagtcagccgtgagactgctgcggagccaccaagacctgaacgccttactgctggaagtggagggacccctgtgcaagaagctgagcctgagcaaggtgatcgactgcgacagcagcgaggcctacgccaaccacagcagcagcttcatcggcagcgcattgcaggaccaagcttctaggctaggcgtccccgttggtatcctgagcgccggtatggtagcaagcagcgtgggccagatttgtaccgcacctgccgagactagccaccccgtgcttctgaccgtggagcagcggaagaagctgagcagcctgctggagttcgcccagtacctgcttgcgcacagcatgttcagccggctgagcttctgccaggagctgtggaagatccagagcagcctgctgctggaagccgtgtggcatctgcacgtgcagggcatcgtgagcctgcaggagctgctggaaagccacccagacatgcacgccgtagggagttggctgttccggaacctgtgctgcctgtgcgagcagatggaggccagctgtcaacacgctgacgtagctcgcgcaatgctgagcgacttcgtgcagatgttcgtgctgcggggcttccagaagaacagcgacctcagacggaccgtggaacccgagaagatgccacaggtgacagtggacgtgctgcagcggatgctgattttcgcgttagacgcactagccgcaggcgttcaggaggagagcagcacccacaagatagtgcggtgctggttcggcgtgttctctggccacacactggggagcgtgatcagcaccgatcccctgaagcggttcttcagccacaccctgacccagatcctgacccatagccccgtgctgaaggcaagcgacgccgtgcagatgcagcgtgagtggagcttcgcacggacccatccgctactgaccagcctataccggcgcctgttcgtgatgctatccgccgaggagttggtcggacacctgcaggaggtgctggagacccaggaggtgcactggcagagagtgctgagcttcgtgagcgccctggtggtgtgctttcccgaggcacagcagctgctggaagactgggtagctcggcttatggcccaggccttcgagagctgccagctggacagcatggtgaccgcctttctcgtggtgaggcaagcagccctagaaggccctagcgcctttctgagctacgccgactggttcaaagccagcttcggctccactcggggttaccacggctgcagcaagaaggccctggtgttcctgttcaccttcctgagcgagctggtgcccttcgagtcaccccggtacctgcaggtgcacatcctgcatccaccactggtccccgggaaataccggagcctgctgaccgactacatcagcctggccaagactcggctggcagacctgaaggtgagcatcgagaacatgggcctgtacgaggatctgagcagcgctggcgacatcacagaaccgcacagccaggccttgcaggacgtggagaaggccatcatggtgttcgagcacaccggcaacatccccgtgaccgtgatggaggccagcattttccgccggccatactacgttagccacttcctgcccgcactgctgactccacgcgtgctgccaaaggtccccgatagcagagtggctttcatcgagagcctgaaacgggccgacaagatcccacctagcctgtacagcacctactgccaagcttgtagcgctgccgaggagaaaccggaagacgctgcactgggtgttagggccgagcccaatagcgcagaggaacccttgggccaactgactgctgcacttggtgagcttcgggcaagcatgaccgacccctctcaacgggacgtgatcagcgcccaagtggccgtgatcagcgagcggttacgtgccgtgctgggccacaacgaggacgacagcagcgtggagatcagcaagatccagctgagcatcaatacgcctcgactggaacctcgggagcacatggccgtggacctgctgctgaccagcttctgccagaacttgatggccgctagcagtgtggctccacccgaacggcaaggaccttgggctgcgctgttcgtcaggaccatgtgtggccgggttcttcccgctgtgttgacccggctttgccagctgctgcggcatcaagggccatctttgtccgcacctcacgttctgggtcttgctgctctggccgtgcatctgggcgagagcagatcggcactgccggaagtggacgtgggacctcctgcacctggtgcaggtcttcccgtgccagccttattcgacagcctgcttacctgccgtactcgggacagcctgttcttctgcctgaagttctgcaccgccgccatcagctacagcctgtgcaagttcagcagccagagccgggacactctgtgtagctgtctgagccctggcctcatcaagaagttccagttcctgatgttcagattatttagcgaggctcggcaacctctgagcgaggaggacgttgccagcctgagttggcgtcccctacaccttcccagcgccgattggcagagggctgctctgtccctgtggactcaccgcactttccgggaggtgctgaaggaggaggacgtgcacctgacctaccaggactggctgcacctggagctcgagatacagcccgaagctgacgccctgtcagacaccgagcggcaggacttccaccagtgggccatccacgagcacttcctgcctgagagcagcgcctcaggagggtgtgacggggacctgcaagcagcctgcaccatcctggtgaacgccctgatggacttccaccagagcagccggagctacgaccacagcgagaacagcgatctagtcttcggcggcagaacaggtaacgaggacatcatcagccggctgcaggagatggtggccgacctggaactgcagcaggacctgattgtccccttgggtcacacccctagccaggagcacttcctgttcgagattttcaggaggcggctgcaagcactgaccagcggctggtctgtcgctgcttcccttcagcgtcagcgggagctgctgatgtacaagcggattctgctgcggctacccagcagtgtgctgtgcggctctagctttcaagccgagcagccaatcaccgctcgctgcgagcagttcttccacctggtgaacagcgagatgcggaatttctgttcccacggaggtgctctgacccaggacatcaccgcccatttcttccggggcctgctgaacgcttgtctccgatcgagagaccccagcctgatggtggacttcatcctggccaagtgccagaccaagtgtcccctgatcctgacctctgccctggtgtggtggccttcactggagcccgtgttgctgtgtcgctggcggagacattgtcaatcaccactgccacgggagctgcagaagctgcaagagggtcggcaattcgcctcggactttctgtctcctgaagctgctagccctgcccctaatcctgactggttgtctgcggccgcactacactttgccatccagcaggtgcgggaggagaacatccggaagcagctgaagaaactggactgcgagcgggaagagctgctggtgttcctgttcttcttcagcctgatgggcctgctgagctcacacctgaccagcaacagcactaccgacctgcccaaagccttccacgtgtgcgctgccatcctggagtgcctggagaagcggaagatcagctggctggccctgttccagctgacagagtctgatcttcggctggggcggttactcctgagagtagcacccgatcagcataccaggctgctccccttcgcattctacagcctgctgagctacttccacgaagacgccgccatacgcgaggaagctttcctgcacgtggccgtggacatgtacctgaagctggtgcagctgttcgttgctggcgacaccagcaccgtgtcacctcctgcaggcaggagcctggagctgaaaggtcagggcaaccccgtggagctgatcaccaaggcccggctgttcctgcttcagctgataccccggtgccccaagaagagctttagccacgtggccgaactgctggcagaccggggtgattgcgacccagaagtgtcagccgcccttcaaagccggcaacaagctgcccctgacgccgacttgagccaggagccgcacctgttctaaagctccccgggggcctcggtggcctagcttcttgccccttgggcctccccccagcccctcctccccttcctgcacccgtacccccgtggtctttgaataaagtctgagtgggcggc

SEQ ID NO: 6-13 - skipped (8 entries)

SEQ ID NO: 14 - RNA, 4365 nt, synthetic construct
  source 1..4365: mol_type = other RNA
atgagcgacagctgggtgcccaacagtgcttccgggcaggatcccggcggtagaagacgggcttgggctgagctgctggccggcagggtgaagcgggagaagtacaaccccgagcgggcccagaagctgaaggagagtgccgtgcggctgctgcggagccaccaggacctgaacgccctcctgctggaggtcgagggcccactgtgcaagaagctcagcctgagcaaggtgatcgactgtgacagcagcgaggcctacgccaaccacagctccagcttcatcggcagcgccctgcaggaccaggccagcagactgggcgtgcccgtgggcatcctgagcgccggcatggtggccagctcggtgggccagatctgcaccgctcccgccgagaccagccaccccgtgctgctcaccgtggagcagcggaagaagctgagcagcctgctggagttcgcccagtacctgctggcccacagcatgttctcccggctgagcttttgccaagagctgtggaagatccagagctccctgctgctggaggccgtgtggcaccttcacgtgcagggcatcgtgagcctgcaggaactgctggagagccaccccgacatgcacgccgtgggcagctggctgttccggaacctgtgctgcctgtgcgagcagatggaggctagctgccagcacgccgacgtggcacgggccatgctgagcgacttcgtgcagatgttcgtgctgcggggcttccagaagaatagcgacctacggcggaccgtggagcccgagaagatgccccaggtgaccgtggacgtgctgcagcggatgctgatcttcgccctggacgctctggccgctggcgtgcaggaggagagcagcacccacaagatcgtgcggtgctggttcggcgtgttcagcggccacaccctgggcagcgtgatcagcaccgacccactgaagcggttcttcagccacaccctcacccagatcctgacccacagccccgtgctgaaggccagcgacgccgtgcagatgcagcgggagtggagcttcgcccggacccacccactgctgactagcctgtaccggcggctgttcgtgatgctgagcgcagaggagctggtgggccacctgcaggaggtgctggagacccaggaggtgcactggcagcgggtgctgagcttcgtgagcgccctggtggtgtgcttccccgaggcccagcagctcctggaggactgggtggcccggctgatggcccaggccttcgagagctgccagctggacagcatggtgaccgccttcctggtggtgcggcaggctgccctggaaggccccagcgctttcctgagctacgccgactggttcaaggccagcttcggctccacccggggctaccacggctgcagcaagaaggccctggtgttcctgttcaccttcctgagcgagctggtgcccttcgagtcaccccggtacctgcaggtgcacatcctgcatcctccgctggtgcccggcaagtaccggagcctgctgaccgactacatcagcctggccaagacccggctggccgacctgaaggtgagcatcgagaacatgggcctgtacgaggacctgagcagcgccggcgacatcaccgagccccacagccaggccctgcaagacgtggagaaggccatcatggtgttcgagcacaccggcaacatccccgtgaccgtcatggaggccagcatcttccggcggccctactacgtgagccacttcctgcccgccctgctgaccccacgggtgctgcccaaggtgcccgacagccgggtggccttcatcgagagcctgaagcgggccgacaagatcccacccagcctgtacagcacctactgccaggcctgctctgccgccgaggagaagcccgaggacgccgcattaggcgtgcgggccgagcccaacagcgccgaggaacccctggggcagctgaccgcagccctgggagagctgcgggccagcatgaccgaccccagccagcgggacgtgatcagcgcccaggtggccgtcatcagcgagcggctgcgggcagtgctgggccacaacgaggacgacagcagcgtggagatcagcaagatccagctgagcatcaacaccccacggctggaacctcgggagcacatggccgtggacctgctgctgaccagcttctgccagaacctgatggccgcctctagcgtggccccaccagaacggcagggtccctgggctgccctgttcgtgcggaccatgtgcggccgggttctgcccgctgtgctgacccggctgtgccagctgctgcggcaccagggccctagcctgagcgctccccacgtgctgggcctagctgccctggccgtgcaccttggggagtcccgaagcgccctgcccgaggtggacgtgggccctccagccccaggtgctggccttcccgtgcccgctctgttcgacagtctgctgacctgccggacccgggacagcctgttcttctgcctgaagttctgcaccgccgccatcagctacagcctgtgcaagttcagcagccagagccgggacaccctgtgcagctgcctgagccccggcctgatcaagaagttccagttcctgatgttccggctgttcagcgaagcccggcagcccctgagcgaggaggacgtcgccagcctgtcctggagacccctgcacctgcccagcgccgactggcagagagccgccctgagcctgtggacccaccggaccttccgggaggtgctgaaggaagaggacgtgcacctgacctaccaggactggctgcacctggagctggagattcagcccgaggccgacgccctgagcgacaccgagcggcaggatttccaccagtgggccatccacgagcatttcctgcccgagagcagcgccagcggcggctgtgacggcgacctgcaggccgcctgcaccatcctggtgaacgccctgatggacttccaccagagcagccggagctacgaccacagcgagaacagcgacctggtgttcggcggacggaccggcaacgaggacatcatcagccggctgcaggagatggtcgccgacctggagttgcagcaggacctgatcgtgcccctgggccacactcccagccaggagcacttcctgttcgagatcttccgccggagactgcaggccctgacctcaggctggagcgtggccgccagcctgcaacggcagcgggagctgctgatgtacaagcggatcctgctgcggctgcccagcagcgtgctgtgcggcagcagcttccaggccgagcagcccatcacggcccggtgcgagcagttcttccacctggtgaacagcgagatgcggaacttctgcagccacggaggcgccctgacccaggacatcaccgcccacttcttccggggcctgctgaacgcctgcctgcggagcagagaccccagcctgatggtggacttcatcctggccaagtgccagaccaagtgtcccctgatcctgacctccgccctggtgtggtggcccagcctggagcccgtgcttctgtgtcggtggcggcggcactgccagagccctctgccccgggagctgcagaagctgcaggagggccggcagttcgccagcgacttcctgtctcccgaggccgctagcccagccccaaaccccgactggctgagcgctgccgccctgcacttcgccatccagcaggtgcgggaggagaacatccggaagcagctgaagaagctggactgcgagcgggaggagctgctcgtgttcctcttcttcttcagcctcatgggccttctgagcagccacctgaccagcaacagcaccaccgacctgcccaaggccttccacgtgtgcgccgccatcctggagtgcctggagaagcggaagatcagctggctggccctgttccagctgaccgagagcgacctgcggctgggccgactgctgctgcgggtggccccagaccagcacacccggctgctgcccttcgccttctacagcctgctgagctacttccacgaggacgctgccatccgggaggaggcctttctgcacgtggcagtggacatgtacctgaagctggtgcagctgttcgtggccggcgacaccagcaccgtgtcaccaccagccggccgctccctggagctgaagggccagggcaaccccgtggagctgatcaccaaggcccggctgttcctgctgcagctgatcccacggtgccccaagaagagcttcagccacgtggccgagctgcttgccgaccggggagactgcgaccccgaggtgagcgccgccttgcagagccggcagcaggccgcacctgacgccgacctgagccaggagccccacctgttc

SEQ ID NO: 15 - RNA, 4365 nt, synthetic construct
  source 1..4365: mol_type = other RNA
atgagcgacagctgggtgcccaacagcgccagtggccaagaccccggcggtcggagacgagcgtgggctgagctgctggccggacgggtgaagcgggagaagtacaaccccgagcgggcccagaagctgaaggagagcgctgtgcggctgctgcggagccaccaggacctgaacgccctgctgctggaggtggagggcccactgtgcaagaagctgagcctgagcaaggtgatcgactgcgacagcagcgaggcctacgccaaccacagcagcagcttcatcggcagcgccctgcaggaccaggccagcagactgggcgtgcccgtgggcatcctgagcgccggcatggtggccagcagcgtgggccagatctgcaccgccccggccgagacaagccaccccgtgctgctgaccgtggagcagcggaagaagctgagcagcctgctggagttcgcccagtacctgctggcccacagcatgttcagccggctgagcttctgccaggagctgtggaagatccagagcagcctgctgctggaggccgtgtggcacctgcacgtgcagggcatcgtgagcctgcaggagctgctggagagccaccccgacatgcacgccgtgggcagctggctgttccggaacctgtgctgcctgtgcgagcagatggaggccagctgccagcacgccgacgtggctcgggccatgctgagcgacttcgtgcagatgttcgtgctgcggggcttccagaagaacagcgacctgcggcggaccgtagagcccgagaagatgccccaggtgaccgtggacgtgctgcagcggatgctgatcttcgcactcgacgccctggccgctggtgtgcaggaggagagcagcacccacaagatcgtgcggtgctggttcggcgtgttcagcggccacaccctgggcagcgtgatcagcaccgatcccctgaagcggttcttcagccacaccctgacccagatcctgacccacagccccgtgctgaaggccagcgacgccgtgcagatgcagcgggagtggagcttcgcccggacccatcccctgctgaccagcctgtaccggcggctgttcgtgatgctgagcgccgaggagctggtgggccacctgcaggaggtgctggagacccaggaggtgcactggcagcgggtgctgagcttcgtgagcgccctggtggtgtgcttccccgaggcccagcagctgctggaggactgggtggcccggctgatggcccaggccttcgagagctgccagctggacagcatggtgaccgccttcctggtggtaaggcaagccgccctggagggtcccagcgccttcctgagctacgccgactggttcaaggccagcttcggcagcacccggggctaccacggctgcagcaagaaggccctggtgttcctgttcaccttcctgagcgagctggtgcccttcgagtcaccccggtacctgcaggtgcacatcctgcacccacccctggtgcccggcaagtaccggagcctgctgaccgactacatcagcctggccaagacccggctggccgacctgaaggtgagcatcgagaacatgggcctgtacgaggacctgagcagcgccggcgacatcaccgagccccacagccaggccctgcaggacgtggagaaggccatcatggtgttcgagcacaccggcaacatccccgtgaccgtgatggaggccagcatcttccggcggccctactacgtgagccacttcctgcccgccctgctgactccccgggtgctgcccaaggtgcccgacagccgggtggccttcatcgagagcctgaagcgggccgacaagattcctcccagcctgtacagcacctactgccaggcctgcagtgccgccgaggagaagcccgaagacgccgctctgggcgtgagagccgagcccaacagcgccgaggagcctttgggccagctgacagcagcccttggcgagctgcgggccagcatgaccgaccccagccagcgggacgtgatcagcgcccaggtggccgtgatctccgagagactgcgggccgtgctgggccacaacgaggacgacagcagcgtggagatcagcaagatccagctgagcatcaacactccccggctggagcccagggagcacatggccgtggacctgctgctgaccagcttctgccagaacctgatggccgcctcaagcgtggcccctccagagcggcaaggcccttgggcggccctgttcgtgcggaccatgtgcggccgggtgttgcctgccgtgctgacccggctgtgccagctgctgcggcaccagggccctagcctgagcgctccccacgtgctggggctggccgccttagcagtgcacctgggcgagtcacggagcgccctgcccgaggtggacgtgggccctcctgcgccaggagcagggcttcccgtgcccgccctgttcgacagcctgctgacctgccggacccgggacagcctgttcttctgcctgaagttctgcaccgccgccatcagctacagcctgtgcaagttcagcagccagagccgggacaccctgtgcagctgcctgagccccggcctgatcaagaagttccagttcctgatgttccggctgttcagcgaggcccggcaacccctgagcgaggaggacgtggccagcctgagctggcggcccctgcaccttcccagcgccgactggcaacgcgccgccctgagcctgtggacccaccggaccttccgggaggtgctgaaggaggaggacgtgcacctgacctaccaggactggctgcacctggagctggagattcagcccgaggccgacgccctgagcgacaccgagcggcaggacttccaccagtgggccatccacgagcacttcctgcccgagagcagtgccagcggcggctgtgacggcgacctgcaggccgcctgcaccatcctggtgaacgccctgatggacttccaccagagcagccggagctacgaccacagcgagaacagcgacctggtgttcggaggccggaccggcaacgaggacatcatcagccggctgcaggagatggtggccgacctggagctgcagcaggacctgatcgtgcccctgggccacacccctagccaggagcacttcctgttcgagatctttcggcggcggctgcaagcgctgaccagcggttggagcgtggccgcctccctgcaacggcagcgggagctgctgatgtacaagcggatcctgctgcggctgcccagcagcgtgctgtgcggcagcagcttccaggccgagcagcccatcaccgcccggtgcgagcagttcttccacctggtgaacagcgagatgcggaacttctgcagccacggtggcgccctgacccaggacatcaccgcccacttcttccggggcctgctgaacgcctgcctgcgtagccgggaccccagcctgatggtggacttcatcctggccaagtgccagaccaagtgcccactgatcctgaccagcgccctggtgtggtggcccagcctggagcccgtgctgctgtgccggtggcgacggcactgccagtcaccgctgccccgggagctgcagaagctgcaggagggccggcagttcgccagcgacttcctgagccccgaggctgccagccctgccccgaaccccgactggctgagtgccgccgccctgcacttcgccatccagcaggtgcgggaggagaacatccggaagcagctgaagaagctggactgcgagcgggaggagctgctggtgttcctgttcttcttcagcctgatgggcctgctgagcagccacctgaccagcaacagcaccaccgacctgcccaaggccttccacgtgtgcgccgccatcctggagtgcctggagaagcggaagatcagctggctggccctgttccagctgaccgagtctgacctgcgcctgggcaggctgctgctgcgggtggctcccgaccagcacacccggctgctgcccttcgccttctacagcctgctgagctacttccacgaggacgccgccatccgggaggaggccttcctgcacgtggccgtggacatgtacctgaagctggtgcagctgttcgtggccggcgacaccagcactgtatctccaccggctgggcggtccctggagctgaagggccagggcaaccccgtggagctgatcaccaaggcccggctgttcctgctgcagctgatcccacggtgccccaagaagagcttcagccacgtggccgagctgctggccgaccggggagactgcgaccccgaggttagtgccgccctccagagccggcagcaagccgcccctgacgccgacctgagccaggagccccacctgttc

SEQ ID NO: 16 - RNA, 4365 nt, synthetic construct
  source 1..4365: mol_type = other RNA
atgtccgactcttgggtccccaactccgcctctggtcaagatccgggaggaaggagacgtgcctgggcggaactgttagccggacgtgtgaagagggagaagtacaatcccgagcgcgcacagaagctcaaggaatccgcagttaggctcctccgctctcaccaggacctcaacgcactgctcctcgaggtcgagggaccactctgcaagaagctctccctctccaaggtcatcgactgcgactcctccgaggcctacgccaaccactcctcctccttcatcggctccgccctccaagatcaagcttcccgcctcggagttcctgtgggcatcctaagcgccgggatggtcgcatcttccgtcggccagatttgcacagcacccgccgagacatcccatcctgtccttctcaccgtcgagcagcgcaagaagctctcctccctcctcgagtttgcccagtacctcctcgcccactccatgttctcccgcctctccttctgccaggagctctggaagatccagtcctccctcctgctcgaagccgtctggcatctccacgtccagggcatcgtctccctccaggagctcctcgaatcccatccagacatgcacgccgtcggttcttggctcttccgcaacctctgctgcctctgcgagcagatggaagcctcctgccaacacgctgacgtcgctcgagccatgctctccgacttcgtccagatgttcgtcctccgcggcttccagaagaattccgacctccgtcgcacagtcgagcctgagaagatgcctcaggtcaccgtcgacgtcctccagagaatgctcatcttcgcactggacgcactagctgcaggcgtccaggaggagtcctccacccacaagatcgtccgctgttggttcggcgtctttagcgggcacaccttggggtcagtcatctccaccgatcccctcaagcgcttcttctcccacaccctcacccagatcctcacccactccccagtcctcaaagcctccgacgccgtccagatgcaacgcgagtggtcctttgcccgaacccacccactcctcacctccctctaccgcaggctcttcgtcatgctctccgccgaagaactcgtcggccacttgcaggaggtcctcgagacccaagaagtccactggcagcgcgtcctctccttcgtctccgccctcgtcgtctgttttccagaggcccagcagctccttgaggattgggtggcacggctcatggctcaggccttcgagtcctgccagctcgactccatggtcaccgcctttcttgttgtccgccaagctgctctcgagggtccttcggcattcctgagctacgccgactggttcaaggcctccttcggtagcacaaggggctaccacggttgctccaagaaggccctcgtcttcctcttcaccttcctctccgagctcgtccccttcgagagtccccgctacctccaggtccacatcctccatcctcctctggtccccggaaagtaccgctccctcctcaccgactacatctccctcgctaagacccgactcgccgacttgaaggtctccatcgagaacatgggcctctacgaagatctctccagtgcaggcgacatcaccgagccacactcccaagccctccaggacgtcgagaaggccatcatggtcttcgagcacaccggcaacatccccgtcaccgtcatggaggcctccatcttcagacgcccctactacgtctcccactttctccctgccctcctaacccctcgggtactccccaaggtccccgactccagagtcgccttcatcgagtccctcaagcgcgcagacaagatcccaccatccctctactccacctattgtcaagcctgttctgccgccgaagagaaacccgaggacgccgcattaggcgtccgtgcagagcccaactcagctgaggagccactgggccagctaacagctgcactaggcgagctgagggcgagtatgaccgacccttcccagcgagacgtcatctccgcccaagtcgccgtcatttccgaacgcttgagggcagtgctgggccacaacgaggacgactcctccgtcgagatctccaagatccagctctccatcaatacacctcgcctggaaccccgagagcacatggccgtcgacctcctcctcacctccttctgccagaacttgatggctgcctcctccgttgctcctccggaaagacaaggtccctgggccgctctctttgtccgcaccatgtgtggacgagtgctcccagctgtcctcactcgcctctgccagttgctccgccaccaaggaccctcactctctgcaccgcacgtactgggactcgcagcactcgcagtccacctcggagaatcccgctctgctctcccagaagtggacgtgggacctcctgcacctggtgctggcttgcccgtaccagctctcttcgactccctcctcacgtgcagaacccgcgactccctcttcttctgcctcaagttctgcaccgccgccatctcctactccctctgcaagttcagctcccagtcacgcgacaccctctgctcctgcttgtcccctggcctcatcaagaagttccagttcctcatgttccgcctcttctccgaggccagacaacccctctccgaggaggacgtggcttccctcagttggagacccctccacttgccttccgccgattggcaaagagccgcgctctctctctggacccatcgcaccttccgcgaggtcctcaaggaggaggacgtccacctcacctaccaggactggctccacctcgagctcgagatccaacccgaagcagacgccctctccgacaccgaacgccaggacttccaccagtgggccatccacgagcacttcctccccgaaagcagtgcttccggtggttgcgacggggatctccaagccgcttgcaccatcctcgtcaacgccctcatggacttccaccagtcctcccgctcctacgaccactccgagaactccgacctagtgtttggcggccgcactggaaacgaggacatcatctcccgcctccaggagatggtggccgatctcgagctccagcaggatctcatcgtccctctcggccacacaccttcgcaggagcacttcctcttcgaaatcttcagaagacgcctccaagcgctcacttccggttggtctgtggccgcaagtctccaacgccagagggaactcctcatgtacaagcgcatcctcctacgcctcccttcctccgtcctctgtgggtcctccttccaggcagaacagcccatcactgcccgctgtgagcagttcttccacctcgtcaactccgagatgcgcaacttctgttcacacggcggtgcactcacccaagacatcaccgcgcacttcttccgcggactcctgaacgcctgcttgagatcccgcgacccatccctcatggtcgacttcatcctcgccaagtgccagaccaagtgtcccctcatcctcaccagtgccctcgtttggtggccctcccttgaacccgtcctcctctgcaggtggagacgccactgtcagtctccgttaccccgcgagctccagaaactccaggagggacgccagttcgcttccgacttcctctcacccgaagctgctagcccagcacccaaccctgactggttatccgccgccgctttgcacttcgccatccagcaagtgcgcgaggagaacatccgcaagcagctcaagaagctcgactgcgagcgagaggagctcctcgttttcctcttcttcttctccctcatgggcctcctctcatcccacctcacctccaactccaccaccgacttgcccaaggcctttcacgtctgtgccgccatcctcgagtgcctcgagaagcgcaagatctcctggctcgccctcttccagctaacagagtccgacctgagactcggtcgcctcctactcagagtcgcacccgaccagcacactcgcctcctccccttcgcattctactccctcctctcctacttccacgaagacgcagcaatccgcgaggaggccttccttcacgtcgccgtcgacatgtacctcaagctcgtccagctctttgtcgcgggcgacacgagtacagttagccctccagcaggtaggtccctcgagcttaaaggccagggcaacccagtcgagctcatcaccaaggcccgcctgttcctgctacagctcatcccacgctgccccaagaagtccttctcccacgtagccgaactcctcgccgatcgcggagattgcgaccccgaagtttccgctgccttgcagtcacggcagcaagcagccccagacgcagacctctcacaggagccccacctcttc

SEQ ID NO: 17 - RNA, 4365 nt, synthetic construct
  source 1..4365: mol_type = other RNA
atgtcagattcctgggtgcccaactccgctagcggacaagatccaggtggccgcaggagagcctgggctgaactgcttgccgggagagtgaagagagagaagtacaacccagagcgcgcccagaagttaaaggagagcgccgtaagactgctgcgcagccaccaagacttaaacgctctgctgctggaggtggaggggcccctgtgcaagaagctgagcctgtccaaggtgatcgattgtgacagcagcgaggcttacgccaatcattcatcttccttcatcggctcagcactgcaggatcaagcaagccggctgggcgttccagttggcatactgagcgccggcatggtggccagcagcgtgggacagatctgcaccgctcccgccgagacctcacatcccgttctgctgacagttgagcaacgcaagaagctgtcaagcctgctggagttcgcacagtacctgttagcccacagcatgttttcccgcttaagcttctgccaggagctgtggaagatccagtcaagtctgctactggaggccgtgtggcacttacacgtgcagggtatcgtgtcactgcaagagctactggagagccatcccgatatgcacgccgtggggtcttggctgttccgcaatctgtgctgcctgtgcgaacagatggaagcctcctgccaacacgccgacgttgctagagcaatgctgagtgacttcgttcagatgttcgtgctgcgcgggttccagaagaactcagatctccggaggaccgtggagcctgagaagatgccccaggtaaccgtggacgtgctgcagcggatgttaatctttgccctggacgctctcgctgccggagtgcaggaggagagctccacccacaagatcgtgcggtgctggttcggcgtgttttccggccataccttaggctccgtgatctccaccgatcccctgaaacggttcttctcccacacactgacccagatactgacccactcccctgtgttaaaggctagcgacgccgtgcaaatgcaacgcgagtggtcctttgcccgcacccatccactgctgaccagtctgtaccggcggctgtttgttatgctgtccgccgaggagttagtgggccacctgcaggaggttctggaaacccaggaggtgcattggcagcgcgtgttaagcttcgtgagcgccctggtggtgtgctttcccgaggcccagcagttactggaggactgggtggcccggctgatggcccaggccttcgagagctgccagctggacagcatggtgaccgccttcctggtggtacgtcaagctgccctggagggcccaagcgccttcctgagttacgctgactggttcaaggccagcttcgggtccacccgcgggtaccacggctgctccaagaaggccctggtgttcctgtttaccttcctgagcgagctggtgccatttgagtctccccgctacctgcaggtgcacatcctgcacccacccctggtccctggaaagtacagatcactgttaaccgactacatctctctggccaagacccggttagccgacctgaaggtgtctatcgagaatatgggcctgtacgaggacctgtccagcgccggcgatatcaccgagccccacagccaagccttacaggacgtggagaaggccattatggtgttcgagcacaccggcaatatccccgtgaccgtaatggaggcttctatcttccggcggccctactacgtgtcccacttcctgcccgccctgttaacacccagagtgttacccaaggtgcccgacagtcgcgttgccttcatagagagcttaaagagagccgacaagatccctcctagtttatactccacctactgccaggcttgctccgccgccgaggagaagcccgaggacgcagccttaggagtaagagctgagcccaactcagccgaggagccactgggccagttaaccgctgccctgggagagttacgggcctctatgaccgaccccagccaacgcgacgttatctctgcccaagtggcagtgataagcgagaggctgcgggcagtattaggacacaacgaggacgacagcagcgtggagatcagtaagatccaattaagcatcaacacacccaggctggagcccagagagcacatggccgtggacctgctgttaacctccttctgtcagaacttaatggccgcaagtagcgtggctcctccagaacgccaaggtccttgggctgccttattcgttcgcaccatgtgtggccgcgtcctgccagcagtgctgacccggctgtgccagttactgcggcaccaagggccctccctgagcgctcctcacgtgcttgggttggccgccctcgctgtacacttaggtgaaagcagaagcgcccttcctgaggtggacgtgggtcctccagccccaggtgccggactccccgtgccagccctgttcgacagcttactgacttgtcgcacccgggactccctgttcttttgtctgaagttctgcaccgccgccatctcctactcactgtgcaagttctctagccagtcccgggataccctgtgctcctgcctgtctccaggcctgatcaagaagttccagttcctgatgttcagactgttctcagaagcaagacagcctctgtccgaggaggacgtggcctccttatcctggcgacccctgcacctgccctccgctgattggcaaagagctgcacttagcctgtggacccaccggacatttcgcgaggtgctgaaagaagaagacgtacacctgacctaccaagactggctgcacctggagctggagatccagcctgaagccgacgccctgagcgacaccgagcgccaagactttcaccagtgggccatacacgagcactttctgcccgagtcaagcgccagtggaggctgcgacggtgatctgcaagcagcctgcaccatcttagtgaacgccctgatggacttccaccaatcttcccgcagctacgaccactccgagaacagcgacctggtgtttggaggccgcactgggaacgaggacatcattagccgcctgcaagaaatggtggctgacctggaactccaacaggacttaatcgtacctctggggcacacaccaagccaggagcacttcttatttgagatatttagaagacgcttacaagcactgacttcaggctggagtgtagctgcaagcctgcaacgccaacgcgagctgctgatgtataagcggatactgctgcgcctgccctcctctgtactgtgcggctcctccttccaggccgagcagcccatcaccgcccggtgtgagcagttcttccacctggtgaacagcgagatgcggaacttctgttctcacggaggcgctctgacccaagacatcacagcccacttcttccgcggcctgctgaacgcctgtctgcggtcaagggaccccagcctgatggtcgacttcatactggcaaagtgtcagaccaagtgtcccctgatcctgaccagcgctctggtttggtggccatcactggagcccgtactcctgtgtcgctggaggcggcattgccagtcacctttaccccgagaactgcagaagctgcaggagggccggcagtttgccagcgacttcctgtcaccagaggcagcctctcccgctcctaaccccgattggctatccgcggccgctctgcacttcgccatccagcaggtgcgggaggagaacatacggaaacaactgaagaaattagactgcgagagagaggagttacttgtgttcctgttcttcttcagtctgatgggcctgctgtcctcccacctgaccagcaattcaaccaccgacctgcccaaggccttccacgtttgtgccgccatcctggagtgcctggagaagcggaagattagctggttagccctgtttcagctgaccgagagtgatttacgcctggggagactgctgttacgcgtggctcccgaccagcacaccaggctgctgcctttcgccttctacagtctgttatcctacttccacgaggacgccgccatcagagaggaggccttcctgcacgtggccgtggacatgtacttaaagctggtgcagctgtttgtggccggcgatacctccacagtaagccctcctgctggcagaagcctggagctgaagggccagggtaaccccgtagagctgattactaaggcccgcctgtttctgctgcaactgatccctcgctgccccaagaagagctttagccacgtggccgaattactggctgacaggggagactgtgatcctgaggtgagcgctgccttacagagccgtcaacaagcggcccctgacgccgatttaagccaagagcctcacctcttc

SEQ ID NO: 18-22 - skipped (5 entries)

SEQ ID NO: 23 - RNA, 4559 nt, synthetic construct
  source 1..4559: mol_type = other RNA
ggaaatcgcaaaatttgctcttcgcgttagatttcttttagttttctcgcaactagcaagctttttgttctcgccatgagcgacagctgggtgcccaacagtgcttccgggcaggatcccggcggtagaagacgggcttgggctgagctgctggccggcagggtgaagcgggagaagtacaaccccgagcgggcccagaagctgaaggagagtgccgtgcggctgctgcggagccaccaggacctgaacgccctcctgctggaggtcgagggcccactgtgcaagaagctcagcctgagcaaggtgatcgactgtgacagcagcgaggcctacgccaaccacagctccagcttcatcggcagcgccctgcaggaccaggccagcagactgggcgtgcccgtgggcatcctgagcgccggcatggtggccagctcggtgggccagatctgcaccgctcccgccgagaccagccaccccgtgctgctcaccgtggagcagcggaagaagctgagcagcctgctggagttcgcccagtacctgctggcccacagcatgttctcccggctgagcttttgccaagagctgtggaagatccagagctccctgctgctggaggccgtgtggcaccttcacgtgcagggcatcgtgagcctgcaggaactgctggagagccaccccgacatgcacgccgtgggcagctggctgttccggaacctgtgctgcctgtgcgagcagatggaggctagctgccagcacgccgacgtggcacgggccatgctgagcgacttcgtgcagatgttcgtgctgcggggcttccagaagaatagcgacctacggcggaccgtggagcccgagaagatgccccaggtgaccgtggacgtgctgcagcggatgctgatcttcgccctggacgctctggccgctggcgtgcaggaggagagcagcacccacaagatcgtgcggtgctggttcggcgtgttcagcggccacaccctgggcagcgtgatcagcaccgacccactgaagcggttcttcagccacaccctcacccagatcctgacccacagccccgtgctgaaggccagcgacgccgtgcagatgcagcgggagtggagcttcgcccggacccacccactgctgactagcctgtaccggcggctgttcgtgatgctgagcgcagaggagctggtgggccacctgcaggaggtgctggagacccaggaggtgcactggcagcgggtgctgagcttcgtgagcgccctggtggtgtgcttccccgaggcccagcagctcctggaggactgggtggcccggctgatggcccaggccttcgagagctgccagctggacagcatggtgaccgccttcctggtggtgcggcaggctgccctggaaggccccagcgctttcctgagctacgccgactggttcaaggccagcttcggctccacccggggctaccacggctgcagcaagaaggccctggtgttcctgttcaccttcctgagcgagctggtgcccttcgagtcaccccggtacctgcaggtgcacatcctgcatcctccgctggtgcccggcaagtaccggagcctgctgaccgactacatcagcctggccaagacccggctggccgacctgaaggtgagcatcgagaacatgggcctgtacgaggacctgagcagcgccggcgacatcaccgagccccacagccaggccctgcaagacgtggagaaggccatcatggtgttcgagcacaccggcaacatccccgtgaccgtcatggaggccagcatcttccggcggccctactacgtgagccacttcctgcccgccctgctgaccccacgggtgctgcccaaggtgcccgacagccgggtggccttcatcgagagcctgaagcgggccgacaagatcccacccagcctgtacagcacctactgccaggcctgctctgccgccgaggagaagcccgaggacgccgcattaggcgtgcgggccgagcccaacagcgccgaggaacccctggggcagctgaccgcagccctgggagagctgcgggccagcatgaccgaccccagccagcgggacgtgatcagcgcccaggtggccgtcatcagcgagcggctgcgggcagtgctgggccacaacgaggacgacagcagcgtggagatcagcaagatccagctgagcatcaacaccccacggctggaacctcgggagcacatggccgtggacctgctgctgaccagcttctgccagaacctgatggccgcctctagcgtggccccaccagaacggcagggtccctgggctgccctgttcgtgcggaccatgtgcggccgggttctgcccgctgtgctgacccggctgtgccagctgctgcggcaccagggccctagcctgagcgctccccacgtgctgggcctagctgccctggccgtgcaccttggggagtcccgaagcgccctgcccgaggtggacgtgggccctccagccccaggtgctggccttcccgtgcccgctctgttcgacagtctgctgacctgccggacccgggacagcctgttcttctgcctgaagttctgcaccgccgccatcagctacagcctgtgcaagttcagcagccagagccgggacaccctgtgcagctgcctgagccccggcctgatcaagaagttccagttcctgatgttccggctgttcagcgaagcccggcagcccctgagcgaggaggacgtcgccagcctgtcctggagacccctgcacctgcccagcgccgactggcagagagccgccctgagcctgtggacccaccggaccttccgggaggtgctgaaggaagaggacgtgcacctgacctaccaggactggctgcacctggagctggagattcagcccgaggccgacgccctgagcgacaccgagcggcaggatttccaccagtgggccatccacgagcatttcctgcccgagagcagcgccagcggcggctgtgacggcgacctgcaggccgcctgcaccatcctggtgaacgccctgatggacttccaccagagcagccggagctacgaccacagcgagaacagcgacctggtgttcggcggacggaccggcaacgaggacatcatcagccggctgcaggagatggtcgccgacctggagttgcagcaggacctgatcgtgcccctgggccacactcccagccaggagcacttcctgttcgagatcttccgccggagactgcaggccctgacctcaggctggagcgtggccgccagcctgcaacggcagcgggagctgctgatgtacaagcggatcctgctgcggctgcccagcagcgtgctgtgcggcagcagcttccaggccgagcagcccatcacggcccggtgcgagcagttcttccacctggtgaacagcgagatgcggaacttctgcagccacggaggcgccctgacccaggacatcaccgcccacttcttccggggcctgctgaacgcctgcctgcggagcagagaccccagcctgatggtggacttcatcctggccaagtgccagaccaagtgtcccctgatcctgacctccgccctggtgtggtggcccagcctggagcccgtgcttctgtgtcggtggcggcggcactgccagagccctctgccccgggagctgcagaagctgcaggagggccggcagttcgccagcgacttcctgtctcccgaggccgctagcccagccccaaaccccgactggctgagcgctgccgccctgcacttcgccatccagcaggtgcgggaggagaacatccggaagcagctgaagaagctggactgcgagcgggaggagctgctcgtgttcctcttcttcttcagcctcatgggccttctgagcagccacctgaccagcaacagcaccaccgacctgcccaaggccttccacgtgtgcgccgccatcctggagtgcctggagaagcggaagatcagctggctggccctgttccagctgaccgagagcgacctgcggctgggccgactgctgctgcgggtggccccagaccagcacacccggctgctgcccttcgccttctacagcctgctgagctacttccacgaggacgctgccatccgggaggaggcctttctgcacgtggcagtggacatgtacctgaagctggtgcagctgttcgtggccggcgacaccagcaccgtgtcaccaccagccggccgctccctggagctgaagggccagggcaaccccgtggagctgatcaccaaggcccggctgttcctgctgcagctgatcccacggtgccccaagaagagcttcagccacgtggccgagctgcttgccgaccggggagactgcgaccccgaggtgagcgccgccttgcagagccggcagcaggccgcacctgacgccgacctgagccaggagccccacctgttctaaagctccccgggggcctcggtggcctagcttcttgccccttgggcctccccccagcccctcctccccttcctgcacccgtacccccgtggtctttgaataaagtctgagtgggcggc

SEQ ID NO: 24 - RNA, 4559 nt, synthetic construct
  source 1..4559: mol_type = other RNA
ggaaatcgcaaaatttgctcttcgcgttagatttcttttagttttctcgcaactagcaagctttttgttctcgccatgagcgacagctgggtgcccaacagcgccagtggccaagaccccggcggtcggagacgagcgtgggctgagctgctggccggacgggtgaagcgggagaagtacaaccccgagcgggcccagaagctgaaggagagcgctgtgcggctgctgcggagccaccaggacctgaacgccctgctgctggaggtggagggcccactgtgcaagaagctgagcctgagcaaggtgatcgactgcgacagcagcgaggcctacgccaaccacagcagcagcttcatcggcagcgccctgcaggaccaggccagcagactgggcgtgcccgtgggcatcctgagcgccggcatggtggccagcagcgtgggccagatctgcaccgccccggccgagacaagccaccccgtgctgctgaccgtggagcagcggaagaagctgagcagcctgctggagttcgcccagtacctgctggcccacagcatgttcagccggctgagcttctgccaggagctgtggaagatccagagcagcctgctgctggaggccgtgtggcacctgcacgtgcagggcatcgtgagcctgcaggagctgctggagagccaccccgacatgcacgccgtgggcagctggctgttccggaacctgtgctgcctgtgcgagcagatggaggccagctgccagcacgccgacgtggctcgggccatgctgagcgacttcgtgcagatgttcgtgctgcggggcttccagaagaacagcgacctgcggcggaccgtagagcccgagaagatgccccaggtgaccgtggacgtgctgcagcggatgctgatcttcgcactcgacgccctggccgctggtgtgcaggaggagagcagcacccacaagatcgtgcggtgctggttcggcgtgttcagcggccacaccctgggcagcgtgatcagcaccgatcccctgaagcggttcttcagccacaccctgacccagatcctgacccacagccccgtgctgaaggccagcgacgccgtgcagatgcagcgggagtggagcttcgcccggacccatcccctgctgaccagcctgtaccggcggctgttcgtgatgctgagcgccgaggagctggtgggccacctgcaggaggtgctggagacccaggaggtgcactggcagcgggtgctgagcttcgtgagcgccctggtggtgtgcttccccgaggcccagcagctgctggaggactgggtggcccggctgatggcccaggccttcgagagctgccagctggacagcatggtgaccgccttcctggtggtaaggcaagccgccctggagggtcccagcgccttcctgagctacgccgactggttcaaggccagcttcggcagcacccggggctaccacggctgcagcaagaaggccctggtgttcctgttcaccttcctgagcgagctggtgcccttcgagtcaccccggtacctgcaggtgcacatcctgcacccacccctggtgcccggcaagtaccggagcctgctgaccgactacatcagcctggccaagacccggctggccgacctgaaggtgagcatcgagaacatgggcctgtacgaggacctgagcagcgccggcgacatcaccgagccccacagccaggccctgcaggacgtggagaaggccatcatggtgttcgagcacaccggcaacatccccgtgaccgtgatggaggccagcatcttccggcggccctactacgtgagccacttcctgcccgccctgctgactccccgggtgctgcccaaggtgcccgacagccgggtggccttcatcgagagcctgaagcgggccgacaagattcctcccagcctgtacagcacctactgccaggcctgcagtgccgccgaggagaagcccgaagacgccgctctgggcgtgagagccgagcccaacagcgccgaggagcctttgggccagctgacagcagcccttggcgagctgcgggccagcatgaccgaccccagccagcgggacgtgatcagcgcccaggtggccgtgatctccgagagactgcgggccgtgctgggccacaacgaggacgacagcagcgtggagatcagcaagatccagctgagcatcaacactccccggctggagcccagggagcacatggccgtggacctgctgctgaccagcttctgccagaacctgatggccgcctcaagcgtggcccctccagagcggcaaggcccttgggcggccctgttcgtgcggaccatgtgcggccgggtgttgcctgccgtgctgacccggctgtgccagctgctgcggcaccagggccctagcctgagcgctccccacgtgctggggctggccgccttagcagtgcacctgggcgagtcacggagcgccctgcccgaggtggacgtgggccctcctgcgccaggagcagggcttcccgtgcccgccctgttcgacagcctgctgacctgccggacccgggacagcctgttcttctgcctgaagttctgcaccgccgccatcagctacagcctgtgcaagttcagcagccagagccgggacaccctgtgcagctgcctgagccccggcctgatcaagaagttccagttcctgatgttccggctgttcagcgaggcccggcaacccctgagcgaggaggacgtggccagcctgagctggcggcccctgcaccttcccagcgccgactggcaacgcgccgccctgagcctgtggacccaccggaccttccgggaggtgctgaaggaggaggacgtgcacctgacctaccaggactggctgcacctggagctggagattcagcccgaggccgacgccctgagcgacaccgagcggcaggacttccaccagtgggccatccacgagcacttcctgcccgagagcagtgccagcggcggctgtgacggcgacctgcaggccgcctgcaccatcctggtgaacgccctgatggacttccaccagagcagccggagctacgaccacagcgagaacagcgacctggtgttcggaggccggaccggcaacgaggacatcatcagccggctgcaggagatggtggccgacctggagctgcagcaggacctgatcgtgcccctgggccacacccctagccaggagcacttcctgttcgagatctttcggcggcggctgcaagcgctgaccagcggttggagcgtggccgcctccctgcaacggcagcgggagctgctgatgtacaagcggatcctgctgcggctgcccagcagcgtgctgtgcggcagcagcttccaggccgagcagcccatcaccgcccggtgcgagcagttcttccacctggtgaacagcgagatgcggaacttctgcagccacggtggcgccctgacccaggacatcaccgcccacttcttccggggcctgctgaacgcctgcctgcgtagccgggaccccagcctgatggtggacttcatcctggccaagtgccagaccaagtgcccactgatcctgaccagcgccctggtgtggtggcccagcctggagcccgtgctgctgtgccggtggcgacggcactgccagtcaccgctgccccgggagctgcagaagctgcaggagggccggcagttcgccagcgacttcctgagccccgaggctgccagccctgccccgaaccccgactggctgagtgccgccgccctgcacttcgccatccagcaggtgcgggaggagaacatccggaagcagctgaagaagctggactgcgagcgggaggagctgctggtgttcctgttcttcttcagcctgatgggcctgctgagcagccacctgaccagcaacagcaccaccgacctgcccaaggccttccacgtgtgcgccgccatcctggagtgcctggagaagcggaagatcagctggctggccctgttccagctgaccgagtctgacctgcgcctgggcaggctgctgctgcgggtggctcccgaccagcacacccggctgctgcccttcgccttctacagcctgctgagctacttccacgaggacgccgccatccgggaggaggccttcctgcacgtggccgtggacatgtacctgaagctggtgcagctgttcgtggccggcgacaccagcactgtatctccaccggctgggcggtccctggagctgaagggccagggcaaccccgtggagctgatcaccaaggcccggctgttcctgctgcagctgatcccacggtgccccaagaagagcttcagccacgtggccgagctgctggccgaccggggagactgcgaccccgaggttagtgccgccctccagagccggcagcaagccgcccctgacgccgacctgagccaggagccccacctgttctaaagctccccgggggcctcggtggcctagcttcttgccccttgggcctccccccagcccctcctccccttcctgcacccgtacccccgtggtctttgaataaagtctgagtgggcggc

SEQ ID NO: 25 - RNA, 4559 nt, synthetic construct
  source 1..4559: mol_type = other RNA
ggaaatcgcaaaatttgctcttcgcgttagatttcttttagttttctcgcaactagcaagctttttgttctcgccatgtccgactcttgggtccccaactccgcctctggtcaagatccgggaggaaggagacgtgcctgggcggaactgttagccggacgtgtgaagagggagaagtacaatcccgagcgcgcacagaagctcaaggaatccgcagttaggctcctccgctctcaccaggacctcaacgcactgctcctcgaggtcgagggaccactctgcaagaagctctccctctccaaggtcatcgactgcgactcctccgaggcctacgccaaccactcctcctccttcatcggctccgccctccaagatcaagcttcccgcctcggagttcctgtgggcatcctaagcgccgggatggtcgcatcttccgtcggccagatttgcacagcacccgccgagacatcccatcctgtccttctcaccgtcgagcagcgcaagaagctctcctccctcctcgagtttgcccagtacctcctcgcccactccatgttctcccgcctctccttctgccaggagctctggaagatccagtcctccctcctgctcgaagccgtctggcatctccacgtccagggcatcgtctccctccaggagctcctcgaatcccatccagacatgcacgccgtcggttcttggctcttccgcaacctctgctgcctctgcgagcagatggaagcctcctgccaacacgctgacgtcgctcgagccatgctctccgacttcgtccagatgttcgtcctccgcggcttccagaagaattccgacctccgtcgcacagtcgagcctgagaagatgcctcaggtcaccgtcgacgtcctccagagaatgctcatcttcgcactggacgcactagctgcaggcgtccaggaggagtcctccacccacaagatcgtccgctgttggttcggcgtctttagcgggcacaccttggggtcagtcatctccaccgatcccctcaagcgcttcttctcccacaccctcacccagatcctcacccactccccagtcctcaaagcctccgacgccgtccagatgcaacgcgagtggtcctttgcccgaacccacccactcctcacctccctctaccgcaggctcttcgtcatgctctccgccgaagaactcgtcggccacttgcaggaggtcctcgagacccaagaagtccactggcagcgcgtcctctccttcgtctccgccctcgtcgtctgttttccagaggcccagcagctccttgaggattgggtggcacggctcatggctcaggccttcgagtcctgccagctcgactccatggtcaccgcctttcttgttgtccgccaagctgctctcgagggtccttcggcattcctgagctacgccgactggttcaaggcctccttcggtagcacaaggggctaccacggttgctccaagaaggccctcgtcttcctcttcaccttcctctccgagctcgtccccttcgagagtccccgctacctccaggtccacatcctccatcctcctctggtccccggaaagtaccgctccctcctcaccgactacatctccctcgctaagacccgactcgccgacttgaaggtctccatcgagaacatgggcctctacgaagatctctccagtgcaggcgacatcaccgagccacactcccaagccctccaggacgtcgagaaggccatcatggtcttcgagcacaccggcaacatccccgtcaccgtcatggaggcctccatcttcagacgcccctactacgtctcccactttctccctgccctcctaacccctcgggtactccccaaggtccccgactccagagtcgccttcatcgagtccctcaagcgcgcagacaagatcccaccatccctctactccacctattgtcaagcctgttctgccgccgaagagaaacccgaggacgccgcattaggcgtccgtgcagagcccaactcagctgaggagccactgggccagctaacagctgcactaggcgagctgagggcgagtatgaccgacccttcccagcgagacgtcatctccgcccaagtcgccgtcatttccgaacgcttgagggcagtgctgggccacaacgaggacgactcctccgtcgagatctccaagatccagctctccatcaatacacctcgcctggaaccccgagagcacatggccgtcgacctcctcctcacctccttctgccagaacttgatggctgcctcctccgttgctcctccggaaagacaaggtccctgggccgctctctttgtccgcaccatgtgtggacgagtgctcccagctgtcctcactcgcctctgccagttgctccgccaccaaggaccctcactctctgcaccgcacgtactgggactcgcagcactcgcagtccacctcggagaatcccgctctgctctcccagaagtggacgtgggacctcctgcacctggtgctggcttgcccgtaccagctctcttcgactccctcctcacgtgcagaacccgcgactccctcttcttctgcctcaagttctgcaccgccgccatctcctactccctctgcaagttcagctcccagtcacgcgacaccctctgctcctgcttgtcccctggcctcatcaagaagttccagttcctcatgttccgcctcttctccgaggccagacaacccctctccgaggaggacgtggcttccctcagttggagacccctccacttgccttccgccgattggcaaagagccgcgctctctctctggacccatcgcaccttccgcgaggtcctcaaggaggaggacgtccacctcacctaccaggactggctccacctcgagctcgagatccaacccgaagcagacgccctctccgacaccgaacgccaggacttccaccagtgggccatccacgagcacttcctccccgaaagcagtgcttccggtggttgcgacggggatctccaagccgcttgcaccatcctcgtcaacgccctcatggacttccaccagtcctcccgctcctacgaccactccgagaactccgacctagtgtttggcggccgcactggaaacgaggacatcatctcccgcctccaggagatggtggccgatctcgagctccagcaggatctcatcgtccctctcggccacacaccttcgcaggagcacttcctcttcgaaatcttcagaagacgcctccaagcgctcacttccggttggtctgtggccgcaagtctccaacgccagagggaactcctcatgtacaagcgcatcctcctacgcctcccttcctccgtcctctgtgggtcctccttccaggcagaacagcccatcactgcccgctgtgagcagttcttccacctcgtcaactccgagatgcgcaacttctgttcacacggcggtgcactcacccaagacatcaccgcgcacttcttccgcggactcctgaacgcctgcttgagatcccgcgacccatccctcatggtcgacttcatcctcgccaagtgccagaccaagtgtcccctcatcctcaccagtgccctcgtttggtggccctcccttgaacccgtcctcctctgcaggtggagacgccactgtcagtctccgttaccccgcgagctccagaaactccaggagggacgccagttcgcttccgacttcctctcacccgaagctgctagcccagcacccaaccctgactggttatccgccgccgctttgcacttcgccatccagcaagtgcgcgaggagaacatccgcaagcagctcaagaagctcgactgcgagcgagaggagctcctcgttttcctcttcttcttctccctcatgggcctcctctcatcccacctcacctccaactccaccaccgacttgcccaaggcctttcacgtctgtgccgccatcctcgagtgcctcgagaagcgcaagatctcctggctcgccctcttccagctaacagagtccgacctgagactcggtcgcctcctactcagagtcgcacccgaccagcacactcgcctcctccccttcgcattctactccctcctctcctacttccacgaagacgcagcaatccgcgaggaggccttccttcacgtcgccgtcgacatgtacctcaagctcgtccagctctttgtcgcgggcgacacgagtacagttagccctccagcaggtaggtccctcgagcttaaaggccagggcaacccagtcgagctcatcaccaaggcccgcctgttcctgctacagctcatcccacgctgccccaagaagtccttctcccacgtagccgaactcctcgccgatcgcggagattgcgaccccgaagtttccgctgccttgcagtcacggcagcaagcagccccagacgcagacctctcacaggagccccacctcttctaaagctccccgggggcctcggtggcctagcttcttgccccttgggcctccccccagcccctcctccccttcctgcacccgtacccccgtggtctttgaataaagtctgagtgggcggc

SEQ ID NO: 26 - RNA, 4559 nt, synthetic construct
  source 1..4559: mol_type = other RNA
ggaaatcgcaaaatttgctcttcgcgttagatttcttttagttttctcgcaactagcaagctttttgttctcgccatgtcagattcctgggtgcccaactccgctagcggacaagatccaggtggccgcaggagagcctgggctgaactgcttgccgggagagtgaagagagagaagtacaacccagagcgcgcccagaagttaaaggagagcgccgtaagactgctgcgcagccaccaagacttaaacgctctgctgctggaggtggaggggcccctgtgcaagaagctgagcctgtccaaggtgatcgattgtgacagcagcgaggcttacgccaatcattcatcttccttcatcggctcagcactgcaggatcaagcaagccggctgggcgttccagttggcatactgagcgccggcatggtggccagcagcgtgggacagatctgcaccgctcccgccgagacctcacatcccgttctgctgacagttgagcaacgcaagaagctgtcaagcctgctggagttcgcacagtacctgttagcccacagcatgttttcccgcttaagcttctgccaggagctgtggaagatccagtcaagtctgctactggaggccgtgtggcacttacacgtgcagggtatcgtgtcactgcaagagctactggagagccatcccgatatgcacgccgtggggtcttggctgttccgcaatctgtgctgcctgtgcgaacagatggaagcctcctgccaacacgccgacgttgctagagcaatgctgagtgacttcgttcagatgttcgtgctgcgcgggttccagaagaactcagatctccggaggaccgtggagcctgagaagatgccccaggtaaccgtggacgtgctgcagcggatgttaatctttgccctggacgctctcgctgccggagtgcaggaggagagctccacccacaagatcgtgcggtgctggttcggcgtgttttccggccataccttaggctccgtgatctccaccgatcccctgaaacggttcttctcccacacactgacccagatactgacccactcccctgtgttaaaggctagcgacgccgtgcaaatgcaacgcgagtggtcctttgcccgcacccatccactgctgaccagtctgtaccggcggctgtttgttatgctgtccgccgaggagttagtgggccacctgcaggaggttctggaaacccaggaggtgcattggcagcgcgtgttaagcttcgtgagcgccctggtggtgtgctttcccgaggcccagcagttactggaggactgggtggcccggctgatggcccaggccttcgagagctgccagctggacagcatggtgaccgccttcctggtggtacgtcaagctgccctggagggcccaagcgccttcctgagttacgctgactggttcaaggccagcttcgggtccacccgcgggtaccacggctgctccaagaaggccctggtgttcctgtttaccttcctgagcgagctggtgccatttgagtctccccgctacctgcaggtgcacatcctgcacccacccctggtccctggaaagtacagatcactgttaaccgactacatctctctggccaagacccggttagccgacctgaaggtgtctatcgagaatatgggcctgtacgaggacctgtccagcgccggcgatatcaccgagccccacagccaagccttacaggacgtggagaaggccattatggtgttcgagcacaccggcaatatccccgtgaccgtaatggaggcttctatcttccggcggccctactacgtgtcccacttcctgcccgccctgttaacacccagagtgttacccaaggtgcccgacagtcgcgttgccttcatagagagcttaaagagagccgacaagatccctcctagtttatactccacctactgccaggcttgctccgccgccgaggagaagcccgaggacgcagccttaggagtaagagctgagcccaactcagccgaggagccactgggccagttaaccgctgccctgggagagttacgggcctctatgaccgaccccagccaacgcgacgttatctctgcccaagtggcagtgataagcgagaggctgcgggcagtattaggacacaacgaggacgacagcagcgtggagatcagtaagatccaattaagcatcaacacacccaggctggagcccagagagcacatggccgtggacctgctgttaacctccttctgtcagaacttaatggccgcaagtagcgtggctcctccagaacgccaaggtccttgggctgccttattcgttcgcaccatgtgtggccgcgtcctgccagcagtgctgacccggctgtgccagttactgcggcaccaagggccctccctgagcgctcctcacgtgcttgggttggccgccctcgctgtacacttaggtgaaagcagaagcgcccttcctgaggtggacgtgggtcctccagccccaggtgccggactccccgtgccagccctgttcgacagcttactgacttgtcgcacccgggactccctgttcttttgtctgaagttctgcaccgccgccatctcctactcactgtgcaagttctctagccagtcccgggataccctgtgctcctgcctgtctccaggcctgatcaagaagttccagttcctgatgttcagactgttctcagaagcaagacagcctctgtccgaggaggacgtggcctccttatcctggcgacccctgcacctgccctccgctgattggcaaagagctgcacttagcctgtggacccaccggacatttcgcgaggtgctgaaagaagaagacgtacacctgacctaccaagactggctgcacctggagctggagatccagcctgaagccgacgccctgagcgacaccgagcgccaagactttcaccagtgggccatacacgagcactttctgcccgagtcaagcgccagtggaggctgcgacggtgatctgcaagcagcctgcaccatcttagtgaacgccctgatggacttccaccaatcttcccgcagctacgaccactccgagaacagcgacctggtgtttggaggccgcactgggaacgaggacatcattagccgcctgcaagaaatggtggctgacctggaactccaacaggacttaatcgtacctctggggcacacaccaagccaggagcacttcttatttgagatatttagaagacgcttacaagcactgacttcaggctggagtgtagctgcaagcctgcaacgccaacgcgagctgctgatgtataagcggatactgctgcgcctgccctcctctgtactgtgcggctcctccttccaggccgagcagcccatcaccgcccggtgtgagcagttcttccacctggtgaacagcgagatgcggaacttctgttctcacggaggcgctctgacccaagacatcacagcccacttcttccgcggcctgctgaacgcctgtctgcggtcaagggaccccagcctgatggtcgacttcatactggcaaagtgtcagaccaagtgtcccctgatcctgaccagcgctctggtttggtggccatcactggagcccgtactcctgtgtcgctggaggcggcattgccagtcacctttaccccgagaactgcagaagctgcaggagggccggcagtttgccagcgacttcctgtcaccagaggcagcctctcccgctcctaaccccgattggctatccgcggccgctctgcacttcgccatccagcaggtgcgggaggagaacatacggaaacaactgaagaaattagactgcgagagagaggagttacttgtgttcctgttcttcttcagtctgatgggcctgctgtcctcccacctgaccagcaattcaaccaccgacctgcccaaggccttccacgtttgtgccgccatcctggagtgcctggagaagcggaagattagctggttagccctgtttcagctgaccgagagtgatttacgcctggggagactgctgttacgcgtggctcccgaccagcacaccaggctgctgcctttcgccttctacagtctgttatcctacttccacgaggacgccgccatcagagaggaggccttcctgcacgtggccgtggacatgtacttaaagctggtgcagctgtttgtggccggcgatacctccacagtaagccctcctgctggcagaagcctggagctgaagggccagggtaaccccgtagagctgattactaaggcccgcctgtttctgctgcaactgatccctcgctgccccaagaagagctttagccacgtggccgaattactggctgacaggggagactgtgatcctgaggtgagcgctgccttacagagccgtcaacaagcggcccctgacgccgatttaagccaagagcctcacctcttctaaagctccccgggggcctcggtggcctagcttcttgccccttgggcctccccccagcccctcctccccttcctgcacccgtacccccgtggtctttgaataaagtctgagtgggcggc

SEQ ID NO: 27 - RNA, 4549 nt, synthetic construct
  source 1..4549: mol_type = other RNA
ggaaatcgcaaaatttgctcttcgcgttagatttcttttagttttctcgcaactagcaagctttttgttctcgccatgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttctgataataggctggagcctccaccgcgttatccgttcctcgtaggctggtcctggggaacgggtcggcgggtacccccgtggtctttgaataaagtctgagtgggcggc

SEQ ID NO: 28 - RNA, 4510 nt, synthetic construct
  source 1..4510: mol_type = other RNA
ggactcactatttgttttcgcgcccagttgcaaaaaatgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttctgataataggctggagcctccaccgcgttatccgttcctcgtaggctggtcctggggaacgggtcggcgggtacccccgtggtctttgaataaagtctgagtgggcggc

SEQ ID NO: 29 - RNA, 4510 nt, synthetic construct
  source 1..4510: mol_type = other RNA
ggactcactatttgttttcgcgcccagttgcaaaaaatgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttctaagcccctccgggggcctccaccgcgttatccgttcctcgtaggctggtcctggggaacgggtcggcgggtacccccgtggtctttgaataaagtctgagtgggcggc

SEQ ID NO: 30 - RNA, 4549 nt, synthetic construct
  source 1..4549: mol_type = other RNA
ggaaatcgcaaaatttgctcttcgcgttagatttcttttagttttctcgcaactagcaagctttttgttctcgccatgagcgacagctgggtgcccaacagtgcttccgggcaggatcccggcggtagaagacgggcttgggctgagctgctggccggcagggtgaagcgggagaagtacaaccccgagcgggcccagaagctgaaggagagtgccgtgcggctgctgcggagccaccaggacctgaacgccctcctgctggaggtcgagggcccactgtgcaagaagctcagcctgagcaaggtgatcgactgtgacagcagcgaggcctacgccaaccacagctccagcttcatcggcagcgccctgcaggaccaggccagcagactgggcgtgcccgtgggcatcctgagcgccggcatggtggccagctcggtgggccagatctgcaccgctcccgccgagaccagccaccccgtgctgctcaccgtggagcagcggaagaagctgagcagcctgctggagttcgcccagtacctgctggcccacagcatgttctcccggctgagcttttgccaagagctgtggaagatccagagctccctgctgctggaggccgtgtggcaccttcacgtgcagggcatcgtgagcctgcaggaactgctggagagccaccccgacatgcacgccgtgggcagctggctgttccggaacctgtgctgcctgtgcgagcagatggaggctagctgccagcacgccgacgtggcacgggccatgctgagcgacttcgtgcagatgttcgtgctgcggggcttccagaagaatagcgacctacggcggaccgtggagcccgagaagatgccccaggtgaccgtggacgtgctgcagcggatgctgatcttcgccctggacgctctggccgctggcgtgcaggaggagagcagcacccacaagatcgtgcggtgctggttcggcgtgttcagcggccacaccctgggcagcgtgatcagcaccgacccactgaagcggttcttcagccacaccctcacccagatcctgacccacagccccgtgctgaaggccagcgacgccgtgcagatgcagcgggagtggagcttcgcccggacccacccactgctgactagcctgtaccggcggctgttcgtgatgctgagcgcagaggagctggtgggccacctgcaggaggtgctggagacccaggaggtgcactggcagcgggtgctgagcttcgtgagcgccctggtggtgtgcttccccgaggcccagcagctcctggaggactgggtggcccggctgatggcccaggccttcgagagctgccagctggacagcatggtgaccgccttcctggtggtgcggcaggctgccctggaaggccccagcgctttcctgagctacgccgactggttcaaggccagcttcggctccacccggggctaccacggctgcagcaagaaggccctggtgttcctgttcaccttcctgagcgagctggtgcccttcgagtcaccccggtacctgcaggtgcacatcctgcatcctccgctggtgcccggcaagtaccggagcctgctgaccgactacatcagcctggccaagacccggctggccgacctgaaggtgagcatcgagaacatgggcctgtacgaggacctgagcagcgccggcgacatcaccgagccccacagccaggccctgcaagacgtggagaaggccatcatggtgttcgagcacaccggcaacatccccgtgaccgtcatggaggccagcatcttccggcggccctactacgtgagccacttcctgcccgccctgctgaccccacgggtgctgcccaaggtgcccgacagccgggtggccttcatcgagagcctgaagcgggccgacaagatcccacccagcctgtacagcacctactgccaggcctgctctgccgccgaggagaagcccgaggacgccgcattaggcgtgcgggccgagcccaacagcgccgaggaacccctggggcagctgaccgcagccctgggagagctgcgggccagcatgaccgaccccagccagcgggacgtgatcagcgcccaggtggccgtcatcagcgagcggctgcgggcagtgctgggccacaacgaggacgacagcagcgtggagatcagcaagatccagctgagcatcaacaccccacggctggaacctcgggagcacatggccgtggacctgctgctgaccagcttctgccagaacctgatggccgcctctagcgtggccccaccagaacggcagggtccctgggctgccctgttcgtgcggaccatgtgcggccgggttctgcccgctgtgctgacccggctgtgccagctgctgcggcaccagggccctagcctgagcgctccccacgtgctgggcctagctgccctggccgtgcaccttggggagtcccgaagcgccctgcccgaggtggacgtgggccctccagccccaggtgctggccttcccgtgcccgctctgttcgacagtctgctgacctgccggacccgggacagcctgttcttctgcctgaagttctgcaccgccgccatcagctacagcctgtgcaagttcagcagccagagccgggacaccctgtgcagctgcctgagccccggcctgatcaagaagttccagttcctgatgttccggctgttcagcgaagcccggcagcccctgagcgaggaggacgtcgccagcctgtcctggagacccctgcacctgcccagcgccgactggcagagagccgccctgagcctgtggacccaccggaccttccgggaggtgctgaaggaagaggacgtgcacctgacctaccaggactggctgcacctggagctggagattcagcccgaggccgacgccctgagcgacaccgagcggcaggatttccaccagtgggccatccacgagcatttcctgcccgagagcagcgccagcggcggctgtgacggcgacctgcaggccgcctgcaccatcctggtgaacgccctgatggacttccaccagagcagccggagctacgaccacagcgagaacagcgacctggtgttcggcggacggaccggcaacgaggacatcatcagccggctgcaggagatggtcgccgacctggagttgcagcaggacctgatcgtgcccctgggccacactcccagccaggagcacttcctgttcgagatcttccgccggagactgcaggccctgacctcaggctggagcgtggccgccagcctgcaacggcagcgggagctgctgatgtacaagcggatcctgctgcggctgcccagcagcgtgctgtgcggcagcagcttccaggccgagcagcccatcacggcccggtgcgagcagttcttccacctggtgaacagcgagatgcggaacttctgcagccacggaggcgccctgacccaggacatcaccgcccacttcttccggggcctgctgaacgcctgcctgcggagcagagaccccagcctgatggtggacttcatcctggccaagtgccagaccaagtgtcccctgatcctgacctccgccctggtgtggtggcccagcctggagcccgtgcttctgtgtcggtggcggcggcactgccagagccctctgccccgggagctgcagaagctgcaggagggccggcagttcgccagcgacttcctgtctcccgaggccgctagcccagccccaaaccccgactggctgagcgctgccgccctgcacttcgccatccagcaggtgcgggaggagaacatccggaagcagctgaagaagctggactgcgagcgggaggagctgctcgtgttcctcttcttcttcagcctcatgggccttctgagcagccacctgaccagcaacagcaccaccgacctgcccaaggccttccacgtgtgcgccgccatcctggagtgcctggagaagcggaagatcagctggctggccctgttccagctgaccgagagcgacctgcggctgggccgactgctgctgcgggtggccccagaccagcacacccggctgctgcccttcgccttctacagcctgctgagctacttccacgaggacgctgccatccgggaggaggcctttctgcacgtggcagtggacatgtacctgaagctggtgcagctgttcgtggccggcgacaccagcaccgtgtcaccaccagccggccgctccctggagctgaagggccagggcaaccccgtggagctgatcaccaaggcccggctgttcctgctgcagctgatcccacggtgccccaagaagagcttcagccacgtggccgagctgcttgccgaccggggagactgcgaccccgaggtgagcgccgccttgcagagccggcagcaggccgcacctgacgccgacctgagccaggagccccacctgttctgataataggctggagcctccaccgcgttatccgttcctcgtaggctggtcctggggaacgggtcggcgggtacccccgtggtctttgaataaagtctgagtgggcggc

SEQ ID NO: 31 - RNA, 4553 nt, synthetic construct
  source 1..4553: mol_type = other RNA
ggaaactttatttagtgttactttattttctgtttatttgtgtttcttcagtgggtttgttctaatttccttggccgccatgtccgactcctgggtgcccaactccgctagcggacaagatccaggtggccgcaggagagcgtgggctgaactgcttgccgggcgagtgaagcgcgagaagtacaaccccgagcgcgcccagaagctgaaggagagcgccgtacggctgctgcgcagccaccaggacctgaacgccctgctgctggaggtggaggggcccctgtgcaagaagctgagcctgtccaaggtgatcgactgcgacagcagcgaggcctacgccaaccacagctcctccttcatcggctccgccctgcaggatcaagctagccggctgggcgttccagtgggcatcctgagcgccggcatggtggccagcagcgtgggccagatctgcaccgctcccgccgagaccagccaccccgtgctgctgaccgtggagcagcgcaagaagctgtcaagcctgctggagttcgcccagtacctgctggcccacagcatgttctcccgcctgagcttctgccaggagctgtggaagatccagtccagcctgctactggaggccgtgtggcacttacacgtgcagggcatcgtgagcctgcaggagctactggagagccaccccgacatgcacgccgtggggtcctggctgttccgcaacctgtgctgcctgtgcgagcagatggaagcctcctgccaacacgccgacgttgcacgggcaatgctgagcgacttcgtgcagatgttcgtgctgcgcgggttccagaagaactcagacctccggaggaccgtggagcctgagaagatgccccaggtgaccgtggacgtgctgcagcggatgctgatctttgccctggacgctctcgctgccggagtgcaggaggagagctccacccacaagatcgtgcggtgctggttcggcgtgttctccggccacaccctgggctccgtgatctccaccgatcccctgaagcggttcttctcccacaccctgacccagatcctgacccactcccctgtgctgaaggccagcgacgccgtgcagatgcagcgcgagtggtccttcgcccgcacccatcccctgctgaccagcctgtaccggcggctgttcgtgatgctgtccgccgaggagctggtgggccacctgcaggaggtgctggagacccaggaggtgcactggcagcgcgtgctgagcttcgtgagcgccctggtggtgtgcttccccgaggcccagcagctcctggaggactgggtggcccggctgatggcccaggccttcgagagctgccagctggacagcatggtgaccgccttcctggtggtgcgtcaagcagccctggagggcccaagcgccttcctgagctacgccgactggttcaaggccagcttcgggtccacccgcgggtaccacggctgctccaagaaggccctggtgttcctgtttaccttcctgagcgagctggtgcccttcgagtcgccccgctacctgcaggtgcacatcctgcacccacccctggtccctggcaagtaccggagcctgctgaccgactacatctccctggccaagacccggctggccgacctgaaggtgagcatcgagaacatgggcctgtacgaggacctgtccagcgccggcgacatcaccgagccccacagccaggccctgcaggacgtggagaaggccatcatggtgttcgagcacaccggcaacatccccgtgaccgtgatggaggctagtatcttccggcggccctactacgtgtcccacttcctgcccgccctgctgacaccccgggtgctgcccaaggtgcccgacagtcgcgtcgccttcatcgagagcctgaagcgggccgacaagatcccgccctccctgtactccacctactgccaggcttgctccgccgccgaggagaagcccgaggacgcagccttaggagtacgcgccgagcccaacagcgccgaggagccactgggccagttgaccgctgccctgggagagctgcgggccagcatgaccgaccccagccaacgcgacgtgatcagcgcccaggtggccgtgatcagcgagaggctgcgggcagtgctgggccacaacgaggacgacagcagcgtggagatcagcaagatccagctgagcatcaacacacccaggctggagcccagagagcacatggccgtggacctgctgctgacctccttctgccagaacctgatggccgcaagcagcgtggctcctccagaacgccaaggcccttgggctgccctgttcgtgcgcaccatgtgtggccgcgtcctgccagcagtgctgacccggctgtgccagcttctgcggcaccaagggccctccctgagcgctccccacgtgcttgggttggccgccctcgctgtgcacctgggcgagagcaggagcgcccttcccgaggtggacgtgggtcctccagccccaggtgccggactccccgtgccagccctgttcgacagcctgctgacttgtcgcacccgggactccctgttcttctgcctgaagttctgcaccgccgccatctcctactccctgtgcaagttctccagccagtcccgggacaccctgtgctcctgcctgtctcccggcctgatcaagaagttccagttcctgatgttccgcctgttcagcgaagcacgccagcccctgtccgaggaggacgtggcctccctgtcctggcgacccctgcacctgccctccgccgattggcagcgcgccgcccttagcctgtggacccaccggaccttccgcgaggtgctgaaggaggaagacgtgcacctgacctaccaggactggctgcacctggagctggagatccagcccgaagccgacgccctgagcgacaccgagcgccaggactttcaccagtgggccatccacgagcactttctgcccgagagcagcgccagtggaggctgcgacggcgacctgcaagccgcctgcaccatcctggtgaacgccctgatggacttccaccagagctcccgcagctacgaccactccgagaacagcgacctggtgtttggaggccgcaccgggaacgaggacatcatcagccgcctgcaggagatggtggccgacctggagctccagcaggacctgatcgtgccactggggcacaccccaagccaggagcacttcctgttcgagatcttccgcagacgcctgcaggcactgacctccggctggagtgtggccgcaagcctgcagcgccaacgcgagctgctgatgtacaagcggatcctgctgcgcctgccctcctccgtgctgtgcggctcctccttccaggccgagcagcccatcaccgcccggtgcgagcagttcttccacctggtgaacagcgagatgcggaacttctgctctcacggcggcgctctgacccaggacatcaccgcccacttcttccgcggcctgctgaacgcctgcctgcggagcagggaccccagcctgatggtcgacttcatcctggccaagtgccagaccaagtgtcccctgatcctgaccagcgccctggtttggtggccctcactggagcccgtgctcctgtgtcgctggaggcggcactgccagtcacccctgccccgagagctgcagaagctgcaggagggccggcagttcgccagcgacttcctgtcaccagaggcagcctctcccgctcctaaccccgattggctatccgcggccgctctgcacttcgccatccagcaggtgcgggaggagaacatccggaagcagctgaagaagctggactgcgagagggaggagctgcttgtgttcctgttcttcttcagcctgatgggcctgctgtcctcccacctgaccagcaactccaccaccgacctgcccaaggccttccacgtgtgcgccgccatcctggagtgcctggagaagcggaagatcagctggctggccctgttccagctgaccgagagcgacctgcgcctggggagactgctgctgcgcgtggctcccgaccagcacaccaggctgctgcccttcgccttctactccctgctgtcctacttccacgaggacgccgccatccgggaggaggccttcctgcacgtggccgtggacatgtacctgaagctggtgcagctgttcgtggccggcgacacctccaccgtgagccctcctgctggccgaagcctggagctgaagggccagggcaaccccgtggagctgatcaccaaggcccgcctgttcctgctgcagctgatccctcgctgccccaagaagagcttcagccacgtggccgagctgctggctgacaggggagactgcgaccccgaggtgagcgctgccctgcagagccgtcaacaagcggcccctgacgccgatctgagccaggagccccacctgttctgataataggctggagcctccaccgcgttatccgttcctcgtaggctggtcctggggaacgggtcggcgggtacccccgtggtctttgaataaagtctgagtgggcggc

SEQ ID NO: 32-49 - skipped (18 entries)

SEQ ID NO: 50 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 51 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 52 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 53 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 54 - RNA, 59 nt, synthetic construct
  source 1..59: mol_type = other RNA
ggagaacttccgcttccgttggcgcaagcgctttcattttttctgctaccgtgactaag

SEQ ID NO: 55 - RNA, 46 nt, synthetic construct
  source 1..46: mol_type = other RNA
ggaaataagagagaaaagaagagtaagaagaaatataagagccacc

SEQ ID NO: 56 - RNA, 56 nt, synthetic construct
  source 1..56: mol_type = other RNA
ggaaataagagagaaaagaagagtaagaagaaatataagaccccggcgccgccacc

SEQ ID NO: 57 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 58 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 59 - RNA, 83 nt, synthetic construct
  source 1..83: mol_type = other RNA
  misc_difference 14..18: note = This region may encompass 0-5 nucleotides
  misc_difference 19: note = This region may be absent
  misc_difference 22: note = This region may be absent
  misc_difference 23..27: note = This region may encompass 0-5 nucleotides
  misc_difference 81..83: note = This region may be absent
ggaaatcgcaaaatttttgctctttttcgcgttagatttcttttagttttctykcaactagcaagctttttgttctcgccrcc

SEQ ID NO: 60 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 61 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 62 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 63 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 64 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 65 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 66 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 67 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 68 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 69 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 70 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 71 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 72 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 73 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 74 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 75 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 76 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 77 - RNA, 57 nt, synthetic construct
  source 1..57: mol_type = other RNA
ggaaataagagagaaaagaagagtaagaagaaatataagagccaaaaaaaaaaaacc

SEQ ID NO: 78 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 79 - skipped

SEQ ID NO: 80 - RNA, 36 nt, synthetic construct
  source 1..36: mol_type = other RNA
ggactcactatttgttttcgcgcccagttgcaaaaa

SEQ ID NO: 81-90 - skipped (10 entries)

SEQ ID NO: 91 - RNA, 50 nt, synthetic construct
  source 1..50: mol_type = other RNA
caccgcgttatccgttcctcgtaggctggtcctggggaacgggtcggcgg

SEQ ID NO: 92 - RNA, 100 nt, synthetic construct
  source 1..100: mol_type = other RNA
ccacccccagcgccaccaccgctgccgtcgccaccgcgttatccgttcctcgtaggctggtcctggggaacgggtcggcggccggtcggcttctgtttta

SEQ ID NO: 93 - RNA, 60 nt, synthetic construct
  source 1..60: mol_type = other RNA
ctgagagacctgtgtgaactattgagaagatcggaacagctccttactctgaggaagttg

SEQ ID NO: 94-116 - skipped (23 entries)

SEQ ID NO: 117 - RNA, 109 nt, synthetic construct
  source 1..109: mol_type = other RNA
tgataataggctggagcctccaccgcgttatccgttcctcgtaggctggtcctggggaacgggtcggcgggtacccccgtggtctttgaataaagtctgagtgggcggc

SEQ ID NO: 118-120 - skipped (3 entries)

SEQ ID NO: 121 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - skipped

SEQ ID NO: 123 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 124-138 - skipped (15 entries)

SEQ ID NO: 139 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 140 - RNA, 119 nt, synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 141 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 142 - RNA, 170 nt, synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 143 - RNA, 141 nt, synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 144 - RNA, 185 nt, synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 145 - RNA, 187 nt, synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 146 - RNA, 170 nt, synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 147 - RNA, 141 nt, synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 148 - RNA, 22 nt, synthetic construct
  source 1..22: mol_type = other RNA
caaacaccattgtcacactcca

SEQ ID NO: 149 - RNA, 23 nt, synthetic construct
  source 1..23: mol_type = other RNA
tccataaagtaggaaacactaca

SEQ ID NO: 150 - RNA, 22 nt, synthetic construct
  source 1..22: mol_type = other RNA
cgcattattactcacggtacga

SEQ ID NO: 151 - RNA, 87 nt, synthetic construct
  source 1..87: mol_type = other RNA
gacagtgcagtcacccataaagtagaaagcactactaacagcactggagggtgtagtgtttcctactttatggatgagtgtactgtg

SEQ ID NO: 152 - RNA, 23 nt, synthetic construct
  source 1..23: mol_type = other RNA
tgtagtgtttcctactttatgga

SEQ ID NO: 153 - RNA, 21 nt, synthetic construct
  source 1..21: mol_type = other RNA
cataaagtagaaagcactact

SEQ ID NO: 154 - RNA, 21 nt, synthetic construct
  source 1..21: mol_type = other RNA
agtagtgctttctactttatg

SEQ ID NO: 155 - RNA, 85 nt, synthetic construct
  source 1..85: mol_type = other RNA
cgctggcgacgggacattattacttttggtacgcgctgtgacacttcaaactcgtaccgtgagtaataatgcgccgtccacggca

SEQ ID NO: 156 - RNA, 21 nt, synthetic construct
  source 1..21: mol_type = other RNA
cattattacttttggtacgcg

SEQ ID NO: 157 - RNA, 21 nt, synthetic construct
  source 1..21: mol_type = other RNA
cgcgtaccaaaagtaataatg

SEQ ID NO: 158-163 - skipped (6 entries)

SEQ ID NO: 164 - RNA, 15 nt, synthetic construct
  source 1..15: mol_type = other RNA
taagcccctccgggg

SEQ ID NO: 165 - RNA, 15 nt, synthetic construct
  source 1..15: mol_type = other RNA
taaagctccccgggg

SEQ ID NO: 166-169 - skipped (4 entries)

SEQ ID NO: 170 - RNA, 18 nt, synthetic construct
  source 1..18: mol_type = other RNA
swstaarymyyyysksss

SEQ ID NO: 171 - RNA, 18 nt, synthetic construct
  source 1..18: mol_type = other RNA
smstgarsmmsssssyys

SEQ ID NO: 172 - RNA, 18 nt, synthetic construct
  source 1..18: mol_type = other RNA
swstagrssrmyymwsyy

SEQ ID NO: 173-179 - skipped (7 entries)

SEQ ID NO: 180 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 181 - RNA, 119 nt, synthetic construct
  source 1..119: mol_type = other RNA
taagcccctccgggggcctcggtggcctagcttcttgccccttgggcctccccccagcccctcctccccttcctgcacccgtacccccgtggtctttgaataaagtctgagtgggcggc

SEQ ID NO: 182 - RNA, 109 nt, synthetic construct
  source 1..109: mol_type = other RNA
taagcccctccgggggcctccaccgcgttatccgttcctcgtaggctggtcctggggaacgggtcggcgggtacccccgtggtctttgaataaagtctgagtgggcggc

SEQ ID NO: 183 - RNA, 125 nt, synthetic construct
  source 1..125: mol_type = other RNA
  modified_base 125: mod_base = OTHER; note = residue 125 has a 3 prime to 3 prime phosphodiester bond to a thymine.
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagaaaaaaaaaaaaaaaaaaaa

SEQ ID NO: 184 - RNA, 150 nt, synthetic construct
  source 1..150: mol_type = other RNA
  misc_difference 1..150: note = This sequence may encompass 80-150 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0313830 A1
(43) Pub. Date: Oct. 9, 2025

(54) MESSENGER RIBONUCLEIC ACIDS WITH EXTENDED HALF-LIFE

(71) Applicant: ModernaTX, Inc., Cambridge, MA (US)

(72) Inventors: David Reid, Medford, MA (US); Michael Albert Zimmer, Wilmington, MA (US)

(21) Appl. No.: 18/849,675

(22) PCT Filed: Mar. 24, 2023
(86) PCT No.: PCT/US2023/016192

§ 371 Date: Sep. 23, 2024

Related U.S. Application Data
(60) Provisional application No. 63/323,748, filed on Mar. 25, 2022.
(60) Provisional application No. 63/405,142, filed on Sep. 9, 2022.
(60) Provisional application No. 63/419,924, filed on Oct. 27, 2022.

(51) Int. Cl. C12N 15/11; A61K 9/51; A61K 31/7125; C12N 15/88

(52) U.S. Cl. CPC C12N 15/11; A61K 9/5123; A61K 31/7125; C12N 15/88

(57) ABSTRACT

The disclosure features a polynucleotide encoding a polypeptide, which polynucleotide comprises a 5′ UTR, a coding region encoding a polypeptide, and a 3 UTR, and lipid nanoparticles comprising the same. The polynucleotides and/or lipid nanoparticles of the present disclosure can increase the level and/or activity of the polypeptide by increasing the half-life and/or duration of expression of the polynucleotide encoding the polypeptide. Also disclosed herein are methods of treating a disease or disorder in a subject using the lipid nanoparticles of the present disclosure.

### CROSS-REFERENCE TO RELATED APPLICATIONS

This application claims the benefit of priority to U.S. Provisional Application No. 63/323,748, filed on Mar. 25, 2022, U.S. Provisional Application No. 63/405,142, filed on Sep. 9, 2022, and U.S. Provisional Application No. 63/419,924, filed on Oct. 27, 2022, the contents of which are hereby incorporated by reference.

### SEQUENCE LISTING

This application contains a Sequence Listing that has been submitted electronically as an XML file named 45817-0128US1_SL.xml. The XML file, created on Apr. 30, 2025, is 178,606 bytes in size. The material in the XML file is hereby incorporated by reference in its entirety.

### BACKGROUND

Efforts to increase messenger ribonucleic acid (mRNA) potency have focused on mRNAs with optimal sequence design for the open reading frame (ORFs). However, there is a need to further improve potency and durability of mRNA expression by exploiting RNA biology.

### SUMMARY

The present disclosure provides, inter alia, polynucleotides encoding a polypeptide (e.g., an mRNA), wherein the polynucleotide comprises: (a) a 5′-UTR (e.g., as described herein); (b) a coding region comprising a stop element (e.g., as described herein); and (c) a 3′-UTR (e.g., as described herein), and LNP compositions comprising the same. In an embodiment, the coding region comprises a polynucleotide sequence, e.g., mRNA, e.g., an open reading frame (ORF) which encodes for a peptide or polypeptide payload, e.g., a therapeutic payload or a prophylactic payload. In an embodiment, the polynucleotide, e.g., mRNA, or polypeptide encoded by the polynucleotide has an increased level and/or activity, e.g., expression or half-life than versions lacking the 5′-UTRs, 3′-UTRs, or stop elements described herein. In an embodiment, the level and/or activity of the polynucleotide, e.g., mRNA, is increased. In an embodiment, the level, activity and/or duration of expression of the polypeptide encoded by the polynucleotide is increased. Also disclosed herein are methods of using an LNP composition comprising a polynucleotide disclosed herein, for treating a disease or disorder, or for promoting a desired biological effect in a subject. It will be understood that any ORF can be combined with the disclosed elements, e.g., ORFs encoding polypeptides or peptides whether, e.g., intracellular, transmembrane, or secreted.

Additional aspects of the disclosure are described in further detail below.

Specifically, provided herein in some embodiments are messenger RNAs (mRNAs) comprising a 5′ UTR, an open reading frame encoding a polypeptide, and a 3′ UTR, wherein the 3′ UTR comprises:
(i) a nucleotide sequence at least 98% identical to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO: 146, or SEQ ID NO:147; or
(ii) a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147, or a deletional variant thereof wherein 1 to 75 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147, wherein the nucleic acid sequence or the deletional variant thereof is modified to include:
   a) one or more miRNA binding sites inserted within the nucleic acid sequence or the deletional variant thereof, and/or
   b) a TENT recruiting sequence, a FUT8 recruiting sequence, one or more Identification and Ratio Determination (IDR) sequences, one or more ribosome engagement detection assay (REDA) sequences, or a combination of one or more IDR sequences and one or more REDA sequences inserted within the nucleic acid sequence or the deletional variant thereof.

In certain embodiments, the disclosure provides a 3′ UTR comprising a nucleotide sequence at least 99% identical to the nucleic acid sequence of SEQ ID NO:139. In certain embodiments, the disclosure provides a 3′ UTR comprising the nucleic acid sequence set forth in SEQ ID NO:139.

In certain embodiments, the disclosure provides a 3′ UTR comprising a nucleotide sequence at least 99% identical to the nucleic acid sequence of SEQ ID NO:140. In certain embodiments, the disclosure provides a 3′ UTR comprising the nucleic acid sequence set forth in SEQ ID NO:140.

In certain embodiments, the disclosure provides a 3′ UTR comprising a nucleotide sequence at least 99% identical to the nucleic acid sequence of SEQ ID NO:141. In certain embodiments, the disclosure provides a 3′ UTR comprising the nucleic acid sequence set forth in SEQ ID NO:141.

In certain embodiments, the disclosure provides a 3′ UTR comprising a nucleotide sequence at least 99% identical to the nucleic acid sequence of SEQ ID NO:142. In certain embodiments, the disclosure provides a 3′ UTR comprising the nucleic acid sequence set forth in SEQ ID NO:142.

In certain embodiments, the disclosure provides a 3′ UTR comprising a nucleotide sequence at least 99% identical to the nucleic acid sequence of SEQ ID NO:143. In certain embodiments, the disclosure provides a 3′ UTR comprising the nucleic acid sequence set forth in SEQ ID NO:143.

In certain embodiments, the disclosure provides a 3′ UTR comprising a nucleotide sequence at least 99% identical to the nucleic acid sequence of SEQ ID NO:144. In certain embodiments, the disclosure provides a 3′ UTR comprising the nucleic acid sequence set forth in SEQ ID NO:144.

In certain embodiments, the disclosure provides a 3′ UTR comprising a nucleotide sequence at least 99% identical to the nucleic acid sequence of SEQ ID NO:145. In certain embodiments, the disclosure provides a 3′ UTR comprising the nucleic acid sequence set forth in SEQ ID NO:145.

In certain embodiments, the disclosure provides a 3′ UTR comprising a nucleotide sequence at least 99% identical to the nucleic acid sequence of SEQ ID NO:146. In certain embodiments, the disclosure provides a 3′ UTR comprising the nucleic acid sequence set forth in SEQ ID NO:146.

In certain embodiments, the disclosure provides a 3′ UTR comprising a nucleotide sequence at least 99% identical to the nucleic acid sequence of SEQ ID NO:147. In certain embodiments, the disclosure provides a 3′ UTR comprising the nucleic acid sequence set forth in SEQ ID NO:147.

In some aspects of the present disclosure, the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include one or more miRNA binding sites inserted within the nucleic acid sequence. In some instances, the one or more miRNA binding sites are selected from SEQ ID NOs:148-157. In some embodiments, the one or more miRNA binding sites comprise at least one copy of SEQ ID NO:149 and at least one copy of SEQ ID NO:150.

In some embodiments, the one or more miRNA binding sites comprise at least three copies of SEQ ID NO:150. In some embodiments, the one or more miRNA binding sites comprise at least two copies of SEQ ID NO:149. In some embodiments, the one or more miRNA binding sites comprise at least two copies of SEQ ID NO:149 and at least one copy of SEQ ID NO:150. In some embodiments, the one or more miRNA binding sites comprise at least three copies of SEQ ID NO:148.

In some aspects provided herein, the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include a TENT recruiting sequence inserted within the nucleic acid sequence.

In some embodiments provided herein, the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include a FUT8 recruiting sequence inserted within the nucleic acid sequence.

In some instances provided herein, the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include one or more IDR sequences inserted within the nucleic acid sequence.

In some aspects provided herein, the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include one or more REDA sequences inserted within the nucleic acid sequence.

In some aspects provided herein, in the deletional variant 1 to 60 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147. In some aspects provided herein, in the deletional variant 1 to 50 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147. In some aspects provided herein, in the deletional variant 1 to 40 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147. In some aspects provided herein, in the deletional variant 1 to 30 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147. In some aspects provided herein, in the deletional variant 1 to 20 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147. In some aspects provided herein, in the deletional variant 1 to 10 consecutive nucleotides are deleted from SEQ ID NO:139, SEQ ID NO:140, SEQ ID NO:141, SEQ ID NO: 142, SEQ ID NO:143, SEQ ID NO:144, SEQ ID NO:145, SEQ ID NO:146, or SEQ ID NO:147. In some aspects provided herein, in the deletional variant less than 10 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147.

In certain embodiments of any of the above described mRNAs, the 5′ UTR comprises a nucleotide sequence at least 80%, 85%, 90%, 95%, 96%, 97%, 98%, 99% or 100% identical to SEQ ID NO:50. In some embodiments, the 5′ UTR comprises the nucleic acid sequence set forth in SEQ ID NO:50.

In certain embodiments provided herein, the 3′ UTR comprises the nucleic acid sequence set forth in SEQ ID NO:139, and wherein the 5′ UTR comprises the nucleic acid sequence set forth in SEQ ID NO:50.

In certain embodiments provided herein, the 3′ UTR comprises the nucleic acid sequence set forth in SEQ ID NO:140, and wherein the 5′ UTR comprises the nucleic acid sequence set forth in SEQ ID NO:50.

In certain embodiments provided herein, the 3′ UTR comprises the nucleic acid sequence set forth in SEQ ID NO:141, and wherein the 5′ UTR comprises the nucleic acid sequence set forth in SEQ ID NO:50.

In certain embodiments provided herein, the 3′ UTR comprises the nucleic acid sequence set forth in SEQ ID NO:142, and wherein the 5′ UTR comprises the nucleic acid sequence set forth in SEQ ID NO:50.

In certain embodiments provided herein, the 3′ UTR comprises the nucleic acid sequence set forth in SEQ ID NO:143, and wherein the 5′ UTR comprises the nucleic acid sequence set forth in SEQ ID NO:50.

In certain embodiments provided herein, the 3′ UTR comprises the nucleic acid sequence set forth in SEQ ID NO:144, and wherein the 5′ UTR comprises the nucleic acid sequence set forth in SEQ ID NO:50.

In certain embodiments provided herein, the 3′ UTR comprises the nucleic acid sequence set forth in SEQ ID NO:145, and wherein the 5′ UTR comprises the nucleic acid sequence set forth in SEQ ID NO:50.

In certain embodiments provided herein, the 3′ UTR comprises the nucleic acid sequence set forth in SEQ ID NO:146, and wherein the 5′ UTR comprises the nucleic acid sequence set forth in SEQ ID NO:50.

In certain embodiments provided herein, the 3′ UTR comprises the nucleic acid sequence set forth in SEQ ID NO:147, and wherein the 5′ UTR comprises the nucleic acid sequence set forth in SEQ ID NO:50.

In certain aspects of any of the above described mRNAs, the mRNA comprises a stop cassette. In some embodiments, the stop cassette is selected from SEQ ID NOs:158-174. In some embodiments, the stop cassette is UAAAGCUCCCCGGGG (SEQ ID NO:165) or UAAGCCCCUCCGGGG (SEQ ID NO:164).

In certain aspects of any of the above described mRNAs, the mRNA comprises a 5′ terminal cap. In some embodiments, the 5′ terminal cap comprises a m⁷GpppG_{2′OMe}, m7G-ppp-Gm-A, m7G-ppp-Gm-AG, CapO, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In certain aspects of any of the above described mRNAs, the mRNA comprises a poly-A region. In some embodiments, the poly-A region is at least about 10, at least about 20, at least about 30, at least about 40, at least about 50, at least about 60, at least about 70, at least about 80, at least about 90 nucleotides in length, or at least about 100 nucleotides in length. In some embodiments, the poly-A region has about 10 to about 200, about 20 to about 180, about 50 to about 160, about 70 to about 140, or about 80 to about 120 nucleotides in length. In some embodiments, the poly-A region comprises A100-UCUAG-A20-inverted deoxy-thymidine (SEQ ID NO:183).

In certain aspects of any of the above described mRNAs, the mRNA comprises at least one chemically modified nucleobase, sugar, backbone, or any combination thereof.

In some embodiments, the at least one chemically modified nucleobase is selected from the group consisting of pseudouracil (ψ), N1-methylpseudouracil (m1ψ), 1-ethylpseudouracil, 2-thiouracil (s2U), 4′-thiouracil, 5-methylcytosine, 5-methyluracil, 5-methoxyuracil, and any combination thereof.

In certain aspects of any of the above described mRNAs, the polypeptide comprises a secreted protein, a membrane-bound protein, or an intercellular protein. In some embodiments, the polypeptide is a cytokine, an antibody, a vaccine, a receptor, an enzyme, a hormone, a transcription factor, a ligand, a membrane transporter, a structural protein, a nuclease, or a component, variant or fragment thereof.

Also provided herein are pharmaceutical compositions comprising any one of the above described mRNAs and a pharmaceutically acceptable carrier.

Also provided herein are lipid nanoparticles comprising any one of the above described mRNAs. In some embodiments, the lipid nanoparticle comprises: (i) an ionizable lipid, (ii) a phospholipid, (iii) a structural lipid, and (iv) a PEG-lipid. In some embodiments, the lipid nanoparticle comprises a compound of Formula (I):

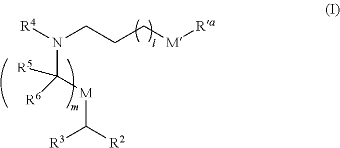

or its N-oxide, or a salt or isomer thereof, wherein R^{′ a} is R^{′branched}; wherein
R^{′branched} is:

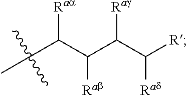

wherein

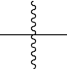

denotes a point of attachment; wherein R^{aα}, R^{aβ}, R^{aγ}, and Raδ are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)˜OH, wherein n is selected from the group consisting of 1, 2, 3, 4, and

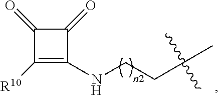

wherein

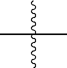

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and
—OC(O)—;
R′ is a C₁₋₁₂ alkyl or C₂₋₁₂ alkenyl; 1 is selected from the group consisting of 1, 2, 3, 4, and 5; and m is selected from the group consisting of 5, 6, 7, 8, 9, 10, 11, 12, and 13.

In some embodiments, the lipid nanoparticle comprises:
(a) (i) Compound II, (ii) Cholesterol, and (iii) PEG-DMG or Compound I;
(b) (i) Compound VI, (ii) Cholesterol, and (iii) PEG-DMG or Compound I;
(c) (i) Compound II, (ii) DSPC or DOPE, (iii) Cholesterol, and (iv) PEG-DMG or Compound I;
(d) (i) Compound VI, (ii) DSPC or DOPE, (iii) Cholesterol, and (iv) PEG-DMG or Compound I;
(e) (i) Compound II, (ii) Cholesterol, and (iii) Compound I;
(f) (i) Compound II, (ii) DSPC or DOPE, (iii) Cholesterol, and (iv) Compound I;
(g) (i) Compound B, (ii) DSPC or DOPE, (iii) Cholesterol, and (iv) PEG-DMG or Compound I;
(h) (i) Compound B, (ii) Cholesterol, and (iii) Compound I; or (i) (i) Compound B, (ii) DSPC or DOPE, (iii) Cholesterol, and (iv) Compound I.

In some embodiments, the lipid nanoparticle comprises Compound II and Compound I. In some embodiments, the lipid nanoparticle comprises Compound B and Compound I. In some embodiments, the lipid nanoparticle comprises Compound II, 10 DSPC, Cholesterol, and Compound I. In some embodiments, the lipid nanoparticle comprises a molar ratio of about 20-60% ionizable lipid: 5-25% phospholipid: 25-55% cholesterol; and 0.5-15% PEG lipid. In some embodiments, the lipid nanoparticle is formulated for intravenous, subcutaneous, intramuscular, intranasal, intraocular, rectal, pulmonary or oral delivery.

Also provided herein are pharmaceutical compositions comprising any one of the lipid nanoparticles described above. Also provided herein are cells comprising any one of the lipid nanoparticles described above.

In some embodiments, also provided herein are methods of increasing expression of a polypeptide, comprising administering to a cell any one of the lipid nanoparticles described above.

Also provided herein are methods of delivering any one of the lipid nanoparticles described above to a cell, comprising contacting the cell in vitro, in vivo or ex vivo with the lipid nanoparticle.

In some instances, also provided herein are methods of delivering any one of the lipid nanoparticles described above to a human subject having a disease or disorder, comprising administering to the human subject in need thereof an effective amount of the lipid nanoparticle.

Other aspects also provided herein are methods of treating, preventing, or preventing a symptom of, a disease or disorder in a human subject in need thereof, comprising administering to the human subject an effective amount of any one of the lipid nanoparticles described above.

Other aspects also provided herein are methods of treating, preventing, or preventing a symptom of, a disease or disorder in a human subject in need thereof, comprising administering to the human subject an effective amount of any one of the lipid nanoparticles described above.

Other features and advantages of the invention will be apparent from the following detailed description and figures, and from the claims.

### BRIEF DESCRIPTION OF DRAWINGS

FIGS. 1A-1G are graphs depicting luciferase or target protein expression encoded by mRNA constructs having the vl.1 5′ UTR (SEQ ID NO: 56) or the v2.0 5′ UTR (SEQ ID NO: 50) in combination with either the alpha 3′UTR or the kappa 3′UTR. FIG. 1A shows whole body ffLuc activity 96 hours post-dose. FIG. 1B shows whole body ffLuc activity 72 hours post-dose. FIG. 1C shows whole body ffLuc activity over 0-4 days post-dose. FIG. 1D shows expression in the liver. FIG. 1E shows expression in the spleen. FIG. 1F shows target protein (i.e., EPO) expression in the serum 96 hours post-dose. FIG. 1G shows target protein (i.e., EPO) expression in the serum 0-4 days post-dose.

FIGS. 2A-2I are graphs depicting overall mean fluorescence intensity or the percentage of mOX40L+ cells as encoded by mRNA constructs having the v2.0 5′ UTR in combination with a control 3′UTR (noted as “triple” in the figure legends), kappa 3′UTR, or iota 3′UTR at 1, 2, and 3 days post-dose in various immune cells. FIG. 2A shows mean fluorescence intensity and % mOX40L+ cells in LSK+ hematopoietic stem and progenitor cells. FIG. 2B shows mean fluorescence intensity and % mOX40L+ cells in splenic dendritic cells. FIG. 2C shows mean fluorescence intensity and % mOX40L+ cells in splenic macrophages. FIG. 2D shows mean fluorescence intensity and % mOX40L+ cells in splenic neutrophils. FIG. 2E shows mean fluorescence intensity and % mOX40L+ cells in splenic monocytes. FIG. 2F shows mean fluorescence intensity and % mOX40L+ cells in splenic eosinophils. FIG. 2G shows mean fluorescence intensity and % mOX40L+ cells in splenic CD4+ T cells. FIG. 2H shows mean fluorescence intensity and % mOX40L+ cells in splenic CD8+ T cells. FIG. 21 shows 5 mean fluorescence intensity and % mOX40L+ cells in splenic B cells.

FIG. 3A is an image showing the protein levels for FANCA (top row for each sample) and Nucleolin (bottom row for each sample) in the FaDu trio cell line at the indicated time points for cells transfected with the indicated constructs. Fold change FANCA expression was calculated normalized to loading control and wild type (WT) average.

FIG. 3B is a graph showing the expression levels of FANCA normalized to Nucleolin and expressed as fold change over the average expression of FANCA in the WT FaDu cell line.

FIG. 4 is a graph showing the percent survival of the FaDu-WT, FaDu-KO transfected with lug GFP mRNA construct or FaDu-KO transfected with lug FANCA constructs and treated with mitomycin C (MMC) at the indicated concentrations 24 hours post transfection. Survival was assessed 5 days post treatment using the cell titer Glo.

FIG. 5A is a graph showing accumulation of cells in G2_M phase of the cell cycle in control (Ctl) or 1, 3-Butadiene Diepoxide (DEB) and MMC treated cells. WT-GFP refers to FaDu WT cells transfected with GFP mRNA; KO-GFP refers to FaDu KO cells transfected with GFP mRNA; FANCA_O1-FANCA-04 refer to FaDu KO cells transfected with FANCA constructs. Transfections occurred 6 hours prior to treatment.

FIG. 5B is a graph showing frequency of G2M for the data of FIG. 5A, presented as mean±SD. Statistical significance is calculated using a student t-test. *P<0.05 **P<0.01, ***P<0.001, ****P<0.0001.

FIG. 6A is a graph showing accumulation of cells in G2_M phase of the cell cycle in control (Ctl) or 1, 3-Butadiene Diepoxide (DEB) and MMC treated cells. WT-GFP refers to FaDu WT cells transfected with GFP mRNA; KO-GFP refers to FaDu KO cells transfected with GFP mRNA; FANCA_O1-FANCA-04 refer to FaDu KO cells transfected with FANCA constructs. Transfections occurred 72 hours prior to treatment.

FIG. 6B is a graph showing frequency of G2M for the data of FIG. 6A, presented as mean±SD. Statistical significance is calculated using a student t-test. *P<0.05 **P<0.01, ***P<0.001, ****P<0.0001.

FIG. 6C is a graph showing expression levels of FANCA normalized to nucleolin and expressed as fold change over the of FANCA in the WT FaDu cell line.

FIG. 7 shows ovalbumin concentration after 6 h and 48h post-dose. The mRNA evaluated had the kappa 3′ UTR (SEQ ID NO:139) and either the v1.1 5′ UTR (SEQ ID NO:56) or the v2.0 5′ UTR (SEQ ID NO:50). Both mRNAs were prepared using the same “alpha” process.

FIG. 8 is a bar graph showing antibody response when an mRNA comprising v2.0 5′ UTR (SEQ ID NO:50) and kappa 3′UTR (SEQ ID NO:139) was utilized.

FIG. 9 is a bar graph showing the effect of a FUT8 sequence in the 3′UTR as compared to when a FUT8 sequence is not present. Shown, from left to right, are: a PBS control, an mRNA containing v1.0 UTRs, an mRNA with v2.0 5′UTR (SEQ ID: NO: 50) and a re-optimized coding sequence, an mRNA with the elements from (2) plus a 3′UTR that bears the ‘delta’ stop cassette and ‘FUT8’ sequence (the 3′ UTR corresponds to SEQ ID NO:140), and two positive controls.

FIG. 10 is a schematic showing that the presence of an v2.0 5′ UTR mRNA encoding hemagglutinin increases immunogenicity in mice.

FIG. 11 are bar graphs showing that presence of v2.0 5′ UTR and v2.0 3′ UTR enhances expression of mRNA encoding hemagglutinin in vitro.

### DETAILED DESCRIPTION

The potency and durability of mRNAs can be optimized by: (1) ensuring that mRNAs delivered to the cytoplasm associate appropriately and productively with ribosomes; and (2) maximizing the time the mRNAs spend actively producing the desired protein product. The sequence of the mRNAs is an important determinant in performance across these aspects.

Disclosed herein, inter alia, is the discovery that the sequence for the 3′ untranslated region (UTR) can be optimized to increase the potency and/or durability of said mRNA. In some embodiments, the combination of the sequence for the 3′ UTR in combination with a 5′ UTR and/or stop element of an mRNA can be optimized to increase the potency and/or durability of said mRNA, for example, by extending the half-life and/or duration of the expression of the mRNA. In some embodiments, the disclosure provides polynucleotides and lipid nanoparticle compositions comprising optimized 3′ UTRs that can increase the efficacy, e.g., level and/or activity, of an mRNA or of a polypeptide encoded by the mRNA.

### 1. Untranslated Regions (UTRs)

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA)), e.g., a mRNA of the invention comprising an open reading frame (ORF) encoding a polypeptide further comprises a UTR (e.g., a 5′ UTR or functional fragment thereof, a 3′ UTR or functional fragment thereof, or a combination thereof).

A UTR can be homologous or heterologous to the coding region in a polynucleotide. In some embodiments, the UTR is homologous to the ORF encoding the therapeutic payload or prophylactic payload. In some embodiments, the UTR is heterologous to the ORF encoding the therapeutic payload or prophylactic payload.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′ UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR.

International Patent Application Publ. No. WO/2014/164253 (incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′ UTR and/or 3′ UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IEl)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nntl); calreticulin (Calr); a procollagen-lysine, 2-oxoglutarate 5-dioxygenase 1 (Plodl); and a nucleobindin (e.g., Nucbl).

In some embodiments, the 5′ UTR is selected from the group consisting of a β-globin 5′ UTR; a 5′ UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see US2010/0129877, the contents of which are incorporated herein by reference in its entirety).

The polynucleotides of the invention can comprise combinations of features. For example, the ORF can be flanked by a 5′ UTR that comprises a strong Kozak translational initiation signal and/or a 3′ UTR comprising an oligo(dT) sequence for templated addition of a poly-A tail. A 5′ UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

a. 5′ UTR sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide encoding a polypeptide comprising, inter alia, a 5′ UTR. In an embodiment, a polynucleotide disclosed herein comprises: (a) a 5′-UTR (e.g., as provided in Table 1 or a variant or fragment thereof); (b) a coding region; and (c) a stop element and 3′-UTR (e.g., as provided in Table 2 or a variant or fragment thereof), and LNP compositions comprising the same. In an embodiment, the polynucleotide comprises a 5′-UTR comprising a sequence provided in Table 1 or a variant or fragment thereof (e.g., a functional variant or fragment thereof). It will be understood that such 5′UTRs are incorporated into constructs not found in nature, e.g., such 5′ UTRs are synthetic, are altered in sequence from naturally occurring 5′UTRs, are truncated or lengthened versions of those found in nature, comprise chemically modified bases, are 5′ of ORF sequences different from those which they may be found in nature, or the like.

In an embodiment, the 5′ UTR comprises a sequence provided in Table 1 or a sequence with at least 80%, 85%, 90%, 95%, 96%, 97%, 98%, 99% or 100% identity to a 5′ UTR sequence provided in Table 1, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 or SEQ ID NO: 58.

In an embodiment, the 5′ UTR comprises a sequence with at least 80%, 85%, 90%, 95%, 96%, 97%, 98%, 99% or 100% identity to SEQ ID NO: 50. In an embodiment, the 5′ UTR comprises a sequence with at least 80%, 85%, 90%, 95%, 96%, 97%, 98%, 99% or 100% identity to SEQ ID NO: 51. In an embodiment, the 5′ UTR comprises a sequence with at least 80%, 85%, 90%, 95%, 96%, 97%, 98%, 99% or 100% identity to SEQ ID NO: 52. In an embodiment, the 5′ UTR comprises a sequence with at least 80%, 85%, 90%, 95%, 96%, 97%, 98%, 99% or 100% identity to SEQ ID NO: 53. In an embodiment, the 5′ UTR comprises a sequence with at least 80%, 85%, 90%, 95%, 96%, 97%, 98%, 99% or 100% identity to SEQ ID NO: 54. In an embodiment, the 5′ UTR comprises a sequence with at least 80%, 85%, 90%, 95%, 96%, 97%, 98%, 99% or 100% identity to SEQ ID NO: 55. In an embodiment, the 5′ UTR comprises a sequence with at least 80%, 85%, 90%, 95%, 96%, 97%, 98%, 99% or 100% identity to SEQ ID NO: 56. In an embodiment, the 5′ UTR comprises a sequence with at least 80%, 85%, 90%, 95%, 96%, 97%, 98%, 99% or 100% identity to SEQ ID NO: 57. In an embodiment, the 5′ UTR comprises a sequence with at least 80%, 85%, 90%, 95%, 96%, 97%, 98%, 99% or 100% identity to SEQ ID NO: 58.

In an embodiment, the 5′ UTR comprises a sequence with at least 80%, 85%, 90%, 95%, 96%, 97%, 98%, 99% or 100% identity to SEQ ID NO: 64.

In an embodiment, the 5′ UTR comprises the sequence of SEQ ID NO:50. In an embodiment, the 5′ UTR consists of the sequence of SEQ ID NO:50.

In an embodiment, the 5′ UTR comprises the sequence of SEQ ID NO:64. In an embodiment, the 5′ UTR consists of the sequence of SEQ ID NO:64.

In an embodiment, a 5′ UTR sequence provided in Table 1 has a first nucleotide which is an A. In an embodiment, a 5′ UTR sequence provided in Table 1 has a first nucleotide which is a G. In an embodiment, a 5′ UTR sequence provided in Table 1 has two first nucleotides which are an AG. In an embodiment, a 5′ UTR sequence provided in Table 1 has two first nucleotides which are a GA.

TABLE 1
5′ UTR sequences

| SEQ | | |
|---|---|---|
| ID | Sequence | |
| NO: | name | Sequence |
| 50 | A1 | GGAAAUCGCAAAAUUUGCUCUUCGCGUUAGAUUUCUUUUA |
| | | GUUUUCUCGCAACUAGCAAGCUUUUUGUUCUCGCC |
| 51 | A5 | GGAAAUCCCCACAACCGCCUCAUAUCCAGGCUCAAGAAUA |
| | | GAGCUCAGUGUUUUGUUGUUUAAUCAUUCCGACGUGUUUU |
| | | GCGAUAUUCGCGCAAAGCAGCCAGUCGCGCGCUUGCUUUU |
| | | AAGUAGAGUUGUUUUUCCACCCGUUUGCCAGGCAUCUUUA |
| | | AUUUAACAUAUUUUUAUUUUUCAGGCUAACCUACGCCGCC |
| | | ACC |
| 52 | A6 | GGAAAUAAGAGAGAAAAGAAGAGUAAGAAGAAAUAUAAGA |
| | | UCUCCCUGAGCUUCAGGGAGCCCCGGCGCCGCCACC |
| 53 | A7 | GGAAACCCCCCACCCCCGUAAGAGAGAAAAGAAGAGUAAG |
| | | AAGAAAUAUAAGAUCUCCCUGAGCUUCAGGGAGCCCCGGC |
| | | GCCGCCACC |
| 54 | A8 | GGAGAACUUCCGCUUCCGUUGGCGCAAGCGCUUUCAUUUU |
| | | UUCUGCUACCGUGACUAAG |
| 55 | A9 | GGAAAUAAGAGAGAAAAGAAGAGUAAGAAGAAAUAUAAGA |
| | | GCCACC |
| 56 | A11 | GGAAAUAAGAGAGAAAAGAAGAGUAAGAAGAAAUAUAAGA |
| | (Refer- | CCCCGGCGCCGCCACC |
| | ence) | |
| 57 | A2 | GGAAAUCGCAAAAUUUGCUCUUCGCGUUAGAUUUCUUUUA |
| | | GUUUUCUCGCAACUAGCAAGCUUUUUGUUCUCGCCGCCGC |
| | | C |
| 58 | A3 | GGAAAUCGCAAAAUUUUCUUUUCGCGUUAGAUUUUUUUA |
| | | GUUUUCUUUCAACUAGCAAGCUUUUUGUUCUCGCCGCCGC |
| | | C |
| 59 | A4 | G G A A A U C G C A A A A (N₂)ₓ (N₃)ₓ C U |
| | | (N₄)ₓ (N₅)ₓ C G C G U U A G A U U U C U U |
| | | U U A G U U U U C U N₆ N₇ C A A C U A G C |
| | | A A G C U U U U U G U U C U C G C C (N₈ |
| | | C C)x |
| | | (N₂)ₓ is a uracil and x is an integer from 0 to 5, e.g., |
| | | wherein x = 3 or 4; |
| | | (N₃)ₓ is a guanine and x is an integer from 0 to 1; |
| | | (N₄)ₓ is a cytosine and x is an integer from 0 to 1; |
| | | (N₅)ₓ is a uracil and x is an integer from 0 to 5, e.g., |
| | | wherein x = 2 or 3; |
| | | N₆ is a uracil or cytosine; |
| | | N₇ is a uracil or guanine; |
| | | N₈ is adenine or guanine and x is an integer from 0 to 1. |
| 60 | A27 | GGAAAAUUUUAGCCUGGAACGUUAGAUAACUGUCCUGUUG |
| | | UCUUUAUAUACUUGGUCCCCAAGUAGUUUGUCUUCCAAA |
| 61 | A12 | GGAAACUUUAUUUAGUGUUACUUUAUUUUCUGUUUAUUUG |
| | | UGUUUCUUCAGUGGGUUUGUUCUAAUUUCCUUGGCCGCC |
| 62 | A13 | GGAAAAUCUGUAUUAGGUUGGCGUGUUCUUUGGUCGGUUG |
| | | UUAGUAUUGUUGUUGAUUCGUUUGUGGUCGGUUGCCGCC |
| 63 | A14 | GGAAAAUUAUUAACAUCUUGGUAUUCUCGAUAACCAUUCG |
| | | UUGGAUUUUAUUGUAUUCGUAGUUUGGGUUCCUGCCGCC |
| 64 | A15 | GGAAAUUAUUAUUAUUUCUAGCUACAAUUUAUCAUUGUAU |
| | | UAUUUUAGCUAUUCAUCAUUAUUUACUUGGUGAUCAACA |
| 65 | A16 | GGAAAUAGGUUGUUAACCAAGUUCAAGCCUAAUAAGCUUG |
| | | GAUUCUGGUGACUUGCUUCACCGUUGGCGGGCACCGAUC |
| 66 | A17 | GGAAAUCGUAGAGAGUCGUACUUAGUACAUAUCGACUAUC |
| | | GGUGGACACCAUCAAGAUUAUAAACCAGGCCAGA |
| 67 | A18 | GGAAACCCGCCCAAGCGACCCCAACAUAUCAGCAGUUGCC |
| | | CAAUCCCAACUCCCAACACAAUCCCCAAGCAACGCCGCC |
| 68 | A19 | GGAAAGCGAUUGAAGGCGUCUUUUCAACUACUCGAUUAAG |
| | | GUUGGGUAUCGUCGUGGGACUUGGAAAUUUGUUGUUUCC |
| 69 | A20 | GGAAACUAAUCGAAAAAAAGAGCCCCGUACUCUUUUAUU |
| | | UCUAUUAGGUUAGGAGCCUUAGCAUUUGUAUCUUAGGUA |
| 70 | A21 | GGAAAUGUGAUUUCCAGCAACUUCUUUUGAAUAUAUUGAA |
| | | UUCCUAAUUCAAAGCGAACAAAUCUACAAGCCAUAUACC |
| 71 | A22 | GGAAAUCGUAGAGAGUCGUACUUACGUGGUCGCCAUUGCA |
| | | UAGCGCGCGAAAGCAACAGGAACAAGAACGCGCC |
| 72 | A23 | GGAAAUCGUAGAGAGUCGUACUUAGAAUAAACAGAGUCGG |
| | | GUCGACUUGUCUCUGAUACUACGACGUCACAAUC |
| 73 | A24 | GGAAAAUUUGCCUUCGGAGUUGCGUAUCCUGAACUGCCCA |
| | | GCCUCCUGAUAUACAACUGUUCCGCUUAUUCGGGCCGCC |
| 74 | A25 | GGAAAUCUGAGCAGGAAUCCUUUGUGCAUUGAAGACUUUA |
| | | GAUUCCUCUCUGCGGUAGACGUGCACUUAUAAGUAUUUG |
| 75 | A26 | GGAAAGCGAUUGAAGGCGUCUUUUCAACUACUCGAUUAAG |
| | | GUUGGGUAUCGUCGUGGGACUUGGAAAUUUGUUGCCACC |
| 76 | A28 | GGAAAUUUUUUUUUGAUAUUAUAAGAGUUUUUUUUUGAUA |
| | | UUAAGAAAAUUUUUUUUUGAUAUUAGAAGAGUAAGAAGAA |
| | | AUAUAAGACCCCGGCGCCGCCACC |
| 77 | A29 | GGAAAUAAGAGAGAAAAGAAGAGUAAGAAGAAAUAUAAGA |
| | | GCCAAAAAAAAAAAACC |
| 78 | A30 | GGAAAUCUCCCUGAGCUUCAGGGAGUAAGAGAGAAAAGAA |
| | | GAGUAAGAAGAAAUAUAAGACCCCGGCGCCGCCACC |
| | A31 | GCCRCC, wherein R = A or G |
| 80 | A32 | GGACUCACUAUUUGUUUUCGCGCCCAGUUGCAAAAA |

In an embodiment, the 5′ UTR comprises a variant of SEQ ID NO:50. In an embodiment, the variant of SEQ ID NO:50 comprises a nucleic acid sequence of Formula A:

(SEQ ID NO: 59)
G G A A A U C G C A A A A(N₂)ₓ(N₃)ₓC U(N₄)ₓ(N₅)ₓC G C
G U U A G A U U U C U U U U A G U U U U C U N₆N₇C A
A C U A G C A A G C U U U U U G U U C U C G C C
(N₈C C)x,

wherein:
(N₂)ₓ is a uracil and x is an integer from 0 to 5, e.g., wherein x=3 or 4;
(N₃)ₓ is a guanine and x is an integer from 0 to 1;
(N₄)ₓ is a cytosine and x is an integer from 0 to 1;
(N₅)ₓ is a uracil and x is an integer from 0 to 5, e.g., wherein x=2 or 3;
N₆ is a uracil or cytosine;
N₇ is a uracil or guanine;
N₈ is adenine or guanine and x is an integer from 0 to 1.

In an embodiment (N₂)ₓ is a uracil and x is 0. In an embodiment (N₂)ₓ is a uracil and x is 1. In an embodiment (N₂)ₓ is a uracil and x is 2. In an embodiment (N₂)ₓ is a uracil and x is 3. In an embodiment, (N₂)ₓ is a uracil and x is 4. In an embodiment (N₂)ₓ is a uracil and x is 5.

In an embodiment, (N₃)ₓ is a guanine and x is 0. In an embodiment, (N₃)ₓ is a guanine and x is 1.

In an embodiment, (N₄)ₓ is a cytosine and x is 0. In an embodiment, (N₄)ₓ is a cytosine and x is 1.

In an embodiment (N₅)ₓ is a uracil and x is 0. In an embodiment (N₅)ₓ is a uracil and x is 1. In an embodiment (N₅)ₓ is a uracil and x is 2. In an embodiment (N₅)ₓ is a uracil and x is 3. In an embodiment, (N₅)ₓ is a uracil and x is 4. In an embodiment (N₅)ₓ is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO:50. In an embodiment, the variant of SEQ ID NO:50 comprises a sequence with at least 50%, 60%, 70%, 80%, 85%, 90%, 95%, 96%, 97%, 98%, or 99% identity to SEQ ID NO:50. In an embodiment, the variant of SEQ ID NO:50 comprises a sequence with at least 50% identity to SEQ ID NO:50. In an embodiment, the variant of SEQ ID NO:50 comprises a sequence with at least 60% identity to SEQ ID NO:50. In an embodiment, the variant of SEQ ID NO:50 comprises a sequence with at least 70% identity to SEQ ID NO:50. In an embodiment, the variant of SEQ ID NO:50 comprises a sequence with at least 80% identity to SEQ ID NO:50. In an embodiment, the variant of SEQ ID NO:50 comprises a sequence with at least 90% identity to SEQ ID NO:50. In an embodiment, the variant of SEQ ID NO:50 comprises a sequence with at least 95% identity to SEQ ID NO:50. In an embodiment, the variant of SEQ ID NO:50 comprises a sequence with at least 96% identity to SEQ ID NO:50. In an embodiment, the variant of SEQ ID NO:50 comprises a sequence with at least 97% identity to SEQ ID NO:50. In an embodiment, the variant of SEQ ID NO:50 comprises a sequence with at least 98% identity to SEQ ID NO:50. In an embodiment, the variant of SEQ ID NO:50 comprises a sequence with at least 99% identity to SEQ ID NO:50.

In an embodiment, the 5′ UTR comprises a variant of SEQ ID NO:64. In an embodiment, the variant of SEQ ID NO: 64 comprises a sequence with at least 64%, 60%, 70%, 80%, 85%, 90%, 95%, 96%, 97%, 98%, or 99% identity to SEQ ID NO: 64.

In an embodiment, the variant of SEQ ID NO: 64 comprises a sequence with at least 64% identity to SEQ ID NO: 64. In an embodiment, the variant of SEQ ID NO: 64 comprises a sequence with at least 60% identity to SEQ ID NO: 64. In an embodiment, the variant of SEQ ID NO: 64 comprises a sequence with at least 70% identity to SEQ ID NO: 64. In an embodiment, the variant of SEQ ID NO: 64 comprises a sequence with at least 80% identity to SEQ ID NO: 64. In an embodiment, the variant of SEQ ID NO: 64 comprises a sequence with at least 90% identity to SEQ ID NO:64. In an embodiment, the variant of SEQ ID NO:64 comprises a sequence with at least 95% identity to SEQ ID NO:64. In an embodiment, the variant of SEQ ID NO:64 comprises a sequence with at least 96% identity to SEQ ID NO:64. In an embodiment, the variant of SEQ ID NO:64 comprises a sequence with at least 97% identity to SEQ ID NO:64. In an embodiment, the variant of SEQ ID NO:64 comprises a sequence with at least 98% identity to SEQ ID NO:64. In an embodiment, the variant of SEQ ID NO:64 comprises a sequence with at least 99% identity to SEQ ID NO:64.

In an embodiment, the 5′ UTR comprises a variant of SEQ ID NO:55. In an embodiment, the variant of SEQ ID NO:55 comprises a sequence with at least 55%, 60%, 70%, 80%, 85%, 90%, 95%, 96%, 97%, 98%, or 99% identity to SEQ ID NO:55. In an embodiment, the variant of SEQ ID NO:55 comprises a sequence with at least 55% identity to SEQ ID NO:55. In an embodiment, the variant of SEQ ID NO:55 comprises a sequence with at least 60% identity to SEQ ID NO:55. In an embodiment, the variant of SEQ ID NO:55 comprises a sequence with at least 70% identity to SEQ ID NO:55. In an embodiment, the variant of SEQ ID NO:55 comprises a sequence with at least 80% identity to SEQ ID NO:55. In an embodiment, the variant of SEQ ID NO:55 comprises a sequence with at least 90% identity to SEQ ID NO:55. In an embodiment, the variant of SEQ ID NO:55 comprises a sequence with at least 95% identity to SEQ ID NO:55. In an embodiment, the variant of SEQ ID NO:55 comprises a sequence with at least 96% identity to SEQ ID NO:55. In an embodiment, the variant of SEQ ID NO:55 comprises a sequence with at least 97% identity to SEQ ID NO:55. In an embodiment, the variant of SEQ ID NO:55 comprises a sequence with at least 98% identity to SEQ ID NO:55. In an embodiment, the variant of SEQ ID NO:55 comprises a sequence with at least 99% identity to SEQ ID NO:55.

In an embodiment, the 5′ UTR comprises a variant of SEQ ID NO:56. In an embodiment, the variant of SEQ ID NO:56 comprises a sequence with at least 56%, 60%, 70%, 80%, 85%, 90%, 95%, 96%, 97%, 98%, or 99% identity to SEQ ID NO:56. In an embodiment, the variant of SEQ ID NO:56 comprises a sequence with at least 56% identity to SEQ ID NO:56. In an embodiment, the variant of SEQ ID NO:56 comprises a sequence with at least 60% identity to SEQ ID NO:56. In an embodiment, the variant of SEQ ID NO:56 comprises a sequence with at least 70% identity to SEQ ID NO:56. In an embodiment, the variant of SEQ ID NO:56 comprises a sequence with at least 80% identity to SEQ ID NO:56. In an embodiment, the variant of SEQ ID NO:56 comprises a sequence with at least 90% identity to SEQ ID NO:56. In an embodiment, the variant of SEQ ID NO:56 comprises a sequence with at least 95% identity to SEQ ID NO:56. In an embodiment, the variant of SEQ ID NO:56 comprises a sequence with at least 96% identity to SEQ ID NO:56. In an embodiment, the variant of SEQ ID NO:56 comprises a sequence with at least 97% identity to SEQ ID NO:56. In an embodiment, the variant of SEQ ID NO:56 comprises a sequence with at least 98% identity to SEQ ID NO:56. In an embodiment, the variant of SEQ ID NO:56 comprises a sequence with at least 99% identity to SEQ ID NO:56.

In an embodiment, the 5′ UTR comprises a variant of SEQ ID NO:58. In an embodiment, the variant of SEQ ID NO:58 comprises a sequence with at least 58%, 60%, 70%, 80%, 85%, 90%, 95%, 96%, 97%, 98%, or 99% identity to SEQ ID NO:58. In an embodiment, the variant of SEQ ID NO:58 comprises a sequence with at least 58% identity to SEQ ID NO:58. In an embodiment, the variant of SEQ ID NO:58 comprises a sequence with at least 60% identity to SEQ ID NO:58. In an embodiment, the variant of SEQ ID NO:58 comprises a sequence with at least 70% identity to SEQ ID NO:58. In an embodiment, the variant of SEQ ID NO:58 comprises a sequence with at least 80% identity to SEQ ID NO:58. In an embodiment, the variant of SEQ ID NO:58 comprises a sequence with at least 90% identity to SEQ ID NO:58. In an embodiment, the variant of SEQ ID NO:58 comprises a sequence with at least 95% identity to SEQ ID NO:58. In an embodiment, the variant of SEQ ID NO:58 comprises a sequence with at least 96% identity to SEQ ID NO:58. In an embodiment, the variant of SEQ ID NO:58 comprises a sequence with at least 97% identity to SEQ ID NO:58. In an embodiment, the variant of SEQ ID NO:58 comprises a sequence with at least 98% identity to SEQ ID NO:58. In an embodiment, the variant of SEQ ID NO:58 comprises a sequence with at least 99% identity to SEQ ID NO:58.

In an embodiment, the 5′ UTR comprises a variant of SEQ ID NO:76. In an embodiment, the variant of SEQ ID NO:76 comprises a sequence with at least 76%, 60%, 70%, 80%, 85%, 90%, 95%, 96%, 97%, 98%, or 99% identity to SEQ ID NO:76. In an embodiment, the variant of SEQ ID NO:76 comprises a sequence with at least 76% identity to SEQ ID NO:76. In an embodiment, the variant of SEQ ID NO:76 comprises a sequence with at least 60% identity to SEQ ID NO:76. In an embodiment, the variant of SEQ ID NO:76 comprises a sequence with at least 70% identity to SEQ ID NO:76. In an embodiment, the variant of SEQ ID NO:76 comprises a sequence with at least 80% identity to SEQ ID NO:76. In an embodiment, the variant of SEQ ID NO:76 comprises a sequence with at least 90% identity to SEQ ID NO:76. In an embodiment, the variant of SEQ ID NO:76 comprises a sequence with at least 95% identity to SEQ ID NO:76. In an embodiment, the variant of SEQ ID NO:76 comprises a sequence with at least 96% identity to SEQ ID NO:76. In an embodiment, the variant of SEQ ID NO:76 comprises a sequence with at least 97% identity to SEQ ID NO:76. In an embodiment, the variant of SEQ ID NO:76 comprises a sequence with at least 98% identity to SEQ ID NO:76. In an embodiment, the variant of SEQ ID NO:76 comprises a sequence with at least 99% identity to SEQ ID NO:76.

In an embodiment, the 5′ UTR comprises a variant of SEQ ID NO:78. In an embodiment, the variant of SEQ ID NO:78 comprises a sequence with at least 78%, 60%, 70%, 80%, 85%, 90%, 95%, 96%, 97%, 98%, or 99% identity to SEQ ID NO:78. In an embodiment, the variant of SEQ ID NO:78 comprises a sequence with at least 78% identity to SEQ ID NO:78. In an embodiment, the variant of SEQ ID NO:78 comprises a sequence with at least 60% identity to SEQ ID NO:78. In an embodiment, the variant of SEQ ID NO:78 comprises a sequence with at least 70% identity to SEQ ID NO:78. In an embodiment, the variant of SEQ ID NO:78 comprises a sequence with at least 80% identity to SEQ ID NO:78. In an embodiment, the variant of SEQ ID NO:78 comprises a sequence with at least 90% identity to SEQ ID NO:78. In an embodiment, the variant of SEQ ID NO:78 comprises a sequence with at least 95% identity to SEQ ID NO:78. In an embodiment, the variant of SEQ ID NO:78 comprises a sequence with at least 96% identity to SEQ ID NO:78. In an embodiment, the variant of SEQ ID NO:78 comprises a sequence with at least 97% identity to SEQ ID NO:78. In an embodiment, the variant of SEQ ID NO:78 comprises a sequence with at least 98% identity to SEQ ID NO:78. In an embodiment, the variant of SEQ ID NO:78 comprises a sequence with at least 99% identity to SEQ ID NO:78.

In an embodiment, the variant of SEQ ID NO:50 comprises a uridine content of at least 5%, 10%, 20%, 30%, 40%, 50%, 60%, 70%, or 80%. In an embodiment, the variant of SEQ ID NO:50 comprises a uridine content of at least 5%. In an embodiment, the variant of SEQ ID NO:50 comprises a uridine content of at least 10%. In an embodiment, the variant of SEQ ID NO:50 comprises a uridine content of at least 20%. In an embodiment, the variant of SEQ ID NO:50 comprises a uridine content of at least 30%.

In an embodiment, the variant of SEQ ID NO:50 comprises a uridine content of at least 40%. In an embodiment, the variant of SEQ ID NO:50 comprises a uridine content of at least 50%. In an embodiment, the variant of SEQ ID NO:50 comprises a uridine content of at least 60%. In an embodiment, the variant of SEQ ID NO:50 comprises a uridine content of at least 70%. In an embodiment, the variant of SEQ ID NO:50 comprises a uridine content of at least 80%.

In an embodiment, the variant of SEQ ID NO:64 comprises a uridine content of at least 5%, 10%, 20%, 30%, 40%, 64%, 60%, 70%, or 80%. In an embodiment, the variant of SEQ ID NO:64 comprises a uridine content of at least 5%. In an embodiment, the variant of SEQ ID NO:64 comprises a uridine content of at least 10%. In an embodiment, the variant of SEQ ID NO:64 comprises a uridine content of at least 20%. In an embodiment, the variant of SEQ ID NO:64 comprises a uridine content of at least 30%.

In an embodiment, the variant of SEQ ID NO:64 comprises a uridine content of at least 40%. In an embodiment, the variant of SEQ ID NO:64 comprises a uridine content of at least 64%. In an embodiment, the variant of SEQ ID NO:64 comprises a uridine content of at least 60%. In an embodiment, the variant of SEQ ID NO:64 comprises a uridine content of at least 70%. In an embodiment, the variant of SEQ ID NO:64 comprises a uridine content of at least 80%.

In an embodiment, the variant of SEQ ID NO:55 comprises a uridine content of at least 5%, 10%, 20%, 30%, 40%, 55%, 60%, 70%, or 80%. In an embodiment, the variant of SEQ ID NO:55 comprises a uridine content of at least 5%. In an embodiment, the variant of SEQ ID NO:55 comprises a uridine content of at least 10%. In an embodiment, the variant of SEQ ID NO:55 comprises a uridine content of at least 20%. In an embodiment, the variant of SEQ ID NO:55 comprises a uridine content of at least 30%.

In an embodiment, the variant of SEQ ID NO:55 comprises a uridine content of at least 40%. In an embodiment, the variant of SEQ ID NO:55 comprises a uridine content of at least 55%. In an embodiment, the variant of SEQ ID NO:55 comprises a uridine content of at least 60%. In an embodiment, the variant of SEQ ID NO:55 comprises a uridine content of at least 70%. In an embodiment, the variant of SEQ ID NO:55 comprises a uridine content of at least 80%.

In an embodiment, the variant of SEQ ID NO:56 comprises a uridine content of at least 5%, 10%, 20%, 30%, 40%, 56%, 60%, 70%, or 80%. In an embodiment, the variant of SEQ ID NO:56 comprises a uridine content of at least 5%. In an embodiment, the variant of SEQ ID NO:56 comprises a uridine content of at least 10%. In an embodiment, the variant of SEQ ID NO:56 comprises a uridine content of at least 20%. In an embodiment, the variant of SEQ ID NO:56 comprises a uridine content of at least 30%.

In an embodiment, the variant of SEQ ID NO:56 comprises a uridine content of at least 40%. In an embodiment, the variant of SEQ ID NO:56 comprises a uridine content of at least 56%. In an embodiment, the variant of SEQ ID NO:56 comprises a uridine content of at least 60%. In an embodiment, the variant of SEQ ID NO:56 comprises a uridine content of at least 70%. In an embodiment, the variant of SEQ ID NO:56 comprises a uridine content of at least 80%.

In an embodiment, the variant of SEQ ID NO:58 comprises a uridine content of at least 5%, 10%, 20%, 30%, 40%, 58%, 60%, 70%, or 80%. In an embodiment, the variant of SEQ ID NO:58 comprises a uridine content of at least 5%. In an embodiment, the variant of SEQ ID NO:58 comprises a uridine content of at least 10%. In an embodiment, the variant of SEQ ID NO:58 comprises a uridine content of at least 20%. In an embodiment, the variant of SEQ ID NO:58 comprises a uridine content of at least 30%.

In an embodiment, the variant of SEQ ID NO:58 comprises a uridine content of at least 40%. In an embodiment, the variant of SEQ ID NO:58 comprises a uridine content of at least 58%. In an embodiment, the variant of SEQ ID NO:58 comprises a uridine content of at least 60%. In an embodiment, the variant of SEQ ID NO:58 comprises a uridine content of at least 70%. In an embodiment, the variant of SEQ ID NO:58 comprises a uridine content of at least 80%.

In an embodiment, the variant of SEQ ID NO:76 comprises a uridine content of at least 5%, 10%, 20%, 30%, 40%, 76%, 60%, 70%, or 80%. In an embodiment, the variant of SEQ ID NO:76 comprises a uridine content of at least 5%. In an embodiment, the variant of SEQ ID NO:76 comprises a uridine content of at least 10%. In an embodiment, the variant of SEQ ID NO:76 comprises a uridine content of at least 20%. In an embodiment, the variant of SEQ ID NO:76 comprises a uridine content of at least 30%.

In an embodiment, the variant of SEQ ID NO:76 comprises a uridine content of at least 40%. In an embodiment, the variant of SEQ ID NO:76 comprises a uridine content of at least 76%. In an embodiment, the variant of SEQ ID NO:76 comprises a uridine content of at least 60%. In an embodiment, the variant of SEQ ID NO:76 comprises a uridine content of at least 70%. In an embodiment, the variant of SEQ ID NO:76 comprises a uridine content of at least 80%.

In an embodiment, the variant of SEQ ID NO:78 comprises a uridine content of at least 5%, 10%, 20%, 30%, 40%, 78%, 60%, 70%, or 80%. In an embodiment, the variant of SEQ ID NO:78 comprises a uridine content of at least 5%. In an embodiment, the variant of SEQ ID NO:78 comprises a uridine content of at least 10%. In an embodiment, the variant of SEQ ID NO:78 comprises a uridine content of at least 20%. In an embodiment, the variant of SEQ ID NO:78 comprises a uridine content of at least 30%.

In an embodiment, the variant of SEQ ID NO:78 comprises a uridine content of at least 40%. In an embodiment, the variant of SEQ ID NO:78 comprises a uridine content of at least 78%. In an embodiment, the variant of SEQ ID NO:78 comprises a uridine content of at least 60%. In an embodiment, the variant of SEQ ID NO:78 comprises a uridine content of at least 70%. In an embodiment, the variant of SEQ ID NO:78 comprises a uridine content of at least 80%.

In an embodiment, the variant of SEQ ID NO:50 comprises at least 2, 3, 4, 5, 6 or 7 consecutive uridines (e.g., a polyuridine tract). In an embodiment, the polyuridine tract in the variant of SEQ ID NO:50 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:50 comprises 4 consecutive uridines. In an embodiment, the polyuridine tract in the variant of SEQ ID NO:50 comprises 5 consecutive uridines.

In an embodiment, the variant of SEQ ID NO:64 comprises at least 2, 3, 4, 5, 6 or 7 consecutive uridines (e.g., a polyuridine tract). In an embodiment, the polyuridine tract in the variant of SEQ ID NO:64 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:64 comprises 4 consecutive uridines. In an embodiment, the polyuridine tract in the variant of SEQ ID NO:64 comprises 5 consecutive uridines.

In an embodiment, the variant of SEQ ID NO:55 comprises at least 2, 3, 4, 5, 6 or 7 consecutive uridines (e.g., a polyuridine tract). In an embodiment, the polyuridine tract in the variant of SEQ ID NO:55 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:55 comprises 4 consecutive uridines. In an embodiment, the polyuridine tract in the variant of SEQ ID NO:55 comprises 5 consecutive uridines.

In an embodiment, the variant of SEQ ID NO:56 comprises at least 2, 3, 4, 5, 6 or 7 consecutive uridines (e.g., a polyuridine tract). In an embodiment, the polyuridine tract in the variant of SEQ ID NO:56 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:56 comprises 4 consecutive uridines. In an embodiment, the polyuridine tract in the variant of SEQ ID NO:56 comprises 5 consecutive uridines. In an embodiment, the variant of SEQ ID NO:58 comprises at least 2, 3, 4, 5, 6 or 7 consecutive uridines (e.g., a polyuridine tract). In an embodiment, the polyuridine tract in the variant of SEQ ID NO:58 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:58 comprises 4 consecutive uridines. In an embodiment, the polyuridine tract in the variant of SEQ ID NO:58 comprises 5 consecutive uridines.

In an embodiment, the variant of SEQ ID NO:76 comprises at least 2, 3, 4, 5, 6 or 7 consecutive uridines (e.g., a polyuridine tract). In an embodiment, the polyuridine tract in the variant of SEQ ID NO:76 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:76 comprises 4 consecutive uridines. In an embodiment, the polyuridine tract in the variant of SEQ ID NO:76 comprises 5 consecutive uridines.

In an embodiment, the variant of SEQ ID NO:78 comprises at least 2, 3, 4, 5, 6 or 7 consecutive uridines (e.g., a polyuridine tract). In an embodiment, the polyuridine tract in the variant of SEQ ID NO:78 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO:78 comprises 4 consecutive uridines. In an embodiment, the polyuridine tract in the variant of SEQ ID NO:78 comprises 5 consecutive uridines.

In an embodiment, the variant of SEQ ID NO:50 comprises 1, 2, 3, 4, 5, 6, 7, 8, 9, 10, 11, 12, 13, 14, or 15 polyuridine tracts. In an embodiment, the variant of SEQ ID NO:50 comprises 3 polyuridine tracts. In an embodiment, the variant of SEQ ID NO:50 comprises 4 polyuridine tracts. In an embodiment, the variant of SEQ ID NO:50 comprises 5 polyuridine tracts.

In an embodiment, the variant of SEQ ID NO:64 comprises 1, 2, 3, 4, 5, 6, 7, 8, 9, 10, 11, 12, 13, 14, or 15 polyuridine tracts. In an embodiment, the variant of SEQ ID NO:64 comprises 3 polyuridine tracts. In an embodiment, the variant of SEQ ID NO:64 comprises 4 polyuridine tracts. In an embodiment, the variant of SEQ ID NO:64 comprises 5 polyuridine tracts.

In an embodiment, the variant of SEQ ID NO:55 comprises 1, 2, 3, 4, 5, 6, 7, 8, 9, 10, 11, 12, 13, 14, or 15 polyuridine tracts. In an embodiment, the variant of SEQ ID NO:55 comprises 3 polyuridine tracts. In an embodiment, the variant of SEQ ID NO:55 comprises 4 polyuridine tracts. In an embodiment, the variant of SEQ ID NO:55 comprises 5 polyuridine tracts.

In an embodiment, the variant of SEQ ID NO:56 comprises 1, 2, 3, 4, 5, 6, 7, 8, 9, 10, 11, 12, 13, 14, or 15 polyuridine tracts. In an embodiment, the variant of SEQ ID NO:56 comprises 3 polyuridine tracts. In an embodiment, the variant of SEQ ID NO:56 comprises 4 polyuridine tracts. In an embodiment, the variant of SEQ ID NO:56 comprises 5 polyuridine tracts.

In an embodiment, the variant of SEQ ID NO:58 comprises 1, 2, 3, 4, 5, 6, 7, 8, 9, 10, 11, 12, 13, 14, or 15 polyuridine tracts. In an embodiment, the variant of SEQ ID NO:58 comprises 3 polyuridine tracts. In an embodiment, the variant of SEQ ID NO:58 comprises 4 polyuridine tracts. In an embodiment, the variant of SEQ ID NO:58 comprises 5 polyuridine tracts.

In an embodiment, the variant of SEQ ID NO:76 comprises 1, 2, 3, 4, 5, 6, 7, 8, 9, 10, 11, 12, 13, 14, or 15 polyuridine tracts. In an embodiment, the variant of SEQ ID NO:76 comprises 3 polyuridine tracts. In an embodiment, the variant of SEQ ID NO:76 comprises 4 polyuridine tracts. In an embodiment, the variant of SEQ ID NO:76 comprises 5 polyuridine tracts.

In an embodiment, the variant of SEQ ID NO:78 comprises 1, 2, 3, 4, 5, 6, 7, 8, 9, 10, 11, 12, 13, 14, or 15 polyuridine tracts. In an embodiment, the variant of SEQ ID NO:78 comprises 3 polyuridine tracts. In an embodiment, the variant of SEQ ID NO:78 comprises 4 polyuridine tracts. In an embodiment, the variant of SEQ ID NO:78 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18. 19, 20, 30, 40, 50 or 60 nucleotides.

In an embodiment, a first polyuridine tract and a second polyuridine tract are adjacent to each other.

In an embodiment, a subsequent, e.g., third, fourth, fifth, sixth or seventh, eighth, ninth, or tenth, polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18. 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence wherein R is an adenine or guanine. In an embodiment, the Kozak sequence is disposed at the 3′ end of the 5′ UTR sequence.

In an embodiment, the polynucleotide comprising a 5′ UTR sequence disclosed herein comprises a coding region which encodes for a payload, e.g., a therapeutic or prophylactic payload.

In an aspect, the polynucleotide (e.g., mRNA) comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

b. Stop Elements+3′ UTR Sequences

Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162). 3′ UTR sequences have been shown to influence translation, half-life, and subcellular localization of mRNAs (Mayr C., Cold Spring Harb Persp Biol 2019 Oct 1;11(10):a034728).

Disclosed herein, inter alia, is a polynucleotide encoding a polypeptide, which polynucleotide has a stop element in combination with a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR; (b) a coding region; and (c) a stop element and 3′-UTR (e.g., as described herein), and LNP compositions comprising the same.

Disclosed herein, inter alia, is a polynucleotide encoding a polypeptide comprising, inter alia, a 3′ UTR. In an embodiment, a polynucleotide disclosed herein comprises: (a) a 5′-UTR (e.g., as provided in Table 1 or a variant or fragment thereof); (b) a coding region; and (c) a stop element and 3′-UTR (e.g., as provided in Table 2 or a variant or fragment thereof), and LNP compositions comprising the same. In an embodiment, the polynucleotide comprises a 3′-UTR comprising a sequence provided in Table 2 or a variant or fragment thereof (e.g., a functional variant or fragment thereof). It will be understood that such 3′UTRs are incorporated into constructs not found in nature, e.g., such 3′ UTRs are synthetic, are altered in sequence from naturally occurring 3′UTRs, are truncated or lengthened versions of those found in nature, comprise chemically modified bases, are 3′ of ORF sequences different from those which they may be found in nature, or the like.

In an embodiment, the 3′ UTR comprises a sequence provided in Table 2 or a sequence with at least 80%, 85%, 90%, 95%, 96%, 97%, 98%, 99% or 100% identity to a 3′ UTR sequence provided in Table 2, or a variant or a fragment thereof. In an embodiment, the 3′ UTR comprises a sequence with at least 80%, 850%, 90%, 95%, 96%, 97%, 98%, 99% or 100% identity to SEQ TD NO: 139, SEQ TD NO: 140, SEQ TD NO: 141, SEQ ID NO: 142, SEQ ID NO: 143, SEQ ID NO: 144, SEQ ID NO: 145, SEQ TD NO: 146, or SEQID NO: 147.

TABLE 2
3′ UTR sequences (stop cassette is italicized; miR binding sites
are boldened)

| SEQ | Sequence | |
|---|---|---|
| ID NO: | name | Sequence |
| SEQ ID NO: | D1 | UAAAGCUCCCCGGGGGCCUCGGUGGCCUAGCUUCUUGCCC |
| 139 | | CUUGGGCCUCCCCCCAGCCCCUCCUCCCCUUCCUGCACCC |
| | | GUACCCCCGUGGUCUUUGAAUAAAGUCUGAGUGGGCGGC |
| SEQ ID NO: | D2 | UAAGUCUAAGCUGGAGCCUCCUGAGAGACCUGUGUGAACU |
| 140 | | AUUGAGAAGAUCGGAACAGCUCCUUACUCUGAGGAAGUUG |
| | | GUACCCCCGUGGUCUUUGAAUAAAGUCUGAGUGGGCGGC |
| SEQ ID NO: | D3 | UAAAGCUCCCCGGGGCAAACACCAUUGUCACACUCCAGCC |
| 141 | | UCGGUGGCCUAGCUUCUUGCCCCUUGGGCCCAAACACCAU |
| | | UGUCACACUCCAUCCCCCCAGCCCCUCCUCCCCUUCCUGC |
| | | ACCCGUACCCCCCAAACACCAUUGUCACACUCCAGUGGUC |
| | | UUUGAAUAAAGUCUGAGUGGGCGGC |
| | | (miR122 binding sites boldened) |
| SEQ ID NO: | D4 | UAAAGCUCCCCGGGGUCCAUAAAGUAGGAAACACUACAGC |
| 142 | | UGGAGCCUCGGUGGCCUAGCUUCUUGCCCCUUGGGCCCAA |
| | | ACACCAUUGUCACACUCCAUCCCCCCAGCCCCUCCUCCCC |
| | | UUCCUGCACCCGUACCCCCGUGGUCUUUGAAUAAAGUCUG |
| | | AGUGGGCGGC |
| | | (miR-142-3p and miR 122 binding sites boldened) |
| SEQ ID NO: | D5 | UAAAGCUCCCCGGGGGCCUCGGUGGCCUAGCUUCUUGCCC |
| 143 | | CUUGGGCCUCCCCCCAGCCCCUCCUCCCCUUCCUGCACCC |
| | | GUACCCCCCAAACACCAUUGUCACACUCCAGUGGUCUUUG |
| | | AAUAAAGUCUGAGUGGGCGGC |
| | | (miR122 binding site boldened) |
| SEQ ID NO: | D6 | UAAGCCCCUCCGGGGCAAACACCAUUGUCACACUCCAGCC |
| 144 | | UCGGUGGCCUAGCUUCUUGCCCCUUGGGCCCAAACACCAU |
| | | UGUCACACUCCAUCCCCCCAGCCCCUCCUCCCCUUCCUGC |
| | | ACCCGUACCCCCCAAACACCAUUGUCACACUCCAGUGGUC |
| | | UUUGAAUAAAGUCUGAGUGGGCGGC |
| | | (miR122 binding sites boldened) |
| SEQ ID NO: | D7 | UAAGCCCCUCCGGGGUCCAUAAAGUAGGAAACACUACAGC |
| 145 | | CUCGGUGGCCUAGCUUCUUGCCCCUUGGGCCUCCAUAAAG |
| | | UAGGAAACACUACAUCCCCCCAGCCCCUCCUCCCCUUCCU |
| | | GCACCCGUACCCCCCGCAUUAUUACUCACGGUACGAGUGG |
| | | UCUUUGAAUAAAGUCUGAGUGGGCGGC |
| | | (miR-142-3p and miR-126-3p binding sites boldened) |
| SEQ ID NO: | D8 | UAAGCCCCUCCGGGGUCCAUAAAGUAGGAAACACUACAGC |
| 146 | | UGGAGCCUCGGUGGCCUAGCUUCUUGCCCCUUGGGCCCAA |
| | | ACACCAUUGUCACACUCCAUCCCCCCAGCCCCUCCUCCCC |
| | | UUCCUGCACCCGUACCCCCGUGGUCUUUGAAUAAAGUCUG |
| | | AGUGGGCGGC |
| | | (miR-142-3p and miR 122 binding sites boldened) |
| SEQ ID NO: | D9 | UAAGCCCCUCCGGGGGCCUCGGUGGCCUAGCUUCUUGCCC |
| 147 | | CUUGGGCCUCCCCCCAGCCCCUCCUCCCCUUCCUGCACCC |
| | | GUACCCCCCAAACACCAUUGUCACACUCCAGUGGUCUUUG |
| | | AAUAAAGUCUGAGUGGGCGGC |
| | | (miR122 binding site boldened) |

In an embodiment, the polynucleotide comprises a stop element and 3′-UTR, wherein the sequence is (stop element is italicized): UAAAGCUCCCCGGGGGCCUCGGUGGCCUAGCUUCUUGCCCCUUGGGCCUCC CCCCAGCCCCUCCUCCCCUUCCUGCACCCGUACCCCCGUGGUCUUUGAAUA AAGUCUGAGUGGGCGGC (SEQ ID NO:139) or a variant or fragment thereof (e.g., a fragment that lacks the first one, two, three, four, five, six, or more nucleotides of nucleotides of SEQ ID NO:139.

In an embodiment, the polynucleotide having a 3′ UTR sequence provided in 10 SEQ ID NO:139 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:139 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:139 or a variant or fragment thereof.

In an embodiment, the polynucleotide comprises a 3′ UTR sequence provided in SEQ ID NO:139 or a sequence with at least 80%, 85%, 90%, 95%, 96%, 97%, 98%, 99% or 100% identity to a 3′ UTR sequence provided in SEQ ID NO:139.

In an embodiment, the polynucleotide comprises a stop element and 3′-UTR, wherein the sequence is (stop element is italicized): UAAGUCUAAGCUGGAGCCUCCUGAGAGACCUGUGUGAACUAUUGAGAAGAU CGGAACAGCUCCUUACUCUGAGGAAGUUGGUACCCCCGUGGUCUUUGAAU AAAGUCUGAGUGGGCGGC (SEQ ID NO:140) or a variant or fragment thereof (e.g., a fragment that lacks the first one, two, three, four, five, six, or more nucleotides of nucleotides of SEQ ID NO:140.

In an embodiment, the polynucleotide having a 3′ UTR sequence provided in SEQ ID NO:140 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:140 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:140 or a variant or fragment thereof.

In an embodiment, the polynucleotide comprises a 3′ UTR sequence provided in SEQ ID NO:140 or a sequence with at least 80%, 85%, 90%, 95%, 96%, 97%, 98%, 99% or 100% identity to a 3′ UTR sequence provided in SEQ ID NO:140.

In an embodiment, the polynucleotide comprises a stop element and 3′-UTR, wherein the sequence is (stop element is italicized): UAAAGCUCCCCGGGGCAAACACCAUUGUCACACUCCAGCCUCGGUGGCCUA GCUUCUUGCCCCUUGGGCCCAAACACCAUUGUCACACUCCAUCCCCCCAGC CCCUCCUCCCCUUCCUGCACCCGUACCCCCCAAACACCAUUGUCACACUCCA GUGGUCUUUGAAUAAAGUCUGAGUGGGCGGC (SEQ ID NO:141) or a variant or fragment thereof (e.g., a fragment that lacks the first one, two, three, four, five, six, or more nucleotides of nucleotides of SEQ ID NO:141.

In an embodiment, the polynucleotide having a 3′ UTR sequence provided in SEQ ID NO:141 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:141 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:141 or a variant or fragment thereof.

In an embodiment, the polynucleotide comprises a 3′ UTR sequence provided in SEQ ID NO:141 or a sequence with at least 80%, 85%, 90%, 95%, 96%, 97%, 98%, 99% or 100% identity to a 3′ UTR sequence provided in SEQ ID NO:141.

In an embodiment, the polynucleotide comprises a stop element and 3′-UTR, wherein the sequence is (stop element is italicized): UAAAGCUCCCCGGGGUCCAUAAAGUAGGAAACACUACAGCUGGAGCCUCGG UGGCCUAGCUUCUUGCCCCUUGGGCCCAAACACCAUUGUCACACUCCAUCC CCCCAGCCCCUCCUCCCCUUCCUGCACCCGUACCCCCGUGGUCUUUGAAUA AAGUCUGAGUGGGCGGC (SEQ ID NO:142) or a variant or fragment thereof (e.g., a fragment that lacks the first one, two, three, four, five, six, or more nucleotides of nucleotides of SEQ ID NO:142.

In an embodiment, the polynucleotide having a 3′ UTR sequence provided in SEQ ID NO:142 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:142 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:142 or a variant or fragment thereof.

In an embodiment, the polynucleotide comprises a 3′ UTR sequence provided in SEQ ID NO:142 or a sequence with at least 80%, 85%, 90%, 95%, 96%, 97%, 98%, 99% or 100% identity to a 3′ UTR sequence provided in SEQ ID NO:142.

In an embodiment, the polynucleotide comprises a stop element and 3′-UTR, wherein the sequence is (stop element is italicized): UAAAGCUCCCCGGGGGCCUCGGUGGCCUAGCUUCUUGCCCCUUGGGCCUCC CCCCAGCCCCUCCUCCCCUUCCUGCACCCGUACCCCCCAAACACCAUUGUCA CACUCCAGUGGUCUUUGAAUAAAGUCUGAGUGGGCGGC (SEQ ID NO:143) or a variant or fragment thereof (e.g., a fragment that lacks the first one, two, three, four, five, six, or more nucleotides of nucleotides of SEQ ID NO:143.

In an embodiment, the polynucleotide having a 3′ UTR sequence provided in SEQ ID NO:143 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:143 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:143 or a variant or fragment thereof.

In an embodiment, the polynucleotide comprises a 3′ UTR sequence provided in SEQ ID NO:143 or a sequence with at least 80%, 85%, 90%, 95%, 96%, 97%, 98%, 99% or 100% identity to a 3′ UTR sequence provided in SEQ ID NO:143.

In an embodiment, the polynucleotide comprises a stop element and 3′-UTR, wherein the sequence is (stop element is italicized): UAAGCCCCUCCGGGGCAAACACCAUUGUCACACUCCAGCCUCGGUGGCCUA GCUUCUUGCCCCUUGGGCCCAAACACCAUUGUCACACUCCAUCCCCCCAGC CCCUCCUCCCCUUCCUGCACCCGUACCCCCCAAACACCAUUGUCACACUCCA GUGGUCUUUGAAUAAAGUCUGAGUGGGCGGC (SEQ ID NO:144) or a variant or fragment thereof (e.g., a fragment that lacks the first one, two, three, four, five, six, or more nucleotides of nucleotides of SEQ ID NO:144.

In an embodiment, the polynucleotide having a 3′ UTR sequence provided in SEQ ID NO:144 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:144 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:144 or a variant or fragment thereof.

In an embodiment, the polynucleotide comprises a 3′ UTR sequence provided in SEQ ID NO:144 or a sequence with at least 80%, 85%, 90%, 95%, 96%, 97%, 98%, 99% or 100% identity to a 3′ UTR sequence provided in SEQ ID NO:144.

In an embodiment, the polynucleotide comprises a stop element and 3′-UTR, wherein the sequence is (stop element is italicized): UAAGCCCCUCCGGGGUCCAUAAAGUAGGAAACACUACAGCCUCGGUGGCCU AGCUUCUUGCCCCUUGGGCCUCCAUAAAGUAGGAAACACUACAUCCCCCCA GCCCCUCCUCCCCUUCCUGCACCCGUACCCCCCGCAUUAUUACUCACGGUA CGAGUGGUCUUUGAAUAAAGUCUGAGUGGGCGGC (SEQ ID NO:145) or a variant or fragment thereof (e.g., a fragment that lacks the first one, two, three, four, five, six, or more nucleotides of nucleotides of SEQ ID NO:145.

In an embodiment, the polynucleotide having a 3′ UTR sequence provided in SEQ ID NO:145 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:145 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:145 or a variant or fragment thereof.

In an embodiment, the polynucleotide comprises a 3′ UTR sequence provided in SEQ ID NO:145 or a sequence with at least 80%, 85%, 90%, 95%, 96%, 97%, 98%, 99% or 100% identity to a 3′ UTR sequence provided in SEQ ID NO:145.

In an embodiment, the polynucleotide comprises a stop element and 3′-UTR, wherein the sequence is (stop element is italicized): UAAGCCCCUCCGGGGUCCAUAAAGUAGGAAACACUACAGCUGGAGCCUCGG UGGCCUAGCUUCUUGCCCCUUGGGCCCAAACACCAUUGUCACACUCCAUCC CCCCAGCCCCUCCUCCCCUUCCUGCACCCGUACCCCCGUGGUCUUUGAAUA AAGUCUGAGUGGGCGGC (SEQ ID NO:146) or a variant or fragment thereof (e.g., a fragment that lacks the first one, two, three, four, five, six, or more nucleotides of nucleotides of SEQ ID NO:146.

In an embodiment, the polynucleotide having a 3′ UTR sequence provided in SEQ ID NO:146 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:146 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:146 or a variant or fragment thereof.

In an embodiment, the polynucleotide comprises a 3′ UTR sequence provided in SEQ ID NO:146 or a sequence with at least 80%, 85%, 90%, 95%, 96%, 97%, 98%, 99% or 100% identity to a 3′ UTR sequence provided in SEQ ID NO:146.

In an embodiment, the polynucleotide comprises a stop element and 3′-UTR, wherein the sequence is (stop element is italicized): UAAGCCCCUCCGGGGGCCUCGGUGGCCUAGCUUCUUGCCCCUUGGGCCUCC CCCCAGCCCCUCCUCCCCUUCCUGCACCCGUACCCCCCAAACACCAUUGUCA CACUCCAGUGGUCUUUGAAUAAAGUCUGAGUGGGCGGC (SEQ ID NO:147) or a variant or fragment thereof (e.g., a fragment that lacks the first one, two, three, four, five, six, or more nucleotides of nucleotides of SEQ ID NO:147.

In an embodiment, the polynucleotide having a 3′ UTR sequence provided in SEQ ID NO:147 or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more.

In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in SEQ ID NO:147 or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of SEQ ID NO:147 or a variant or fragment thereof.

In an embodiment, the polynucleotide comprises a 3′ UTR sequence provided in SEQ ID NO:147 or a sequence with at least 80%, 85%, 90%, 95%, 96%, 97%, 98%, 99% or 100% identity to a 3′ UTR sequence provided in SEQ ID NO:147. 2. 3′ stabilizing region Disclosed herein, inter alia, is a polynucleotide encoding a polypeptide, wherein the polynucleotide comprises: (a) a 5′-UTR (e.g., as described herein); (b) a coding region comprising a stop element (e.g., as described herein); (c) a 3′-UTR (e.g., as described herein), and (d) a 3′ stabilizing region. Also disclosed herein are LNP compositions comprising the same.

In an embodiment, the polynucleotide comprises a 3′ stabilizing region, e.g., a stabilized tail e.g., as described herein. A polynucleotide containing a 3′-stabilizing region (e.g., a 3′-stabilizing region including an alternative nucleobase, sugar, and/or backbone) may be particularly effective for use in therapeutic compositions, because they may benefit from increased stability, high expression levels.

In an embodiment, the 3′ stabilizing region comprises a poly A tail, e.g., a poly A tail comprising 80-150 (SEQ ID NO:184), e.g., 120, adenines (SEQ ID NO:123). In an embodiment, the poly A tail comprises one or more non-adenosine residues, e.g., one or more guanosines, e.g., as described herein. In an embodiment, the poly A tail comprises a UCUAG sequence. In an embodiment, the poly A tail comprises about 80-120, e.g., 100, adenines upstream of SEQ ID NO: 44. In an embodiment, the poly A tail comprises about 1-40, e.g., 20, adenines downstream of SEQ ID NO: 44.

In an embodiment, the 3′ stabilizing region comprises at least one alternative nucleoside. In an embodiment, the alternative nucleoside is an inverted thymidine (idT).

In an embodiment, the alternative nucleoside is disposed at the 3′ end of the 3′ stabilizing region.

In an embodiment, the 3′ stabilizing region comprises a structure of Formula VII:

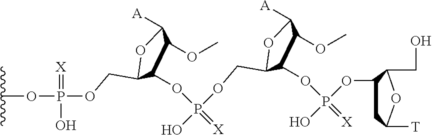

or a salt thereof, wherein each X is independently O or S, and A represents adenine and T represents thymine.

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); (c) a 3′-UTR (e.g., as described herein) and; (d) a 3′ stabilizing region, e.g., as described herein.

In an aspect, an LNP composition comprising a polynucleotide comprising a stabilizing region disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition comprising a polynucleotide disclosed herein encoding a therapeutic payload or prophylactic payload, e.g., as described herein, can be administered with an additional agent, e.g., as described herein.

3. MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh KK, Johnston WK, Garrett-Engele P, Lim LP, Bartel DP; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′ UTR and/or 3′ UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the nucleic acid molecule (e.g., RNA, e.g., mRNA).

For example, one of skill in the art would understand that one or more miR binding sites can be included in a nucleic acid molecule (e.g., RNA, e.g., mRNA) to minimize expression in cell types other than lymphoid cells. In one embodiment, a miR122 binding site can be used. In another embodiment, a miR126 binding site can be used. In still another embodiment, multiple copies of these miR binding sites or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety). miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation.

Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′ UTR and/or 3′ UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response.

Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7α-2-3p, hsa-let-7α-3p, hsa-7α-5p, hsa-let-7c, hsa-let-7e-3p, hsa-let-7e-5p, hsa-let-7g-3p, hsa-let-7g-5p, hsa-let-7i-3p, hsa-let-7i-5p, miR-10α-3p, miR-10α-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130α-3p, miR-130α-5p, miR-132-3p, miR-132-5p, miR-142-3p, miR-142-5p, miR-143-3p, miR-143-5p, miR-146α-3p, miR-146α-5p, miR-146b-3p, miR-146b-5p, miR-147a, miR-147b, miR-148α-5p, miR-148α-3p, miR-150-3p, miR-150-5p, miR-151b, 15 miR-155-3p, miR-155-5p, miR-15α-3p, miR-15α-5p, miR-15b-5p, miR-15b-3p, miR-16-1-3p, miR-16-2-3p, miR-16-5p, miR-17-5p, miR-181α-3p, miR-181α-5p, miR-181α-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p,miR-24-2-5p, miR-24-3p, miR-26α-1-3p, miR-26α-2-3p, miR-26α-5p, miR-26b-3p, miR-26b-5p, miR-27α-3p, miR-27α-5p, miR-27b-3p,miR-27b-5p, miR-28-3p, miR-28-5p, miR-2909, miR-29α-3p, miR-29α-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34α-3p, miR-34α-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99α-3p, miR-99α-5p, miR-99b-3p, and miR-99b-5p.

Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.) In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure. miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′ UTR and/or 3′ UTR. As a non-limiting example, a non-human 3′ UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′ UTR in order to enhance microRNA mediated gene regulation.

At least one miRNA binding site can be engineered into the 3′ UTR of a polynucleotide of the disclosure. In this context, at least two, at least three, at least four, at least five, at least six, at least seven, at least eight, at least nine, at least ten, or more miRNA binding sites can be engineered into a 3′ UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′ UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′ UTR, about halfway between the 5′ terminus and 3′ terminus of the 3′ UTR and/or near the 3′ terminus of the 3′ UTR in a nucleic acid molecule (e.g., RNA, e.g., mRNA) of the disclosure. As a non-limiting example, a miRNA binding site can be engineered near the 5′ terminus of the 3′ UTR and about halfway between the 5′ terminus and 3′ terminus of the 3′ UTR. As another non-limiting example, a miRNA binding site can be engineered near the 3′ terminus of the 3′ UTR and about halfway between the 5′ terminus and 3′ terminus of the 3′ UTR. As yet another non-limiting example, a miRNA binding site can be engineered near the 5′ terminus of the 3′ UTR and near the 3′ terminus of the 3′ UTR.

In another embodiment, a 3′ UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition. In some embodiments, a nucleic acid molecule (e.g., RNA, e.g., mRNA) of the disclosure can comprise at least one miRNA binding site in the 3′ UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs are shown in Table 3 below.

TABLE 3
miRNA binding site sequences

| | SEQ | |
|---|---|---|
| | ID | |
| Sequence name | NO: | Sequence |
| miR122 bs | 148 | CAAACACCAUUGUCACACUCCA |
| miR-142-3p bs | 149 | UCCAUAAAGUAGGAAACACUACA |
| miR-126-3p bs | 150 | CGCAUUAUUACUCACGGUACGA |
| miR-142 | 151 | GACAGUGCAGUCACCCAUAAAGUAGAAAG |
| | | CACUACUAACAGCACUGGAGGGUGUAGUG |
| | | UUUCCUACUUUAUGGAUGAGUGUACUGUG |
| miR-142-3p | 152 | UGUAGUGUUUCCUACUUUAUGGA |
| miR-142-5p | 153 | CAUAAAGUAGAAAGCACUACU |
| miR-142-5p | 154 | AGUAGUGCUUUCUACUUUAUG |
| binding site | | |
| miR-126 | 155 | CGCUGGCGACGGGACAUUAUUACUUUUGG |
| | | UACGCGCUGUGACACUUCAAACUCGUACC |
| | | GUGAGUAAUAAUGCGCCGUCCACGGCA |
| miR-126-5p | 156 | CAUUAUUACUUUUGGUACGCG |
| miR-126-5p | 157 | CGCGUACCAAAAGUAAUAAUG |
| binding site | | |

In some embodiments, the 3′UTRs of the nucleic acid molecules described herein comprise miR122 bs (i.e., SEQ TD NO: 148 as shown in Table 3 above). In some embodiments, the 3′UTRs of the nucleic acid molecules described herein comprise miR-142-3p bs (i.e., SEQ ID NO: 149 as shown in Table 3 above). In some embodiments, the 3′UTRs of the nucleic acid molecules described herein comprise miR-126-3p bs (i.e., SEQ ID NO: 150 as shown in Table 3 above).

In some embodiments, the 3′UTRs of the nucleic acid molecules described herein comprise more than one miRNA binding site. In some embodiments, the 3′UTRs of the nucleic acid molecules described herein comprise 1, 2, 3, 4, 5, 6, 7, 8, 9, or 10 miRNA binding sites. In some embodiments, where more than one miRNA binding sites are present, the miRNA binding sites are the same. In some where more than one miRNA binding sites are present, the miRNA binding sites (e.g., any combination of any of the miRNA binding sites listed in Table 3 above). In some embodiments, where more than one miRNA binding sites are present, about 1-25 nucleotides may be present in between each of the miRNA binding sites. For example, 1, 2, 3, 4, 5, 6, 7, 8, 9, 10, 11, 12, 13, 14, 15, 16, 17, 18, 19, 20, 21, 22, 23, 24, or 25 nucleotides may be present in between each of the miRNA bindings.

In some embodiments, the 3′UTRS of the nucleic acid molecules described herein comprise both miR142-3p bs and miR-126-3p bs. In some embodiments, the 3′UTRS of the nucleic acid molecules described herein comprise three copies of miR-142-3p bs. In some embodiments, the 3′UTRS of the nucleic acid molecules described herein comprise two copies of miR-142-3p bs. In some embodiments, the 3′UTRS of the nucleic acid molecules described herein comprise two copies of miR-142-3p bs and one copy of miR-126-3p bs. In some embodiments, the 3′UTRS of the nucleic acid molecules described herein comprise three copies of miR122bs.

### 4. Nucleotide Caps

The disclosure also includes a polynucleotide that comprises both a 5′ Cap and a polynucleotide of the present invention (e.g., a polynucleotide comprising a nucleotide sequence encoding a polypeptide to be expressed).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a polypeptide) incorporate a cap moiety.

In some embodiments, polynucleotides of the present invention comprise a non-hydrolyzable cap structure preventing decapping and thus increasing mRNA half-life.

Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m⁷G(5′)ppp(5′)G). The 3′-0 atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

Another exemplary cap is m⁷GpppG_{2′OMe} or m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein.

Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

Polynucleotides of the invention can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature.

That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N1pN2p (cap 0), 7mG(5′)ppp(5′)N1mpNp (cap 1), and 7mG(5′)-ppp(5′)N1mpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced 10 in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

As used here the term “cap” includes the inverted G nucleotide and can comprise 15 one or more additional nucleotides 3′ of the inverted G nucleotide, e.g., 1, 2, 3, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group.

In one embodiment, a cap comprises a compound of formula (I)

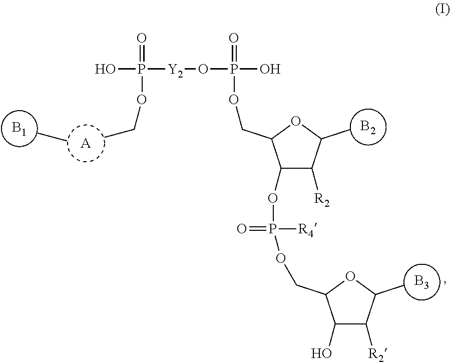

stereoisomer, tautomer or salt thereof, wherein

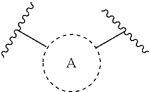

is

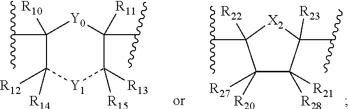

ring B₁ is a modified or unmodified Guanine;
ring B₂ and ring B₃ each independently is a nucleobase or a modified nucleobase;
X₂ is O, S(O)ₚ, NR₂₄ or CR₂₅R₂₆ in which p is 0, 1, or 2;
Y₀ is O or CR₆R₇;
Y1 is O, S(O)n, CR₆R₇, or NR₈, in which n is 0, 1, or 2;
each — is a single bond or absent, wherein when each — is a single bond, Y₁ is O, S(O)ₙ, CR₆R₇, or NR₈; and when each — is absent, Y₁ is void;
Y₂ is (OP(O)R₄)ₘ in which m is 0, 1, or 2, or —O—(CR₄₀R₄₁)u—Q₀—(CR₄₂R₄₃)v—, in which Q₀ is a bond, O, S(O)ᵣ, NR₄₄, or CR₄₅R₄₆, r is 0, 1, or 2, and each of u and v independently is 1, 2, 3 or 4;
each R₂ and R₂′ independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R₄′ independently is H, halo, C₁-C₆ alkyl, OH, SH, SeH, or BH₃—;
each of R₆, R₇, and R₈, independently, is —Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)*, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)*, C₃-C₈ cycloalkyl, C₆-C₁₀ aryl, 4 to 12-membered heterocycloalkyl, and 5- or 6-membered heteroaryl;
each of R₁₀, R₁₁, R₁₂, R₁₃ R₁₄, and R₁₅, independently, is —Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)*, 4 to 12-membered heterocycloalkyl, or 5- or 6-membered
heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)*, C₃-C₈ cycloalkyl, C₆-C₁₀ aryl, 4 to 12-membered
heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is —Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl; each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃—.

It should be understood that a cap analog, as provided herein, may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a cap comprises the following structure:

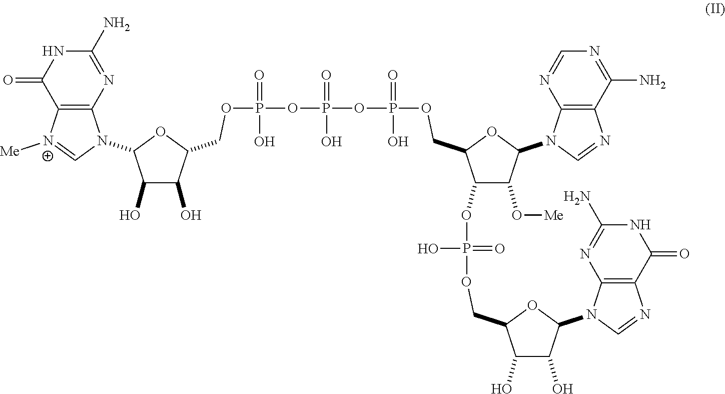 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)(O)OP(=O)(O)OP(=O)(O)OC[C@H]2O[C@@H](n3c[n+](C)c4c(=O)[nH]c(N)nc43)[C@H](O)[C@@H]2O)O[C@H]1n1cnc2c(N)ncnc21]

In other embodiments, a cap comprises the following structure:

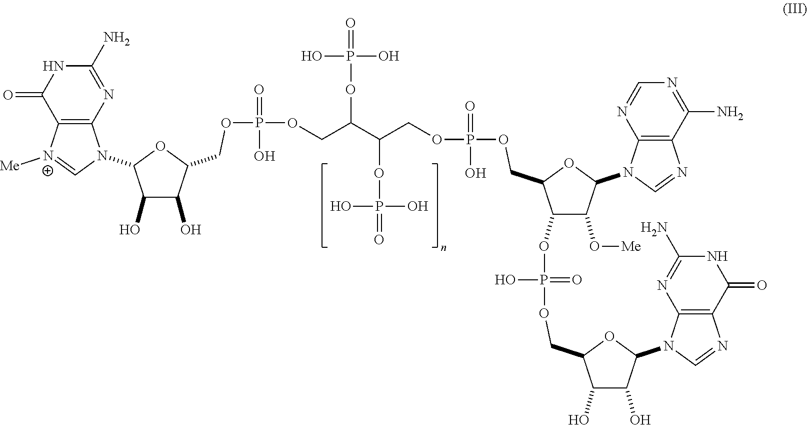

In yet other embodiments, a cap comprises the following structure:

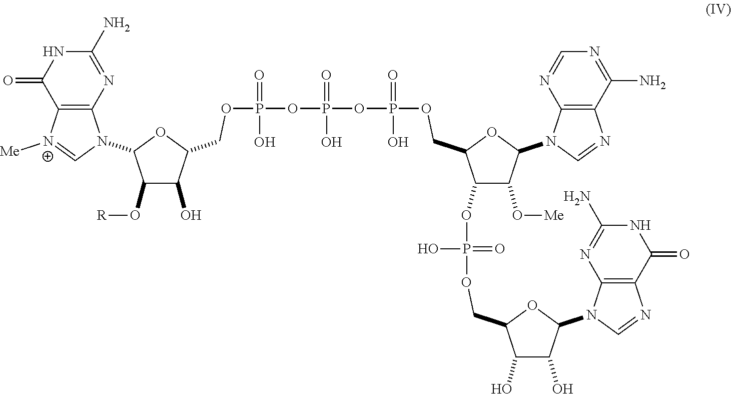

In still other embodiments, a cap comprises the following structure:

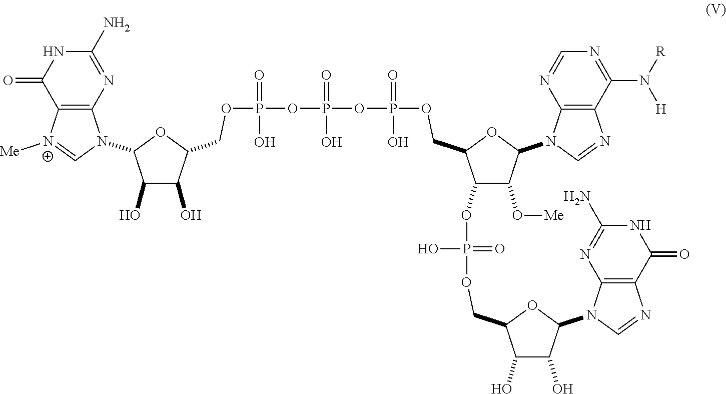

In some embodiments, R is an alkyl (e.g., C₁-C₆ alkyl). In some embodiments, R is a methyl group (e.g., C₁ alkyl). In some embodiments, R is an ethyl group (e.g., C₂ alkyl).

In some embodiments, a cap comprises a sequence selected from the following sequences: GAA, GAC, GAG, GAU, GCA, GCC, GCG, GCU, GGA, GGC, GGG, GGU, GUA, GUC, GUG, and GUUl. In some embodiments, a cap comprises GAA. In some embodiments, a cap comprises GAC. In some embodiments, a cap comprises GAG. In some embodiments, a cap comprises GAU. In some embodiments, a cap comprises GCA.

In some embodiments, a cap comprises GCC. In some embodiments, a cap comprises GCG. In some embodiments, a cap comprises GCU. In some embodiments, a cap comprises GGA. In some embodiments, a cap comprises GGC. In some embodiments, a cap comprises GGG. In some embodiments, a cap comprises GGU. In some embodiments, a cap comprises GUA. In some embodiments, a cap comprises GUC. In some embodiments, a cap comprises GUG. In some embodiments, a cap comprises GUUl.

In some embodiments, a cap comprises a sequence selected from the following sequences: m⁷GpppG, m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a cap comprises m⁷GpppApA. In some embodiments, a cap comprises m⁷GpppApC. In some embodiments, a cap comprises m⁷GpppApG. In some embodiments, a cap comprises m⁷GpppApU. In some embodiments, a cap comprises m⁷GpppCpA. In some embodiments, a cap comprises m⁷GpppCpC. In some embodiments, a cap comprises m⁷GpppCpG. In some embodiments, a cap comprises m⁷GpppCpU. In some embodiments, a cap comprises m⁷GpppGpA. In some embodiments, a cap comprises m⁷GpppGpC. In some embodiments, a cap comprises m⁷GpppGpG. In some embodiments, a cap comprises m⁷GpppGpU. In some embodiments, a cap comprises m⁷GpppUpA. In some embodiments, a cap comprises m⁷GpppUpC. In some embodiments, a cap comprises m⁷GpppUpG. In some embodiments, a cap comprises m⁷GpppUpU.

A cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G₃′OMepppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a cap comprises m⁷G_{3′OMe}pppApA. In some embodiments, a cap comprises m⁷G_{3′OMe}pppApC. In some embodiments, a cap comprises m⁷G_{3′OMe}pppApG. In some embodiments, a cap comprises m⁷G_{3′OMe}pppApU. In some embodiments, a cap comprises m⁷G_{3′OMe}pppCpA. In some embodiments, a cap comprises m⁷G_{3′OMe}pppCpC. In some embodiments, a cap comprises m⁷G_{3′OMe}pppCpG. In some embodiments, a cap comprises m⁷G_{3′OMe}pppCpU. In some embodiments, a cap comprises m⁷G_{3′OMe}pppGpA. In some embodiments, a cap comprises m⁷G_{3′OMe}pppGpC.

In some embodiments, a cap comprises m⁷G_{3′OMe}pppGpG. In some embodiments, a cap comprises m⁷G_{3′OMe}pppGpU. In some embodiments, a cap comprises m⁷G_{3′OMe}pppUpA. In some embodiments, a cap comprises m⁷G_{3′OMe}pppUpC. In some embodiments, a cap comprises m⁷G_{3′OMe}pppUpG. In some embodiments, a cap comprises m⁷G_{3′OMe}pppUpU.

A cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a cap comprises m⁷G_{3′OMe}pppA_{2′OMe}pA. In some embodiments, a cap comprises m⁷G_{3′OMe}pppA_{2′OMe}pC. In some embodiments, a cap comprises m⁷G_{3′OMe}pppA_{2′OMe}pG. In some embodiments, a cap comprises m⁷G_{3′OMe}pppA_{2′OMe}pU. In some embodiments, a cap comprises m⁷G_{3′OMe}pppC_{2′OMe}pA. In some embodiments, a cap comprises m⁷G_{3′OMe}pppC_{2′OMe}pC. In some embodiments, a cap comprises m⁷G_{3′OMe}pppC_{2′OMe}pG. In some embodiments, a cap comprises m⁷G_{3′OMe}ppPC_{2′OMe}pU. In some embodiments, a cap comprises m⁷G_{3′OMe}pppG_{2′OMe}pA. In some embodiments, a cap comprises m⁷G_{3′OMe}pppG_{2′OMe}pC. In some embodiments, a cap comprises m⁷G_{3′OMe}pppG_{2′OMe}pG. In some embodiments, a cap comprises m⁷G_{3′OMe}pppG_{2′OMe}pU. In some embodiments, a cap comprises m⁷G_{3′OMe}pppU_{2′OMe}pA. In some embodiments, a cap comprises m⁷G_{3′OMe}pppU_{2′OMe}pC. In some embodiments, a cap comprises m⁷G_{3′OMe}pppU_{2′OMe}pG. In some embodiments, a cap comprises m⁷G_{3′OMe}pppU_{2′OMe}pU.

A cap, in still other embodiments, comprises a sequence selected from the following sequences: m⁷GpppG_{2′OMe}, m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a cap comprises m⁷GpppA_{2′OMe}pA. In some embodiments, a cap comprises m⁷GpppA_{2′OMe}pC. In some embodiments, a cap comprises m⁷GpppA_{2′OMe}pG. In some embodiments, a cap comprises m⁷GpppA_{2′OMe}pU. In some embodiments, a cap comprises m⁷GpppC_{2′OMe}pA. In some embodiments, a cap comprises m⁷GpppC_{2′OMe}pC. In some embodiments, a cap comprises m⁷GpppC_{2′OMe}pG.

In some embodiments, a cap comprises m⁷GpppC_{2′OMe}pU. In some embodiments, a cap comprises m⁷GpppG_{2′OMe}pA. In some embodiments, a cap comprises m⁷GpppG_{2′OMe}pC.

In some embodiments, a cap comprises m⁷GpppG_{2′OMe}pG. In some embodiments, a cap comprises m7GpppG_{2′OMe}pU. In some embodiments, a cap comprises m⁷GpppU_{2′OMe}pA. In some embodiments, a cap comprises m⁷GpppU_{2′OMe}pC. In some embodiments, a cap comprises m⁷GpppU_{2′OMe}pG. In some embodiments, a cap comprises m⁷GpppU_{2′OMe}pU.

In some embodiments, a cap comprises m⁷Gpppm⁶A_{2′OMe}pG. In some embodiments, a cap comprises m⁷Gpppe⁶A_{2′OMe}pG.

In some embodiments, a cap comprises GAG. In some embodiments, a cap comprises GCG. In some embodiments, a cap comprises GUG. In some embodiments, a cap comprises GGG.

In some embodiments, a cap comprises any one of the following structures:

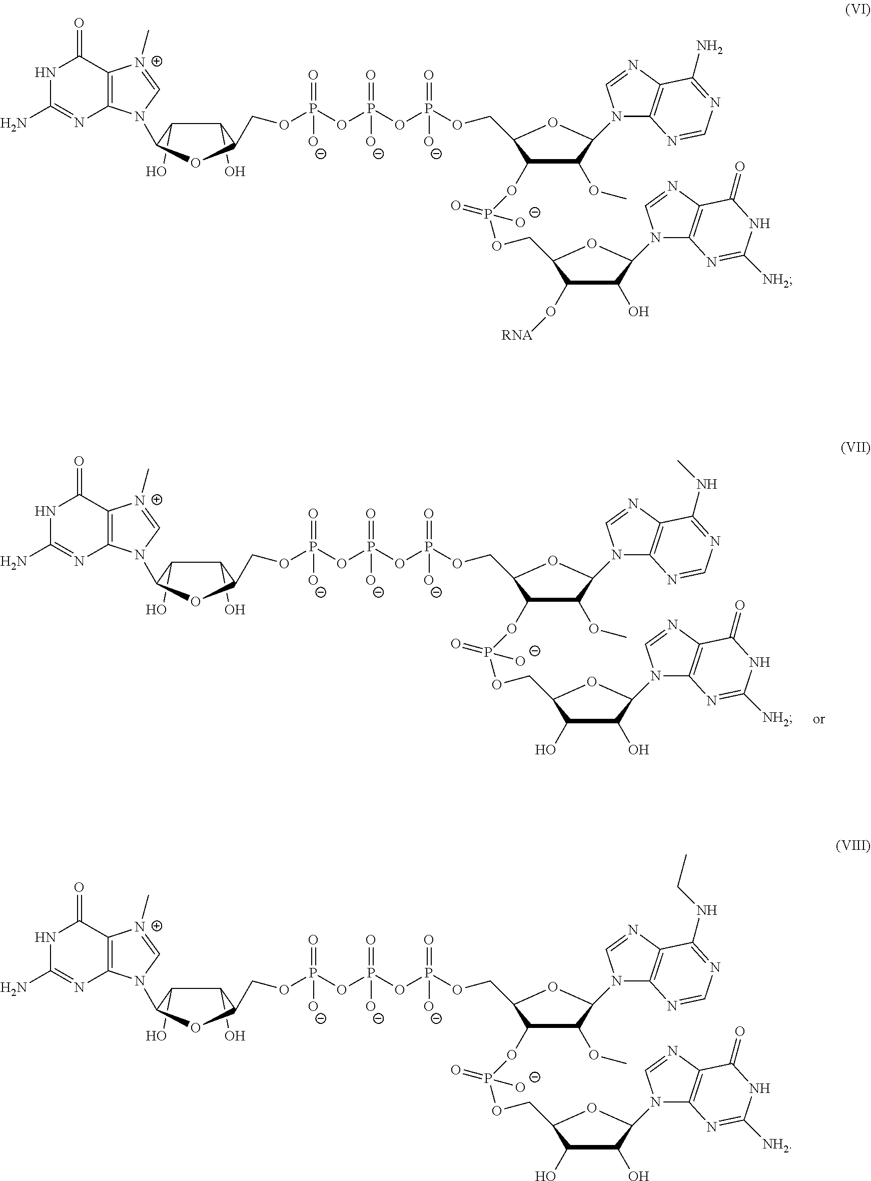

In some embodiments, the cap comprises ^{m7}GpppN₁N₂N₃, where N₁, N₂, and N₃ are optional (i.e., can be absent or one or more can be present) and are independently a natural, a modified, or an unnatural nucleoside base. In some embodiments, m⁷G is further methylated, e.g., at the 3′ position. In some embodiments, the m⁷G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position.

In some embodiments, the cap comprises the following structure:

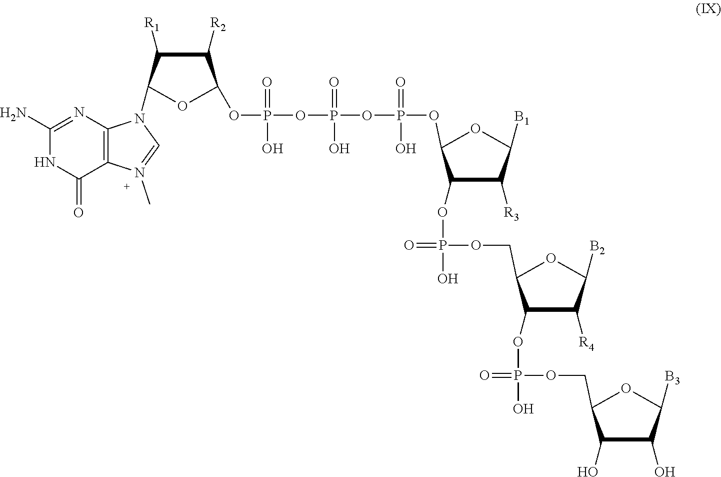

wherein B₁, B₂, and B₃ are independently a natural, a modified, or an unnatural nucleoside based; and R₁, R₂, R₃, and R₄ are independently OH or O-methyl. In some 5 embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B₁ is adenine, cytosine, thymine, oruracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU. In some embodiments the cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A cap, in some embodiments, comprises a sequence selected from the following sequences: m⁷G_{3′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{3′OMe}pppU_{2′OMe}pUpN, where N is a natural, a modified, or an unnatural nucleoside base.

A cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m⁷GpppC_{2′OMe}pUpN, m⁷GpppG_{2′OMe}pApN, m⁷GpppG_{2′OMe}pCpN, m⁷GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{2′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}pN, m⁷G_{3′OMe}pppG_{2′OMe}pA_{2′OMe}pN, m⁷G_{3′OMe}pppG_{2′OMe}pC_{2′OMe}pN, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base.

In some embodiments, a cap comprises GGAG. In some embodiments, a cap comprises the following structure:

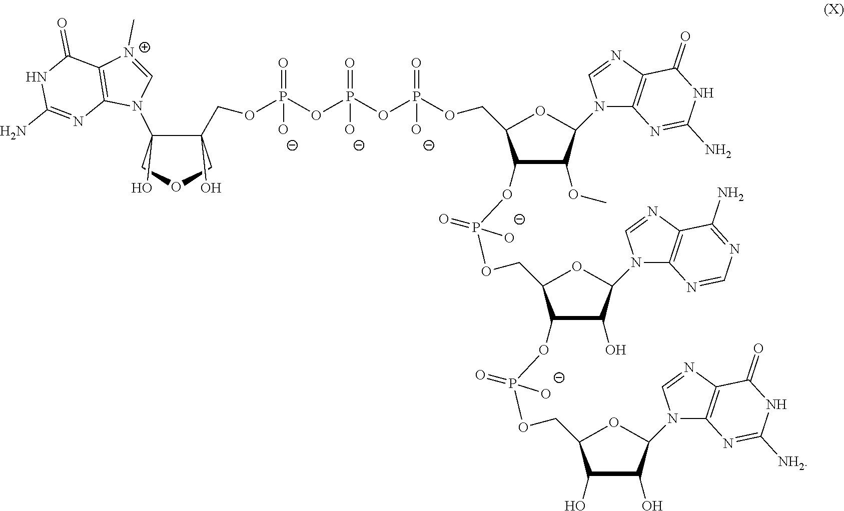

### 5. Stop Element

Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162).

Disclosed herein, inter alia, is a polynucleotide encoding a polypeptide, which polynucleotide has a coding region comprising a stop element which confers an increased half-life, increased expression and/or increased activity of the polypeptide encoded by said polynucleotide, or of the polynucleotide itself In an embodiment, the polynucleotide comprises: (a) a 5′-UTR (e.g., as described herein); (b) a coding region comprising a stop element (e.g., as described herein); and (c) a 3′-UTR (e.g., as described herein), and LNP compositions comprising the same. In an embodiment, the polynucleotide comprises a coding region comprising a stop element provided in Table 4.

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In an embodiment, the stop element comprises a plurality of the same stop codon. In an embodiment, the stop element comprises a plurality of different stop codons.

In an embodiment, a stop element further comprises at least 1, 2, 3, 4, 5, or 10 nucleotides upstream and/or downstream of the one or more stop codons. In an embodiment, a stop element further comprises at least 1, 2, 3, 4, 5, or 10 nucleotides upstream of the one or more stop codons. In an embodiment, a stop element further comprises at least 1, 2, 3, 4, 5, or 10 nucleotides downstream of the one or more stop codons.

The invention also includes a polynucleotide that comprises both a stop codon element and the polynucleotide described herein. In some embodiments, a stop codon element comprises a stop codon region. In some embodiments, the coding region of the polynucleotide comprises the stop element. In some embodiments, the stop element is upstream, e.g., before, the 3′ UTR sequence in the polynucleotide.

In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

It has been observed that stop elements comprising a sequence provided in Table 4 can result in increased half-life of the polynucleotide and/or increased level or activity of the polypeptide encoded by the polynucleotide.

In an embodiment, the polynucleotide having a stop element provided in Table 4 results in an increased half-life of the polynucleotide or an increased level and/or activity, e.g., output, of the polypeptide encoded by the polynucleotide. In an embodiment, the increase in half-life is about 1.5-20-fold. In an embodiment, the increase in half-life is about 1.5, 2, 3, 4, 5, 6, 7, 8, 9, 10, 11, 12, 13, 14, 15, 16, 17, 18, 19 or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold. In an embodiment, the increase in half life is about 5-fold or more.

In an embodiment, the polynucleotide having a stop element provided in Table 4 results in an increased level and/or activity, e.g., output or duration of expression, of the polypeptide encoded by the polynucleotide. In an embodiment, the stop element results in about 1.5-20-fold increase in level and/or activity, e.g., detectable level or activity, of the polypeptide encoded by the polynucleotide for about 1, 2, 3, 4, 5, 6, 7, 8, 9, 10, or 14 days. In an embodiment, the stop element results in detectable level or activity of the polypeptide encoded by the polynucleotide for about 1, 2, 3, 4, 5, 6, 7, 8, 9, 10, or 14 days.

In an embodiment, the increase in activity is about 1.5, 2, 3, 4, 5, 6, 7, 8, 9, 10, 11, 12, 13, 14, 15, 16, 17, 18, 19 or 20-fold, or more. In an embodiment, the increase in activity is about 1.5-fold or more. In an embodiment, the increase in activity is about 2-fold or more. In an embodiment, the increase in activity is about 3-fold or more. In an embodiment, the increase in activity is about 4-fold or more. In an embodiment, the increase in activity is about 5-fold or more.

In an embodiment, the increase is compared to an otherwise similar polynucleotide which does not have a stop element, has a different stop element, or does not have a stop element provided in Table 4.

In an embodiment, the stop element comprises a sequence provided in Table 4. In an embodiment, the stop element comprises the sequence of SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, SEQ ID NO; 169, SEQ ID NO: 173 or SEQ ID NO: 174. In an embodiment, the stop element comprises the sequence of SEQ ID NO: 158. In an embodiment, the stop element comprises the sequence of SEQ ID NO: 159. In an embodiment, the stop element comprises the sequence of SEQ ID NO: 160. In an embodiment, the stop element comprises the sequence of SEQ ID NO: 161. In an embodiment, the stop element comprises the sequence of SEQ ID NO: 162. In an embodiment, the stop element comprises the sequence of SEQ ID NO: 163. In an embodiment, the stop element comprises the sequence of SEQ ID NO: 164. In an embodiment, the stop element comprises the sequence of SEQ ID NO: 165. In an embodiment, the stop element comprises the sequence of SEQ ID NO: 166. In an embodiment, the stop element comprises the sequence of SEQ ID NO: 167. In an embodiment, the stop element comprises the sequence of SEQ ID NO: 168. In an embodiment, the stop element comprises the sequence of SEQ ID NO: 169. In an embodiment, the stop element comprises the sequence of SEQ ID NO: 173. In an embodiment, the stop element comprises the sequence of SEQ ID NO: 174.

In some embodiments the polynucleotide includes a kappa stop cassette (i.e., UAAAGCUCCCCGGGG (SEQ ID NO: 165) or an iota stop cassette (i.e., UAAGCCCCUCCGGGG (SEQ ID NO: 164).

In an embodiment, the coding region of (b) comprises a stop element comprising a consensus sequence of Formula B:

(SEQ ID NO: 170)
X₋₃-X₋₂-X₋₁-U-A-A-X₁-X₂-X₃-X₄-X₅-X₆-X₇-X₈-X₉-X₁₀-X₁₁-X₁₂

wherein:
X₁ is a G or A;
X₂, X₄, X₅ X₆ or X₇ is each independently C or U;
X₃ is C or A;
X₈, X₁₀, X₁₁, X₁₂X₁ or X₃ is each independently C or G;
X₉ is G or U; and/or
X₂ is A or U.

In an embodiment, X₁ is a G. In an embodiment, X₁ is an A.

In an embodiment, X₂ is a C. In an embodiment, X₂ is a U.

In an embodiment, X₄ is a C. In an embodiment, X₄ is a U.

In an embodiment, X₅ is a C. In an embodiment, X₅ is a U.

In an embodiment, X₆ is a C. In an embodiment, X₆ is a U.

In an embodiment, X₇ is a C. In an embodiment, X₇ is a U.

In an embodiment, X₃ is a C. In an embodiment, X₃ is an A.

In an embodiment, X₈ is a C. In an embodiment, X₈ is a G.

In an embodiment, X₁₀ is a C. In an embodiment, X₁₀ is a G.

In an embodiment, X₁₁ is a C. In an embodiment, X₁₁ is a G.

In an embodiment, X₁₂ is a C. In an embodiment, X₁₂ is a G.

In an embodiment, X₋₁ is a C. In an embodiment, X₁ is a G.

In an embodiment, X₋₃ is a C. In an embodiment, X₋₃ is a G.

In an embodiment, X₉ is a G. In an embodiment, X₉ is a U.

In an embodiment, X₋₂ is an A. In an embodiment, X₋₂ is a U.

In an embodiment, the consensus sequence of Formula B (SEQ ID NO: 170) has a high GC content, e.g., GC content of about 50%, 60%, 70%, 80%, 90% or 99%. In an embodiment, the GC content is about 50%. In an embodiment, the GC content is about 60%. In an embodiment, the GC content is about 70%. In an embodiment, the GC content is about 80%. In an embodiment, the GC content is about 90%. In an embodiment, the GC content is about 99%.

In an embodiment, the coding region of (b) comprises a stop element comprising a consensus sequence of Formula C:

(SEQ ID NO: 171)
X₋₃-X₋₂-X₋₁-U-G-A-X₁-X₂-X₃-X₄-X₅-X₆-X₇-X₈-X₉-X₁₀-X₁₁-X₁₂

wherein:
X₋₃, X₁₁, X₂, X₅, X₆, X₇, X₈, X₉, or X₁₂ is each independently G or C; X₋₂, X₃, or X₄ is each independent A or C; X₁ is A or G; and/or X₁₀ or X₁₁ is each independently C or U.

In an embodiment, X₋₃ is a G. In an embodiment, X₋₃ is a C.

In an embodiment, X₋₁ is a G. In an embodiment, X₋₁ is a C.

In an embodiment, X₂ is a G. In an embodiment, X₂ is a C.

In an embodiment, X₅ is a G. In an embodiment, X₅ is a C.

In an embodiment, X₆ is a G. In an embodiment, X₆ is a C.

In an embodiment, X₇ is a G. In an embodiment, X₇ is a C.

In an embodiment, X₈ is a G. In an embodiment, X₈ is a C.

In an embodiment, X₉ is a G. In an embodiment, X₉ is a C.

In an embodiment, X₁₂ is a G. In an embodiment, X₁₂ is a C.

In an embodiment, X₋₂ is an A. In an embodiment, X₋₂ is a C.

In an embodiment, X₃ is an A. In an embodiment, X₃ is a C.

In an embodiment, X₄ is an A. In an embodiment, X₄ is a C.

In an embodiment, X₁ is an A. In an embodiment, X₁ is a G.

In an embodiment, X₁₀ is a C. In an embodiment, X₁₀ is a U.

In an embodiment, X₁₁ is a C. In an embodiment, X₁₁ is a U.

In an embodiment, the consensus sequence of Formula C (SEQ ID NO: 171) has a high GC content, e.g., GC content of about 50%, 60%, 70%, 80%, 90% or 99%. In an embodiment, the GC content is about 50%. In an embodiment, the GC content is about 60%. In an embodiment, the GC content is about 70%. In an embodiment, the GC content is about 80%. In an embodiment, the GC content is about 90%. In an embodiment, the GC content is about 99%.

In an embodiment, the coding region of (b) comprises a stop element comprising a consensus sequence of Formula D:

(SEQ ID NO: 172)
X₋₃-X₋₂-X₋₁-U-A-G-X₁-X₂-X₃-X₄-X₅-X₆-X₇-X₈-X₉-X₁₀-X₁₁-X₁₂

wherein:
X₋₃, X₁, X₂, X₃, X₁₀ is each independently G or C;
X₋₂ or X₉ is each independently A or U;
X₁ or X₄ is each independently A or G;
X₅ or X₈ is each independently A or C; and/or
X₆, X₇, X₁₁ or X₁₂ is each independently C or U.
In an embodiment, X₋₃ is a G. In an embodiment, X₋₃ is a C.
In an embodiment, X₋₁ is a G. In an embodiment, X₋₁ is a C.
In an embodiment, X₂ is a G. In an embodiment, X₂ is a C.
In an embodiment, X₃ is a G. In an embodiment, X₃ is a C.
In an embodiment, X₁₀ is a G. In an embodiment, X₁₀ is a C.
In an embodiment, X₋₂ is an A. In an embodiment, X₋₂ is a U.
In an embodiment, X₉ is an A. In an embodiment, X₉ is a U.
In an embodiment, Xᵢ is an A. In an embodiment, X₁ is a G.
In an embodiment, X₄is an A. In an embodiment, X₄ is a G.
In an embodiment, X₅ is an A. In an embodiment, X₅ is a C.
In an embodiment, X₈ is an A. In an embodiment, X₈ is a C.
In an embodiment, X₆ is a C. In an embodiment, X₆ is a U.
In an embodiment, X₇ is a C. In an embodiment, X₇ is a U.
In an embodiment, X₁₁ is a C. In an embodiment, X₁₁ is a U.
In an embodiment, X₁₂ is a C. In an embodiment, X₁₂ is a U.

In an embodiment, the consensus sequence of Formula D (SEQ ID NO: 172) has a high GC content, e.g., GC content of about 50%, 60%, 70%, 80%, 90% or 99%. In an embodiment, the GC content is about 50%. In an embodiment, the GC content is about 60%. In an embodiment, the GC content is about 70%. In an embodiment, the GC content is about 80%. In an embodiment, the GC content is about 90%. In an embodiment, the GC content is about 99%.

TABLE 4
Stop elements

| SEQ | Sequence | |
|---|---|---|
| ID | informa- | |
| NO | tion | Sequence |
| 164 | C1 (refer- | UGAUAAUAG |
| | ence) | |
| | C2 | UAAUAGUAA |
| | C3 | UAAGUCUAA |
| | C4 | UAAAGCUAA |
| | C5 | UAAGUCUCC |
| | C6 | UAAGGCUAA |
| | C7 | UAAGCCCCUCCGGGG |
| 165 | C8 | UAAAGCUCCCCGGGG |
| | C9 | UAAGCCCCU |
| | C10 | UAAAGCUCC |
| | C11 | UAGGGUUAA |
| | C15 | UAAGCACCC |
| 170 | C12 (UAA | X₋₃-X₋₂-X₋₁-U-A-A-X₁-X₂-X₃-X₄-X₅-X₆-X₇-X₈-X₉-X₁₀-X₁₁-X₁₂ |
| | consensus) | wherein: |
| | | X₁ is a G or A; |
| | | X₂, X₄, X₅ X₆ or X₇ is each independently C or U; |
| | | X₃ is C or A; |
| | | X₈, X₁₀, X₁₁, X₁₂ X₋₁ or X₋₃ is each independently C or G; |
| | | X₉ is G or U; and/or |
| | | X₋₂ is A or U. |
| 171 | C13 (UGA | X₋₃-X₋₂-X₋₁-U-G-A-X₁-X₂-X₃-X₄-X₅-X₆-X₇-X₈-X₉-X₁₀-X₁₁-X₁₂ |
| | consensus) | wherein: |
| | | X₋₃, X₋₁, X₂, X₅, X₆, X₇, X₈, X₉, or X₁₂ is each |
| | | independently G or C; |
| | | X₋₂, X₃, or X₄ is each independent A or C; |
| | | X₁ is A or G; and/or |
| | | X₁₀ or X₁₁ is each independently C or U. |
| 172 | C14 (UAG | X₋₃-X₋₂-X₋₁-U-A-G-X₁-X₂-X₃-X₄-X₅-X₆-X₇-X₈-X₉-X₁₀-X₁₁-X₁₂ |
| | consensus) | wherein: |
| | | X₋₃, X₋₁, X₂, X₃, X₁₀ is each independently G or C; |
| | | X₋₂ or X₉ is each independently A or U; |
| | | X₁ or X₄ is each independently A or G; |
| | | X₅ or X₈ is each independently A or C; and/or |
| | | X₆, X₇, X₁₁ or X₁₂ is each independently C or U. |
| | C16 | UGAUAGUAA |
| | C17 | UAAAGCGCU |

In an aspect, disclosed herein is a polynucleotide encoding a polypeptide, wherein the polynucleotide comprises: (a) a 5′-UTR, e.g., as described herein; (b) a coding region comprising a stop element (e.g., as provided in Table 4); and (c) a 3′-UTR (e.g., as described herein).

6. Poly A tails

In some embodiments, the polynucleotides of the present disclosure further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO: 121).

(SEQ ID NO: 121)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA.

In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:123).

(SEQ ID NO: 123)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

In some embodiments, the poly-A tail is a mixed poly-A tail with intermittent non-adenosine residues (e.g., guanosine). In some embodiments, the poly-A tail is guanylated. Without wishing to be bound by theory, it is believed that in some embodiments the mixed poly-A tail can shield mRNA from rapid deadenylation.

In some embodiments, the poly-A tail comprises one or more non-adenosine residues. In some embodiments, the non-adenosine residue is guanosine. In some embodiments, the poly-A tail comprises 1-20, e.g., 1-15, 1-10, 1-5, 15-20, 10-20, 5-20, 2-15, 5-10, 1-5, 2-10, or 5-15, non-adenosine residues (e.g., guanosine). For example, the poly-A tail can comprise 1, 2, 3, 4, 5, 6. 7, 8, 9, 10, 11, 12, 13, 14, 15, 16, 17, 18, 19, 20, or more. non-adenosine residues (e.g., guanosine). In some embodiments, at least 1%, e.g., at least 2%, 5%, 10%, 15%, 20%, or 25%, of the residues in the poly-A tail are non-adenosine residues (e.g., guanosine). In some embodiments, the poly-A tail is guanylated, e.g., comprising one or more guanosine residues.

In an embodiment, the poly-A tail comprising one or more non-adenosine residues is chemically synthesized

In an embodiment, the 3′ UTR comprises a TENT recruiting sequence, e.g., as described herein, which recruits one or more terminal nucleotidyl transferases (TENTs) to the polynucleotide comprising the 3′ UTR. In an embodiment, the TENT is TENT4, e.g., TENT4A and/or TENT4B. Without wishing to be bound by theory, it is believed that in some embodiments one or more TENTs (e.g., TENT4A and/or TENT4B) generates a mixed poly-A tail with intermittent non-adenosine residues (e.g., guanosine), which shields mRNA from rapid deadenylation.

Exemplary TENT recruiting sequences include, but are not limited to,

(SEQ ID NO: 91)
CACCGCGUUAUCCGUUCCUCGUAGGCUGGUCCUGGGGAACGGGUCGGCGG
and
(SEQ ID NO: 92)
CCACCCCCAGCGCCACCACCGCUGCCGUCGCCACCGCGUUAUCCGUUCCU
CGUAGGCUGGUCCUGGGGAACGGGUCGGCGGCCGGUCGGCUUCUGUUUUA

In an embodiment, the TENT recruiting sequence comprises the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the TENT recruiting sequence comprises the nucleotide sequence of SEQ ID NO: 91.

In an embodiment, the TENT recruiting sequence comprises the nucleotide sequence of SEQ ID NO: 92, or a nucleotide sequence having at least 80%, 85%, 90%, 95%, 96%, 97%, 98%, or 99% identity thereto, or differing by no more than 1, 2, 3, 4, 5, 6, 7, 8, 9, 10, 11, 12, 13, 14, 15, 16, 17, 18, 19, or 20 nucleotides therefrom. In an embodiment, the TENT recruiting sequence comprises the nucleotide sequence of SEQ ID NO: 92.

In an embodiment, the 3′ UTR comprises one or more (e.g., 2, 3, 4, 5, or more) TENT recruiting sequences, e.g., one or more TENT recruiting sequences described herein. In an embodiment the 3′ UTR comprises one TENT recruiting sequence. In an embodiment the 3′ UTR comprises two TENT recruiting sequences. In an embodiment the 3′ UTR comprises three TENT recruiting sequences. In an embodiment the 3′ UTR comprises four TENT recruiting sequences. In an embodiment the 3′ UTR comprises five TENT recruiting sequences. For example, the multiple TENT recruiting sequences in the 3′ UTR can be identical or different.

In an embodiment, the 3′ UTR comprises a TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises a TENT recruiting sequence comprising the nucleotide sequence of SEQ ID NO: 91.

In an embodiment, the 3′ UTR comprises one or more (e.g., 2, 3, 4, 5, or more) of a TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises one TENT recruiting sequence comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises two TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises three TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises four TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In an embodiment, the 3′ UTR comprises five TENT recruiting sequences, each comprising the nucleotide sequence of SEQ ID NO: 91, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom.

In an embodiment, the 3′ UTR comprises one or more (e.g., 2, 3, 4, 5, or more) of a TENT recruiting sequence comprising the nucleotide sequence of SEQ ID NO: 91. In an embodiment, the 3′ UTR comprises two TENT recruiting sequences, each comprising the nucleotide sequence of SEQ ID NO: 91. In an embodiment, the 3′ UTR comprises three TENT recruiting sequences, each comprising the nucleotide sequence of SEQ ID NO: 91. In an embodiment, the 3′ UTR comprises four TENT recruiting sequences, each comprising the nucleotide sequence of SEQ ID NO: 91. In an embodiment, the 3′ UTR comprises five TENT recruiting sequences, each comprising the nucleotide sequence of SEQ ID NO: 91.

7. Additional 3′UTR elements

### A) Identification and Ratio Determination (IDR)

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### B) FUT8

In some embodiments, the 3′UTR comprises a FUT8 sequence. For example, the FUT8 sequence comprises the following sequence: CUGAGAGACCUGUGUGAACUAUUGAGAAGAUCGGAACAGCUCCUUACUCUGAGGAAGUU G SEQ ID NO: 93. In an embodiment, the 3′ UTR comprises a FUT8 sequence comprising the nucleotide sequence of SEQ ID NO: 93, or a nucleotide sequence having at least 80%, 85%, 90%, 95%, 96%, 97%, 98%, or 99% identity thereto, or differing by no more than 1, 2, 3, 4, 5, 6, 7, 8, 9, or 10 nucleotides therefrom. In some embodiments, the FUT8 sequence can be combined with any of the miRNA binding sites present in the 3′UTR and as described here.

C) Ribosome Engagement Detection Assay (REDA) REDA can be used to assess potency and effectiveness of cellular lipid nanoparticle-nucleic acid uptake and translation of mRNA of a manufactured nucleic acid. The assay incorporates some aspects of a Ribosome Engagement Detection Assay (REDA) in order to measure mRNA bound to ribosomes during the translation step in the cell. The assay does not need to involve actual protein expression, but rather, is representative of the effectiveness of a nucleic acid such as an mRNA in producing protein in a cell by demonstrating effective mRNA uptake and association with ribosomes, and thus effective intracellular translation.

Accordingly, in some embodiments, any of the 3′ UTR sequences, as described herein, comprise a sequence that can be detected by qPCR in REDA.

An RNA species (e.g., RNA having a given coding sequence) may comprise a REDA sequence that differs from the REDA sequence of other RNA species (e.g., RNA(s) having different coding sequence(s)). Each REDA sequence thus identifies a particular RNA species, and so the abundance of REDA sequences may be measured to determine the abundance of each RNA species in a composition. Use of distinct REDA sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise a REDA sequence that is not a sequence isomer of a REDA sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another REDA sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise a REDA sequence having a mass that differs from the mass of REDA sequences of each other RNA species in a multivalent RNA composition. For example, the mass of each REDA sequence may differ from the mass of other REDA sequences by at least 9 Da, at least 25 Da, at least 25 Da, or at least 50 Da. Use of REDA sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises a REDA sequence with a different length. For example, each IDR sequence may have a length independently selected from 0 to 25 nucleotides. The length of a nucleic acid influences the rate at which the nucleic acid traverses a chromatography column, and so the use of REDA sequences of different lengths on different RNA species allows RNA fragments having different REDA sequences to be distinguished using chromatography-based methods (e.g., LC-UV).

REDA sequences may be chosen such that no REDA sequence comprises a start codon, ‘AUG’. Lack of a start codon in an IDR sequence prevents undesired translation of nucleotide sequences within and/or downstream from the REDA sequence.

REDA sequences may be chosen such that no REDA sequence comprises a recognition site for a restriction enzyme. In one example, no REDA sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### 8. Start Codon Region

The invention also includes a polynucleotide that comprises both a start codon region and the polynucleotide described herein. In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence in order to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

9. Combination of mRNA Elements

Any of the polynucleotides disclosed herein can comprise one, two, three, or all of the following elements: a 5′-UTR, e.g., as described herein; a coding region; a stop element+3′-UTR (e.g., as described herein) and; optionally a 3′ stabilizing region, e.g., as described herein. Also disclosed herein are LNP compositions comprising the same.

In an embodiment, a polynucleotide of the disclosure comprises a 5′ UTR described in Table 1 or a variant or fragment thereof and a stop element+3′ UTR comprising the sequence of SEQ ID NO:139 or a variant or fragment thereof. In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In an embodiment, a polynucleotide of the disclosure comprises a 5′ UTR comprising the sequence of SEQ ID NO: 50 or a variant or fragment thereof and a stop element+3′ UTR comprising the sequence of SEQ ID NO: 139 or a variant or fragment thereof In an embodiment, the polynucleotide further comprises a cap structure, e.g., as described herein, or a poly A tail, e.g., as described herein. In an embodiment, the polynucleotide further comprises a 3′ stabilizing region, e.g., as described herein. In an embodiment, a polynucleotide of the disclosure comprises a 5′ UTR comprising the sequence of SEQ ID NO: 56 or a variant or fragment thereof and a stop element+3′ UTR comprising the sequence of SEQ ID NO: 139 or a variant or fragment thereof In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In some embodiments, the 5′UTR is SEQ ID NO: 50 and the 3′UTR is SEQ ID NO: 139 or SEQ ID NO: 144. For example, the 5′UTR is SEQ ID NO: 50 and the 3′UTR is SEQ ID NO: 139, or the 5′UTR is SEQ ID NO: 50 and the 3′UTR is SEQ ID NO: 144.

In an embodiment, the polynucleotide further comprises a cap structure, e.g., as described herein, or a poly A tail, e.g., as described herein. In an embodiment, the polynucleotide further comprises a 3′ stabilizing region, e.g., as described herein.

In some embodiments, any one or more of the miRNA binding site sequences selected from Table 3 can be combined with any one of the stop cassettes as shown in Table 4. Additionally, in some embodiments, a Tent recruiting sequence can be combined with any one or more of the miRNA binding site sequences selected from Table 3 and any one of the stop cassette as shown in Table 4. Further, in some embodiments, the FUT8 sequence can be combined with any one or more of the miRNA binding site sequences selected from Table 3and any one of the stop cassettes as shown in Table 4.

### 10. Therapeutic Payload or Prophylactic Payload

Disclosed herein, inter alia, is a polynucleotide having a 5′ UTR described herein, a 3′ UTR described herein, and/or a coding region comprising a stop element, which coding region further comprises a sequence that encodes for a payload, e.g., a therapeutic payload or a prophylactic payload. In an embodiment, the coding region encodes for one payload. In an embodiment, the coding region encodes for more than one payload, e.g., 2, 3, 4, 5, 6, or more payloads, e.g., same or different payloads. In an embodiment, the sequence encoding each payload is contiguous in the polynucleotide. In an embodiment, the sequence encoding each payload is separated by at least 1-1000 nucleotides. In some embodiments, the therapeutic payload or prophylactic payload comprises an mRNA encoding: a secreted protein; a membrane-bound protein; or an intercellular protein, or peptides, polypeptides or biologically active fragments thereof.

Also disclosed herein is an LNP comprising a polynucleotide comprising a coding region which encodes for a payload, e.g., a therapeutic payload or a prophylactic payload. In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, or a peptide, a polypeptide or a biologically active fragment thereof In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof In some embodiments, the secreted protein comprises an immune modulator, e.g., an immune checkpoint agonist or antagonist.

In some embodiments, the therapeutic payload or prophylactic payload comprises an mRNA encoding a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the therapeutic payload or prophylactic payload comprises an mRNA encoding an intracellular protein, or a peptide, a polypeptide or a biologically active fragment thereof. In some embodiments, the intracellular protein comprises an enzyme, or a variant or fragment (e.g., a biologically active fragment) thereof In some embodiments, the intracellular protein comprises a transcription factor, or a variant or fragment (e.g., a biologically active fragment) thereof In some embodiments, the intracellular protein comprises a nuclease, or a variant or fragment (e.g., a biologically active fragment) thereof. In some embodiments, the intracellular protein comprises a structural protein, or a variant or fragment (e.g., a biologically active fragment) thereof.

In some embodiments, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the therapeutic payload or prophylactic payload comprises a protein or peptide.

It will be understood that the regulatory elements disclosed herein (e.g., 5′UTRs, stop elements, 3′UTRs, stabilizing regions (e.g., idT or modified poly A tails) can be used with ORFs encoding a payload described herein. It will further be understood that the regulatory elements disclosed herein can be used in a modular fashion, i.e., can be used in an mRNA construct in combination with other regulatory elements from the art (e.g., a 5′UTR of the instant invention in combination with an ORF and other regulatory regions from the art), or can be used in combination with the other regulatory elements disclosed herein (e.g., a 5′UTR of the instant invention and a 3′UTR of the instant invention, et cetera). It will further be understood that a stop element of the present invention can be used in combination with a desired ORF that lacks a stop codon. It will also be understood that when a desired ORF comprises a stop codon, an additional stop codon or stop element will not be included in the final construct. In some embodiments, the stop codon in the desired ORF can be replaced with a stop element described herein.

### 11. Methods of Making Polynucleotides

The present disclosure also provides methods for making a polynucleotide disclosed herein or a complement thereof. In some aspects, a polynucleotide (e.g., an mRNA) disclosed herein can be constructed using in vitro transcription.

In other aspects, a polynucleotide (e.g., an mRNA) disclosed herein can be constructed by chemical synthesis using an oligonucleotide synthesizer. In other aspects, a polynucleotide (e.g., an mRNA) disclosed herein is made by using a host cell. In certain aspects, a polynucleotide (e.g., an mRNA) disclosed herein is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a therapeutic payload or prophylactic payload. The resultant mRNAs can then be examined for their ability to produce protein and/or produce a therapeutic outcome.

While RNA can be made synthetically using methods well known in the art, in one embodiment an RNA transcript (e.g., mRNA transcript) is synthesized by contacting a DNA template with a RNA polymerase (e.g., a T7 RNA polymerase or a T7 RNA polymerase variant) under conditions that result in the production of RNA transcript.

In some aspects, the present disclosure provides methods of performing an IVT (in vitro transcription) reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects of the present disclosure provide capping methods, e.g., co-transcriptional capping methods or other methods known in the art. In one embodiment, a capping method comprises reacting a polynucleotide template with a T7 RNA polymerase variant, nucleoside triphosphates, and a cap analog under in vitro transcription reaction conditions to produce RNA transcript.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application.

Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A deoxyribonucleic acid (DNA) is simply a nucleic acid template for RNA polymerase. A DNA template may include a polynucleotide encoding a polypeptide of interest (e.g., an antigenic polypeptide). A DNA template, in some embodiments, includes a RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) tail located at the 3′ end of the gene of interest.

Polypeptides of interest include, but are not limited to, biologics, antibodies, antigens (vaccines), and therapeutic proteins. The term “protein” encompasses peptides.

A RNA transcript, in some embodiments, is the product of an IVT reaction and, as will be understood by one of ordinary skill in the art, the DNA template for making an RNA molecule is known based on base complementarity. A RNA transcript, in some embodiments, is a messenger RNA (mRNA) that includes a nucleotide sequence encoding a polypeptide of interest linked to a polyA tail. In some embodiments, the mRNA is modified mRNA (mmRNA), which includes at least one modified nucleotide.

A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4 or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap) to nucleoside triphosphates in the reaction is less than 1:1. For example, the molar ratio of cap analog (e.g., trinucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

The composition of NTPs in an IVT reaction may also vary. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. The same IVT reaction may include 3.75 millimolar cap analog (e.g., trinucleotide cap). In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5.

In some embodiments, a RNA transcript (e.g., mRNA transcript) includes a modified nucleobase selected from pseudouridine (yr), 1-methylpseudouridine (m¹ψ), 5-methoxyuridine (mo⁵U), 5-methylcytidine (m⁵C), α-thio-guanosine and α-thio-adenosine.

In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m¹ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above.

Alternatively, the polynucleotide (e.g., RNA polynucleotide, such as mRNA polynucleotide) may not be uniformly modified (e.g., partially modified, part of the sequence is modified). Each possibility represents a separate embodiment of the present invention.

In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl₂) present in an IVT reaction is 1:1 to 1:5. For example, the molar ratio of NTP+ trinucleotide cap (e.g., GAG) to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

The addition of nucleoside triphosphates (NTPs) to the 3′ end of a growing RNA strand is catalyzed by a polymerase, such as T7 RNA polymerase, for example, any one or more of the T7 RNA polymerase variants (e.g., G47A) of the present disclosure. In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, the polynucleotide of the present disclosure is an IVT polynucleotide. Traditionally, the basic components of an mRNA molecule include at least a coding region, a 5′ UTR, a 3′ UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded therapeutic payload or prophylactic payload. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a therapeutic payload or prophylactic payload can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a therapeutic payload or prophylactic payload, or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture and methods of making a polynucleotide are disclosed in International PCT application WO 2017/201325, filed on 18 May 2017, the entire contents of which are hereby incorporated by reference.

### 12. Purification

In other aspects, a polynucleotide (e.g., an mRNA) disclosed herein can be purified. Purification of the polynucleotides (e.g., mRNA) described herein can include, but is not limited to, polynucleotide clean-up, quality assurance and quality control.

Clean-up can be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) of the disclosure removes impurities that can reduce or remove an unwanted immune response, e.g., reducing cytokine activity.

In some embodiments, the polynucleotide (e.g., mRNA) of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a therapeutic payload or prophylactic payload disclosed herein increases expression of the therapeutic payload or prophylactic payload, compared to polynucleotides encoding the therapeutic payload or prophylactic payload, purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a therapeutic payload or prophylactic payload. In some embodiments, the purified polynucleotide encodes a therapeutic payload or prophylactic payload.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### 13. Chemical Modifications of Polynucleotides

As described above, modified nucleosides and nucleotides of a nucleic acid (e.g., RNA nucleic acids, such as mRNA nucleic acids) may be included in a polynucleotide of the invention. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N₁-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an RNA nucleic acid of the disclosure comprises N1-methyl-pseudouridine (m1ψ) substitutions at one or more or all uridine positions of the nucleic acid.

In some embodiments, an RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises pseudouridine (ψ) substitutions at one or more or all uridine positions of the nucleic acid.

In some embodiments, an RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N₁-methyl-pseudouridine, meaning that all uridine residues in the mRNA sequence are replaced with N₁-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### 14. Sequence Optimization and Methods Thereof

In some embodiments, a polynucleotide of the disclosure comprises a sequence-optimized nucleotide sequence encoding a polypeptide disclosed herein, e.g., a polynucleotide encoding a therapeutic payload or prophylactic payload. In some embodiments, the polynucleotide of the disclosure comprises an open reading frame (ORF) encoding a therapeutic payload or prophylactic payload, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence of the disclosure has a reduced number of consecutive uracils with respect to the corresponding wild-type nucleic acid sequence. For example, two consecutive leucines can be encoded by the sequence CUUUUG, which includes a four-uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence of the disclosure has a reduced number of uracil triplets (UUU) with respect to the wild-type nucleic acid sequence. In some embodiments, a uracil-modified sequence has a reduced number of uracil pairs (UU) with respect to the number of uracil pairs (UU) in the wild-type nucleic acid sequence. In some embodiments, a uracil-modified sequence of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence has a % UUwt between below 100%.

In some embodiments, the polynucleotide of the disclosure comprises a uracil-modified sequence. In some embodiments, the uracil-modified sequence comprises at least one chemically modified nucleobase, e.g., 5-methoxyuracil. In some embodiments, at least 95% of a nucleobase (e.g., uracil) in a uracil-modified sequence of the disclosure are modified nucleobases. In some embodiments, at least 95% of uracil in a uracil-modified sequence is 5-methoxyuracil.

In some embodiments, a polynucleotide of the disclosure is sequence optimized.

A sequence optimized nucleotide sequence (nucleotide sequence is also referred to as “nucleic acid” herein) comprises at least one codon modification with respect to a reference sequence (e.g., a wild-type sequence encoding a therapeutic payload or prophylactic payload). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence-optimized nucleic acids (e.g., an RNA, e.g., an mRNA) can be administered to a subject in need thereof to facilitate in vivo expression of functionally active encoding a therapeutic payload or prophylactic payload.

Additional and exemplary methods of sequence optimization are disclosed in International PCT application WO 2017/201325, filed on 18 May 2017, the entire contents of which are hereby incorporated by reference.

### 15. Lipid Content of Lipid Nanoparticles

As set forth above, with respect to lipids, LNPs for use as delivery vehicles disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below.

In some embodiments, nucleic acids of the invention are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, ²-15%, ²-10%, ²-5%, 5-15%, 5-10%, or 10-15% PEG lipid. In some embodiments, the lipid nanoparticle comprises a molar ratio of 0.5%, 1%, 2%, 3%, 4%, 5%, 6%, 7% 8%, 9% 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Ionizable Amino Lipids

In some aspects, the disclosure relates to a compound of Formula (I):

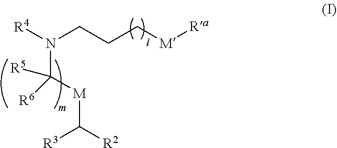

or its N-oxide, or a salt or isomer thereof,
wherein R^{′ a} is R^{′branched}; wherein
R′branched is:

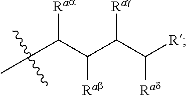

wherein

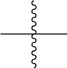

denotes a point of attachment;
wherein Raα, Raβ, Raγ, and Raδ are each independently selected from the group consisting of H, C2-12 alkyl, and C2-12 alkenyl;
R2 and R3 are each independently selected from the group consisting of C₁-14 alkyl and
C2-14 alkenyl;
R4 is selected from the group consisting of —(CH2)nOH, wherein n is selected from the group consisting of 1, 2, 3, 4, and 5, and

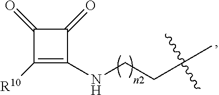

wherein

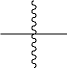

denotes a point of attachment; wherein
R₁₀ is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are each independently selected from the group consisting of —C(O)O— and

—OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.
In some embodiments of the compounds of Formula (I), R^{′ a} is R^{′branched};
R′branched is

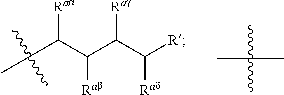

denotes a point of attachment; Raα, Raβ, Raγ, and Raδ are each H; R2 and R3 are each C1-14 alkyl; R4 is —(CH2)nOH; n is 2; each R5 is H; each R6 is H; M and M′ are each —C(O)O—; R′ is a C₁₋₁₂ alkyl; 1 is 5; and m is 7.
In some embodiments of the compounds of Formula (I), R^{′ a} is R^{′branched};
R′branched is

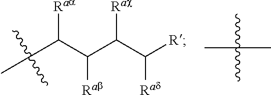

denotes a point of attachment; Raα, Raβ, Raγ, and Raδ are each H; R2 and R3 are each C1-14 alkyl; R4 is —(CH2)nOH; n is 2; each R5 is H; each R6 is H; M and M′ are each —C(O)O—; R′ is a C₁₋₁₂ alkyl; 1 is 3; and m is 7.

In some embodiments of the compounds of Formula (I), R^{′ a} is R^{′branched};
R′branched is

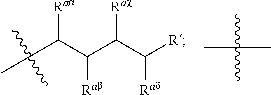

denotes a point of attachment; Raα is C2-12 alkyl; Raβ, Raγ, and Raδ are each H; R2 and R3 are each C1-14 alkyl; R4 is

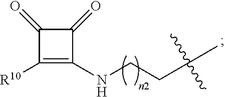

R₁₀ NH(C1-6 alkyl); n2 is 2; R5 is H; each R6 is H; M and M′ are each —C(O)O—; R′ is a C₁₋₁₂ alkyl; 1 is 5; and m is 7.

In some embodiments of the compounds of Formula (I), R^{′ a} is R^{′branched};
R′branched is

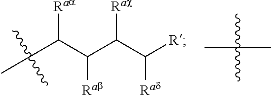

denotes a point of attachment; Raα, Raβ, and Raδ are each H; Ray is C2-12 alkyl; R2 and R3 are each C1-14 alkyl; R4 is —(CH2)nOH; n is 2; each R5 is H; each R6 is H; M and M′ are each —C(O)O—; R′ is a C1-12 alkyl; 1 is 5; and m is 7.

In some embodiments, the compound of Formula (I) is selected from:

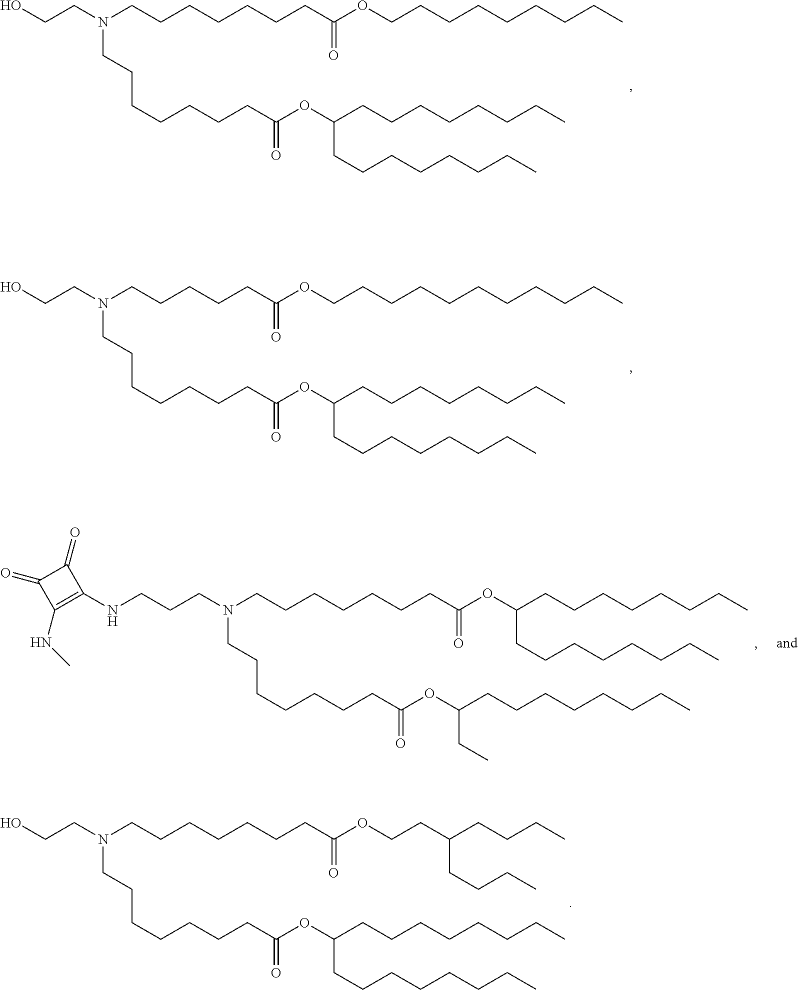 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

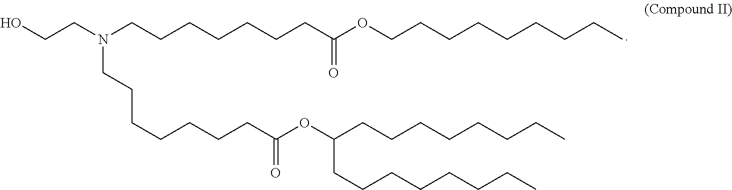 [SMILES: CCCCCCCCCOC(=O)CCCCCCCN(CCO)CCCCCCCC(=O)OC(CCCCCCCC)CCCCCCCC]

In some embodiments, the compound of Formula (I) is:

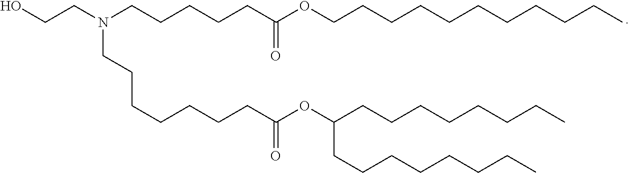 [SMILES: CCCCCCCCCCCOC(=O)CCCCCN(CCO)CCCCCCCC(=O)OC(CCCCCCCC)CCCCCCCC]

In some embodiments, the compound of Formula (I) is:

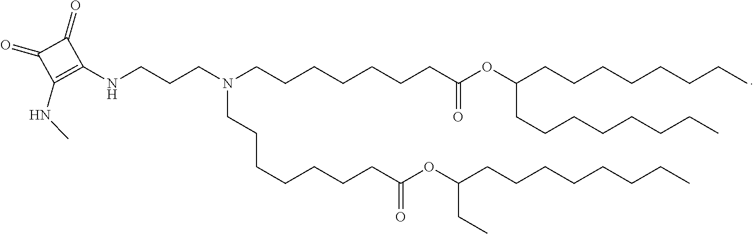 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

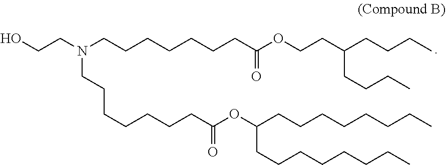 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (Ia):

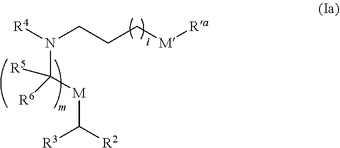

or a salt or isomer thereof,
wherein R^{′ a} is R^{′branched}; wherein
R′branched is

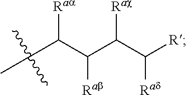

wherein

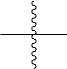

denotes a point of attachment;
wherein Raβ, Raγ, and Raδ are each independently selected from the group consisting of H, C2-12 alkyl, and C2-12 alkenyl;
R2 and R3 are each independently selected from the group consisting of C1-14 alkyl and

C2-14 alkenyl;
R4 is selected from the group consisting of —(CH2)nOH wherein n is selected from the group consisting of 1, 2, 3, 4, and

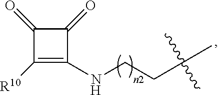

wherein

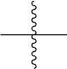

denotes a point of attachment; wherein
R10 is N(R)2; each R is independently selected from the group consisting of C1-6 alkyl, C2-3 alkenyl, and H; and n2 is selected from the group consisting of 1, 2, 3, 4, 5, 6, 15 7, 8, 9, and 10;
each R5 is independently selected from the group consisting of C1-3 alkyl, C2-3 alkenyl, and H;
each R6 is independently selected from the group consisting of C1-3 alkyl, C2-3 alkenyl, and H; M and M′ are each independently selected from the group consisting of —C(O)O— and

—OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some aspects, the disclosure relates to a compound of Formula (Ib):

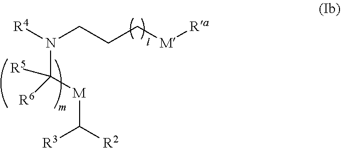

or its N-oxide, or a salt or isomer thereof,
wherein R^{′ a} is R^{′branched}; wherein
R′branched is

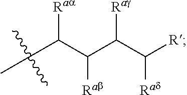

wherein

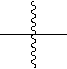

denotes a point of attachment;
wherein Raα, Raβ, Raγ, and Raδ are each independently selected from the group consisting of H, C2-12 alkyl, and C2-12 alkenyl;
R2 and R3 are each independently selected from the group consisting of C1-14 alkyl and C2-14 alkenyl;
R4 is —(CH2)nOH, wherein n is selected from the group consisting of 1, 2, 3, 4, and 5;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are each independently selected from the group consisting of —C(O)O— and

—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.
In some embodiments of Formula (I) or (Ib), R^{′ a} is R^{′branched}; R′branched is

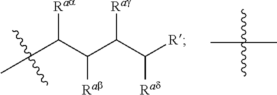

denotes a point of attachment; Raβ, Raγ, and Raδ are each H; R2 and R3 are each C1-14 alkyl; R4 is —(CH2)nOH; n is 2; each R5 is H; each R6 is H; M and M′ are each —C(O)O—; R′ is a C₁₋₁₂ alkyl; 1 is 5; and m is 7.

In some embodiments of Formula (I) or (Ib), R^{′ a} is R^{′branched}; R′branched is

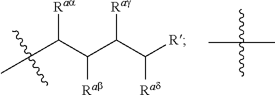

denotes a point of attachment; Raβ, Raγ, and Raδ are each H; R2 and R3 are each C1-14 alkyl; R4 is —(CH2)nOH; n is 2; each R5 is H; each R6 is H; M and M′ are each —C(O)O—; R′ is a C₁₋₁₂ alkyl; 1 is 3; and m is 7.

In some embodiments of Formula (I) or (Ib), R^{′ a} is R^{′branched}; R′branched is

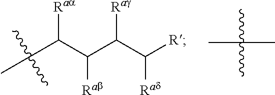

denotes a point of attachment; Rap and Raδ are each H; Ray is C2-12 alkyl; R2 and R3 are each C1-14 alkyl; R4 is —(CH2)nOH; n is 2; each R5 is H; each R6 is H; M and M′ are each —C(O)O—; R′ is a C₁₋₁₂ alkyl; 1 is 5; and m is 7.

In some aspects the disclosure relates to a compound of Formula (Ic):

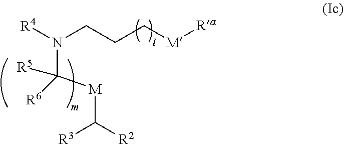

or its N-oxide, or a salt or isomer thereof,
wherein R^{′ a} is R^{′branched}; wherein
R′branched is

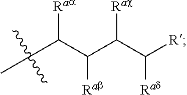

wherein

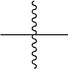

denotes a point of attachment;
wherein Raα, Raβ, Raγ, and Raδ are each independently selected from the group consisting of H, C2-12 alkyl, and C2-12 alkenyl;
R2 and R3 are each independently selected from the group consisting of C1-14 alkyl and

C2-14 alkenyl;
R4 is

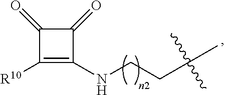

wherein

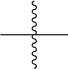

denotes a point of attachment; wherein
R10 is N(R)2; each R is independently selected from the group consisting of C1-6 alkyl, C2-3 alkenyl, and H; n2 is selected from the group consisting of 1, 2, 3, 4, 5, 6, 7, 8, 9, and 10;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are each independently selected from the group consisting of —C(O)O— and

—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.
In some embodiments, R^{′ a} is R^{′branched}; R′branched is

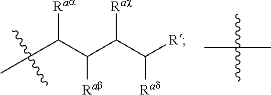

denotes a point of attachment; Raβ, Raγ, and Raδ are each H; Raα is C2-12 alkyl; R2 and R3 are each C1-14 alkyl; R4 is

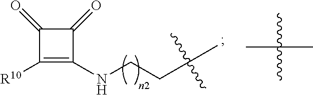

denotes a point of attachment; R10 is NH(C1-6 alkyl); n2 is 2; each R5 is H; each R6 is H; M and M′ are each —C(O)O—; R′ is a C₁₋₁₂ alkyl; 1 is 5; and m is 7. In some embodiments, the compound of Formula (Ic) is:

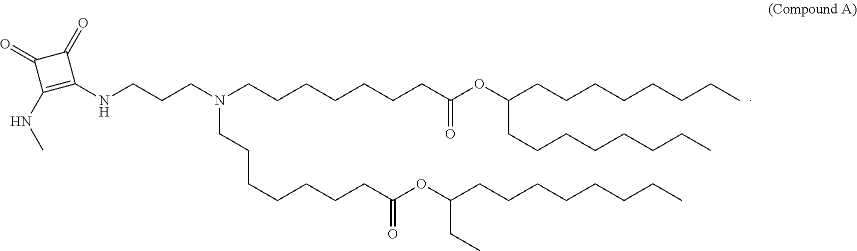 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (II):

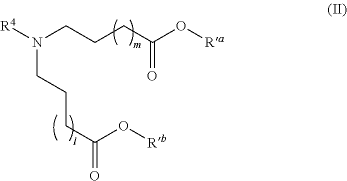

or its N-oxide, or a salt or isomer thereof,
wherein R^{′ a} is R^{′branched} or R′cyclic; wherein
R′branched is

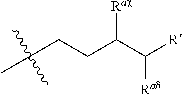

### and R′cyclic is:

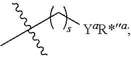

and

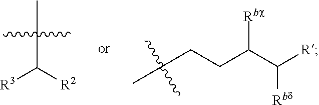

wherein

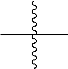

denotes a point of attachment;
Raγ and Raδ are each independently selected from the group consisting of H, C1-12 alkyl, and C2-12 alkenyl, wherein at least one of Raγ and Raδ is selected from the group consisting of C1-12 alkyl and C2-12 alkenyl;
Rbγ and Rbδ are each independently selected from the group consisting of H, C1-12 alkyl, and C2-12 alkenyl, wherein at least one of Rbγ and Rbδ is selected from the group consisting of C1-12 alkyl and C2-12 alkenyl;
R2 and R3 are each independently selected from the group consisting of C1-14 alkyl and C2-14 alkenyl;
R4 is selected from the group consisting of —(CH2)nOH wherein n is selected from the group consisting of 1, 2, 3, 4, and

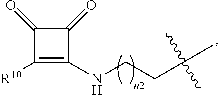

wherein

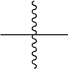

denotes a point of attachment; wherein
R₁₀ is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Ya is a C3-6 carbocycle;
R*″ a is selected from the group consisting of C1-15 alkyl and C2-15 alkenyl; and
s is 2 or 3;
m is selected from 1, 2, 3, 4, 5, 6, 7, 8, and 9;
l is selected from 1, 2, 3, 4, 5, 6, 7, 8, and 9.
In some aspects, the disclosure relates to a compound of Formula (II-a):

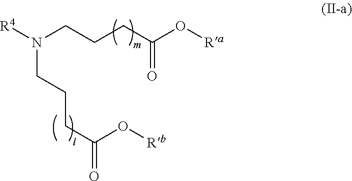

or its N-oxide, or a salt or isomer thereof,
wherein R^{′ a} is R^{′branched} or R′cyclic; wherein
R′branched is:

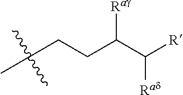

### and R′b is:

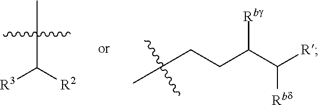

wherein

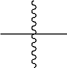

denotes a point of attachment;

Raγ and Raδ are each independently selected from the group consisting of H, C1-12 alkyl, and C2-12 alkenyl, wherein at least one of Raγ and Raδ is selected from the group consisting of C1-12 alkyl and C2-12 alkenyl;
Rbδ and Rbδ are each independently selected from the group consisting of H, C1-12 alkyl, and C2-12 alkenyl, wherein at least one of Rbδ and Rbδ is selected from the group consisting of C1-12 alkyl and C2-12 alkenyl;
R2 and R3 are each independently selected from the group consisting of C1-14 alkyl and

C2-14 alkenyl;
R4 is selected from the group consisting of —(CH2)nOH wherein n is selected from the group consisting of 1, 2, 3, 4, and

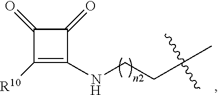

wherein

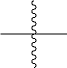

denotes a point of attachment; wherein
R₁₀ is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.
In some aspects, the disclosure relates to a compound of Formula (II-b):

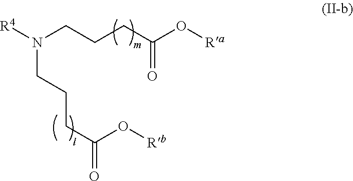

or its N-oxide, or a salt or isomer thereof,
wherein R^{′ a} is R^{′branched} or R′cyclic; wherein
R^{′branched} is:

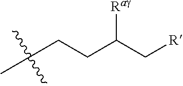

### and R′b is:

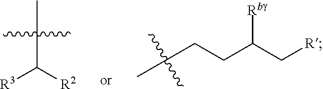

wherein

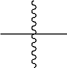

denotes a point of attachment;
Raγ and Rbδ are each independently selected from the group consisting of C₁₋₁₂ alkyl and C2-12 alkenyl;
R2 and R3 are each independently selected from the group consisting of C1-14 alkyl and

C2-14 alkenyl;
R4 is selected from the group consisting of —(CH2)nOH wherein n is selected from the group consisting of 1, 2, 3, 4, and

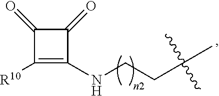

wherein

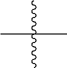

denotes a point of attachment; wherein
R₁₀ is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.
In some aspects, the disclosure relates to a compound of Formula (II-c):

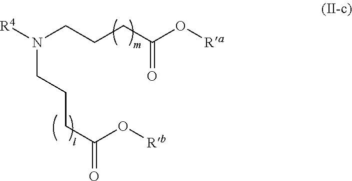

or its N-oxide, or a salt or isomer thereof,
wherein R^{′ a} is R^{′branched} or R′cyclic; wherein
R^{′branched} is:

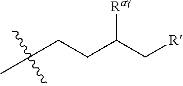

### and R′b is:

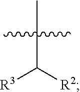

wherein

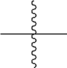

denotes a point of attachment;
wherein Raγ is selected from the group consisting of C1-12 alkyl and C2-12 alkenyl;
R2 and R3 are each independently selected from the group consisting of C1-14 alkyl and

C2-14 alkenyl;
R4 is selected from the group consisting of —(CH2)nOH wherein n is selected from the group consisting of 1, 2, 3, 4, and

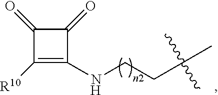

wherein

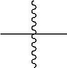

denotes a point of attachment; wherein
R10 is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.
In some aspects, the disclosure relates to a compound of Formula (II-d):

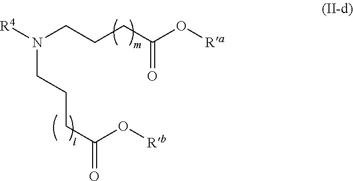

or its N-oxide, or a salt or isomer thereof,
wherein R^{′ a} is R^{′branched} or R′cyclic; wherein R′branched is:

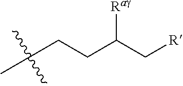

### and R′b is:

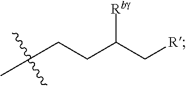

wherein

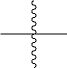

denotes a point of attachment;
wherein Raγ and Rbδ are each independently selected from the group consisting of C1-12 alkyl and C2-12 alkenyl;
R4 is selected from the group consisting of —(CH2)nOH wherein n is selected from the group consisting of 1, 2, 3, 4, and 5, and H n

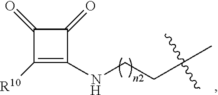

wherein

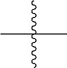

denotes a point of attachment; wherein
R₁₀ is N(R)2; each R is independently selected from the group consisting of C1-6 alkyl, C2-3 alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.
In some aspects, the disclosure relates to a compound of Formula (II-e):

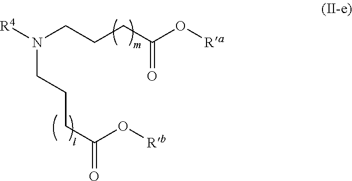

or its N-oxide, or a salt or isomer thereof,
wherein R^{′ a} is R^{′branched} or R′cyclic; wherein
R^{′branched} is:

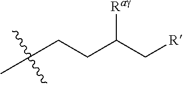

### and R′b is:

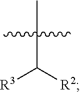

wherein

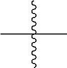

denotes a point of attachment;
wherein Raγ is selected from the group consisting of C1-12 alkyl and C2-12 alkenyl;
R2 and R3 are each independently selected from the group consisting of C1-14 alkyl and

C2-14 alkenyl;
R4 is —(CH2)nOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (II), (II-a), (II-b), (II-c), (II-d), or (II-e), m and l are each independently selected from 4, 5, and 6. In some embodiments of the compound of Formula (II), (II-a), (II-b), (II-c), (II-d), or (II-e), m and l are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C2-5 alkyl.

In some embodiments of the compound of Formula (II), (II-a), (II-b), (II-c), (II-d), or (II-e), R′b is:

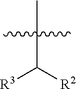

and R2 and R3 are each independently a C1-14 alkyl. In some embodiments of the compound of Formula (II), (II-a), (II-b), (II-c), (II-d), or (II-e), R′b is:

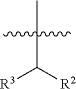

and R3 are each independently a C6-10 alkyl. In some embodiments of the compound of Formula (II), (II-a), (II-b), (II-c), (II-d), or (II-e), R′b is:

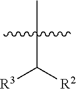

and R2 and R3 are each a C8 alkyl.

In some embodiments of the compound of Formula (II), (II-a), (II-b), (II-c), (II-d), or (II-e), R′branched is:

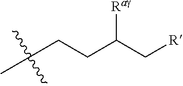

### and R′b is:

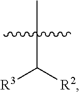

Raγ is a C₁₋₁₂ alkyl and R2 and R3 are each independently a C6-10 alkyl. In some embodiments of the compound of Formula (II), (II-a), (II-b), (II-c), (II-d), or (II-e), R′branched is:

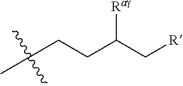

### and R′b is:

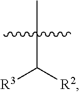

Raγ is a C2-6 alkyl and R2 and R3 are each independently a C6-10 alkyl. In some embodiments of the compound of Formula (II), (II-a), (II-b), (II-c), (II-d), or (II-e), R′branched is

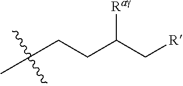

### and R′b is:

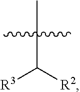

Raγ is a C2-6 alkyl, and R2 and R3 are each a C8 alkyl.

In some embodiments of the compound of Formula (II), (II-a), (II-b), (II-c), (II-d), or (II-e), R′branched is:

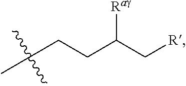

### R′b is:

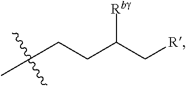

and Raγ and Rbδ are each a C₁₋₁₂ alkyl. In some embodiments of the compound of Formula (II), (II-a), (II-b), (II-c), (II-d), or (II-e), R′branched is:

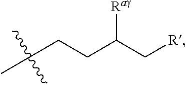

### R′b is:

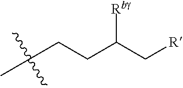

and Raγ and Rbδ are each a C2-6 alkyl.

In some embodiments of the compound of Formula (II), (II-a), (II-b), (II-c), (II-d), or (II-e), m and l are each independently selected from 4, 5, and 6 and each R′ independently is a C₁₋₁₂ alkyl. In some embodiments of the compound of Formula (II), (II-a), (II-b), (II-c), (II-d), or (II-e), m and l are each 5 and each R′ independently is a C2-5 alkyl.

In some embodiments of the compound of Formula (II), (II-a), (II-b), (II-c), (II-d), or (II-e), R′branched is:

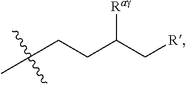

### R′b is:

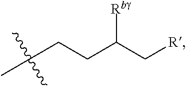

m and l are each independently selected from 4, 5, and 6, each R′ independently is a C₁₋₁₂ alkyl, and Raγ and Rbδ are each a C₁₋₁₂ alkyl. In some embodiments of the compound of Formula (II), (II-a), (II-b), (II-c), (II-d), or (II-e), R′branched is:

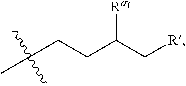

### R′b is:

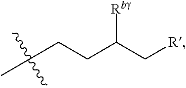

m and l are each 5, each R′ independently is a C2-5 alkyl, and Raγ and Rbγ are each a C2-6 alkyl.

In some embodiments of the compound of Formula (II), (II-a), (II-b), (II-c), (II-d), or (II-e), R′branched is:

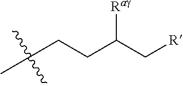

### and R′b is:

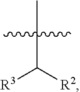

m and l are each independently selected from 4, 5, and 6, R′ is a C₁₋₁₂ alkyl, Raγ is a C₁₋₁₂ alkyl and R2 and R3 are each independently a C6-10 alkyl. In some embodiments of the compound of

Formula (II), II-a), (II-b), (II-c), (II-d), or (II-e), R′branched is:

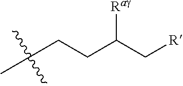

and

R′b is:

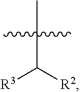

m and l are each 5, R′ is a C2-5 alkyl, Raγ is a C2-6 alkyl, and R2 and R3 are each a C8 alkyl.

In some embodiments of the compound of Formula (II), (II-a), (II-b), (II-c), (II-d), or (II-e),

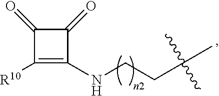

wherein R₁₀ is NH(C1-6 alkyl) and n2 is 2. In some embodiments of the compound of Formula (II), (II-a), (II-b), (II-c), (II-d), or (II-e), R4 is

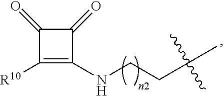

wherein R₁₀ is NH(CH3) and n2 is 2.

In some embodiments of the compound of Formula (II), (II-a), (II-b), (II-c), (II-d), or (II-e), R′branched is:

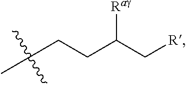

### R′b is:

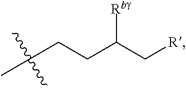

m and l are each independently selected from 4, 5, and 6, each R′ independently is a C₁₋₁₂ alkyl, Raγ and Rbδ are each a C₁₋₁₂ alkyl, and R4 is

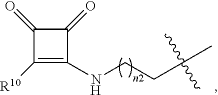

wherein R₁₀ is NH(C1-6 alkyl), and n2 is 2. In some embodiments of the compound of Formula II), (II-a), (II-b), (II-c), (II-d), or (II-e), R′branched is:

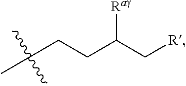

### R′b is:

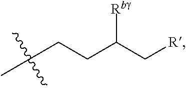

m and 1 are each 5, each R′ independently is a C2-5 alkyl, Raγ and Rbδ are each a C2-6 alkyl, and R4 is

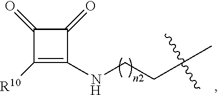

wherein R₁₀ is NH(CH3) and n2 is 2.

In some embodiments of the compound of Formula (II), (II-a), (II-b), (II-c), (II-d), or (II-e), R′branched is:

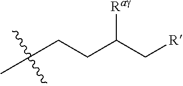

### and R′b is:

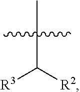

m and l are each independently selected from 4, 5, and 6, R′ is a C₁₋₁₂ alkyl, R2 and R3 are each independently a C6-10 alkyl, Raγ is a C₁₋₁₂ alkyl, and R4 is

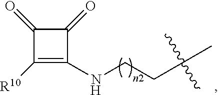

wherein R₁₀ is NH(C1-6 alkyl) and n2 is 2. In some embodiments of the compound of Formula (II), (II-a), (II-b), (II-c), (II-d), or (II-e), R′branched is:

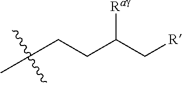

### and R′b is:

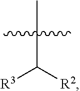

m and l are each 5, R′ is a C2-5 alkyl, Raγ is a C2-6 alkyl, R2 and R3 are each a C8 alkyl, and R4 is

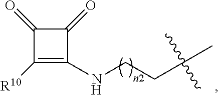

wherein R₁₀ is NH(CH3) and n2 is 2.

In some embodiments of the compound of Formula (II), (II-a), (II-b), (II-c), (II-d), or (II-e), R4 is —(CH2)nOH and n is 2, 3, or 4. In some embodiments of the compound of Formula (II), (II-a), (II-b), (II-c), (II-d), or (II-e), R4 is —(CH2)nOH and n is 2.

In some embodiments of the compound of Formula (II), (II-a), (II-b), (II-c), (II-d), or (II-e), R′branched is:

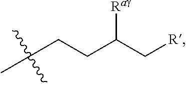

### R′b is:

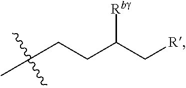

m and l are each independently selected from 4, 5, and 6, each R′ independently is a C₁₋₁₂ alkyl, Raγ and Rbγ are each a C₁₋₁₂ alkyl, R4 is —(CH2)nOH, and n is 2, 3, or 4. In some embodiments of the compound of Formula (II), (II-a), (II-b), (II-c), (II-d), or (II-e), R′branched is:

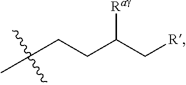

### R′b is:

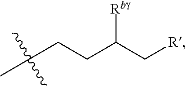

m and l are each 5, each R′ independently is a C2-5 alkyl, Raγ and Rbγ are each a C2-6 alkyl, R4 is —(CH2)nOH, and n is 2.

In some aspects, the disclosure relates to a compound of Formula (II-f):

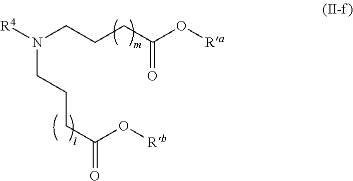

or its N-oxide, or a salt or isomer thereof, wherein R^{′ a} is R^{′branched} or R′cyclic; wherein
R′branched is:

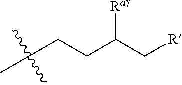

### and R′b is:

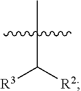

wherein

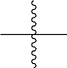

denotes a point of attachment;
Raγ is a C₁₋₁₂ alkyl;
R2 and R3 are each independently a C1-14 alkyl;
R4 is —(CH2)nOH wherein n is selected from the group consisting of 1, 2, 3, 4, and 5;
R′ is a C₁₋₁₂ alkyl;
m is selected from 4, 5, and 6; and
1 is selected from 4, 5, and 6.

In some embodiments of the compound of Formula (II-f), m and l are each 5, and
n is 2, 3, or 4.

In some embodiments of the compound of Formula (II-f) R′ is a C2-5 alkyl, Raγ is a C2-6 alkyl, and R2 and R3 are each a C6-10 alkyl.

In some embodiments of the compound of Formula (II-f), m and l are each 5, n is 2, 3, or 4, R′ is a C2-5 alkyl, Raγ is a C2-6 alkyl, and R2 and R3 are each a C6-10 alkyl.

In some aspects, the disclosure relates to a compound of Formula (II-g):

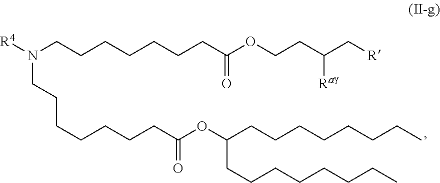

wherein
Raγ is a C2-6 alkyl;
R′ is a C2-5 alkyl; and
R4 is selected from the group consisting of —(CH2)nOH wherein n is selected from the group consisting of 3, 4, and

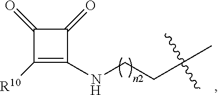

wherein

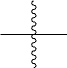

denotes a point of attachment, R₁₀ is NH(C1-6 alkyl), and n2 is selected from the group consisting of 1, 2, and 3.

In some aspects, the disclosure relates to a compound of Formula (II-h):

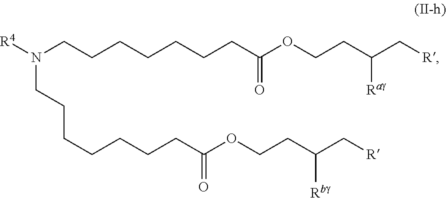

R (II-h), wherein
Raγ and Rbγ are each independently a C2-6 alkyl;
each R′ independently is a C2-5 alkyl; and
R4 is selected from the group consisting of —(CH2)nOH wherein n is selected from the group consisting of 3, 4, and

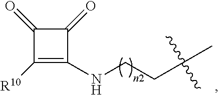

wherein

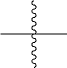

denotes a point of attachment, R₁₀ is NH(C1-6 alkyl), and n2 is selected from the group consisting of 1, 2, and 3.

In some embodiments of the compound of Formula (II-g) or (II-h), R4 is

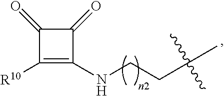

wherein
R₁₀ is NH(CH3) and n2 is 2.

In some embodiments of the compound of Formula (II-g) or (II-h), R4 is —(CH2)2OH.

In some aspects, the disclosure relates to a compound having the Formula (III):

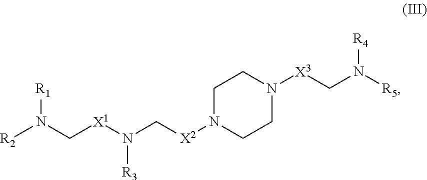

or a salt or isomer thereof, wherein
R1, R2, R3, R4, and R5 are independently selected from the group consisting of C5-20 alkyl, C5-20 alkenyl, —R″MR′, —R*YR″, —YR″, and —R*OR″;
each M is independently selected from the group consisting of —C(O)O—, —OC(O)—, —OC(O)O—, —C(O)N(R′)-, —N(R′)C(O)-, —C(O)—, —C(S)—, —C(S)S—, —S C(S)—, —CH(OH)—, —P(O)(OR′)O-, —S(O)2—, an aryl group, and a heteroaryl group;
X1, X2, and X3 are independently selected from the group consisting of a bond, —CH2—, —(CH2)2—, —CHR—, —CHY—, —C(O)—, —C(O)O—, —OC(O)—, —C(O)—CH2—, —CH2—C(O)—, —C(O)O—CH2—, —OC(O)—CH2—, —CH2—C(O)O—, —CH2—OC(O)—, —CH(OH)—, —C(S)—, and —CH(SH)—;
each Y is independently a C3-6 carbocycle;
each R* is independently selected from the group consisting of C1-12 alkyl and C2-12 alkenyl;
each R is independently selected from the group consisting of C1-3 alkyl and a C3-6 carbocycle;
each R′ is independently selected from the group consisting of C1-12 alkyl, C2-12 alkenyl, and H; and
each R″ is independently selected from the group consisting of C3-12 alkyl and C3-12 alkenyl, and wherein:
i) at least one of X1, X2, and X3 is not —CH2—; and/or
ii) at least one of R1, R2, R3, R4, and R5 is —R″MR′.

In some embodiments, R1, R2, R3, R4, and R5 are each C5-20 alkyl; X₁ is —CH2—; and X2 and X3 are each —C(O)-.

In some embodiments, the compound of Formula (III) is:

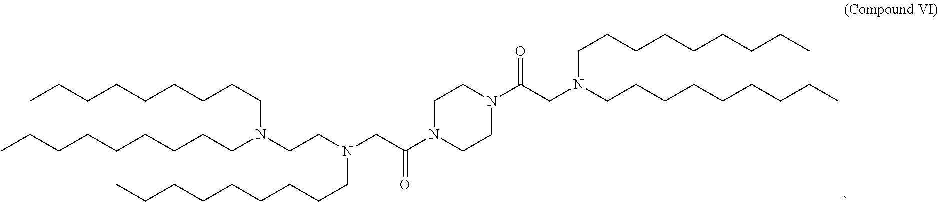 [SMILES: CCCCCCCCCN(CCCCCCCCC)CCN(CCCCCCCCC)CC(=O)N1CCN(C(=O)CN(CCCCCCCCC)CCCCCCCCC)CC1]

a salt or isomer thereof.

Phospholipids The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

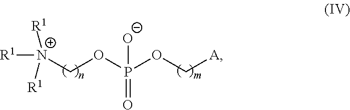

or a salt thereof, wherein:
each R1 is independently optionally substituted alkyl; or optionally two R1 are joined together with the intervening atoms to form optionally substituted monocyclic carbocyclyl or optionally substituted monocyclic heterocyclyl; or optionally three R1 are joined together with the intervening atoms to form optionally substituted bicyclic carbocyclyl or optionally substitute bicyclic heterocyclyl;
n is 1,2,3,4,5,6,7,8,9, or 10;
m is 0, 1, 2, 3, 4, 5, 6, 7, 8, 9, or 10;
A is of the Formula:

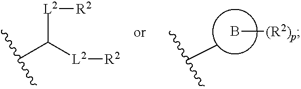

each instance of L2 is independently a bond or optionally substituted C1-6 alkylene, wherein one methylene unit of the optionally substituted C1-6 alkylene is optionally replaced with O, N(RN), S, C(O), C(O)N(RN), NRNC(O), C(O)O, OC(O), —OC(O)O, OC(O)N(RN), NRNC(O)O, or NRNC(O)N(RN);
each instance of R2 is independently optionally substituted C₁-30 alkyl, optionally substituted C1-30 alkenyl, or optionally substituted C₁₋₃₀ alkynyl; optionally wherein one or more methylene units of R2 are independently replaced with optionally substituted carbocyclylene, optionally substituted heterocyclylene, optionally substituted arylene, optionally substituted heteroarylene, N(RN), O, S, C(O), C(O)N(RN), —NRNC(O), NRNC(O)N(RN), C(O)O, OC(O), OC(O)O, OC(O)N(RN), NRNC(O)O, —C(O)S, SC(O), C(═NRN), C(═NRN)N(RN), NRNC(═NRN), NRNC(═NRN)N(RN), —C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), —N(RN)S(O)O, S(O)2, N(RN)S(O)2, S(O)2N(RN), N(RN)S(O)2N(RN), OS(O)2N(RN), or N(RN)S(O)20;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the Formula:

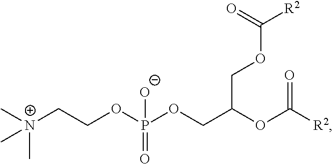

wherein each instance of R2 is independently unsubstituted alkyl, unsubstituted alkenyl, or unsubstituted alkynyl.

In some embodiments, the phospholipids may be one or more of the phospholipids described in U.S. Application No. 62/520,530.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R1 is not methyl. In certain embodiments, at least one of R1 is not hydrogen or methyl. In certain embodiments, the compound of Formula (IV) is of one of the following Formulae:

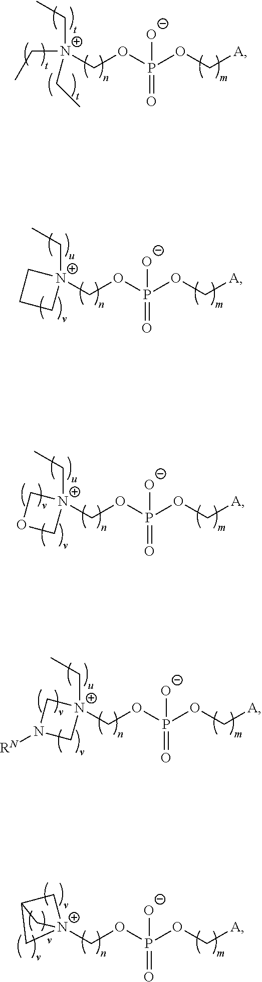

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

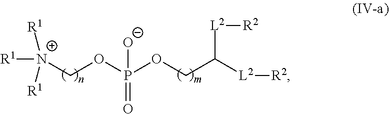

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

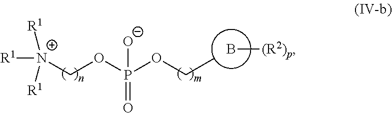

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R2 is each instance of R2 is optionally substituted C₁-30 alkyl, wherein one or more methylene units of R2 are independently replaced with optionally substituted carbocyclylene, optionally substituted heterocyclylene, optionally substituted arylene, optionally substituted heteroarylene, N(RN), O, S, C(O), C(O)N(RN), NRNC(O), —NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), N(RN)S(O)O, S(O)2, —N(RN)S(O)2, S(O)2N(RN), N(RN)S(O)2N(RN), OS(O)2N(RN), or N(RN)S(O)20.

In certain embodiments, the compound of Formula (IV) is of Formula (IV-c):

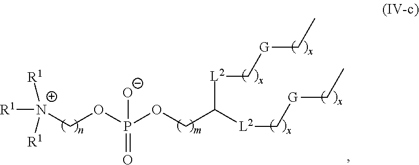

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(RN), O, S, C(O), —C(O)N(RN), NRNC(O), NRNC(O)N(RN), C(O)O, OC(O), OC(O)O, OC(O)N(RN), —NRNC(O)O, C(O)S, SC(O), C(═NRN), C(═NRN)N(RN), NRNC(═NRN), —NRNC(═NRN)N(RN), C(S), C(S)N(RN), NRNC(S), NRNC(S)N(RN), S(O), OS(O), —S(O)O, OS(O)O, OS(O)2, S(O)2O, OS(O)2O, N(RN)S(O), S(O)N(RN), —N(RN)S(O)N(RN), OS(O)N(RN), N(RN)S(O)O, S(O)2, N(RN)S(O)2, S(O)2N(RN), —N(RN)S(O)2N(RN), OS(O)2N(RN), or N(RN)S(O)20. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following Formulae:

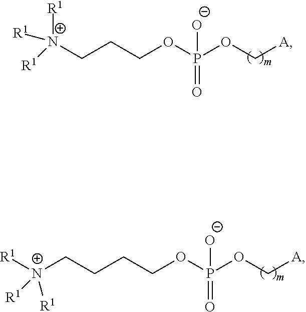

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

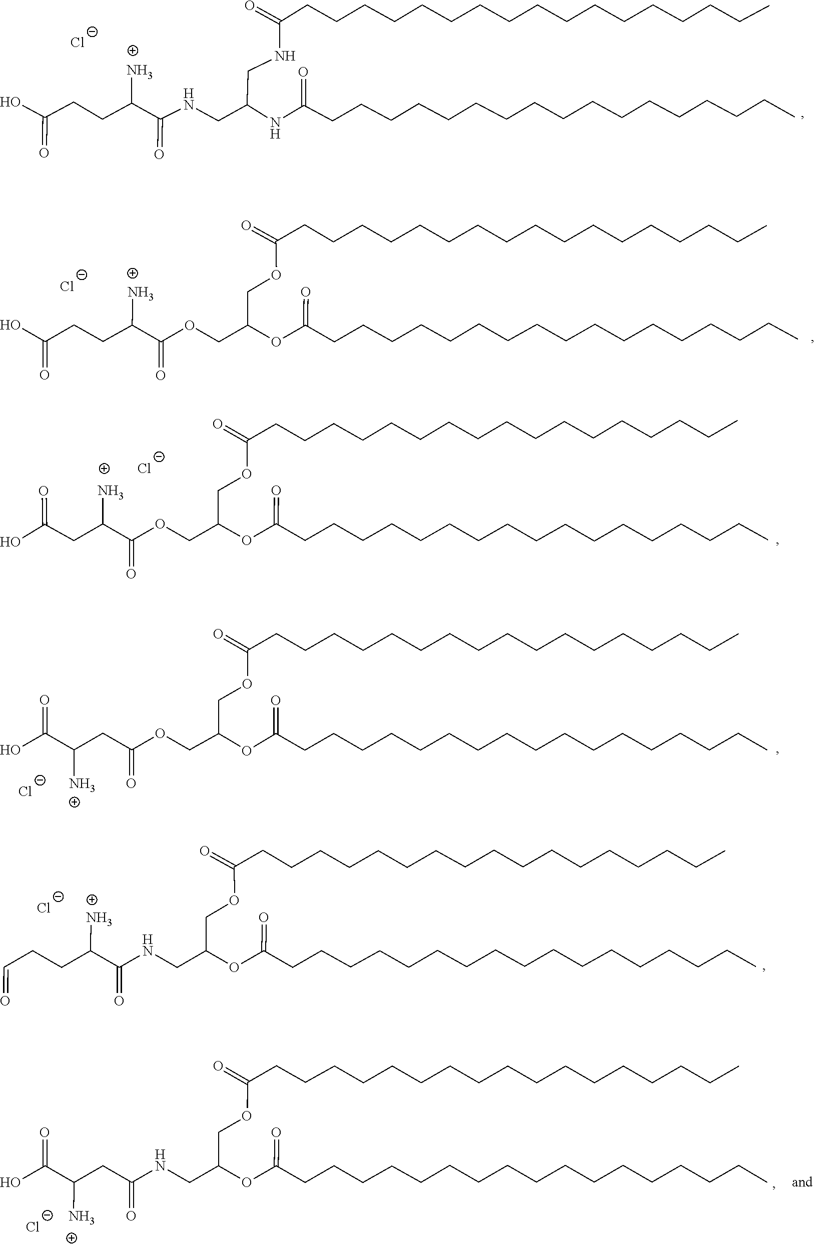

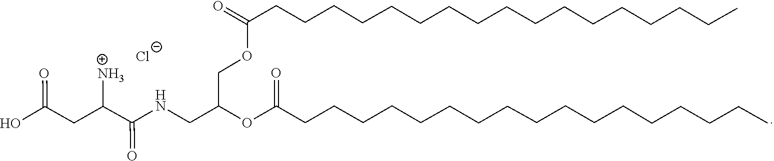 [SMILES: CCCCCCCCCCCCCCCCCC(=O)OCC(CNC(=O)C([NH3+])CC(=O)O)OC(=O)CCCCCCCCCCCCCCCCC ; [Cl-]]

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

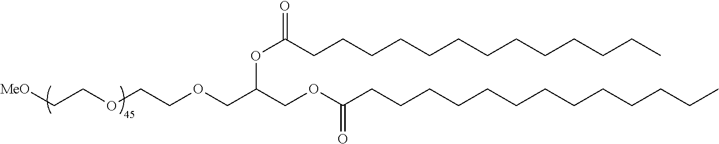

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

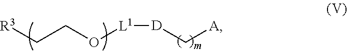

or salts thereof, wherein:
R3 is —ORO;
RO is hydrogen, optionally substituted alkyl, or an oxygen protecting group;
r is an integer between 1 and 100, inclusive;
L1 is optionally substituted C1-10 alkylene, wherein at least one methylene of the optionally substituted C1-10 alkylene is independently replaced with optionally substituted carbocyclylene, optionally substituted heterocyclylene, optionally substituted arylene, optionally substituted heteroarylene, O, N(RN), S, C(O), C(O)N(RN), —NRNC(O), C(O)O, OC(O), OC(O)O, OC(O)N(RN), NRNC(O)O, or NRNC(O)N(RN);
D is a moiety obtained by click chemistry or a moiety cleavable under physiological conditions;
m is 0,1,2,3,4,5,6 7 8 9 or 10;
A is of the Formula:

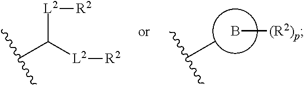

each instance of L2 is independently a bond or optionally substituted C1-6 alkylene, wherein one methylene unit of the optionally substituted C1-6 alkylene is optionally replaced with O, N(RN), S, C(O), C(O)N(RN), NRNC(O), C(O)O, OC(O), —OC(O)O, OC(O)N(RN), NRNC(O)O, or NRNC(O)N(RN);
each instance of R2 is independently optionally substituted C₁-30 alkyl, optionally substituted C1-30 alkenyl, or optionally substituted C₁₋₃₀ alkynyl; optionally wherein one or more methylene units of R2 are independently replaced with optionally substituted carbocyclylene, optionally substituted heterocyclylene, optionally substituted arylene, optionally substituted heteroarylene, N(RN), O, S, C(O), C(O)N(RN), —NRNC(O), NRNC(O)N(RN), C(O)O, OC(O), OC(O)O, OC(O)N(RN), NRNC(O)O, —C(O)S, SC(O), C(═NRN), C(═NRN)N(RN), NRNC(═NRN), NRNC(═NRN)N(RN), —C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), —N(RN)S(O)O, S(O)2, N(RN)S(O)2, S(O)2N(RN), N(RN)S(O)2N(RN), OS(O)2N(RN), or N(RN)S(O)2O;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R3 is —ORO, and RO is hydrogen). In certain embodiments, the compound of Formula (V) is of Formula (V-OH):

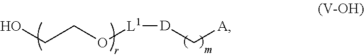

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

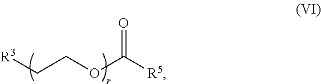

or a salts thereof, wherein:
R3 is-ORO;
RO is hydrogen, optionally substituted alkyl or an oxygen protecting group;
r is an integer between 1 and 100, inclusive;
R5 is optionally substituted C10-40 alkyl, optionally substituted C10-40 alkenyl, or optionally substituted C10-40 alkynyl; and optionally one or more methylene groups of R5 are replaced with optionally substituted carbocyclylene, optionally substituted heterocyclylene, optionally substituted arylene, optionally substituted heteroarylene, —N(RN), O, S, C(O), C(O)N(RN), NRNC(O), NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), —S(O)O, OS(O)O, OS(O)2, S(O)2O, OS(O)2O, N(RN)S(O), S(O)N(RN), —N(RN)S(O)N(RN), OS(O)N(RN), N(RN)S(O)O, S(O)2, N(RN)S(O)2, S(O)2N(RN), —N(RN)S(O)2N(RN), OS(O)2N(RN), or N(RN)S(O)20; and each instance of RN is independently hydrogen, optionally substituted alkyl, or a nitrogen protecting group.

In certain embodiments, the compound of Formula (VI) is of Formula (VI-OH):

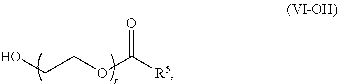

or a salt thereof. In some embodiments, r is 45.

In yet other embodiments the compound of Formula (VI) is:

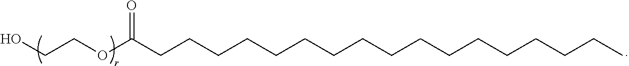

or a salt thereof.

In one embodiment, the compound of Formula (VI) is

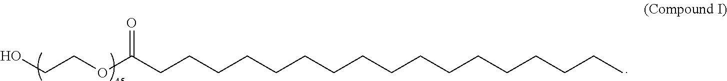

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic

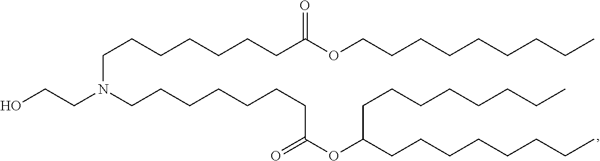 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

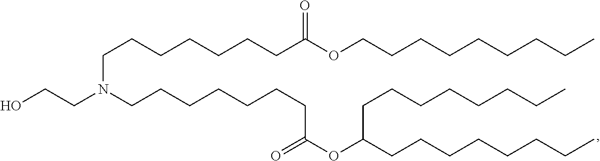 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

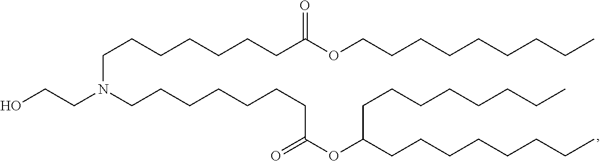 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

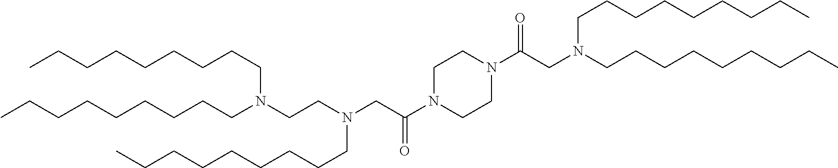 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of

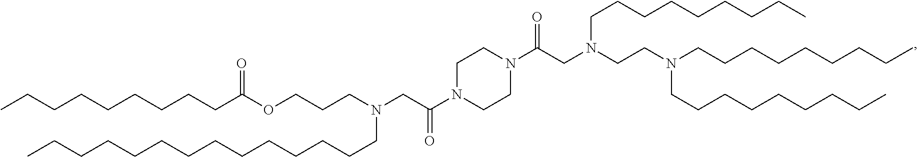 [SMILES: CCCCCCCCCCCCCCN(CCCOC(=O)CCCCCCCCC)CC(=O)N1CCN(C(=O)CN(CCCCCCCCC)CCN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C1-14 alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C2-14 alkenyl” means an optionally substituted linear or branched hydrocarbon including 2-14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group.

Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C2-14 alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms.

Rings may be three, four, five, six, seven, eight, nine, ten, eleven, twelve, thirteen, fourteen, fifteen, sixteen, seventeen, eighteen, nineteen, or twenty membered rings. The notation “C3-6 carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)2—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the Formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)43—), a thiol (e.g., SH), a sulfoxide (e.g., S(O)R), a sulfinic acid (e.g., S(O)OH), a sulfonic acid (e.g., S(O)2OH), a thial (e.g., C(S)H), a sulfate (e.g., S(O)42—), a sulfonyl (e.g., S(O)2), an amide (e.g., C(O)NR2, or N(R)C(O)R), an azido (e.g., N₃), a nitro (e.g., NO2), a cyano (e.g., CN), an isocyano (e.g., NC), an acyloxy (e.g., OC(O)R), an amino (e.g., NR2, NRH, or NH2), a carbamoyl (e.g., OC(O)NR2, OC(O)NRH, or OC(O)NH2), a sulfonamide (e.g., S(O)2NR2, S(O)2NRH, S(O)2NH2, N(R)S(O)2R, N(H)S(O)2R, N(R)S(O)2H, or N(H)S(O)2H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C1-6 alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N→O or N+—O—). Furthermore, in other instances, the nitrogens in the compounds of the disclosure can be converted to N-hydroxy or N-alkoxy compounds.

For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C1-C6 alkyl, C1-C6 alkenyl, C1-C6 alkynyl, 3-14-membered carbocycle or 3-14-membered heterocycle) derivatives.

mRNA-Lipid Adducts

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP-IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP-TP HPLC.

Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP-IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm.

Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (0-(2,3,4,5,6-Pentafluorobenzyl)hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9.

In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### 16. Exemplary Additional LNP Components

### 20 Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer.

For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10⁻⁴ M in water at about 30° C. and atmospheric pressure.

For example, amphiphilic polymer has a critical micelle concentration (CMC) ranging between about 0.1×10⁻⁴ M and about 1.3×10⁻⁴ M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

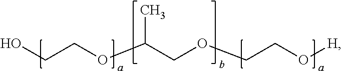

wherein a is an integer between 10 and 150 and b is an integer between 20 and 60.

For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

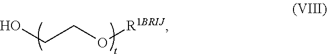

or a salt or isomer thereof, wherein:
t is an integer between 1 and 100;
R^{1BRIJ} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—NR^{N}C(O)N(R^{N})—, —C(O)O—, —OC(O)—, —OC(O)O—, —OC(O)N(R^{N})—, —NRNC(O)O—C(O)S—, —SC(O)—, —C(═NR^{N})—, —C(═NR^{N})N(R^{N})—, —NR^{N}C(═NRN—NRNC(═NRN)N(RN)—, —C(S)—, —C(S)N(RN)—, —NRNC(S)—, —NRNC(S)N(RN)-S(O)—, —OS(O)—, —S(O)O—, —OS(O)O—, —OS(O)₂—, —S(O)₂0—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂0-; and
each instance of R^{N} is independently hydrogen, C₁₋₆ alkyl, or a nitrogen protecting group

In some embodiment, R^{1BRIJ} is C₁₈ alkyl. For example, the polyethylene glycol ether is a compound of Formula (VIII-a):

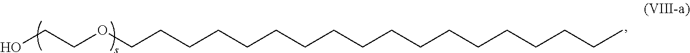

or a salt or isomer thereof.

In some embodiments, R^{1BRIJ} is C₁₈ alkenyl. For example, the polyethylene glycol ether is a compound of Formula (VIII-b):

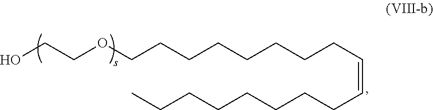

or a salt or isomer thereof

In some embodiments, the poloxamer is selected from the group consisting of 10 poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 20 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

Other components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 04, dornase alfa, neltenexine, and erdosteine), and DNases (e.g., rhDNase). A surface altering agent may be disposed within a nanoparticle and/or on the surface of an LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of an LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

Additional and exemplary lipid nanoparticles and compounds are disclosed in International Application No. PCT/US2020/051609, filed Sep. 18, 2020, the entire contents of which are hereby incorporated by reference.

### 17. Methods of Using the LNP Compositions

The present disclosure provides LNP compositions, which can be delivered to cells, e.g., target cells, e.g., in vitro or in vivo. For in vitro protein expression, the cell is contacted with the LNP by incubating the LNP and the cell ex vivo. Such cells may subsequently be introduced in vivo. For in vivo protein expression, the cell is contacted with the LNP by administering the LNP to a subject to thereby increase or induce protein expression in or on the cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the cell is contacted with the LNP by incubating the LNP and the target cell ex vivo. In one embodiment, the cell is a human cell. Various types of cells have been demonstrated to be transfectable by the LNP.

In another embodiment, the cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the cell is contacted with the LNP for a single treatment/transfection. In another embodiment, the cell is contacted with the LNP for multiple treatments/transfections (e.g., two, three, four or more treatments/transfections of the same cells).

In another embodiment, for in vivo delivery, the cell is contacted with the LNP by administering the LNP to a subject to thereby deliver the nucleic acid to cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In an aspect, provided herein is a method of increasing expression of a therapeutic payload or prophylactic payload in a cell, comprising administering to the cell an LNP composition disclosed herein.

In a related aspect, provided herein is an LNP composition for use in a method of increasing expression of a therapeutic payload or prophylactic payload in a cell.

In another aspect, the disclosure provides a method of increasing expression of a therapeutic payload or prophylactic payload, in a subject, comprising administering to the subject an effective amount of an LNP composition disclosed herein.

In a related aspect, provided herein is an LNP composition for use in a method of increasing expression of a therapeutic payload or prophylactic payload in a subject.

In yet another aspect, provided herein is a method of delivering an LNP composition disclosed herein.

In a related aspect, provided herein is an LNP composition for use in a method of delivering the LNP composition to a cell.

In an embodiment, the method or use, comprises contacting the cell in vitro, in vivo or ex vivo with the LNP composition.

In an embodiment, the LNP compositions of the present disclosure are contacted with cells, e.g., ex vivo or in vivo and can be used to deliver a secreted polypeptide, an intracellular polypeptide or a transmembrane polypeptide to a subject.

In an aspect, the disclosure provides a method of delivering an LNP composition disclosed herein to a subject having a disease or disorder, e.g., as described herein.

In a related aspect, provided herein is an LNP composition for use in a method of delivering the LNP composition to a subject having a disease or disorder, e.g., as described herein.

In another aspect, provided herein is a method of modulating an immune response in a subject, comprising administering to the subject in need thereof an effective amount of an LNP composition disclosed herein.

In a related aspect, provided herein is an LNP composition for use in a method of modulating an immune response in a subject, comprising administering to the subject an effective amount of the LNP composition.

In another aspect, provided herein is a method of delivering a secreted polypeptide, an intracellular polypeptide or a transmembrane polypeptide to a subject.

In an aspect, provided herein is a method of treating, preventing, or preventing a symptom of, a disease or disorder comprising administering to a subject in need thereof an effective amount of an LNP composition disclosed herein.

In a related aspect, provided herein is an LNP composition for use in a method of treating, preventing, or preventing a symptom of, a disease or disorder in a subject, comprising administering to the subject in need thereof an effective amount of the LNP composition.

In some embodiments, the methods or composition for use result in an increased expression and/or level of mRNA encoding the therapeutic payload or prophylactic payload.

In some embodiments, the methods or composition for use result in sustained expression and/or level of mRNA encoding the therapeutic payload or prophylactic payload.

In some embodiments, the methods or composition for use result in increased expression and/or level of therapeutic payload or prophylactic payload.

In some embodiments, the methods or composition for use result in sustained expression and/or level of therapeutic payload or prophylactic payload.

In some embodiments, any one of the functional effects described herein is compared to a cell which:
(a) has not been contacted with the LNP composition disclosed herein; or
(b) has not been contacted with an LNP comprising a polynucleotide comprising a 5′ UTR described herein, a 3′ UTR described herein and/or a coding region comprising a stop element described herein.

### 18. Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA.

In some aspects, the subject for the present methods or compositions has been treated with one or more standard of care therapies. In other aspects, the subject for the present methods or compositions has not been responsive to one or more standard of care therapies or anti-cancer therapies.

### 19. Pharmaceutical Compositions

The present disclosure provides pharmaceutical formulations comprising any of the LNP compositions disclosed herein.

In some embodiments of the disclosure, the polynucleotide is formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### 20. Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient.

The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1⁻³0%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotide is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### 21. Definitions

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art, such interval of accuracy is +10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Stop element. A “stop element” as that term is used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons. In an embodiment, a stop element further comprises at least 1, 2, 3, 4, 5, 6, 7, 8, 9, 10, 11 or 12 nucleotides upstream and/or downstream of the one or more stop codons.

3′stabilizing region. A “3′ stabilizing region” as that term is used herein, refers to a region that is made or becomes stable. A 3′ stabilizing region can be present at the 3′ terminus of a nucleic acids sequence. In an embodiment, a 3′ stabilizing region comprises a poly A tail, e.g., as described herein. In an embodiment, a 3′ stabilizing region comprises an alternative nucleoside, e.g., an inverted thymidine.

Sequence Identity: Calculations of sequence identity between sequences can be performed as follows. To determine the percent identity of two nucleotide sequences, the sequences are aligned for optimal comparison purposes (e.g., gaps can be introduced in one or both of a first and a second nucleotide sequence for optimal alignment). In some embodiments, the length of a reference sequence aligned for comparison purposes is at least 50%, e.g., at least 60%, 70%, 80%, 90%, or 100% of the length of the reference sequence. The nucleotides at corresponding nucleotide positions are compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The percent identity typically refers to the ratio of the number of matching residues to the total length of the alignment. The comparison of sequences and determination of percent identity between two sequences can be accomplished using a mathematical algorithm. In some embodiments, the percent identity between two nucleotide sequences is determined using a pairwise sequence alignment program or a multiple sequence alignment program. Exemplary sequence alignment programs include, but are not limited to, the lalign program (embnet.vital-it.ch; Huang and Miller, (1991) Adv. Appl. Math. 12:337-357); the Clustal Omega program (www.ebi.ac.uk; Sievers et al. (2011) Mol. Syst. Biol. 7:539). In some embodiments, the default parameters of the program are used. The nucleotide sequences described herein can be used as a “query sequence” to perform a search against public databases to, for example, identify other family members or related sequences.

Such searches can be performed using the BLAST® programs (blast.ncbi.nlm.nhi.gov; Altschul, et al. (1990) J. Mol. Biol. 215:403-10). For example, BLAST nucleotide searches can be performed with the blastn program to obtain nucleotide sequences identical or similar to a nucleotide sequence described herein. In some embodiments, the default parameters of the program are used.

Alternative nucleoside. An “alternative nucleoside” as that term is use herein, in reference to a nucleotide, nucleoside, or polynucleotide (such as the polynucleotides of the invention, e.g., mRNA molecule), refers to alteration with respect to A, G, U or C ribonucleotides. Generally, herein, these terms are not intended to refer to the ribonucleotide alterations in naturally occurring 5′-terminal mRNA cap moieties. The alterations may be various distinct alterations. In some embodiments, where the polynucleotide is an mRNA, the coding region, the flanking regions and/or the terminal regions (e.g., a 3′-stabilizing region) may contain one, two, or more (optionally different) nucleoside or nucleotide alterations. In some embodiments, an alternative polynucleotide introduced to a cell may exhibit reduced degradation in the cell, as compared to an unaltered polynucleotide.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Antibody molecule: In one embodiment, antibody molecules can be used for targeting to desired cell types. As used herein, “antibody molecule” refers to a naturally occurring antibody, an engineered antibody, or a fragment thereof, e.g., an antigen binding portion of a naturally occurring antibody or an engineered antibody. An antibody molecule can include, e.g., an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CHi domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Exemplary antibody molecules include, but are not limited to, humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering an LNP including the therapeutic and/or prophylactic to the subject (e.g., by an intravenous, intramuscular, intradermal, pulmonary or subcutaneous route). Administration of an LNP to a mammal or mammalian cell may involve contacting one or more cells with the lipid nanoparticle.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated.

Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.) Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. In an embodiment, the mRNA is a circular mRNA.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 n, from about 50 nm to about 200 nm or from about 70 to about 120 nm In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids. Unless otherwise specified, the nucleobase sequence of a SEQ ID NO described herein encompasses both natural nucleobases and chemically modified nucleobases (e.g., a “U” designation in a SEQ ID NO encompasses both uracil and chemically modified uracil).

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, PharmaceuticalSalts: Properties, Selection, and Use, P.H. Stahl and C.G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a molecule having at least 50%, 60%, 70%, 80%, 85%, 90%, 95%, 96%, 97%, 98%, 99% or 100% activity of, or structural similarity to, the wild type molecule, e.g., as measured by an art-recognized assay.

### 22. Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The invention is further described in the following examples, which do not limit the scope of the invention described in the claims.

Sequence information related to the 5′ and 3′ UTRs for Examples 1 and 2 is provided in Tables 5 and 6 below.

TABLE 5
5′UTR sequences

| Name: | Sequence: |
|---|---|
| v1.1 5′ UTR | GGAAAUAAGAGAGAAAAGAAGAGUAAGAAGAAAUAUA |
| | AGACCCCGGCGCCGCCACC (SEQ ID NO: 56) |
| v2.0 5′ UTR | GGAAAUCGCAAAAUUUGCUCUUCGCGUUAGAUUUCUU |
| | UUAGUUUUCUCGCAACUAGCAAGCUUUUUGUUCUCGC |
| | C (SEQ ID NO: 50) |

TABLE 6
3′UTR with stop element sequences underlined

| Name: | Sequence: |
|---|---|
| alpha 3'UTR/ | UGAUAAUAGGCUGGAGCCUCGGUGGCCUAGCUUCUUG |
| control | CCCCUUGGGCCUCCCCCCAGCCCCUCCUCCCCUUCCU |
| 3′UTR | GCACCCGUACCCCCGUGGUCUUUGAAUAAAGUCUGAG |
| | UGGGCGGC (SEQ ID NO: 180) |
| kappa 3′UTR | UAAAGCUCCCCGGGGGCCUCGGUGGCCUAGCUUCUUG |
| | CCCCUUGGGCCUCCCCCCAGCCCCUCCUCCCCUUCCU |
| | GCACCCGUACCCCCGUGGUCUUUGAAUAAAGUCUGAG |
| | UGGGCGGC (SEQ ID NO: 139) |
| iota 3′UTR | UAAGCCCCUCCGGGGGCCUCGGUGGCCUAGCUUCUUG |
| | CCCCUUGGGCCUCCCCCCAGCCCCUCCUCCCCUUCCU |
| | GCACCCGUACCCCCGUGGUCUUUGAAUAAAGUCUGAG |
| | UGGGCGGC (SEQ ID NO: 181) |

### Example 1. In vivo Effect of mRNA Having the v1.1 5′ UTR or the v2.0 5′ UTR in Combination with Either the Alpha 3′ UTR or the Kappa 3′ UTR

This Example describes in vivo assessments of firefly luciferase luminescence (ffluc) and/or a target protein encoded by mRNAs having the v1.1 5′ UTR or the v2.0 5′ UTR in combination with either the alpha 3′UTR (also referred to herein as “control 3′ UTR) or the kappa 3′UTR.

For evaluating the effect of the two 3′UTRs (“alpha” or “kappa”) in vivo in mice, CD-1 mice were intravenously dosed with 0.25 mg/kg of formulated ffLuc mRNA or hEPO mRNA Compound II/DMG (50% Compound II, 10% DSPC, 38.5 cholesterol, 1.5% PEG-DMG 2500 MW) by bolus intravenous tail vein injections] The mRNA constructs either had the v1.1 5′ UTR or the v2.0 5′ UTR. The animals were imaged 0-4 days (or 0-96 hours) post-dosing. There were 10 animals in each group. Serum was also collected and analyzed for hEPO protein level by ELISA (12 hours, 48 hours, and 96 hours). Mice were sacrificed on day 4 (i.e., 96 hours); liver and spleen were dissected and analyzed for luminescence.

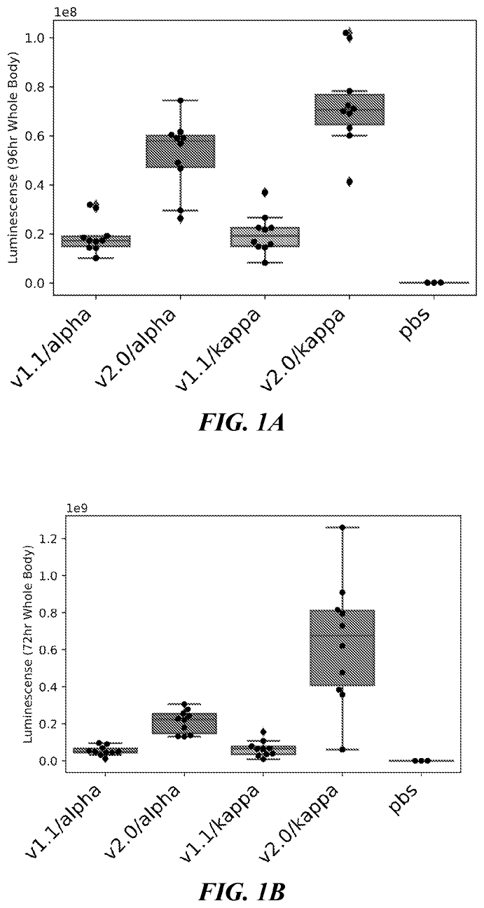

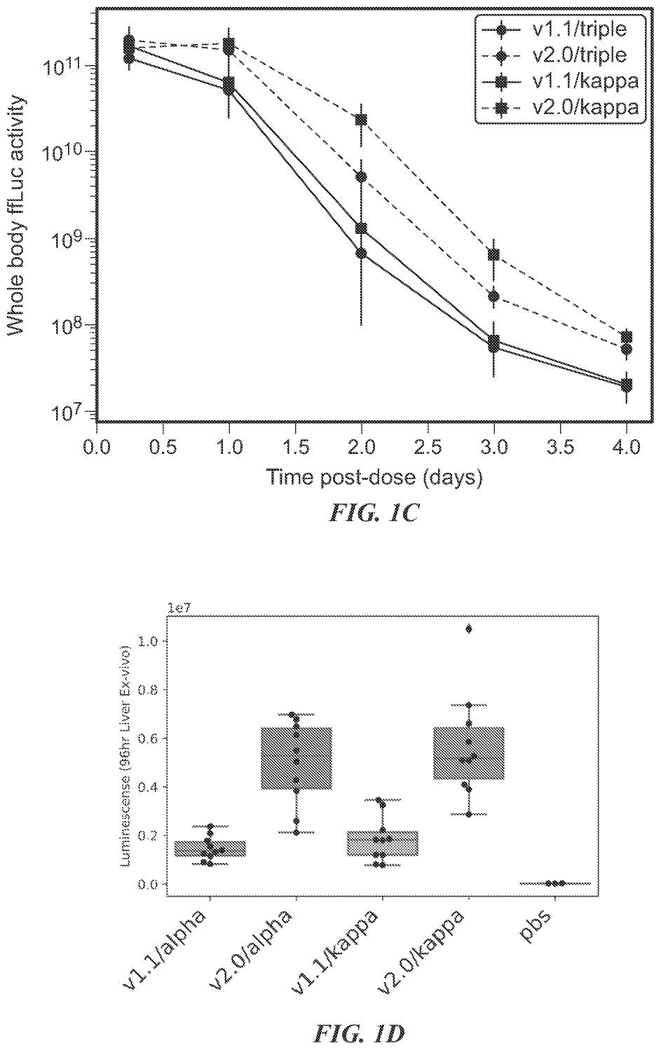

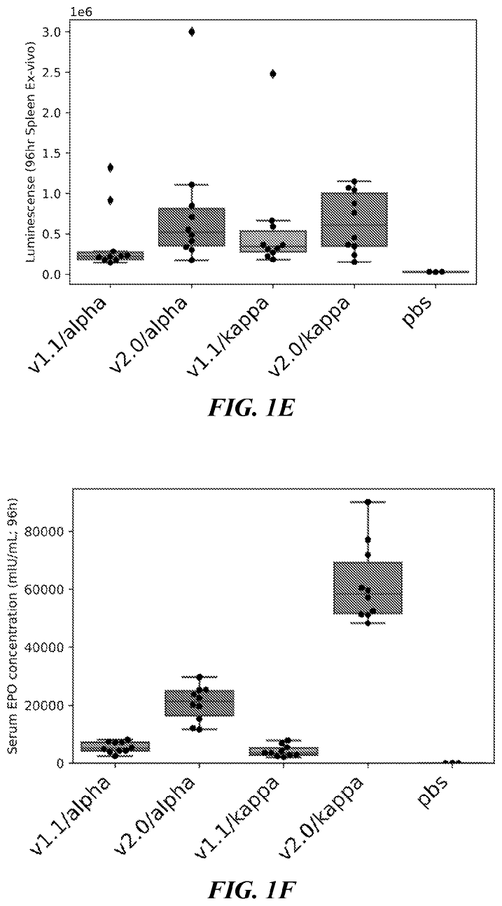

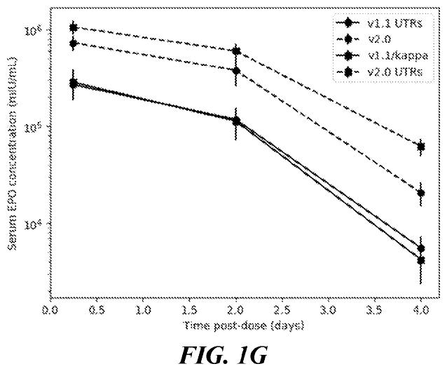

The results of this experiment are shown in FIGS. 1A-1G. The mRNA construct having both the v2.0 5′ UTR and the kappa 3′ UTR resulted in highest whole body luminescence (FIGS. 1A-1C), liver luminescence (FIG. 1D), and spleen luminescence (FIG. 1E). A similar effect was observed for target protein (i.e., EPO) expression in serum (FIGS. 1F-1G).

### Example 2. In vivo Effect of mRNA Having the v2.0 5′ UTR in Combination with Either the Kappa 3′ UTR or the Iota 3′ UTR

This Example describes in vivo assessments of target protein expression (codon optimized mOX40L) encoded by mRNAs having the v2.0 5′ UTR in combination with: a control 3′ UTR (v1.1), the kappa 3′UTR, or the iota 3′UTR in immune cells.

For evaluating the effect of two 3′UTRs (“kappa” or “iota”) in comparison to a control 3′UTR in vivo in mice, C57BL/6 mice were intravenously dosed with 0.50 mg/kg mOX40L_D99K mRNA formulated in LNPs containing Compound II and Compound I.

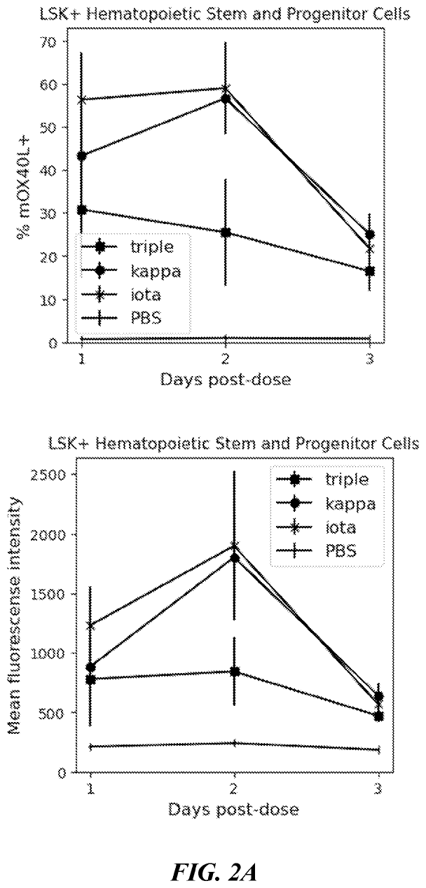

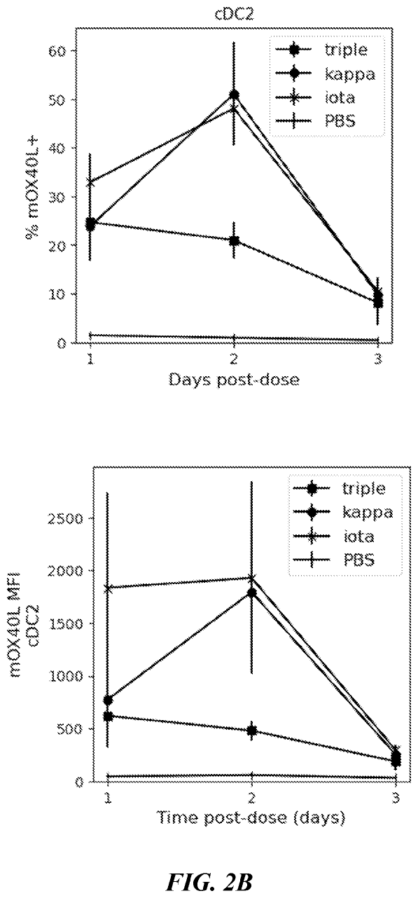

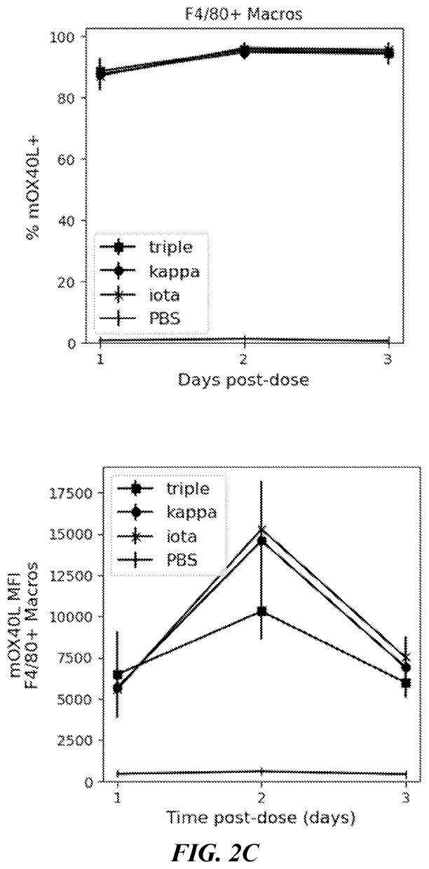

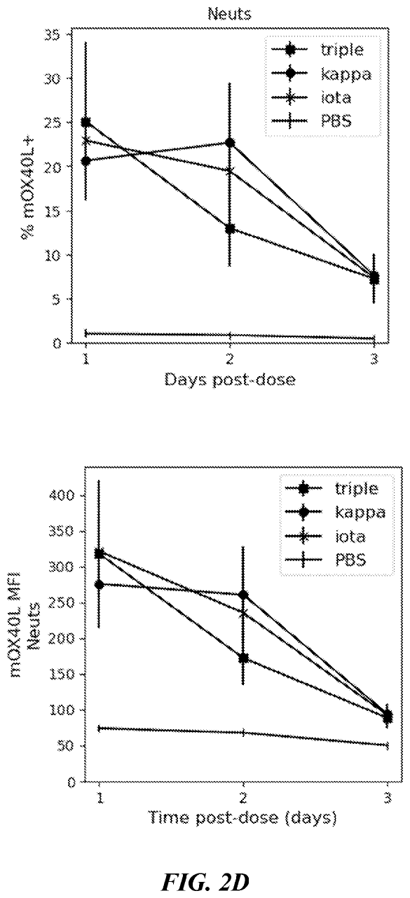

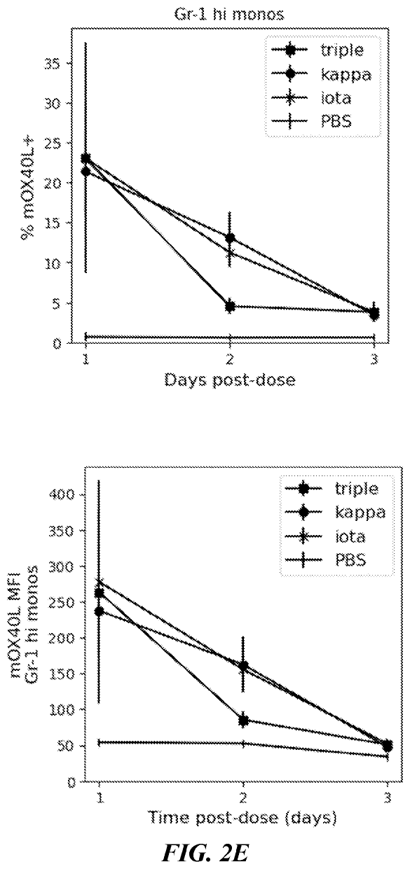

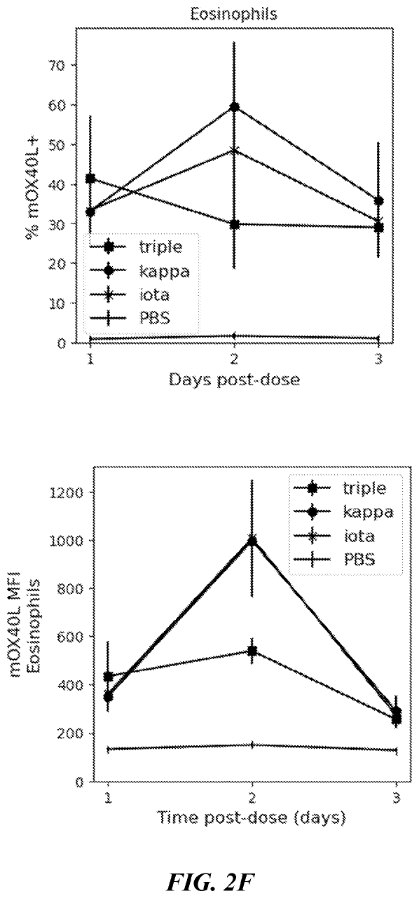

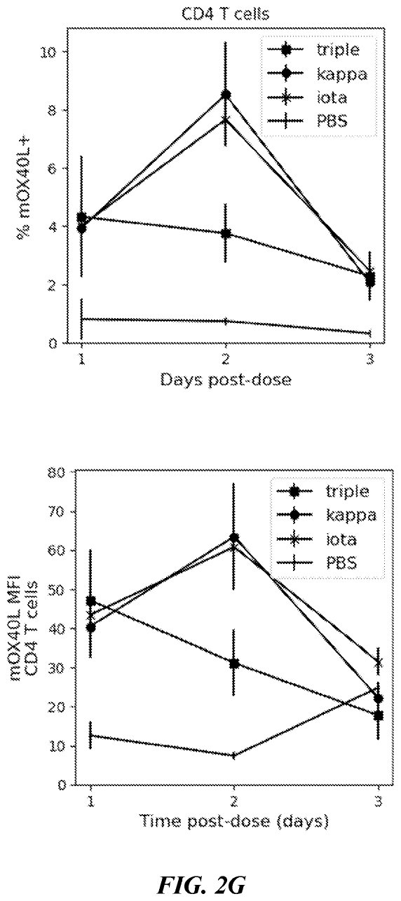

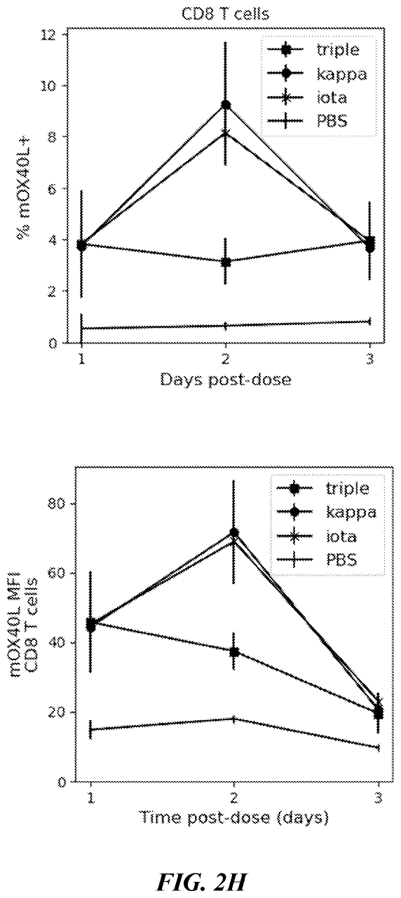

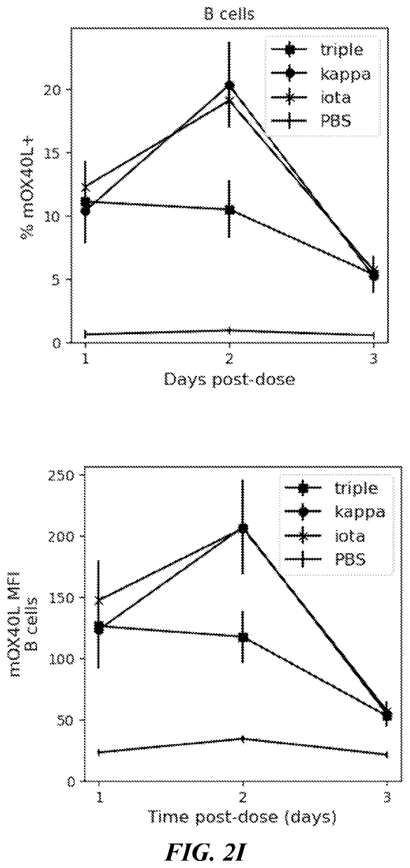

The mRNA constructs had the v2.0 5′ UTR, codon optimized mOX40L_D99K ORF, and either the kappa 3′UTR or the iota 3′UTR. The mean fluorescence intensity of OX40L and the percentage of mOX40L+ cells were assessed at 1, 2, and 3 days post-dose for LSK+ hematopoietic stem and progenitor cells (FIG. 2A), splenic dendritic cells (FIG. 2B), splenic macrophages (FIG. 2C), splenic neutrophils (FIG. 2D), splenic monocytes (FIG. 2E), splenic eosinophils (FIG. 2F), splenic CD4+ T cells (FIG. 2G), splenic CD8+ T cells (FIG. 211), and splenic B cells (FIG. 2I). mRNAs containing either the iota 3′UTR or the kappa 3′UTR were associated with increased expression of mOX40L in a majority of immune cells.

### Example 3. Synthesis of mRNA Encoding FANCA

An mRNA encoding human Fanconi anemia complementation group A (FANCA) polypeptide can be constructed, e.g., by using the ORF sequence (amino acid) provided in SEQ ID NO:1. See, Table 7 below.

An exemplary sequence optimized nucleotide sequence encoding the amino acid sequence of SEQ ID NO:1 is provided in SEQ ID NO:2. Another exemplary sequence optimized nucleotide sequence encoding the amino acid sequence of SEQ ID NO:1 is provided in SEQ ID NO:3. Additional exemplary sequence optimized nucleotide sequences encoding the amino acid sequence of SEQ ID NO:1 are provided in SEQ ID NOs:14-17. See, Table 7 below.

The mRNA sequence includes both 5′ and 3′ UTR regions flanking the ORF sequence (nucleotide). In an exemplary construct, the 5′ UTR and 3′ UTR sequences are SEQ ID NOs:64 and 139, respectively.

5′ UTR:
(SEQ ID NO: 64)
GGAAAUUAUUAUUAUUUCUAGCUACAAUUUAUCAUUGUAUUAUUUUAGCU
AUUCAUCAUUAUUUACUUGGUGAUCAACA
3′ UTR:
(SEQ ID NO: 139)
UAAAGCUCCCCGGGGGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC

In another exemplary construct, the 5′ UTR and 3′ UTR sequences are SEQ ID NOs:50 and 139, respectively.

5′ UTR:
(SEQ ID NO: 50)
GGAAAUCGCAAAAUUUGCUCUUCGCGUUAGAUUUCUUUUAGUUUUCUCGC
AACUAGCAAGCUUUUUGUUCUCGCC
3′ UTR:
(SEQ ID NO: 139)
UAAAGCUCCCCGGGGGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC

The FANCA mRNA sequence is prepared as modified mRNA. Specifically, during in vitro transcription, modified mRNA can be generated using N₁-methylpseudouridine-5′-Triphosphate to ensure that the mRNAs contain 100% N₁-methylpseudouridine instead of uridine. Alternatively, during in vitro transcription, modified mRNA can be generated using N₁-methoxyuridine-5′-Triphosphate to ensure that the mRNAs contain 100% 5-methoxyuridine instead of uridine. Further, FANCA-mRNA can be synthesized with a primer that introduces a polyA-tail, and a cap structure is generated on both mRNAs using co-transcriptional capping via m⁷G-ppp-Gm-AG tetranucleotide to incorporate a m⁷G-ppp-Gm-AG 5′ cap1. Alternatively, FANCA-mRNA can be synthesized and the polyA-tail introduced during Gibson assembly of the DNA template.

FANCA construct sequences used in the following examples are described below. By “G5” is meant that all uracils (U) in the mRNA are replaced by N₁-methylpseudouracils.

TABLE 7
FANCA Sequences

| | ORF Sequence | ORF Sequence | 5′ UTR | 3′ UTR | Construct |
|---|---|---|---|---|---|
| mRNA Name | (Amino Acid) | (Nucleotide) | Sequence | Sequence | Sequence |
| | SEQ ID NO: | | | | |
| | 1 | 2 | 64 | 139 | 4 |
| FANCA_01 | MSDSWVPNSA | AUGUCCGACUCCUGG | GGAAA | UAAAGC | SEQ ID |
| Chemistry: G5 | SGQDPGGRRRA | GUGCCCAACUCCGCU | UUAUU | UCCCCG | NO: 4 |
| Cap: C1 | WAELLAGRVK | AGCGGACAAGAUCCA | AUUAU | GGGGCC | consists |
| Poly A tail: | REKYNPERAQK | GGUGGCCGCAGGAG | UUCUAG | UCGGUG | from 5′ to |
| 100 nt (SEQ ID | LKESAVRLLRS | AGCGUGGGCUGAAC | CUACAA | GCCUAG | 3′ end: 5′ |
| NO: 121) | HQDLNALLLEV | UGCUUGCCGGGCGAG | UUUAUC | CUUCUU | UTR of |
| | EGPLCKKLSLS | UGAAGCGCGAGAAG | AUUGU | GCCCCU | SEQ ID |
| | KVIDCDSSEAY | UACAACCCCGAGCGC | AUUAU | UGGGCC | NO: 64, |
| | ANHSSSFIGSAL | GCCCAGAAGCUGAAG | UUUAGC | UCCCCC | ORF |
| | QDQASRLGVPV | GAGAGCGCCGUACGG | UAUUCA | CAGCCC | Sequence |
| | GILSAGMVASS | CUGCUGCGCAGCCAC | UCAUUA | CUCCUC | of SEQ ID |
| | VGQICTAPAET | CAGGACCUGAACGCC | UUUACU | CCCUUC | NO: 2, and |
| | SHPVLLTVEQR | CUGCUGCUGGAGGU | UGGUG | CUGCAC | 3′ UTR of |
| | KKLSSLLEFAQ | GGAGGGGCCCCUGUG | AUCAAC | CCGUAC | SEQ ID |
| | YLLAHSMFSRL | CAAGAAGCUGAGCCU | A | CCCCGU | NO: 139 |
| | SFCQELWKIQS | GUCCAAGGUGAUCG | | GGUCUU | |
| | SLLLEAVWHLH | ACUGCGACAGCAGCG | | UGAAU | |
| | VQGIVSLQELL | AGGCCUACGCCAACC | | AAAGUC | |
| | ESHPDMHAVG | ACAGCUCCUCCUUCA | | UGAGU | |
| | SWLFRNLCCLC | UCGGCUCCGCCCUGC | | GGGCGG | |
| | EQMEASCQHA | AGGAUCAAGCUAGCC | | C | |
| | DVARAMLSDF | GGCUGGGCGUUCCAG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCUCCCG | | | |
| | LAAGVQEESST | CCGAGACCAGCCACC | | | |
| | HKIVRCWFGVF | CCGUGCUGCUGACCG | | | |
| | SGHTLGSVISTD | UGGAGCAGCGCAAG | | | |
| | PLKRFFSHTLT | AAGCUGUCAAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCUCCCGC | | | |
| | YRRLFVMLSAE | CUGAGCUUCUGCCAG | | | |
| | ELVGHLQEVLE | GAGCUGUGGAAGAU | | | |
| | TQEVHWQRVL | CCAGUCCAGCCUGCU | | | |
| | SFVSALVVCFP | ACUGGAGGCCGUGU | | | |
| | EAQQLLEDWV | GGCACUUACACGUGC | | | |
| | ARLMAQAFESC | AGGGCAUCGUGAGCC | | | |
| | QLDSMVTAFLV | UGCAGGAGCUACUG | | | |
| | VRQAALEGPSA | GAGAGCCACCCCGAC | | | |
| | FLSYADWFKAS | AUGCACGCCGUGGGG | | | |
| | FGSTRGYHGCS | UCCUGGCUGUUCCGC | | | |
| | KKALVFLFTFL | AACCUGUGCUGCCUG | | | |
| | SELVPFESPRYL | UGCGAGCAGAUGGA | | | |
| | QVHILHPPLVP | AGCCUCCUGCCAACA | | | |
| | GKYRSLLTDYI | CGCCGACGUUGCACG | | | |
| | SLAKTRLADLK | GGCAAUGCUGAGCG | | | |
| | VSIENMGLYED | ACUUCGUGCAGAUG | | | |
| | LSSAGDITEPHS | UUCGUGCUGCGCGGG | | | |
| | QALQDVEKAI | UUCCAGAAGAACUCA | | | |
| | MVFEHTGNIPV | GACCUCCGGAGGACC | | | |
| | TVMEASIFRRP | GUGGAGCCUGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UUGCCCUGGACGCUC | | | |
| | QACSAAEEKPE | UCGCUGCCGGAGUGC | | | |
| | DAALGVRAEP | AGGAGGAGAGCUCC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCUCCGGCCAC | | | |
| | VAVISERLRAV | ACCCUGGGCUCCGUG | | | |
| | LGHNEDDSSVE | AUCUCCACCGAUCCC | | | |
| | ISKIQLSINTPRL | CUGAAGCGGUUCUUC | | | |
| | EPREHMAVDLL | UCCCACACCCUGACC | | | |
| | LTSFCQNLMAA | CAGAUCCUGACCCAC | | | |
| | SSVAPPERQGP | UCCCCUGUGCUGAAG | | | |
| | WAALFVRTMC | GCCAGCGACGCCGUG | | | |
| | GRVLPAVLTRL | CAGAUGCAGCGCGAG | | | |
| | CQLLRHQGPSL | UGGUCCUUCGCCCGC | | | |
| | SAPHVLGLAAL | ACCCAUCCCCUGCUG | | | |
| | AVHLGESRSAL | ACCAGCCUGUACCGG | | | |
| | PEVDVGPPAPG | CGGCUGUUCGUGAU | | | |
| | AGLPVPALFDS | GCUGUCCGCCGAGGA | | | |
| | LLTCRTRDSLFF | GCUGGUGGGCCACCU | | | |
| | CLKFCTAAISYS | GCAGGAGGUGCUGG | | | |
| | LCKFSSQSRDT | AGACCCAGGAGGUGC | | | |
| | LCSCLSPGLIKK | ACUGGCAGCGCGUGC | | | |
| | FQFLMFRLFSE | UGAGCUUCGUGAGC | | | |
| | ARQPLSEEDVA | GCCCUGGUGGUGUGC | | | |
| | SLSWRPLHLPS | UUCCCCGAGGCCCAG | | | |
| | ADWQRAALSL | CAGCUCCUGGAGGAC | | | |
| | WTHRTFREVLK | UGGGUGGCCCGGCUG | | | |
| | EEDVHLTYQD | AUGGCCCAGGCCUUC | | | |
| | WLHLELEIQPE | GAGAGCUGCCAGCUG | | | |
| | ADALSDTERQD | GACAGCAUGGUGACC | | | |
| | FHQWAIHEHFL | GCCUUCCUGGUGGUG | | | |
| | PESSASGGCDG | CGUCAAGCAGCCCUG | | | |
| | DLQAACTILVN | GAGGGCCCAAGCGCC | | | |
| | ALMDFHQSSRS | UUCCUGAGCUACGCC | | | |
| | YDHSENSDLVF | GACUGGUUCAAGGCC | | | |
| | GGRTGNEDIISR | AGCUUCGGGUCCACC | | | |
| | LQEMVADLEL | CGCGGGUACCACGGC | | | |
| | QQDLIVPLGHT | UGCUCCAAGAAGGCC | | | |
| | PSQEHFLFEIFR | CUGGUGUUCCUGUU | | | |
| | RRLQALTSGWS | UACCUUCCUGAGCGA | | | |
| | VAASLQRQREL | GCUGGUGCCCUUCGA | | | |
| | LMYKRILLRLP | GUCGCCCCGCUACCU | | | |
| | SSVLCGSSFQA | GCAGGUGCACAUCCU | | | |
| | EQPITARCEQFF | GCACCCACCCCUGGU | | | |
| | HLVNSEMRNFC | CCCUGGCAAGUACCG | | | |
| | SHGGALTQDIT | GAGCCUGCUGACCGA | | | |
| | AHFFRGLLNAC | CUACAUCUCCCUGGC | | | |
| | LRSRDPSLMVD | CAAGACCCGGCUGGC | | | |
| | FILAKCQTKCP | CGACCUGAAGGUGA | | | |
| | LILTSALVWWP | GCAUCGAGAACAUG | | | |
| | SLEPVLLCRWR | GGCCUGUACGAGGAC | | | |
| | RHCQSPLPREL | CUGUCCAGCGCCGGC | | | |
| | QKLQEGRQFAS | GACAUCACCGAGCCC | | | |
| | DFLSPEAASPAP | CACAGCCAGGCCCUG | | | |
| | NPDWLSAAAL | CAGGACGUGGAGAA | | | |
| | HFAIQQVREENI | GGCCAUCAUGGUGU | | | |
| | RKQLKKLDCER | UCGAGCACACCGGCA | | | |
| | EELLVFLFFFSL | ACAUCCCCGUGACCG | | | |
| | MGLLSSHLTSN | UGAUGGAGGCUAGU | | | |
| | STTDLPKAFHV | AUCUUCCGGCGGCCC | | | |
| | CAAILECLEKR | UACUACGUGUCCCAC | | | |
| | KISWLALFQLT | UUCCUGCCCGCCCUG | | | |
| | ESDLRLGRLLL | CUGACACCCCGGGUG | | | |
| | RVAPDQHTRLL | CUGCCCAAGGUGCCC | | | |
| | PFAFYSLLSYFH | GACAGUCGCGUCGCC | | | |
| | EDAAIREEAFL | UUCAUCGAGAGCCUG | | | |
| | HVAVDMYLKL | AAGCGGGCCGACAAG | | | |
| | VQLFVAGDTST | AUCCCGCCCUCCCUG | | | |
| | VSPPAGRSLEL | UACUCCACCUACUGC | | | |
| | KGQGNPVELIT | CAGGCUUGCUCCGCC | | | |
| | KARLFLLQLIPR | GCCGAGGAGAAGCCC | | | |
| | CPKKSFSHVAE | GAGGACGCAGCCUUA | | | |
| | LLADRGDCDPE | GGAGUACGCGCCGAG | | | |
| | VSAALQSRQQA | CCCAACAGCGCCGAG | | | |
| | APDADLSQEPH | GAGCCACUGGGCCAG | | | |
| | LF | UUGACCGCUGCCCUG | | | |
| | | GGAGAGCUGCGGGCC | | | |
| | | AGCAUGACCGACCCC | | | |
| | | AGCCAACGCGACGUG | | | |
| | | AUCAGCGCCCAGGUG | | | |
| | | GCCGUGAUCAGCGAG | | | |
| | | AGGCUGCGGGCAGU | | | |
| | | GCUGGGCCACAACGA | | | |
| | | GGACGACAGCAGCGU | | | |
| | | GGAGAUCAGCAAGA | | | |
| | | UCCAGCUGAGCAUCA | | | |
| | | ACACACCCAGGCUGG | | | |
| | | AGCCCAGAGAGCACA | | | |
| | | UGGCCGUGGACCUGC | | | |
| | | UGCUGACCUCCUUCU | | | |
| | | GCCAGAACCUGAUGG | | | |
| | | CCGCAAGCAGCGUGG | | | |
| | | CUCCUCCAGAACGCC | | | |
| | | AAGGCCCUUGGGCUG | | | |
| | | CCCUGUUCGUGCGCA | | | |
| | | CCAUGUGUGGCCGCG | | | |
| | | UCCUGCCAGCAGUGC | | | |
| | | UGACCCGGCUGUGCC | | | |
| | | AGCUUCUGCGGCACC | | | |
| | | AAGGGCCCUCCCUGA | | | |
| | | GCGCUCCCCACGUGC | | | |
| | | UUGGGUUGGCCGCCC | | | |
| | | UCGCUGUGCACCUGG | | | |
| | | GCGAGAGCAGGAGC | | | |
| | | GCCCUUCCCGAGGUG | | | |
| | | GACGUGGGUCCUCCA | | | |
| | | GCCCCAGGUGCCGGA | | | |
| | | CUCCCCGUGCCAGCC | | | |
| | | CUGUUCGACAGCCUG | | | |
| | | CUGACUUGUCGCACC | | | |
| | | CGGGACUCCCUGUUC | | | |
| | | UUCUGCCUGAAGUUC | | | |
| | | UGCACCGCCGCCAUC | | | |
| | | UCCUACUCCCUGUGC | | | |
| | | AAGUUCUCCAGCCAG | | | |
| | | UCCCGGGACACCCUG | | | |
| | | UGCUCCUGCCUGUCU | | | |
| | | CCCGGCCUGAUCAAG | | | |
| | | AAGUUCCAGUUCCUG | | | |
| | | AUGUUCCGCCUGUUC | | | |
| | | AGCGAAGCACGCCAG | | | |
| | | CCCCUGUCCGAGGAG | | | |
| | | GACGUGGCCUCCCUG | | | |
| | | UCCUGGCGACCCCUG | | | |
| | | CACCUGCCCUCCGCC | | | |
| | | GAUUGGCAGCGCGCC | | | |
| | | GCCCUUAGCCUGUGG | | | |
| | | ACCCACCGGACCUUC | | | |
| | | CGCGAGGUGCUGAA | | | |
| | | GGAGGAAGACGUGC | | | |
| | | ACCUGACCUACCAGG | | | |
| | | ACUGGCUGCACCUGG | | | |
| | | AGCUGGAGAUCCAGC | | | |
| | | CCGAAGCCGACGCCC | | | |
| | | UGAGCGACACCGAGC | | | |
| | | GCCAGGACUUUCACC | | | |
| | | AGUGGGCCAUCCACG | | | |
| | | AGCACUUUCUGCCCG | | | |
| | | AGAGCAGCGCCAGUG | | | |
| | | GAGGCUGCGACGGCG | | | |
| | | ACCUGCAAGCCGCCU | | | |
| | | GCACCAUCCUGGUGA | | | |
| | | ACGCCCUGAUGGACU | | | |
| | | UCCACCAGAGCUCCC | | | |
| | | GCAGCUACGACCACU | | | |
| | | CCGAGAACAGCGACC | | | |
| | | UGGUGUUUGGAGGC | | | |
| | | CGCACCGGGAACGAG | | | |
| | | GACAUCAUCAGCCGC | | | |
| | | CUGCAGGAGAUGGU | | | |
| | | GGCCGACCUGGAGCU | | | |
| | | CCAGCAGGACCUGAU | | | |
| | | CGUGCCACUGGGGCA | | | |
| | | CACCCCAAGCCAGGA | | | |
| | | GCACUUCCUGUUCGA | | | |
| | | GAUCUUCCGCAGACG | | | |
| | | CCUGCAGGCACUGAC | | | |
| | | CUCCGGCUGGAGUGU | | | |
| | | GGCCGCAAGCCUGCA | | | |
| | | GCGCCAACGCGAGCU | | | |
| | | GCUGAUGUACAAGC | | | |
| | | GGAUCCUGCUGCGCC | | | |
| | | UGCCCUCCUCCGUGC | | | |
| | | UGUGCGGCUCCUCCU | | | |
| | | UCCAGGCCGAGCAGC | | | |
| | | CCAUCACCGCCCGGU | | | |
| | | GCGAGCAGUUCUUCC | | | |
| | | ACCUGGUGAACAGCG | | | |
| | | AGAUGCGGAACUUC | | | |
| | | UGCUCUCACGGCGGC | | | |
| | | GCUCUGACCCAGGAC | | | |
| | | AUCACCGCCCACUUC | | | |
| | | UUCCGCGGCCUGCUG | | | |
| | | AACGCCUGCCUGCGG | | | |
| | | AGCAGGGACCCCAGC | | | |
| | | CUGAUGGUCGACUUC | | | |
| | | AUCCUGGCCAAGUGC | | | |
| | | CAGACCAAGUGUCCC | | | |
| | | CUGAUCCUGACCAGC | | | |
| | | GCCCUGGUUUGGUG | | | |
| | | GCCCUCACUGGAGCC | | | |
| | | CGUGCUCCUGUGUCG | | | |
| | | CUGGAGGCGGCACUG | | | |
| | | CCAGUCACCCCUGCC | | | |
| | | CCGAGAGCUGCAGAA | | | |
| | | GCUGCAGGAGGGCCG | | | |
| | | GCAGUUCGCCAGCGA | | | |
| | | CUUCCUGUCACCAGA | | | |
| | | GGCAGCCUCUCCCGC | | | |
| | | UCCUAACCCCGAUUG | | | |
| | | GCUAUCCGCGGCCGC | | | |
| | | UCUGCACUUCGCCAU | | | |
| | | CCAGCAGGUGCGGGA | | | |
| | | GGAGAACAUCCGGA | | | |
| | | AGCAGCUGAAGAAG | | | |
| | | CUGGACUGCGAGAG | | | |
| | | GGAGGAGCUGCUUG | | | |
| | | UGUUCCUGUUCUUCU | | | |
| | | UCAGCCUGAUGGGCC | | | |
| | | UGCUGUCCUCCCACC | | | |
| | | UGACCAGCAACUCCA | | | |
| | | CCACCGACCUGCCCA | | | |
| | | AGGCCUUCCACGUGU | | | |
| | | GCGCCGCCAUCCUGG | | | |
| | | AGUGCCUGGAGAAG | | | |
| | | CGGAAGAUCAGCUG | | | |
| | | GCUGGCCCUGUUCCA | | | |
| | | GCUGACCGAGAGCGA | | | |
| | | CCUGCGCCUGGGGAG | | | |
| | | ACUGCUGCUGCGCGU | | | |
| | | GGCUCCCGACCAGCA | | | |
| | | CACCAGGCUGCUGCC | | | |
| | | CUUCGCCUUCUACUC | | | |
| | | CCUGCUGUCCUACUU | | | |
| | | CCACGAGGACGCCGC | | | |
| | | CAUCCGGGAGGAGGC | | | |
| | | CUUCCUGCACGUGGC | | | |
| | | CGUGGACAUGUACCU | | | |
| | | GAAGCUGGUGCAGC | | | |
| | | UGUUCGUGGCCGGCG | | | |
| | | ACACCUCCACCGUGA | | | |
| | | GCCCUCCUGCUGGCC | | | |
| | | GAAGCCUGGAGCUG | | | |
| | | AAGGGCCAGGGCAAC | | | |
| | | CCCGUGGAGCUGAUC | | | |
| | | ACCAAGGCCCGCCUG | | | |
| | | UUCCUGCUGCAGCUG | | | |
| | | AUCCCUCGCUGCCCC | | | |
| | | AAGAAGAGCUUCAG | | | |
| | | CCACGUGGCCGAGCU | | | |
| | | GCUGGCUGACAGGG | | | |
| | | GAGACUGCGACCCCG | | | |
| | | AGGUGAGCGCUGCCC | | | |
| | | UGCAGAGCCGUCAAC | | | |
| | | AAGCGGCCCCUGACG | | | |
| | | CCGAUCUGAGCCAGG | | | |
| | | AGCCCCACCUGUUC | | | |
| | SEQ ID NO | | | | |
| | 1 | 3 | 50 | 139 | 5 |
| FANCA_02 | MSDSWVPNSA | AUGAGCGACAGCUG | GGAAA | UAAAGC | SEQ ID |
| Chemistry: G5 | SGQDPGGRRRA | GGUGCCUAAUAGCGC | UCGCAA | UCCCCG | NO: 5 |
| Cap: m7Gp- | WAELLAGRVK | CAGCGGACAGGAUCC | AAUUU | GGGGCC | consists |
| ppGm-A | REKYNPERAQK | UGGUGGAAGGAGAA | GCUCUU | UCGGUG | from 5′ to |
| PolyA tail: | LKESAVRLLRS | GGGCGUGGGCAGAG | CGCGUU | GCCUAG | 3′ end: 5′ |
| 100 nt (SEQ ID | HQDLNALLLEV | CUACUGGCUGGACGG | AGAUU | CUUCUU | UTR of |
| NO: 121) | EGPLCKKLSLS | GUGAAGCGAGAGAA | UCUUUU | GCCCCU | SEQ ID |
| | KVIDCDSSEAY | GUACAACCCCGAGCG | AGUUU | UGGGCC | NO: 50, |
| | ANHSSSFIGSAL | AGCACAGAAGCUGA | UCUCGC | UCCCCC | ORF |
| | QDQASRLGVPV | AGGAGUCAGCCGUG | AACUAG | CAGCCC | Sequence |
| | GILSAGMVASS | AGACUGCUGCGGAGC | CAAGCU | CUCCUC | of SEQ ID |
| | VGQICTAPAET | CACCAAGACCUGAAC | UUUUG | CCCUUC | NO: 3, and |
| | SHPVLLTVEQR | GCCUUACUGCUGGAA | UUCUCG | CUGCAC | 3′ UTR of |
| | KKLSSLLEFAQ | GUGGAGGGACCCCUG | CC | CCGUAC | SEQ ID |
| | YLLAHSMFSRL | UGCAAGAAGCUGAG | | CCCCGU | NO: 139 |
| | SFCQELWKIQS | CCUGAGCAAGGUGA | | GGUCUU | |
| | SLLLEAVWHLH | UCGACUGCGACAGCA | | UGAAU | |
| | VQGIVSLQELL | GCGAGGCCUACGCCA | | AAAGUC | |
| | ESHPDMHAVG | ACCACAGCAGCAGCU | | UGAGU | |
| | SWLFRNLCCLC | UCAUCGGCAGCGCAU | | GGGCGG | |
| | EQMEASCQHA | UGCAGGACCAAGCUU | | C | |
| | DVARAMLSDF | CUAGGCUAGGCGUCC | | | |
| | VQMFVLRGFQ | CCGUUGGUAUCCUGA | | | |
| | KNSDLRRTVEP | GCGCCGGUAUGGUA | | | |
| | EKMPQVTVDV | GCAAGCAGCGUGGGC | | | |
| | LQRMLIFALDA | CAGAUUUGUACCGCA | | | |
| | LAAGVQEESST | CCUGCCGAGACUAGC | | | |
| | HKIVRCWFGVF | CACCCCGUGCUUCUG | | | |
| | SGHTLGSVISTD | ACCGUGGAGCAGCGG | | | |
| | PLKRFFSHTLT | AAGAAGCUGAGCAG | | | |
| | QILTHSPVLKAS | CCUGCUGGAGUUCGC | | | |
| | DAVQMQREWS | CCAGUACCUGCUUGC | | | |
| | FARTHPLLTSL | GCACAGCAUGUUCAG | | | |
| | YRRLFVMLSAE | CCGGCUGAGCUUCUG | | | |
| | ELVGHLQEVLE | CCAGGAGCUGUGGA | | | |
| | TQEVHWQRVL | AGAUCCAGAGCAGCC | | | |
| | SFVSALVVCFP | UGCUGCUGGAAGCCG | | | |
| | EAQQLLEDWV | UGUGGCAUCUGCACG | | | |
| | ARLMAQAFESC | UGCAGGGCAUCGUG | | | |
| | QLDSMVTAFLV | AGCCUGCAGGAGCUG | | | |
| | VRQAALEGPSA | CUGGAAAGCCACCCA | | | |
| | FLSYADWFKAS | GACAUGCACGCCGUA | | | |
| | FGSTRGYHGCS | GGGAGUUGGCUGUU | | | |
| | KKALVFLFTFL | CCGGAACCUGUGCUG | | | |
| | SELVPFESPRYL | CCUGUGCGAGCAGAU | | | |
| | QVHILHPPLVP | GGAGGCCAGCUGUCA | | | |
| | GKYRSLLTDYI | ACACGCUGACGUAGC | | | |
| | SLAKTRLADLK | UCGCGCAAUGCUGAG | | | |
| | VSIENMGLYED | CGACUUCGUGCAGAU | | | |
| | LSSAGDITEPHS | GUUCGUGCUGCGGG | | | |
| | QALQDVEKAI | GCUUCCAGAAGAACA | | | |
| | MVFEHTGNIPV | GCGACCUCAGACGGA | | | |
| | TVMEASIFRRP | CCGUGGAACCCGAGA | | | |
| | YYVSHFLPALL | AGAUGCCACAGGUG | | | |
| | TPRVLPKVPDS | ACAGUGGACGUGCU | | | |
| | RVAFIESLKRA | GCAGCGGAUGCUGA | | | |
| | DKIPPSLYSTYC | UUUUCGCGUUAGAC | | | |
| | QACSAAEEKPE | GCACUAGCCGCAGGC | | | |
| | DAALGVRAEP | GUUCAGGAGGAGAG | | | |
| | NSAEEPLGQLT | CAGCACCCACAAGAU | | | |
| | AALGELRASMT | AGUGCGGUGCUGGU | | | |
| | DPSQRDVISAQ | UCGGCGUGUUCUCUG | | | |
| | VAVISERLRAV | GCCACACACUGGGGA | | | |
| | LGHNEDDSSVE | GCGUGAUCAGCACCG | | | |
| | ISKIQLSINTPRL | AUCCCCUGAAGCGGU | | | |
| | EPREHMAVDLL | UCUUCAGCCACACCC | | | |
| | LTSFCQNLMAA | UGACCCAGAUCCUGA | | | |
| | SSVAPPERQGP | CCCAUAGCCCCGUGC | | | |
| | WAALFVRTMC | UGAAGGCAAGCGAC | | | |
| | GRVLPAVLTRL | GCCGUGCAGAUGCAG | | | |
| | CQLLRHQGPSL | CGUGAGUGGAGCUU | | | |
| | SAPHVLGLAAL | CGCACGGACCCAUCC | | | |
| | AVHLGESRSAL | GCUACUGACCAGCCU | | | |
| | PEVDVGPPAPG | AUACCGGCGCCUGUU | | | |
| | AGLPVPALFDS | CGUGAUGCUAUCCGC | | | |
| | LLTCRTRDSLFF | CGAGGAGUUGGUCG | | | |
| | CLKFCTAAISYS | GACACCUGCAGGAGG | | | |
| | LCKFSSQSRDT | UGCUGGAGACCCAGG | | | |
| | LCSCLSPGLIKK | AGGUGCACUGGCAG | | | |
| | FQFLMFRLFSE | AGAGUGCUGAGCUU | | | |
| | ARQPLSEEDVA | CGUGAGCGCCCUGGU | | | |
| | SLSWRPLHLPS | GGUGUGCUUUCCCGA | | | |
| | ADWQRAALSL | GGCACAGCAGCUGCU | | | |
| | WTHRTFREVLK | GGAAGACUGGGUAG | | | |
| | EEDVHLTYQD | CUCGGCUUAUGGCCC | | | |
| | WLHLELEIQPE | AGGCCUUCGAGAGCU | | | |
| | ADALSDTERQD | GCCAGCUGGACAGCA | | | |
| | FHQWAIHEHFL | UGGUGACCGCCUUUC | | | |
| | PESSASGGCDG | UCGUGGUGAGGCAA | | | |
| | DLQAACTILVN | GCAGCCCUAGAAGGC | | | |
| | ALMDFHQSSRS | CCUAGCGCCUUUCUG | | | |
| | YDHSENSDLVF | AGCUACGCCGACUGG | | | |
| | GGRTGNEDIISR | UUCAAAGCCAGCUUC | | | |
| | LQEMVADLEL | GGCUCCACUCGGGGU | | | |
| | QQDLIVPLGHT | UACCACGGCUGCAGC | | | |
| | PSQEHFLFEIFR | AAGAAGGCCCUGGU | | | |
| | RRLQALTSGWS | GUUCCUGUUCACCUU | | | |
| | VAASLQRQREL | CCUGAGCGAGCUGGU | | | |
| | LMYKRILLRLP | GCCCUUCGAGUCACC | | | |
| | SSVLCGSSFQA | CCGGUACCUGCAGGU | | | |
| | EQPITARCEQFF | GCACAUCCUGCAUCC | | | |
| | HLVNSEMRNFC | ACCACUGGUCCCCGG | | | |
| | SHGGALTQDIT | GAAAUACCGGAGCCU | | | |
| | AHFFRGLLNAC | GCUGACCGACUACAU | | | |
| | LRSRDPSLMVD | CAGCCUGGCCAAGAC | | | |
| | FILAKCQTKCP | UCGGCUGGCAGACCU | | | |
| | LILTSALVWWP | GAAGGUGAGCAUCG | | | |
| | SLEPVLLCRWR | AGAACAUGGGCCUG | | | |
| | RHCQSPLPREL | UACGAGGAUCUGAG | | | |
| | QKLQEGRQFAS | CAGCGCUGGCGACAU | | | |
| | DFLSPEAASPAP | CACAGAACCGCACAG | | | |
| | NPDWLSAAAL | CCAGGCCUUGCAGGA | | | |
| | HFAIQQVREENI | CGUGGAGAAGGCCA | | | |
| | RKQLKKLDCER | UCAUGGUGUUCGAG | | | |
| | EELLVFLFFFSL | CACACCGGCAACAUC | | | |
| | MGLLSSHLTSN | CCCGUGACCGUGAUG | | | |
| | STTDLPKAFHV | GAGGCCAGCAUUUUC | | | |
| | CAAILECLEKR | CGCCGGCCAUACUAC | | | |
| | KISWLALFQLT | GUUAGCCACUUCCUG | | | |
| | ESDLRLGRLLL | CCCGCACUGCUGACU | | | |
| | RVAPDQHTRLL | CCACGCGUGCUGCCA | | | |
| | PFAFYSLLSYFH | AAGGUCCCCGAUAGC | | | |
| | EDAAIREEAFL | AGAGUGGCUUUCAU | | | |
| | HVAVDMYLKL | CGAGAGCCUGAAACG | | | |
| | VQLFVAGDTST | GGCCGACAAGAUCCC | | | |
| | VSPPAGRSLEL | ACCUAGCCUGUACAG | | | |
| | KGQGNPVELIT | CACCUACUGCCAAGC | | | |
| | KARLFLLQLIPR | UUGUAGCGCUGCCGA | | | |
| | CPKKSFSHVAE | GGAGAAACCGGAAG | | | |
| | LLADRGDCDPE | ACGCUGCACUGGGUG | | | |
| | VSAALQSRQQA | UUAGGGCCGAGCCCA | | | |
| | APDADLSQEPH | AUAGCGCAGAGGAA | | | |
| | LF | CCCUUGGGCCAACUG | | | |
| | | ACUGCUGCACUUGGU | | | |
| | | GAGCUUCGGGCAAGC | | | |
| | | AUGACCGACCCCUCU | | | |
| | | CAACGGGACGUGAUC | | | |
| | | AGCGCCCAAGUGGCC | | | |
| | | GUGAUCAGCGAGCG | | | |
| | | GUUACGUGCCGUGCU | | | |
| | | GGGCCACAACGAGGA | | | |
| | | CGACAGCAGCGUGGA | | | |
| | | GAUCAGCAAGAUCCA | | | |
| | | GCUGAGCAUCAAUAC | | | |
| | | GCCUCGACUGGAACC | | | |
| | | UCGGGAGCACAUGGC | | | |
| | | CGUGGACCUGCUGCU | | | |
| | | GACCAGCUUCUGCCA | | | |
| | | GAACUUGAUGGCCGC | | | |
| | | UAGCAGUGUGGCUCC | | | |
| | | ACCCGAACGGCAAGG | | | |
| | | ACCUUGGGCUGCGCU | | | |
| | | GUUCGUCAGGACCAU | | | |
| | | GUGUGGCCGGGUUC | | | |
| | | UUCCCGCUGUGUUGA | | | |
| | | CCCGGCUUUGCCAGC | | | |
| | | UGCUGCGGCAUCAAG | | | |
| | | GGCCAUCUUUGUCCG | | | |
| | | CACCUCACGUUCUGG | | | |
| | | GUCUUGCUGCUCUGG | | | |
| | | CCGUGCAUCUGGGCG | | | |
| | | AGAGCAGAUCGGCAC | | | |
| | | UGCCGGAAGUGGAC | | | |
| | | GUGGGACCUCCUGCA | | | |
| | | CCUGGUGCAGGUCUU | | | |
| | | CCCGUGCCAGCCUUA | | | |
| | | UUCGACAGCCUGCUU | | | |
| | | ACCUGCCGUACUCGG | | | |
| | | GACAGCCUGUUCUUC | | | |
| | | UGCCUGAAGUUCUGC | | | |
| | | ACCGCCGCCAUCAGC | | | |
| | | UACAGCCUGUGCAAG | | | |
| | | UUCAGCAGCCAGAGC | | | |
| | | CGGGACACUCUGUGU | | | |
| | | AGCUGUCUGAGCCCU | | | |
| | | GGCCUCAUCAAGAAG | | | |
| | | UUCCAGUUCCUGAUG | | | |
| | | UUCAGAUUAUUUAG | | | |
| | | CGAGGCUCGGCAACC | | | |
| | | UCUGAGCGAGGAGG | | | |
| | | ACGUUGCCAGCCUGA | | | |
| | | GUUGGCGUCCCCUAC | | | |
| | | ACCUUCCCAGCGCCG | | | |
| | | AUUGGCAGAGGGCU | | | |
| | | GCUCUGUCCCUGUGG | | | |
| | | ACUCACCGCACUUUC | | | |
| | | CGGGAGGUGCUGAA | | | |
| | | GGAGGAGGACGUGC | | | |
| | | ACCUGACCUACCAGG | | | |
| | | ACUGGCUGCACCUGG | | | |
| | | AGCUCGAGAUACAGC | | | |
| | | CCGAAGCUGACGCCC | | | |
| | | UGUCAGACACCGAGC | | | |
| | | GGCAGGACUUCCACC | | | |
| | | AGUGGGCCAUCCACG | | | |
| | | AGCACUUCCUGCCUG | | | |
| | | AGAGCAGCGCCUCAG | | | |
| | | GAGGGUGUGACGGG | | | |
| | | GACCUGCAAGCAGCC | | | |
| | | UGCACCAUCCUGGUG | | | |
| | | AACGCCCUGAUGGAC | | | |
| | | UUCCACCAGAGCAGC | | | |
| | | CGGAGCUACGACCAC | | | |
| | | AGCGAGAACAGCGA | | | |
| | | UCUAGUCUUCGGCGG | | | |
| | | CAGAACAGGUAACG | | | |
| | | AGGACAUCAUCAGCC | | | |
| | | GGCUGCAGGAGAUG | | | |
| | | GUGGCCGACCUGGAA | | | |
| | | CUGCAGCAGGACCUG | | | |
| | | AUUGUCCCCUUGGGU | | | |
| | | CACACCCCUAGCCAG | | | |
| | | GAGCACUUCCUGUUC | | | |
| | | GAGAUUUUCAGGAG | | | |
| | | GCGGCUGCAAGCACU | | | |
| | | GACCAGCGGCUGGUC | | | |
| | | UGUCGCUGCUUCCCU | | | |
| | | UCAGCGUCAGCGGGA | | | |
| | | GCUGCUGAUGUACA | | | |
| | | AGCGGAUUCUGCUGC | | | |
| | | GGCUACCCAGCAGUG | | | |
| | | UGCUGUGCGGCUCUA | | | |
| | | GCUUUCAAGCCGAGC | | | |
| | | AGCCAAUCACCGCUC | | | |
| | | GCUGCGAGCAGUUCU | | | |
| | | UCCACCUGGUGAACA | | | |
| | | GCGAGAUGCGGAAU | | | |
| | | UUCUGUUCCCACGGA | | | |
| | | GGUGCUCUGACCCAG | | | |
| | | GACAUCACCGCCCAU | | | |
| | | UUCUUCCGGGGCCUG | | | |
| | | CUGAACGCUUGUCUC | | | |
| | | CGAUCGAGAGACCCC | | | |
| | | AGCCUGAUGGUGGA | | | |
| | | CUUCAUCCUGGCCAA | | | |
| | | GUGCCAGACCAAGUG | | | |
| | | UCCCCUGAUCCUGAC | | | |
| | | CUCUGCCCUGGUGUG | | | |
| | | GUGGCCUUCACUGGA | | | |
| | | GCCCGUGUUGCUGUG | | | |
| | | UCGCUGGCGGAGACA | | | |
| | | UUGUCAAUCACCACU | | | |
| | | GCCACGGGAGCUGCA | | | |
| | | GAAGCUGCAAGAGG | | | |
| | | GUCGGCAAUUCGCCU | | | |
| | | CGGACUUUCUGUCUC | | | |
| | | CUGAAGCUGCUAGCC | | | |
| | | CUGCCCCUAAUCCUG | | | |
| | | ACUGGUUGUCUGCG | | | |
| | | GCCGCACUACACUUU | | | |
| | | GCCAUCCAGCAGGUG | | | |
| | | CGGGAGGAGAACAU | | | |
| | | CCGGAAGCAGCUGAA | | | |
| | | GAAACUGGACUGCG | | | |
| | | AGCGGGAAGAGCUG | | | |
| | | CUGGUGUUCCUGUUC | | | |
| | | UUCUUCAGCCUGAUG | | | |
| | | GGCCUGCUGAGCUCA | | | |
| | | CACCUGACCAGCAAC | | | |
| | | AGCACUACCGACCUG | | | |
| | | CCCAAAGCCUUCCAC | | | |
| | | GUGUGCGCUGCCAUC | | | |
| | | CUGGAGUGCCUGGA | | | |
| | | GAAGCGGAAGAUCA | | | |
| | | GCUGGCUGGCCCUGU | | | |
| | | UCCAGCUGACAGAGU | | | |
| | | CUGAUCUUCGGCUGG | | | |
| | | GGCGGUUACUCCUGA | | | |
| | | GAGUAGCACCCGAUC | | | |
| | | AGCAUACCAGGCUGC | | | |
| | | UCCCCUUCGCAUUCU | | | |
| | | ACAGCCUGCUGAGCU | | | |
| | | ACUUCCACGAAGACG | | | |
| | | CCGCCAUACGCGAGG | | | |
| | | AAGCUUUCCUGCACG | | | |
| | | UGGCCGUGGACAUG | | | |
| | | UACCUGAAGCUGGU | | | |
| | | GCAGCUGUUCGUUGC | | | |
| | | UGGCGACACCAGCAC | | | |
| | | CGUGUCACCUCCUGC | | | |
| | | AGGCAGGAGCCUGG | | | |
| | | AGCUGAAAGGUCAG | | | |
| | | GGCAACCCCGUGGAG | | | |
| | | CUGAUCACCAAGGCC | | | |
| | | CGGCUGUUCCUGCUU | | | |
| | | CAGCUGAUACCCCGG | | | |
| | | UGCCCCAAGAAGAGC | | | |
| | | UUUAGCCACGUGGCC | | | |
| | | GAACUGCUGGCAGAC | | | |
| | | CGGGGUGAUUGCGA | | | |
| | | CCCAGAAGUGUCAGC | | | |
| | | CGCCCUUCAAAGCCG | | | |
| | | GCAACAAGCUGCCCC | | | |
| | | UGACGCCGACUUGAG | | | |
| | | CCAGGAGCCGCACCU | | | |
| | | GUUC | | | |
| | SEQ ID NO: | | | | |
| | 1 | 14 | 50 | 139 | 23 |
| FANCA_03 | MSDSWVPNSA | AUGAGCGACAGCUG | GGAAA | UAAAGC | SEQ ID |
| Chemistry: G5 | SGQDPGGRRRA | GGUGCCCAACAGUGC | UCGCAA | UCCCCG | NO: 23 |
| Cap: C1 | WAELLAGRVK | UUCCGGGCAGGAUCC | AAUUU | GGGGCC | consists |
| PolyA tail: | REKYNPERAQK | CGGCGGUAGAAGAC | GCUCUU | UCGGUG | from 5′ to |
| 100 nt (SEQ ID | LKESAVRLLRS | GGGCUUGGGCUGAG | CGCGUU | GCCUAG | 3′ end: 5′ |
| NO: 121) | HQDLNALLLEV | CUGCUGGCCGGCAGG | AGAUU | CUUCUU | UTR of |
| | EGPLCKKLSLS | GUGAAGCGGGAGAA | UCUUUU | GCCCCU | SEQ ID |
| | KVIDCDSSEAY | GUACAACCCCGAGCG | AGUUU | UGGGCC | NO: 50, |
| | ANHSSSFIGSAL | GGCCCAGAAGCUGAA | UCUCGC | UCCCCC | ORF |
| | QDQASRLGVPV | GGAGAGUGCCGUGC | AACUAG | CAGCCC | Sequence |
| | GILSAGMVASS | GGCUGCUGCGGAGCC | CAAGCU | CUCCUC | of SEQ ID |
| | VGQICTAPAET | ACCAGGACCUGAACG | UUUUG | CCCUUC | NO: 14, |
| | SHPVLLTVEQR | CCCUCCUGCUGGAGG | UUCUCG | CUGCAC | and 3′ |
| | KKLSSLLEFAQ | UCGAGGGCCCACUGU | CC | CCGUAC | UTR of |
| | YLLAHSMFSRL | GCAAGAAGCUCAGCC | | CCCCGU | SEQ ID |
| | SFCQELWKIQS | UGAGCAAGGUGAUC | | GGUCUU | NO: 139 |
| | SLLLEAVWHLH | GACUGUGACAGCAGC | | UGAAU | |
| | VQGIVSLQELL | GAGGCCUACGCCAAC | | AAAGUC | |
| | ESHPDMHAVG | CACAGCUCCAGCUUC | | UGAGU | |
| | SWLFRNLCCLC | AUCGGCAGCGCCCUG | | GGGCGG | |
| | EQMEASCQHA | CAGGACCAGGCCAGC | | C | |
| | DVARAMLSDF | AGACUGGGCGUGCCC | | | |
| | VQMFVLRGFQ | GUGGGCAUCCUGAGC | | | |
| | KNSDLRRTVEP | GCCGGCAUGGUGGCC | | | |
| | EKMPQVTVDV | AGCUCGGUGGGCCAG | | | |
| | LQRMLIFALDA | AUCUGCACCGCUCCC | | | |
| | LAAGVQEESST | GCCGAGACCAGCCAC | | | |
| | HKIVRCWFGVF | CCCGUGCUGCUCACC | | | |
| | SGHTLGSVISTD | GUGGAGCAGCGGAA | | | |
| | PLKRFFSHTLT | GAAGCUGAGCAGCCU | | | |
| | QILTHSPVLKAS | GCUGGAGUUCGCCCA | | | |
| | DAVQMQREWS | GUACCUGCUGGCCCA | | | |
| | FARTHPLLTSL | CAGCAUGUUCUCCCG | | | |
| | YRRLFVMLSAE | GCUGAGCUUUUGCCA | | | |
| | ELVGHLQEVLE | AGAGCUGUGGAAGA | | | |
| | TQEVHWQRVL | UCCAGAGCUCCCUGC | | | |
| | SFVSALVVCFP | UGCUGGAGGCCGUG | | | |
| | EAQQLLEDWV | UGGCACCUUCACGUG | | | |
| | ARLMAQAFESC | CAGGGCAUCGUGAGC | | | |
| | QLDSMVTAFLV | CUGCAGGAACUGCUG | | | |
| | VRQAALEGPSA | GAGAGCCACCCCGAC | | | |
| | FLSYADWFKAS | AUGCACGCCGUGGGC | | | |
| | FGSTRGYHGCS | AGCUGGCUGUUCCGG | | | |
| | KKALVFLFTFL | AACCUGUGCUGCCUG | | | |
| | SELVPFESPRYL | UGCGAGCAGAUGGA | | | |
| | QVHILHPPLVP | GGCUAGCUGCCAGCA | | | |
| | GKYRSLLTDYI | CGCCGACGUGGCACG | | | |
| | SLAKTRLADLK | GGCCAUGCUGAGCGA | | | |
| | VSIENMGLYED | CUUCGUGCAGAUGU | | | |
| | LSSAGDITEPHS | UCGUGCUGCGGGGCU | | | |
| | QALQDVEKAI | UCCAGAAGAAUAGC | | | |
| | MVFEHTGNIPV | GACCUACGGCGGACC | | | |
| | TVMEASIFRRP | GUGGAGCCCGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UCGCCCUGGACGCUC | | | |
| | QACSAAEEKPE | UGGCCGCUGGCGUGC | | | |
| | DAALGVRAEP | AGGAGGAGAGCAGC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCAGCGGCCAC | | | |
| | VAVISERLRAV | ACCCUGGGCAGCGUG | | | |
| | LGHNEDDSSVE | AUCAGCACCGACCCA | | | |
| | ISKIQLSINTPRL | CUGAAGCGGUUCUUC | | | |
| | EPREHMAVDLL | AGCCACACCCUCACC | | | |
| | LTSFCQNLMAA | CAGAUCCUGACCCAC | | | |
| | SSVAPPERQGP | AGCCCCGUGCUGAAG | | | |
| | WAALFVRTMC | GCCAGCGACGCCGUG | | | |
| | GRVLPAVLTRL | CAGAUGCAGCGGGA | | | |
| | CQLLRHQGPSL | GUGGAGCUUCGCCCG | | | |
| | SAPHVLGLAAL | GACCCACCCACUGCU | | | |
| | AVHLGESRSAL | GACUAGCCUGUACCG | | | |
| | PEVDVGPPAPG | GCGGCUGUUCGUGA | | | |
| | AGLPVPALFDS | UGCUGAGCGCAGAG | | | |
| | LLTCRTRDSLFF | GAGCUGGUGGGCCAC | | | |
| | CLKFCTAAISYS | CUGCAGGAGGUGCU | | | |
| | LCKFSSQSRDT | GGAGACCCAGGAGG | | | |
| | LCSCLSPGLIKK | UGCACUGGCAGCGGG | | | |
| | FQFLMFRLFSE | UGCUGAGCUUCGUG | | | |
| | ARQPLSEEDVA | AGCGCCCUGGUGGUG | | | |
| | SLSWRPLHLPS | UGCUUCCCCGAGGCC | | | |
| | ADWQRAALSL | CAGCAGCUCCUGGAG | | | |
| | WTHRTFREVLK | GACUGGGUGGCCCGG | | | |
| | EEDVHLTYQD | CUGAUGGCCCAGGCC | | | |
| | WLHLELEIQPE | UUCGAGAGCUGCCAG | | | |
| | ADALSDTERQD | CUGGACAGCAUGGU | | | |
| | FHQWAIHEHFL | GACCGCCUUCCUGGU | | | |
| | PESSASGGCDG | GGUGCGGCAGGCUGC | | | |
| | DLQAACTILVN | CCUGGAAGGCCCCAG | | | |
| | ALMDFHQSSRS | CGCUUUCCUGAGCUA | | | |
| | YDHSENSDLVF | CGCCGACUGGUUCAA | | | |
| | GGRTGNEDIISR | GGCCAGCUUCGGCUC | | | |
| | LQEMVADLEL | CACCCGGGGCUACCA | | | |
| | QQDLIVPLGHT | CGGCUGCAGCAAGAA | | | |
| | PSQEHFLFEIFR | GGCCCUGGUGUUCCU | | | |
| | RRLQALTSGWS | GUUCACCUUCCUGAG | | | |
| | VAASLQRQREL | CGAGCUGGUGCCCUU | | | |
| | LMYKRILLRLP | CGAGUCACCCCGGUA | | | |
| | SSVLCGSSFQA | CCUGCAGGUGCACAU | | | |
| | EQPITARCEQFF | CCUGCAUCCUCCGCU | | | |
| | HLVNSEMRNFC | GGUGCCCGGCAAGUA | | | |
| | SHGGALTQDIT | CCGGAGCCUGCUGAC | | | |
| | AHFFRGLLNAC | CGACUACAUCAGCCU | | | |
| | LRSRDPSLMVD | GGCCAAGACCCGGCU | | | |
| | FILAKCQTKCP | GGCCGACCUGAAGGU | | | |
| | LILTSALVWWP | GAGCAUCGAGAACA | | | |
| | SLEPVLLCRWR | UGGGCCUGUACGAG | | | |
| | RHCQSPLPREL | GACCUGAGCAGCGCC | | | |
| | QKLQEGRQFAS | GGCGACAUCACCGAG | | | |
| | DFLSPEAASPAP | CCCCACAGCCAGGCC | | | |
| | NPDWLSAAAL | CUGCAAGACGUGGA | | | |
| | HFAIQQVREENI | GAAGGCCAUCAUGG | | | |
| | RKQLKKLDCER | UGUUCGAGCACACCG | | | |
| | EELLVFLFFFSL | GCAACAUCCCCGUGA | | | |
| | MGLLSSHLTSN | CCGUCAUGGAGGCCA | | | |
| | STTDLPKAFHV | GCAUCUUCCGGCGGC | | | |
| | CAAILECLEKR | CCUACUACGUGAGCC | | | |
| | KISWLALFQLT | ACUUCCUGCCCGCCC | | | |
| | ESDLRLGRLLL | UGCUGACCCCACGGG | | | |
| | RVAPDQHTRLL | UGCUGCCCAAGGUGC | | | |
| | PFAFYSLLSYFH | CCGACAGCCGGGUGG | | | |
| | EDAAIREEAFL | CCUUCAUCGAGAGCC | | | |
| | HVAVDMYLKL | UGAAGCGGGCCGACA | | | |
| | VQLFVAGDTST | AGAUCCCACCCAGCC | | | |
| | VSPPAGRSLEL | UGUACAGCACCUACU | | | |
| | KGQGNPVELIT | GCCAGGCCUGCUCUG | | | |
| | KARLFLLQLIPR | CCGCCGAGGAGAAGC | | | |
| | CPKKSFSHVAE | CCGAGGACGCCGCAU | | | |
| | LLADRGDCDPE | UAGGCGUGCGGGCCG | | | |
| | VSAALQSRQQA | AGCCCAACAGCGCCG | | | |
| | APDADLSQEPH | AGGAACCCCUGGGGC | | | |
| | LF | AGCUGACCGCAGCCC | | | |
| | | UGGGAGAGCUGCGG | | | |
| | | GCCAGCAUGACCGAC | | | |
| | | CCCAGCCAGCGGGAC | | | |
| | | GUGAUCAGCGCCCAG | | | |
| | | GUGGCCGUCAUCAGC | | | |
| | | GAGCGGCUGCGGGCA | | | |
| | | GUGCUGGGCCACAAC | | | |
| | | GAGGACGACAGCAGC | | | |
| | | GUGGAGAUCAGCAA | | | |
| | | GAUCCAGCUGAGCAU | | | |
| | | CAACACCCCACGGCU | | | |
| | | GGAACCUCGGGAGCA | | | |
| | | CAUGGCCGUGGACCU | | | |
| | | GCUGCUGACCAGCUU | | | |
| | | CUGCCAGAACCUGAU | | | |
| | | GGCCGCCUCUAGCGU | | | |
| | | GGCCCCACCAGAACG | | | |
| | | GCAGGGUCCCUGGGC | | | |
| | | UGCCCUGUUCGUGCG | | | |
| | | GACCAUGUGCGGCCG | | | |
| | | GGUUCUGCCCGCUGU | | | |
| | | GCUGACCCGGCUGUG | | | |
| | | CCAGCUGCUGCGGCA | | | |
| | | CCAGGGCCCUAGCCU | | | |
| | | GAGCGCUCCCCACGU | | | |
| | | GCUGGGCCUAGCUGC | | | |
| | | CCUGGCCGUGCACCU | | | |
| | | UGGGGAGUCCCGAA | | | |
| | | GCGCCCUGCCCGAGG | | | |
| | | UGGACGUGGGCCCUC | | | |
| | | CAGCCCCAGGUGCUG | | | |
| | | GCCUUCCCGUGCCCG | | | |
| | | CUCUGUUCGACAGUC | | | |
| | | UGCUGACCUGCCGGA | | | |
| | | CCCGGGACAGCCUGU | | | |
| | | UCUUCUGCCUGAAGU | | | |
| | | UCUGCACCGCCGCCA | | | |
| | | UCAGCUACAGCCUGU | | | |
| | | GCAAGUUCAGCAGCC | | | |
| | | AGAGCCGGGACACCC | | | |
| | | UGUGCAGCUGCCUGA | | | |
| | | GCCCCGGCCUGAUCA | | | |
| | | AGAAGUUCCAGUUCC | | | |
| | | UGAUGUUCCGGCUG | | | |
| | | UUCAGCGAAGCCCGG | | | |
| | | CAGCCCCUGAGCGAG | | | |
| | | GAGGACGUCGCCAGC | | | |
| | | CUGUCCUGGAGACCC | | | |
| | | CUGCACCUGCCCAGC | | | |
| | | GCCGACUGGCAGAGA | | | |
| | | GCCGCCCUGAGCCUG | | | |
| | | UGGACCCACCGGACC | | | |
| | | UUCCGGGAGGUGCU | | | |
| | | GAAGGAAGAGGACG | | | |
| | | UGCACCUGACCUACC | | | |
| | | AGGACUGGCUGCACC | | | |
| | | UGGAGCUGGAGAUU | | | |
| | | CAGCCCGAGGCCGAC | | | |
| | | GCCCUGAGCGACACC | | | |
| | | GAGCGGCAGGAUUU | | | |
| | | CCACCAGUGGGCCAU | | | |
| | | CCACGAGCAUUUCCU | | | |
| | | GCCCGAGAGCAGCGC | | | |
| | | CAGCGGCGGCUGUGA | | | |
| | | CGGCGACCUGCAGGC | | | |
| | | CGCCUGCACCAUCCU | | | |
| | | GGUGAACGCCCUGAU | | | |
| | | GGACUUCCACCAGAG | | | |
| | | CAGCCGGAGCUACGA | | | |
| | | CCACAGCGAGAACAG | | | |
| | | CGACCUGGUGUUCGG | | | |
| | | CGGACGGACCGGCAA | | | |
| | | CGAGGACAUCAUCAG | | | |
| | | CCGGCUGCAGGAGAU | | | |
| | | GGUCGCCGACCUGGA | | | |
| | | GUUGCAGCAGGACCU | | | |
| | | GAUCGUGCCCCUGGG | | | |
| | | CCACACUCCCAGCCA | | | |
| | | GGAGCACUUCCUGUU | | | |
| | | CGAGAUCUUCCGCCG | | | |
| | | GAGACUGCAGGCCCU | | | |
| | | GACCUCAGGCUGGAG | | | |
| | | CGUGGCCGCCAGCCU | | | |
| | | GCAACGGCAGCGGGA | | | |
| | | GCUGCUGAUGUACA | | | |
| | | AGCGGAUCCUGCUGC | | | |
| | | GGCUGCCCAGCAGCG | | | |
| | | UGCUGUGCGGCAGCA | | | |
| | | GCUUCCAGGCCGAGC | | | |
| | | AGCCCAUCACGGCCC | | | |
| | | GGUGCGAGCAGUUC | | | |
| | | UUCCACCUGGUGAAC | | | |
| | | AGCGAGAUGCGGAA | | | |
| | | CUUCUGCAGCCACGG | | | |
| | | AGGCGCCCUGACCCA | | | |
| | | GGACAUCACCGCCCA | | | |
| | | CUUCUUCCGGGGCCU | | | |
| | | GCUGAACGCCUGCCU | | | |
| | | GCGGAGCAGAGACCC | | | |
| | | CAGCCUGAUGGUGG | | | |
| | | ACUUCAUCCUGGCCA | | | |
| | | AGUGCCAGACCAAGU | | | |
| | | GUCCCCUGAUCCUGA | | | |
| | | CCUCCGCCCUGGUGU | | | |
| | | GGUGGCCCAGCCUGG | | | |
| | | AGCCCGUGCUUCUGU | | | |
| | | GUCGGUGGCGGCGGC | | | |
| | | ACUGCCAGAGCCCUC | | | |
| | | UGCCCCGGGAGCUGC | | | |
| | | AGAAGCUGCAGGAG | | | |
| | | GGCCGGCAGUUCGCC | | | |
| | | AGCGACUUCCUGUCU | | | |
| | | CCCGAGGCCGCUAGC | | | |
| | | CCAGCCCCAAACCCC | | | |
| | | GACUGGCUGAGCGCU | | | |
| | | GCCGCCCUGCACUUC | | | |
| | | GCCAUCCAGCAGGUG | | | |
| | | CGGGAGGAGAACAU | | | |
| | | CCGGAAGCAGCUGAA | | | |
| | | GAAGCUGGACUGCG | | | |
| | | AGCGGGAGGAGCUG | | | |
| | | CUCGUGUUCCUCUUC | | | |
| | | UUCUUCAGCCUCAUG | | | |
| | | GGCCUUCUGAGCAGC | | | |
| | | CACCUGACCAGCAAC | | | |
| | | AGCACCACCGACCUG | | | |
| | | CCCAAGGCCUUCCAC | | | |
| | | GUGUGCGCCGCCAUC | | | |
| | | CUGGAGUGCCUGGA | | | |
| | | GAAGCGGAAGAUCA | | | |
| | | GCUGGCUGGCCCUGU | | | |
| | | UCCAGCUGACCGAGA | | | |
| | | GCGACCUGCGGCUGG | | | |
| | | GCCGACUGCUGCUGC | | | |
| | | GGGUGGCCCCAGACC | | | |
| | | AGCACACCCGGCUGC | | | |
| | | UGCCCUUCGCCUUCU | | | |
| | | ACAGCCUGCUGAGCU | | | |
| | | ACUUCCACGAGGACG | | | |
| | | CUGCCAUCCGGGAGG | | | |
| | | AGGCCUUUCUGCACG | | | |
| | | UGGCAGUGGACAUG | | | |
| | | UACCUGAAGCUGGU | | | |
| | | GCAGCUGUUCGUGGC | | | |
| | | CGGCGACACCAGCAC | | | |
| | | CGUGUCACCACCAGC | | | |
| | | CGGCCGCUCCCUGGA | | | |
| | | GCUGAAGGGCCAGG | | | |
| | | GCAACCCCGUGGAGC | | | |
| | | UGAUCACCAAGGCCC | | | |
| | | GGCUGUUCCUGCUGC | | | |
| | | AGCUGAUCCCACGGU | | | |
| | | GCCCCAAGAAGAGCU | | | |
| | | UCAGCCACGUGGCCG | | | |
| | | AGCUGCUUGCCGACC | | | |
| | | GGGGAGACUGCGACC | | | |
| | | CCGAGGUGAGCGCCG | | | |
| | | CCUUGCAGAGCCGGC | | | |
| | | AGCAGGCCGCACCUG | | | |
| | | ACGCCGACCUGAGCC | | | |
| | | AGGAGCCCCACCUGU | | | |
| | | UC | | | |
| | SEQ ID NO: | | | | |
| | 1 | 15 | 50 | 139 | 24 |
| FANCA_04 | MSDSWVPNSA | AUGAGCGACAGCUG | GGAAA | UAAAGC | SEQ ID |
| Chemistry: G5 | SGQDPGGRRRA | GGUGCCCAACAGCGC | UCGCAA | UCCCCG | NO: 24 |
| Cap: C1 | WAELLAGRVK | CAGUGGCCAAGACCC | AAUUU | GGGGCC | consists |
| PolyA tail: | REKYNPERAQK | CGGCGGUCGGAGACG | GCUCUU | UCGGUG | from 5′ to |
| 100 nt (SEQ ID | LKESAVRLLRS | AGCGUGGGCUGAGC | CGCGUU | GCCUAG | 3′ end: 5′ |
| NO: 121) | HQDLNALLLEV | UGCUGGCCGGACGGG | AGAUU | CUUCUU | UTR of |
| | EGPLCKKLSLS | UGAAGCGGGAGAAG | UCUUUU | GCCCCU | SEQ ID |
| | KVIDCDSSEAY | UACAACCCCGAGCGG | AGUUU | UGGGCC | NO: 50, |
| | ANHSSSFIGSAL | GCCCAGAAGCUGAAG | UCUCGC | UCCCCC | ORF |
| | QDQASRLGVPV | GAGAGCGCUGUGCG | AACUAG | CAGCCC | Sequence |
| | GILSAGMVASS | GCUGCUGCGGAGCCA | CAAGCU | CUCCUC | of SEQ ID |
| | VGQICTAPAET | CCAGGACCUGAACGC | UUUUG | CCCUUC | NO: 15, |
| | SHPVLLTVEQR | CCUGCUGCUGGAGGU | UUCUCG | CUGCAC | and 3′ |
| | KKLSSLLEFAQ | GGAGGGCCCACUGUG | CC | CCGUAC | UTR of |
| | YLLAHSMFSRL | CAAGAAGCUGAGCCU | | CCCCGU | SEQ ID |
| | SFCQELWKIQS | GAGCAAGGUGAUCG | | GGUCUU | NO: 139 |
| | SLLLEAVWHLH | ACUGCGACAGCAGCG | | UGAAU | |
| | VQGIVSLQELL | AGGCCUACGCCAACC | | AAAGUC | |
| | ESHPDMHAVG | ACAGCAGCAGCUUCA | | UGAGU | |
| | SWLFRNLCCLC | UCGGCAGCGCCCUGC | | GGGCGG | |
| | EQMEASCQHA | AGGACCAGGCCAGCA | | C | |
| | DVARAMLSDF | GACUGGGCGUGCCCG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCCCCGG | | | |
| | LAAGVQEESST | CCGAGACAAGCCACC | | | |
| | HKIVRCWFGVF | CCGUGCUGCUGACCG | | | |
| | SGHTLGSVISTD | UGGAGCAGCGGAAG | | | |
| | PLKRFFSHTLT | AAGCUGAGCAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCAGCCGG | | | |
| | YRRLFVMLSAE | CUGAGCUUCUGCCAG | | | |
| | ELVGHLQEVLE | GAGCUGUGGAAGAU | | | |
| | TQEVHWQRVL | CCAGAGCAGCCUGCU | | | |
| | SFVSALVVCFP | GCUGGAGGCCGUGU | | | |
| | EAQQLLEDWV | GGCACCUGCACGUGC | | | |
| | ARLMAQAFESC | AGGGCAUCGUGAGCC | | | |
| | QLDSMVTAFLV | UGCAGGAGCUGCUG | | | |
| | VRQAALEGPSA | GAGAGCCACCCCGAC | | | |
| | FLSYADWFKAS | AUGCACGCCGUGGGC | | | |
| | FGSTRGYHGCS | AGCUGGCUGUUCCGG | | | |
| | KKALVFLFTFL | AACCUGUGCUGCCUG | | | |
| | SELVPFESPRYL | UGCGAGCAGAUGGA | | | |
| | QVHILHPPLVP | GGCCAGCUGCCAGCA | | | |
| | GKYRSLLTDYI | CGCCGACGUGGCUCG | | | |
| | SLAKTRLADLK | GGCCAUGCUGAGCGA | | | |
| | VSIENMGLYED | CUUCGUGCAGAUGU | | | |
| | LSSAGDITEPHS | UCGUGCUGCGGGGCU | | | |
| | QALQDVEKAI | UCCAGAAGAACAGCG | | | |
| | MVFEHTGNIPV | ACCUGCGGCGGACCG | | | |
| | TVMEASIFRRP | UAGAGCCCGAGAAG | | | |
| | YYVSHFLPALL | AUGCCCCAGGUGACC | | | |
| | TPRVLPKVPDS | GUGGACGUGCUGCA | | | |
| | RVAFIESLKRA | GCGGAUGCUGAUCU | | | |
| | DKIPPSLYSTYC | UCGCACUCGACGCCC | | | |
| | QACSAAEEKPE | UGGCCGCUGGUGUGC | | | |
| | DAALGVRAEP | AGGAGGAGAGCAGC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCAGCGGCCAC | | | |
| | VAVISERLRAV | ACCCUGGGCAGCGUG | | | |
| | LGHNEDDSSVE | AUCAGCACCGAUCCC | | | |
| | ISKIQLSINTPRL | CUGAAGCGGUUCUUC | | | |
| | EPREHMAVDLL | AGCCACACCCUGACC | | | |
| | LTSFCQNLMAA | CAGAUCCUGACCCAC | | | |
| | SSVAPPERQGP | AGCCCCGUGCUGAAG | | | |
| | WAALFVRTMC | GCCAGCGACGCCGUG | | | |
| | GRVLPAVLTRL | CAGAUGCAGCGGGA | | | |
| | CQLLRHQGPSL | GUGGAGCUUCGCCCG | | | |
| | SAPHVLGLAAL | GACCCAUCCCCUGCU | | | |
| | AVHLGESRSAL | GACCAGCCUGUACCG | | | |
| | PEVDVGPPAPG | GCGGCUGUUCGUGA | | | |
| | AGLPVPALFDS | UGCUGAGCGCCGAGG | | | |
| | LLTCRTRDSLFF | AGCUGGUGGGCCACC | | | |
| | CLKFCTAAISYS | UGCAGGAGGUGCUG | | | |
| | LCKFSSQSRDT | GAGACCCAGGAGGU | | | |
| | LCSCLSPGLIKK | GCACUGGCAGCGGGU | | | |
| | FQFLMFRLFSE | GCUGAGCUUCGUGA | | | |
| | ARQPLSEEDVA | GCGCCCUGGUGGUGU | | | |
| | SLSWRPLHLPS | GCUUCCCCGAGGCCC | | | |
| | ADWQRAALSL | AGCAGCUGCUGGAG | | | |
| | WTHRTFREVLK | GACUGGGUGGCCCGG | | | |
| | EEDVHLTYQD | CUGAUGGCCCAGGCC | | | |
| | WLHLELEIQPE | UUCGAGAGCUGCCAG | | | |
| | ADALSDTERQD | CUGGACAGCAUGGU | | | |
| | FHQWAIHEHFL | GACCGCCUUCCUGGU | | | |
| | PESSASGGCDG | GGUAAGGCAAGCCGC | | | |
| | DLQAACTILVN | CCUGGAGGGUCCCAG | | | |
| | ALMDFHQSSRS | CGCCUUCCUGAGCUA | | | |
| | YDHSENSDLVF | CGCCGACUGGUUCAA | | | |
| | GGRTGNEDIISR | GGCCAGCUUCGGCAG | | | |
| | LQEMVADLEL | CACCCGGGGCUACCA | | | |
| | QQDLIVPLGHT | CGGCUGCAGCAAGAA | | | |
| | PSQEHFLFEIFR | GGCCCUGGUGUUCCU | | | |
| | RRLQALTSGWS | GUUCACCUUCCUGAG | | | |
| | VAASLQRQREL | CGAGCUGGUGCCCUU | | | |
| | LMYKRILLRLP | CGAGUCACCCCGGUA | | | |
| | SSVLCGSSFQA | CCUGCAGGUGCACAU | | | |
| | EQPITARCEQFF | CCUGCACCCACCCCU | | | |
| | HLVNSEMRNFC | GGUGCCCGGCAAGUA | | | |
| | SHGGALTQDIT | CCGGAGCCUGCUGAC | | | |
| | AHFFRGLLNAC | CGACUACAUCAGCCU | | | |
| | LRSRDPSLMVD | GGCCAAGACCCGGCU | | | |
| | FILAKCQTKCP | GGCCGACCUGAAGGU | | | |
| | LILTSALVWWP | GAGCAUCGAGAACA | | | |
| | SLEPVLLCRWR | UGGGCCUGUACGAG | | | |
| | RHCQSPLPREL | GACCUGAGCAGCGCC | | | |
| | QKLQEGRQFAS | GGCGACAUCACCGAG | | | |
| | DFLSPEAASPAP | CCCCACAGCCAGGCC | | | |
| | NPDWLSAAAL | CUGCAGGACGUGGA | | | |
| | HFAIQQVREENI | GAAGGCCAUCAUGG | | | |
| | RKQLKKLDCER | UGUUCGAGCACACCG | | | |
| | EELLVFLFFFSL | GCAACAUCCCCGUGA | | | |
| | MGLLSSHLTSN | CCGUGAUGGAGGCCA | | | |
| | STTDLPKAFHV | GCAUCUUCCGGCGGC | | | |
| | CAAILECLEKR | CCUACUACGUGAGCC | | | |
| | KISWLALFQLT | ACUUCCUGCCCGCCC | | | |
| | ESDLRLGRLLL | UGCUGACUCCCCGGG | | | |
| | RVAPDQHTRLL | UGCUGCCCAAGGUGC | | | |
| | PFAFYSLLSYFH | CCGACAGCCGGGUGG | | | |
| | EDAAIREEAFL | CCUUCAUCGAGAGCC | | | |
| | HVAVDMYLKL | UGAAGCGGGCCGACA | | | |
| | VQLFVAGDTST | AGAUUCCUCCCAGCC | | | |
| | VSPPAGRSLEL | UGUACAGCACCUACU | | | |
| | KGQGNPVELIT | GCCAGGCCUGCAGUG | | | |
| | KARLFLLQLIPR | CCGCCGAGGAGAAGC | | | |
| | CPKKSFSHVAE | CCGAAGACGCCGCUC | | | |
| | LLADRGDCDPE | UGGGCGUGAGAGCC | | | |
| | VSAALQSRQQA | GAGCCCAACAGCGCC | | | |
| | APDADLSQEPH | GAGGAGCCUUUGGG | | | |
| | LF | CCAGCUGACAGCAGC | | | |
| | | CCUUGGCGAGCUGCG | | | |
| | | GGCCAGCAUGACCGA | | | |
| | | CCCCAGCCAGCGGGA | | | |
| | | CGUGAUCAGCGCCCA | | | |
| | | GGUGGCCGUGAUCUC | | | |
| | | CGAGAGACUGCGGGC | | | |
| | | CGUGCUGGGCCACAA | | | |
| | | CGAGGACGACAGCAG | | | |
| | | CGUGGAGAUCAGCA | | | |
| | | AGAUCCAGCUGAGCA | | | |
| | | UCAACACUCCCCGGC | | | |
| | | UGGAGCCCAGGGAGC | | | |
| | | ACAUGGCCGUGGACC | | | |
| | | UGCUGCUGACCAGCU | | | |
| | | UCUGCCAGAACCUGA | | | |
| | | UGGCCGCCUCAAGCG | | | |
| | | UGGCCCCUCCAGAGC | | | |
| | | GGCAAGGCCCUUGGG | | | |
| | | CGGCCCUGUUCGUGC | | | |
| | | GGACCAUGUGCGGCC | | | |
| | | GGGUGUUGCCUGCCG | | | |
| | | UGCUGACCCGGCUGU | | | |
| | | GCCAGCUGCUGCGGC | | | |
| | | ACCAGGGCCCUAGCC | | | |
| | | UGAGCGCUCCCCACG | | | |
| | | UGCUGGGGCUGGCCG | | | |
| | | CCUUAGCAGUGCACC | | | |
| | | UGGGCGAGUCACGG | | | |
| | | AGCGCCCUGCCCGAG | | | |
| | | GUGGACGUGGGCCCU | | | |
| | | CCUGCGCCAGGAGCA | | | |
| | | GGGCUUCCCGUGCCC | | | |
| | | GCCCUGUUCGACAGC | | | |
| | | CUGCUGACCUGCCGG | | | |
| | | ACCCGGGACAGCCUG | | | |
| | | UUCUUCUGCCUGAAG | | | |
| | | UUCUGCACCGCCGCC | | | |
| | | AUCAGCUACAGCCUG | | | |
| | | UGCAAGUUCAGCAGC | | | |
| | | CAGAGCCGGGACACC | | | |
| | | CUGUGCAGCUGCCUG | | | |
| | | AGCCCCGGCCUGAUC | | | |
| | | AAGAAGUUCCAGUU | | | |
| | | CCUGAUGUUCCGGCU | | | |
| | | GUUCAGCGAGGCCCG | | | |
| | | GCAACCCCUGAGCGA | | | |
| | | GGAGGACGUGGCCA | | | |
| | | GCCUGAGCUGGCGGC | | | |
| | | CCCUGCACCUUCCCA | | | |
| | | GCGCCGACUGGCAAC | | | |
| | | GCGCCGCCCUGAGCC | | | |
| | | UGUGGACCCACCGGA | | | |
| | | CCUUCCGGGAGGUGC | | | |
| | | UGAAGGAGGAGGAC | | | |
| | | GUGCACCUGACCUAC | | | |
| | | CAGGACUGGCUGCAC | | | |
| | | CUGGAGCUGGAGAU | | | |
| | | UCAGCCCGAGGCCGA | | | |
| | | CGCCCUGAGCGACAC | | | |
| | | CGAGCGGCAGGACUU | | | |
| | | CCACCAGUGGGCCAU | | | |
| | | CCACGAGCACUUCCU | | | |
| | | GCCCGAGAGCAGUGC | | | |
| | | CAGCGGCGGCUGUGA | | | |
| | | CGGCGACCUGCAGGC | | | |
| | | CGCCUGCACCAUCCU | | | |
| | | GGUGAACGCCCUGAU | | | |
| | | GGACUUCCACCAGAG | | | |
| | | CAGCCGGAGCUACGA | | | |
| | | CCACAGCGAGAACAG | | | |
| | | CGACCUGGUGUUCGG | | | |
| | | AGGCCGGACCGGCAA | | | |
| | | CGAGGACAUCAUCAG | | | |
| | | CCGGCUGCAGGAGAU | | | |
| | | GGUGGCCGACCUGGA | | | |
| | | GCUGCAGCAGGACCU | | | |
| | | GAUCGUGCCCCUGGG | | | |
| | | CCACACCCCUAGCCA | | | |
| | | GGAGCACUUCCUGUU | | | |
| | | CGAGAUCUUUCGGCG | | | |
| | | GCGGCUGCAAGCGCU | | | |
| | | GACCAGCGGUUGGA | | | |
| | | GCGUGGCCGCCUCCC | | | |
| | | UGCAACGGCAGCGGG | | | |
| | | AGCUGCUGAUGUAC | | | |
| | | AAGCGGAUCCUGCUG | | | |
| | | CGGCUGCCCAGCAGC | | | |
| | | GUGCUGUGCGGCAGC | | | |
| | | AGCUUCCAGGCCGAG | | | |
| | | CAGCCCAUCACCGCC | | | |
| | | CGGUGCGAGCAGUUC | | | |
| | | UUCCACCUGGUGAAC | | | |
| | | AGCGAGAUGCGGAA | | | |
| | | CUUCUGCAGCCACGG | | | |
| | | UGGCGCCCUGACCCA | | | |
| | | GGACAUCACCGCCCA | | | |
| | | CUUCUUCCGGGGCCU | | | |
| | | GCUGAACGCCUGCCU | | | |
| | | GCGUAGCCGGGACCC | | | |
| | | CAGCCUGAUGGUGG | | | |
| | | ACUUCAUCCUGGCCA | | | |
| | | AGUGCCAGACCAAGU | | | |
| | | GCCCACUGAUCCUGA | | | |
| | | CCAGCGCCCUGGUGU | | | |
| | | GGUGGCCCAGCCUGG | | | |
| | | AGCCCGUGCUGCUGU | | | |
| | | GCCGGUGGCGACGGC | | | |
| | | ACUGCCAGUCACCGC | | | |
| | | UGCCCCGGGAGCUGC | | | |
| | | AGAAGCUGCAGGAG | | | |
| | | GGCCGGCAGUUCGCC | | | |
| | | AGCGACUUCCUGAGC | | | |
| | | CCCGAGGCUGCCAGC | | | |
| | | CCUGCCCCGAACCCC | | | |
| | | GACUGGCUGAGUGCC | | | |
| | | GCCGCCCUGCACUUC | | | |
| | | GCCAUCCAGCAGGUG | | | |
| | | CGGGAGGAGAACAU | | | |
| | | CCGGAAGCAGCUGAA | | | |
| | | GAAGCUGGACUGCG | | | |
| | | AGCGGGAGGAGCUG | | | |
| | | CUGGUGUUCCUGUUC | | | |
| | | UUCUUCAGCCUGAUG | | | |
| | | GGCCUGCUGAGCAGC | | | |
| | | CACCUGACCAGCAAC | | | |
| | | AGCACCACCGACCUG | | | |
| | | CCCAAGGCCUUCCAC | | | |
| | | GUGUGCGCCGCCAUC | | | |
| | | CUGGAGUGCCUGGA | | | |
| | | GAAGCGGAAGAUCA | | | |
| | | GCUGGCUGGCCCUGU | | | |
| | | UCCAGCUGACCGAGU | | | |
| | | CUGACCUGCGCCUGG | | | |
| | | GCAGGCUGCUGCUGC | | | |
| | | GGGUGGCUCCCGACC | | | |
| | | AGCACACCCGGCUGC | | | |
| | | UGCCCUUCGCCUUCU | | | |
| | | ACAGCCUGCUGAGCU | | | |
| | | ACUUCCACGAGGACG | | | |
| | | CCGCCAUCCGGGAGG | | | |
| | | AGGCCUUCCUGCACG | | | |
| | | UGGCCGUGGACAUG | | | |
| | | UACCUGAAGCUGGU | | | |
| | | GCAGCUGUUCGUGGC | | | |
| | | CGGCGACACCAGCAC | | | |
| | | UGUAUCUCCACCGGC | | | |
| | | UGGGCGGUCCCUGGA | | | |
| | | GCUGAAGGGCCAGG | | | |
| | | GCAACCCCGUGGAGC | | | |
| | | UGAUCACCAAGGCCC | | | |
| | | GGCUGUUCCUGCUGC | | | |
| | | AGCUGAUCCCACGGU | | | |
| | | GCCCCAAGAAGAGCU | | | |
| | | UCAGCCACGUGGCCG | | | |
| | | AGCUGCUGGCCGACC | | | |
| | | GGGGAGACUGCGACC | | | |
| | | CCGAGGUUAGUGCCG | | | |
| | | CCCUCCAGAGCCGGC | | | |
| | | AGCAAGCCGCCCCUG | | | |
| | | ACGCCGACCUGAGCC | | | |
| | | AGGAGCCCCACCUGU | | | |
| | | UC | | | |
| | SEQ ID NO: | | | | |
| | 1 | 16 | 50 | 139 | 25 |
| FANCA_05 | MSDSWVPNSA | AUGUCCGACUCUUGG | GGAAA | UAAAGC | SEQ ID |
| Chemistry: G5 | SGQDPGGRRRA | GUCCCCAACUCCGCC | UCGCAA | UCCCCG | NO: 25 |
| Cap: C1 | WAELLAGRVK | UCUGGUCAAGAUCCG | AAUUU | GGGGCC | consists |
| PolyA tail: | REKYNPERAQK | GGAGGAAGGAGACG | GCUCUU | UCGGUG | from 5′ to |
| 100 nt (SEQ ID | LKESAVRLLRS | UGCCUGGGCGGAACU | CGCGUU | GCCUAG | 3′ end: 5′ |
| NO: 121) | HQDLNALLLEV | GUUAGCCGGACGUG | AGAUU | CUUCUU | UTR of |
| | EGPLCKKLSLS | UGAAGAGGGAGAAG | UCUUUU | GCCCCU | SEQ ID |
| | KVIDCDSSEAY | UACAAUCCCGAGCGC | AGUUU | UGGGCC | NO: 50, |
| | ANHSSSFIGSAL | GCACAGAAGCUCAAG | UCUCGC | UCCCCC | ORF |
| | QDQASRLGVPV | GAAUCCGCAGUUAG | AACUAG | CAGCCC | Sequence |
| | GILSAGMVASS | GCUCCUCCGCUCUCA | CAAGCU | CUCCUC | of SEQ ID |
| | VGQICTAPAET | CCAGGACCUCAACGC | UUUUG | CCCUUC | NO: 16, |
| | SHPVLLTVEQR | ACUGCUCCUCGAGGU | UUCUCG | CUGCAC | and 3′ |
| | KKLSSLLEFAQ | CGAGGGACCACUCUG | CC | CCGUAC | UTR of |
| | YLLAHSMFSRL | CAAGAAGCUCUCCCU | | CCCCGU | SEQ ID |
| | SFCQELWKIQS | CUCCAAGGUCAUCGA | | GGUCUU | NO: 139 |
| | SLLLEAVWHLH | CUGCGACUCCUCCGA | | UGAAU | |
| | VQGIVSLQELL | GGCCUACGCCAACCA | | AAAGUC | |
| | ESHPDMHAVG | CUCCUCCUCCUUCAU | | UGAGU | |
| | SWLFRNLCCLC | CGGCUCCGCCCUCCA | | GGGCGG | |
| | EQMEASCQHA | AGAUCAAGCUUCCCG | | C | |
| | DVARAMLSDF | CCUCGGAGUUCCUGU | | | |
| | VQMFVLRGFQ | GGGCAUCCUAAGCGC | | | |
| | KNSDLRRTVEP | CGGGAUGGUCGCAUC | | | |
| | EKMPQVTVDV | UUCCGUCGGCCAGAU | | | |
| | LQRMLIFALDA | UUGCACAGCACCCGC | | | |
| | LAAGVQEESST | CGAGACAUCCCAUCC | | | |
| | HKIVRCWFGVF | UGUCCUUCUCACCGU | | | |
| | SGHTLGSVISTD | CGAGCAGCGCAAGAA | | | |
| | PLKRFFSHTLT | GCUCUCCUCCCUCCU | | | |
| | QILTHSPVLKAS | CGAGUUUGCCCAGUA | | | |
| | DAVQMQREWS | CCUCCUCGCCCACUC | | | |
| | FARTHPLLTSL | CAUGUUCUCCCGCCU | | | |
| | YRRLFVMLSAE | CUCCUUCUGCCAGGA | | | |
| | ELVGHLQEVLE | GCUCUGGAAGAUCCA | | | |
| | TQEVHWQRVL | GUCCUCCCUCCUGCU | | | |
| | SFVSALVVCFP | CGAAGCCGUCUGGCA | | | |
| | EAQQLLEDWV | UCUCCACGUCCAGGG | | | |
| | ARLMAQAFESC | CAUCGUCUCCCUCCA | | | |
| | QLDSMVTAFLV | GGAGCUCCUCGAAUC | | | |
| | VRQAALEGPSA | CCAUCCAGACAUGCA | | | |
| | FLSYADWFKAS | CGCCGUCGGUUCUUG | | | |
| | FGSTRGYHGCS | GCUCUUCCGCAACCU | | | |
| | KKALVFLFTFL | CUGCUGCCUCUGCGA | | | |
| | SELVPFESPRYL | GCAGAUGGAAGCCUC | | | |
| | QVHILHPPLVP | CUGCCAACACGCUGA | | | |
| | GKYRSLLTDYI | CGUCGCUCGAGCCAU | | | |
| | SLAKTRLADLK | GCUCUCCGACUUCGU | | | |
| | VSIENMGLYED | CCAGAUGUUCGUCCU | | | |
| | LSSAGDITEPHS | CCGCGGCUUCCAGAA | | | |
| | QALQDVEKAI | GAAUUCCGACCUCCG | | | |
| | MVFEHTGNIPV | UCGCACAGUCGAGCC | | | |
| | TVMEASIFRRP | UGAGAAGAUGCCUC | | | |
| | YYVSHFLPALL | AGGUCACCGUCGACG | | | |
| | TPRVLPKVPDS | UCCUCCAGAGAAUGC | | | |
| | RVAFIESLKRA | UCAUCUUCGCACUGG | | | |
| | DKIPPSLYSTYC | ACGCACUAGCUGCAG | | | |
| | QACSAAEEKPE | GCGUCCAGGAGGAG | | | |
| | DAALGVRAEP | UCCUCCACCCACAAG | | | |
| | NSAEEPLGQLT | AUCGUCCGCUGUUGG | | | |
| | AALGELRASMT | UUCGGCGUCUUUAGC | | | |
| | DPSQRDVISAQ | GGGCACACCUUGGGG | | | |
| | VAVISERLRAV | UCAGUCAUCUCCACC | | | |
| | LGHNEDDSSVE | GAUCCCCUCAAGCGC | | | |
| | ISKIQLSINTPRL | UUCUUCUCCCACACC | | | |
| | EPREHMAVDLL | CUCACCCAGAUCCUC | | | |
| | LTSFCQNLMAA | ACCCACUCCCCAGUC | | | |
| | SSVAPPERQGP | CUCAAAGCCUCCGAC | | | |
| | WAALFVRTMC | GCCGUCCAGAUGCAA | | | |
| | GRVLPAVLTRL | CGCGAGUGGUCCUUU | | | |
| | CQLLRHQGPSL | GCCCGAACCCACCCA | | | |
| | SAPHVLGLAAL | CUCCUCACCUCCCUC | | | |
| | AVHLGESRSAL | UACCGCAGGCUCUUC | | | |
| | PEVDVGPPAPG | GUCAUGCUCUCCGCC | | | |
| | AGLPVPALFDS | GAAGAACUCGUCGGC | | | |
| | LLTCRTRDSLFF | CACUUGCAGGAGGUC | | | |
| | CLKFCTAAISYS | CUCGAGACCCAAGAA | | | |
| | LCKFSSQSRDT | GUCCACUGGCAGCGC | | | |
| | LCSCLSPGLIKK | GUCCUCUCCUUCGUC | | | |
| | FQFLMFRLFSE | UCCGCCCUCGUCGUC | | | |
| | ARQPLSEEDVA | UGUUUUCCAGAGGCC | | | |
| | SLSWRPLHLPS | CAGCAGCUCCUUGAG | | | |
| | ADWQRAALSL | GAUUGGGUGGCACG | | | |
| | WTHRTFREVLK | GCUCAUGGCUCAGGC | | | |
| | EEDVHLTYQD | CUUCGAGUCCUGCCA | | | |
| | WLHLELEIQPE | GCUCGACUCCAUGGU | | | |
| | ADALSDTERQD | CACCGCCUUUCUUGU | | | |
| | FHQWAIHEHFL | UGUCCGCCAAGCUGC | | | |
| | PESSASGGCDG | UCUCGAGGGUCCUUC | | | |
| | DLQAACTILVN | GGCAUUCCUGAGCUA | | | |
| | ALMDFHQSSRS | CGCCGACUGGUUCAA | | | |
| | YDHSENSDLVF | GGCCUCCUUCGGUAG | | | |
| | GGRTGNEDIISR | CACAAGGGGCUACCA | | | |
| | LQEMVADLEL | CGGUUGCUCCAAGAA | | | |
| | QQDLIVPLGHT | GGCCCUCGUCUUCCU | | | |
| | PSQEHFLFEIFR | CUUCACCUUCCUCUC | | | |
| | RRLQALTSGWS | CGAGCUCGUCCCCUU | | | |
| | VAASLQRQREL | CGAGAGUCCCCGCUA | | | |
| | LMYKRILLRLP | CCUCCAGGUCCACAU | | | |
| | SSVLCGSSFQA | CCUCCAUCCUCCUCU | | | |
| | EQPITARCEQFF | GGUCCCCGGAAAGUA | | | |
| | HLVNSEMRNFC | CCGCUCCCUCCUCAC | | | |
| | SHGGALTQDIT | CGACUACAUCUCCCU | | | |
| | AHFFRGLLNAC | CGCUAAGACCCGACU | | | |
| | LRSRDPSLMVD | CGCCGACUUGAAGGU | | | |
| | FILAKCQTKCP | CUCCAUCGAGAACAU | | | |
| | LILTSALVWWP | GGGCCUCUACGAAGA | | | |
| | SLEPVLLCRWR | UCUCUCCAGUGCAGG | | | |
| | RHCQSPLPREL | CGACAUCACCGAGCC | | | |
| | QKLQEGRQFAS | ACACUCCCAAGCCCU | | | |
| | DFLSPEAASPAP | CCAGGACGUCGAGAA | | | |
| | NPDWLSAAAL | GGCCAUCAUGGUCUU | | | |
| | HFAIQQVREENI | CGAGCACACCGGCAA | | | |
| | RKQLKKLDCER | CAUCCCCGUCACCGU | | | |
| | EELLVFLFFFSL | CAUGGAGGCCUCCAU | | | |
| | MGLLSSHLTSN | CUUCAGACGCCCCUA | | | |
| | STTDLPKAFHV | CUACGUCUCCCACUU | | | |
| | CAAILECLEKR | UCUCCCUGCCCUCCU | | | |
| | KISWLALFQLT | AACCCCUCGGGUACU | | | |
| | ESDLRLGRLLL | CCCCAAGGUCCCCGA | | | |
| | RVAPDQHTRLL | CUCCAGAGUCGCCUU | | | |
| | PFAFYSLLSYFH | CAUCGAGUCCCUCAA | | | |
| | EDAAIREEAFL | GCGCGCAGACAAGAU | | | |
| | HVAVDMYLKL | CCCACCAUCCCUCUA | | | |
| | VQLFVAGDTST | CUCCACCUAUUGUCA | | | |
| | VSPPAGRSLEL | AGCCUGUUCUGCCGC | | | |
| | KGQGNPVELIT | CGAAGAGAAACCCGA | | | |
| | KARLFLLQLIPR | GGACGCCGCAUUAGG | | | |
| | CPKKSFSHVAE | CGUCCGUGCAGAGCC | | | |
| | LLADRGDCDPE | CAACUCAGCUGAGGA | | | |
| | VSAALQSRQQA | GCCACUGGGCCAGCU | | | |
| | APDADLSQEPH | AACAGCUGCACUAGG | | | |
| | LF | CGAGCUGAGGGCGA | | | |
| | | GUAUGACCGACCCUU | | | |
| | | CCCAGCGAGACGUCA | | | |
| | | UCUCCGCCCAAGUCG | | | |
| | | CCGUCAUUUCCGAAC | | | |
| | | GCUUGAGGGCAGUG | | | |
| | | CUGGGCCACAACGAG | | | |
| | | GACGACUCCUCCGUC | | | |
| | | GAGAUCUCCAAGAUC | | | |
| | | CAGCUCUCCAUCAAU | | | |
| | | ACACCUCGCCUGGAA | | | |
| | | CCCCGAGAGCACAUG | | | |
| | | GCCGUCGACCUCCUC | | | |
| | | CUCACCUCCUUCUGC | | | |
| | | CAGAACUUGAUGGC | | | |
| | | UGCCUCCUCCGUUGC | | | |
| | | UCCUCCGGAAAGACA | | | |
| | | AGGUCCCUGGGCCGC | | | |
| | | UCUCUUUGUCCGCAC | | | |
| | | CAUGUGUGGACGAG | | | |
| | | UGCUCCCAGCUGUCC | | | |
| | | UCACUCGCCUCUGCC | | | |
| | | AGUUGCUCCGCCACC | | | |
| | | AAGGACCCUCACUCU | | | |
| | | CUGCACCGCACGUAC | | | |
| | | UGGGACUCGCAGCAC | | | |
| | | UCGCAGUCCACCUCG | | | |
| | | GAGAAUCCCGCUCUG | | | |
| | | CUCUCCCAGAAGUGG | | | |
| | | ACGUGGGACCUCCUG | | | |
| | | CACCUGGUGCUGGCU | | | |
| | | UGCCCGUACCAGCUC | | | |
| | | UCUUCGACUCCCUCC | | | |
| | | UCACGUGCAGAACCC | | | |
| | | GCGACUCCCUCUUCU | | | |
| | | UCUGCCUCAAGUUCU | | | |
| | | GCACCGCCGCCAUCU | | | |
| | | CCUACUCCCUCUGCA | | | |
| | | AGUUCAGCUCCCAGU | | | |
| | | CACGCGACACCCUCU | | | |
| | | GCUCCUGCUUGUCCC | | | |
| | | CUGGCCUCAUCAAGA | | | |
| | | AGUUCCAGUUCCUCA | | | |
| | | UGUUCCGCCUCUUCU | | | |
| | | CCGAGGCCAGACAAC | | | |
| | | CCCUCUCCGAGGAGG | | | |
| | | ACGUGGCUUCCCUCA | | | |
| | | GUUGGAGACCCCUCC | | | |
| | | ACUUGCCUUCCGCCG | | | |
| | | AUUGGCAAAGAGCC | | | |
| | | GCGCUCUCUCUCUGG | | | |
| | | ACCCAUCGCACCUUC | | | |
| | | CGCGAGGUCCUCAAG | | | |
| | | GAGGAGGACGUCCAC | | | |
| | | CUCACCUACCAGGAC | | | |
| | | UGGCUCCACCUCGAG | | | |
| | | CUCGAGAUCCAACCC | | | |
| | | GAAGCAGACGCCCUC | | | |
| | | UCCGACACCGAACGC | | | |
| | | CAGGACUUCCACCAG | | | |
| | | UGGGCCAUCCACGAG | | | |
| | | CACUUCCUCCCCGAA | | | |
| | | AGCAGUGCUUCCGGU | | | |
| | | GGUUGCGACGGGGA | | | |
| | | UCUCCAAGCCGCUUG | | | |
| | | CACCAUCCUCGUCAA | | | |
| | | CGCCCUCAUGGACUU | | | |
| | | CCACCAGUCCUCCCG | | | |
| | | CUCCUACGACCACUC | | | |
| | | CGAGAACUCCGACCU | | | |
| | | AGUGUUUGGCGGCC | | | |
| | | GCACUGGAAACGAG | | | |
| | | GACAUCAUCUCCCGC | | | |
| | | CUCCAGGAGAUGGU | | | |
| | | GGCCGAUCUCGAGCU | | | |
| | | CCAGCAGGAUCUCAU | | | |
| | | CGUCCCUCUCGGCCA | | | |
| | | CACACCUUCGCAGGA | | | |
| | | GCACUUCCUCUUCGA | | | |
| | | AAUCUUCAGAAGAC | | | |
| | | GCCUCCAAGCGCUCA | | | |
| | | CUUCCGGUUGGUCUG | | | |
| | | UGGCCGCAAGUCUCC | | | |
| | | AACGCCAGAGGGAAC | | | |
| | | UCCUCAUGUACAAGC | | | |
| | | GCAUCCUCCUACGCC | | | |
| | | UCCCUUCCUCCGUCC | | | |
| | | UCUGUGGGUCCUCCU | | | |
| | | UCCAGGCAGAACAGC | | | |
| | | CCAUCACUGCCCGCU | | | |
| | | GUGAGCAGUUCUUCC | | | |
| | | ACCUCGUCAACUCCG | | | |
| | | AGAUGCGCAACUUCU | | | |
| | | GUUCACACGGCGGUG | | | |
| | | CACUCACCCAAGACA | | | |
| | | UCACCGCGCACUUCU | | | |
| | | UCCGCGGACUCCUGA | | | |
| | | ACGCCUGCUUGAGAU | | | |
| | | CCCGCGACCCAUCCC | | | |
| | | UCAUGGUCGACUUCA | | | |
| | | UCCUCGCCAAGUGCC | | | |
| | | AGACCAAGUGUCCCC | | | |
| | | UCAUCCUCACCAGUG | | | |
| | | CCCUCGUUUGGUGGC | | | |
| | | CCUCCCUUGAACCCG | | | |
| | | UCCUCCUCUGCAGGU | | | |
| | | GGAGACGCCACUGUC | | | |
| | | AGUCUCCGUUACCCC | | | |
| | | GCGAGCUCCAGAAAC | | | |
| | | UCCAGGAGGGACGCC | | | |
| | | AGUUCGCUUCCGACU | | | |
| | | UCCUCUCACCCGAAG | | | |
| | | CUGCUAGCCCAGCAC | | | |
| | | CCAACCCUGACUGGU | | | |
| | | UAUCCGCCGCCGCUU | | | |
| | | UGCACUUCGCCAUCC | | | |
| | | AGCAAGUGCGCGAG | | | |
| | | GAGAACAUCCGCAAG | | | |
| | | CAGCUCAAGAAGCUC | | | |
| | | GACUGCGAGCGAGA | | | |
| | | GGAGCUCCUCGUUUU | | | |
| | | CCUCUUCUUCUUCUC | | | |
| | | CCUCAUGGGCCUCCU | | | |
| | | CUCAUCCCACCUCAC | | | |
| | | CUCCAACUCCACCAC | | | |
| | | CGACUUGCCCAAGGC | | | |
| | | CUUUCACGUCUGUGC | | | |
| | | CGCCAUCCUCGAGUG | | | |
| | | CCUCGAGAAGCGCAA | | | |
| | | GAUCUCCUGGCUCGC | | | |
| | | CCUCUUCCAGCUAAC | | | |
| | | AGAGUCCGACCUGAG | | | |
| | | ACUCGGUCGCCUCCU | | | |
| | | ACUCAGAGUCGCACC | | | |
| | | CGACCAGCACACUCG | | | |
| | | CCUCCUCCCCUUCGC | | | |
| | | AUUCUACUCCCUCCU | | | |
| | | CUCCUACUUCCACGA | | | |
| | | AGACGCAGCAAUCCG | | | |
| | | CGAGGAGGCCUUCCU | | | |
| | | UCACGUCGCCGUCGA | | | |
| | | CAUGUACCUCAAGCU | | | |
| | | CGUCCAGCUCUUUGU | | | |
| | | CGCGGGCGACACGAG | | | |
| | | UACAGUUAGCCCUCC | | | |
| | | AGCAGGUAGGUCCCU | | | |
| | | CGAGCUUAAAGGCCA | | | |
| | | GGGCAACCCAGUCGA | | | |
| | | GCUCAUCACCAAGGC | | | |
| | | CCGCCUGUUCCUGCU | | | |
| | | ACAGCUCAUCCCACG | | | |
| | | CUGCCCCAAGAAGUC | | | |
| | | CUUCUCCCACGUAGC | | | |
| | | CGAACUCCUCGCCGA | | | |
| | | UCGCGGAGAUUGCG | | | |
| | | ACCCCGAAGUUUCCG | | | |
| | | CUGCCUUGCAGUCAC | | | |
| | | GGCAGCAAGCAGCCC | | | |
| | | CAGACGCAGACCUCU | | | |
| | | CACAGGAGCCCCACC | | | |
| | | UCUUC | | | |
| | SEQ ID NO: | | | | |
| | 1 | 17 | 50 | 139 | 26 |
| FANCA_06 | MSDSWVPNSA | AUGUCAGAUUCCUG | GGAAA | UAAAGC | SEQ ID |
| Chemistry: G5 | SGQDPGGRRRA | GGUGCCCAACUCCGC | UCGCAA | UCCCCG | NO: 26 |
| Cap: C1 | WAELLAGRVK | UAGCGGACAAGAUCC | AAUUU | GGGGCC | consists |
| PolyA tail: | REKYNPERAQK | AGGUGGCCGCAGGA | GCUCUU | UCGGUG | from 5′ to |
| 100 nt (SEQ ID | LKESAVRLLRS | GAGCCUGGGCUGAAC | CGCGUU | GCCUAG | 3′ end: 5′ |
| NO: 121) | HQDLNALLLEV | UGCUUGCCGGGAGA | AGAUU | CUUCUU | UTR of |
| | EGPLCKKLSLS | GUGAAGAGAGAGAA | UCUUUU | GCCCCU | SEQ ID |
| | KVIDCDSSEAY | GUACAACCCAGAGCG | AGUUU | UGGGCC | NO: 50, |
| | ANHSSSFIGSAL | CGCCCAGAAGUUAAA | UCUCGC | UCCCCC | ORF |
| | QDQASRLGVPV | GGAGAGCGCCGUAA | AACUAG | CAGCCC | Sequence |
| | GILSAGMVASS | GACUGCUGCGCAGCC | CAAGCU | CUCCUC | of SEQ ID |
| | VGQICTAPAET | ACCAAGACUUAAACG | UUUUG | CCCUUC | NO: 17, |
| | SHPVLLTVEQR | CUCUGCUGCUGGAGG | UUCUCG | CUGCAC | and 3′ |
| | KKLSSLLEFAQ | UGGAGGGGCCCCUGU | CC | CCGUAC | UTR of |
| | YLLAHSMFSRL | GCAAGAAGCUGAGCC | | CCCCGU | SEQ ID |
| | SFCQELWKIQS | UGUCCAAGGUGAUC | | GGUCUU | NO: 139 |
| | SLLLEAVWHLH | GAUUGUGACAGCAG | | UGAAU | |
| | VQGIVSLQELL | CGAGGCUUACGCCAA | | AAAGUC | |
| | ESHPDMHAVG | UCAUUCAUCUUCCUU | | UGAGU | |
| | SWLFRNLCCLC | CAUCGGCUCAGCACU | | GGGCGG | |
| | EQMEASCQHA | GCAGGAUCAAGCAA | | C | |
| | DVARAMLSDF | GCCGGCUGGGCGUUC | | | |
| | VQMFVLRGFQ | CAGUUGGCAUACUG | | | |
| | KNSDLRRTVEP | AGCGCCGGCAUGGUG | | | |
| | EKMPQVTVDV | GCCAGCAGCGUGGGA | | | |
| | LQRMLIFALDA | CAGAUCUGCACCGCU | | | |
| | LAAGVQEESST | CCCGCCGAGACCUCA | | | |
| | HKIVRCWFGVF | CAUCCCGUUCUGCUG | | | |
| | SGHTLGSVISTD | ACAGUUGAGCAACGC | | | |
| | PLKRFFSHTLT | AAGAAGCUGUCAAG | | | |
| | QILTHSPVLKAS | CCUGCUGGAGUUCGC | | | |
| | DAVQMQREWS | ACAGUACCUGUUAGC | | | |
| | FARTHPLLTSL | CCACAGCAUGUUUUC | | | |
| | YRRLFVMLSAE | CCGCUUAAGCUUCUG | | | |
| | ELVGHLQEVLE | CCAGGAGCUGUGGA | | | |
| | TQEVHWQRVL | AGAUCCAGUCAAGUC | | | |
| | SFVSALVVCFP | UGCUACUGGAGGCCG | | | |
| | EAQQLLEDWV | UGUGGCACUUACACG | | | |
| | ARLMAQAFESC | UGCAGGGUAUCGUG | | | |
| | QLDSMVTAFLV | UCACUGCAAGAGCUA | | | |
| | VRQAALEGPSA | CUGGAGAGCCAUCCC | | | |
| | FLSYADWFKAS | GAUAUGCACGCCGUG | | | |
| | FGSTRGYHGCS | GGGUCUUGGCUGUU | | | |
| | KKALVFLFTFL | CCGCAAUCUGUGCUG | | | |
| | SELVPFESPRYL | CCUGUGCGAACAGAU | | | |
| | QVHILHPPLVP | GGAAGCCUCCUGCCA | | | |
| | GKYRSLLTDYI | ACACGCCGACGUUGC | | | |
| | SLAKTRLADLK | UAGAGCAAUGCUGA | | | |
| | VSIENMGLYED | GUGACUUCGUUCAG | | | |
| | LSSAGDITEPHS | AUGUUCGUGCUGCGC | | | |
| | QALQDVEKAI | GGGUUCCAGAAGAA | | | |
| | MVFEHTGNIPV | CUCAGAUCUCCGGAG | | | |
| | TVMEASIFRRP | GACCGUGGAGCCUGA | | | |
| | YYVSHFLPALL | GAAGAUGCCCCAGGU | | | |
| | TPRVLPKVPDS | AACCGUGGACGUGCU | | | |
| | RVAFIESLKRA | GCAGCGGAUGUUAA | | | |
| | DKIPPSLYSTYC | UCUUUGCCCUGGACG | | | |
| | QACSAAEEKPE | CUCUCGCUGCCGGAG | | | |
| | DAALGVRAEP | UGCAGGAGGAGAGC | | | |
| | NSAEEPLGQLT | UCCACCCACAAGAUC | | | |
| | AALGELRASMT | GUGCGGUGCUGGUU | | | |
| | DPSQRDVISAQ | CGGCGUGUUUUCCGG | | | |
| | VAVISERLRAV | CCAUACCUUAGGCUC | | | |
| | LGHNEDDSSVE | CGUGAUCUCCACCGA | | | |
| | ISKIQLSINTPRL | UCCCCUGAAACGGUU | | | |
| | EPREHMAVDLL | CUUCUCCCACACACU | | | |
| | LTSFCQNLMAA | GACCCAGAUACUGAC | | | |
| | SSVAPPERQGP | CCACUCCCCUGUGUU | | | |
| | WAALFVRTMC | AAAGGCUAGCGACGC | | | |
| | GRVLPAVLTRL | CGUGCAAAUGCAACG | | | |
| | CQLLRHQGPSL | CGAGUGGUCCUUUGC | | | |
| | SAPHVLGLAAL | CCGCACCCAUCCACU | | | |
| | AVHLGESRSAL | GCUGACCAGUCUGUA | | | |
| | PEVDVGPPAPG | CCGGCGGCUGUUUGU | | | |
| | AGLPVPALFDS | UAUGCUGUCCGCCGA | | | |
| | LLTCRTRDSLFF | GGAGUUAGUGGGCC | | | |
| | CLKFCTAAISYS | ACCUGCAGGAGGUUC | | | |
| | LCKFSSQSRDT | UGGAAACCCAGGAG | | | |
| | LCSCLSPGLIKK | GUGCAUUGGCAGCGC | | | |
| | FQFLMFRLFSE | GUGUUAAGCUUCGU | | | |
| | ARQPLSEEDVA | GAGCGCCCUGGUGGU | | | |
| | SLSWRPLHLPS | GUGCUUUCCCGAGGC | | | |
| | ADWQRAALSL | CCAGCAGUUACUGGA | | | |
| | WTHRTFREVLK | GGACUGGGUGGCCCG | | | |
| | EEDVHLTYQD | GCUGAUGGCCCAGGC | | | |
| | WLHLELEIQPE | CUUCGAGAGCUGCCA | | | |
| | ADALSDTERQD | GCUGGACAGCAUGG | | | |
| | FHQWAIHEHFL | UGACCGCCUUCCUGG | | | |
| | PESSASGGCDG | UGGUACGUCAAGCU | | | |
| | DLQAACTILVN | GCCCUGGAGGGCCCA | | | |
| | ALMDFHQSSRS | AGCGCCUUCCUGAGU | | | |
| | YDHSENSDLVF | UACGCUGACUGGUUC | | | |
| | GGRTGNEDIISR | AAGGCCAGCUUCGGG | | | |
| | LQEMVADLEL | UCCACCCGCGGGUAC | | | |
| | QQDLIVPLGHT | CACGGCUGCUCCAAG | | | |
| | PSQEHFLFEIFR | AAGGCCCUGGUGUUC | | | |
| | RRLQALTSGWS | CUGUUUACCUUCCUG | | | |
| | VAASLQRQREL | AGCGAGCUGGUGCCA | | | |
| | LMYKRILLRLP | UUUGAGUCUCCCCGC | | | |
| | SSVLCGSSFQA | UACCUGCAGGUGCAC | | | |
| | EQPITARCEQFF | AUCCUGCACCCACCC | | | |
| | HLVNSEMRNFC | CUGGUCCCUGGAAAG | | | |
| | SHGGALTQDIT | UACAGAUCACUGUU | | | |
| | AHFFRGLLNAC | AACCGACUACAUCUC | | | |
| | LRSRDPSLMVD | UCUGGCCAAGACCCG | | | |
| | FILAKCQTKCP | GUUAGCCGACCUGAA | | | |
| | LILTSALVWWP | GGUGUCUAUCGAGA | | | |
| | SLEPVLLCRWR | AUAUGGGCCUGUAC | | | |
| | RHCQSPLPREL | GAGGACCUGUCCAGC | | | |
| | QKLQEGRQFAS | GCCGGCGAUAUCACC | | | |
| | DFLSPEAASPAP | GAGCCCCACAGCCAA | | | |
| | NPDWLSAAAL | GCCUUACAGGACGUG | | | |
| | HFAIQQVREENI | GAGAAGGCCAUUAU | | | |
| | RKQLKKLDCER | GGUGUUCGAGCACAC | | | |
| | EELLVFLFFFSL | CGGCAAUAUCCCCGU | | | |
| | MGLLSSHLTSN | GACCGUAAUGGAGG | | | |
| | STTDLPKAFHV | CUUCUAUCUUCCGGC | | | |
| | CAAILECLEKR | GGCCCUACUACGUGU | | | |
| | KISWLALFQLT | CCCACUUCCUGCCCG | | | |
| | ESDLRLGRLLL | CCCUGUUAACACCCA | | | |
| | RVAPDQHTRLL | GAGUGUUACCCAAG | | | |
| | PFAFYSLLSYFH | GUGCCCGACAGUCGC | | | |
| | EDAAIREEAFL | GUUGCCUUCAUAGA | | | |
| | HVAVDMYLKL | GAGCUUAAAGAGAG | | | |
| | VQLFVAGDTST | CCGACAAGAUCCCUC | | | |
| | VSPPAGRSLEL | CUAGUUUAUACUCCA | | | |
| | KGQGNPVELIT | CCUACUGCCAGGCUU | | | |
| | KARLFLLQLIPR | GCUCCGCCGCCGAGG | | | |
| | CPKKSFSHVAE | AGAAGCCCGAGGACG | | | |
| | LLADRGDCDPE | CAGCCUUAGGAGUA | | | |
| | VSAALQSRQQA | AGAGCUGAGCCCAAC | | | |
| | APDADLSQEPH | UCAGCCGAGGAGCCA | | | |
| | LF | CUGGGCCAGUUAACC | | | |
| | | GCUGCCCUGGGAGAG | | | |
| | | UUACGGGCCUCUAUG | | | |
| | | ACCGACCCCAGCCAA | | | |
| | | CGCGACGUUAUCUCU | | | |
| | | GCCCAAGUGGCAGUG | | | |
| | | AUAAGCGAGAGGCU | | | |
| | | GCGGGCAGUAUUAG | | | |
| | | GACACAACGAGGACG | | | |
| | | ACAGCAGCGUGGAG | | | |
| | | AUCAGUAAGAUCCA | | | |
| | | AUUAAGCAUCAACAC | | | |
| | | ACCCAGGCUGGAGCC | | | |
| | | CAGAGAGCACAUGGC | | | |
| | | CGUGGACCUGCUGUU | | | |
| | | AACCUCCUUCUGUCA | | | |
| | | GAACUUAAUGGCCGC | | | |
| | | AAGUAGCGUGGCUCC | | | |
| | | UCCAGAACGCCAAGG | | | |
| | | UCCUUGGGCUGCCUU | | | |
| | | AUUCGUUCGCACCAU | | | |
| | | GUGUGGCCGCGUCCU | | | |
| | | GCCAGCAGUGCUGAC | | | |
| | | CCGGCUGUGCCAGUU | | | |
| | | ACUGCGGCACCAAGG | | | |
| | | GCCCUCCCUGAGCGC | | | |
| | | UCCUCACGUGCUUGG | | | |
| | | GUUGGCCGCCCUCGC | | | |
| | | UGUACACUUAGGUG | | | |
| | | AAAGCAGAAGCGCCC | | | |
| | | UUCCUGAGGUGGAC | | | |
| | | GUGGGUCCUCCAGCC | | | |
| | | CCAGGUGCCGGACUC | | | |
| | | CCCGUGCCAGCCCUG | | | |
| | | UUCGACAGCUUACUG | | | |
| | | ACUUGUCGCACCCGG | | | |
| | | GACUCCCUGUUCUUU | | | |
| | | UGUCUGAAGUUCUG | | | |
| | | CACCGCCGCCAUCUC | | | |
| | | CUACUCACUGUGCAA | | | |
| | | GUUCUCUAGCCAGUC | | | |
| | | CCGGGAUACCCUGUG | | | |
| | | CUCCUGCCUGUCUCC | | | |
| | | AGGCCUGAUCAAGA | | | |
| | | AGUUCCAGUUCCUGA | | | |
| | | UGUUCAGACUGUUC | | | |
| | | UCAGAAGCAAGACA | | | |
| | | GCCUCUGUCCGAGGA | | | |
| | | GGACGUGGCCUCCUU | | | |
| | | AUCCUGGCGACCCCU | | | |
| | | GCACCUGCCCUCCGC | | | |
| | | UGAUUGGCAAAGAG | | | |
| | | CUGCACUUAGCCUGU | | | |
| | | GGACCCACCGGACAU | | | |
| | | UUCGCGAGGUGCUG | | | |
| | | AAAGAAGAAGACGU | | | |
| | | ACACCUGACCUACCA | | | |
| | | AGACUGGCUGCACCU | | | |
| | | GGAGCUGGAGAUCC | | | |
| | | AGCCUGAAGCCGACG | | | |
| | | CCCUGAGCGACACCG | | | |
| | | AGCGCCAAGACUUUC | | | |
| | | ACCAGUGGGCCAUAC | | | |
| | | ACGAGCACUUUCUGC | | | |
| | | CCGAGUCAAGCGCCA | | | |
| | | GUGGAGGCUGCGAC | | | |
| | | GGUGAUCUGCAAGC | | | |
| | | AGCCUGCACCAUCUU | | | |
| | | AGUGAACGCCCUGAU | | | |
| | | GGACUUCCACCAAUC | | | |
| | | UUCCCGCAGCUACGA | | | |
| | | CCACUCCGAGAACAG | | | |
| | | CGACCUGGUGUUUG | | | |
| | | GAGGCCGCACUGGGA | | | |
| | | ACGAGGACAUCAUU | | | |
| | | AGCCGCCUGCAAGAA | | | |
| | | AUGGUGGCUGACCU | | | |
| | | GGAACUCCAACAGGA | | | |
| | | CUUAAUCGUACCUCU | | | |
| | | GGGGCACACACCAAG | | | |
| | | CCAGGAGCACUUCUU | | | |
| | | AUUUGAGAUAUUUA | | | |
| | | GAAGACGCUUACAA | | | |
| | | GCACUGACUUCAGGC | | | |
| | | UGGAGUGUAGCUGC | | | |
| | | AAGCCUGCAACGCCA | | | |
| | | ACGCGAGCUGCUGAU | | | |
| | | GUAUAAGCGGAUAC | | | |
| | | UGCUGCGCCUGCCCU | | | |
| | | CCUCUGUACUGUGCG | | | |
| | | GCUCCUCCUUCCAGG | | | |
| | | CCGAGCAGCCCAUCA | | | |
| | | CCGCCCGGUGUGAGC | | | |
| | | AGUUCUUCCACCUGG | | | |
| | | UGAACAGCGAGAUG | | | |
| | | CGGAACUUCUGUUCU | | | |
| | | CACGGAGGCGCUCUG | | | |
| | | ACCCAAGACAUCACA | | | |
| | | GCCCACUUCUUCCGC | | | |
| | | GGCCUGCUGAACGCC | | | |
| | | UGUCUGCGGUCAAG | | | |
| | | GGACCCCAGCCUGAU | | | |
| | | GGUCGACUUCAUACU | | | |
| | | GGCAAAGUGUCAGA | | | |
| | | CCAAGUGUCCCCUGA | | | |
| | | UCCUGACCAGCGCUC | | | |
| | | UGGUUUGGUGGCCA | | | |
| | | UCACUGGAGCCCGUA | | | |
| | | CUCCUGUGUCGCUGG | | | |
| | | AGGCGGCAUUGCCAG | | | |
| | | UCACCUUUACCCCGA | | | |
| | | GAACUGCAGAAGCU | | | |
| | | GCAGGAGGGCCGGCA | | | |
| | | GUUUGCCAGCGACUU | | | |
| | | CCUGUCACCAGAGGC | | | |
| | | AGCCUCUCCCGCUCC | | | |
| | | UAACCCCGAUUGGCU | | | |
| | | AUCCGCGGCCGCUCU | | | |
| | | GCACUUCGCCAUCCA | | | |
| | | GCAGGUGCGGGAGG | | | |
| | | AGAACAUACGGAAA | | | |
| | | CAACUGAAGAAAUU | | | |
| | | AGACUGCGAGAGAG | | | |
| | | AGGAGUUACUUGUG | | | |
| | | UUCCUGUUCUUCUUC | | | |
| | | AGUCUGAUGGGCCU | | | |
| | | GCUGUCCUCCCACCU | | | |
| | | GACCAGCAAUUCAAC | | | |
| | | CACCGACCUGCCCAA | | | |
| | | GGCCUUCCACGUUUG | | | |
| | | UGCCGCCAUCCUGGA | | | |
| | | GUGCCUGGAGAAGC | | | |
| | | GGAAGAUUAGCUGG | | | |
| | | UUAGCCCUGUUUCAG | | | |
| | | CUGACCGAGAGUGA | | | |
| | | UUUACGCCUGGGGA | | | |
| | | GACUGCUGUUACGCG | | | |
| | | UGGCUCCCGACCAGC | | | |
| | | ACACCAGGCUGCUGC | | | |
| | | CUUUCGCCUUCUACA | | | |
| | | GUCUGUUAUCCUACU | | | |
| | | UCCACGAGGACGCCG | | | |
| | | CCAUCAGAGAGGAG | | | |
| | | GCCUUCCUGCACGUG | | | |
| | | GCCGUGGACAUGUAC | | | |
| | | UUAAAGCUGGUGCA | | | |
| | | GCUGUUUGUGGCCG | | | |
| | | GCGAUACCUCCACAG | | | |
| | | UAAGCCCUCCUGCUG | | | |
| | | GCAGAAGCCUGGAGC | | | |
| | | UGAAGGGCCAGGGU | | | |
| | | AACCCCGUAGAGCUG | | | |
| | | AUUACUAAGGCCCGC | | | |
| | | CUGUUUCUGCUGCAA | | | |
| | | CUGAUCCCUCGCUGC | | | |
| | | CCCAAGAAGAGCUUU | | | |
| | | AGCCACGUGGCCGAA | | | |
| | | UUACUGGCUGACAG | | | |
| | | GGGAGACUGUGAUC | | | |
| | | CUGAGGUGAGCGCU | | | |
| | | GCCUUACAGAGCCGU | | | |
| | | CAACAAGCGGCCCCU | | | |
| | | GACGCCGAUUUAAGC | | | |
| | | CAAGAGCCUCACCUC | | | |
| | | UUC | | | |
| | SEQ ID NO: | | | | |
| | 1 | 2 | 50 | 117 | 27 |
| FANCA_07 | MSDSWVPNSA | AUGUCCGACUCCUGG | GGAAA | UGAUA | SEQ ID |
| Chemistry: G5 | SGQDPGGRRRA | GUGCCCAACUCCGCU | UCGCAA | AUAGGC | NO: 27 |
| Cap: C1 | WAELLAGRVK | AGCGGACAAGAUCCA | AAUUU | UGGAGC | consists |
| Poly A tail: | REKYNPERAQK | GGUGGCCGCAGGAG | GCUCUU | CUCCAC | from 5′ to |
| 100 nt (SEQ ID | LKESAVRLLRS | AGCGUGGGCUGAAC | CGCGUU | CGCGUU | 3′ end: 5′ |
| NO: 121) | HQDLNALLLEV | UGCUUGCCGGGCGAG | AGAUU | AUCCGU | UTR of |
| | EGPLCKKLSLS | UGAAGCGCGAGAAG | UCUUUU | UCCUCG | SEQ ID |
| | KVIDCDSSEAY | UACAACCCCGAGCGC | AGUUU | UAGGCU | NO: 50, |
| | ANHSSSFIGSAL | GCCCAGAAGCUGAAG | UCUCGC | GGUCCU | ORF |
| | QDQASRLGVPV | GAGAGCGCCGUACGG | AACUAG | GGGGA | Sequence |
| | GILSAGMVASS | CUGCUGCGCAGCCAC | CAAGCU | ACGGGU | of SEQ ID |
| | VGQICTAPAET | CAGGACCUGAACGCC | UUUUG | CGGCGG | NO: 2, and |
| | SHPVLLTVEQR | CUGCUGCUGGAGGU | UUCUCG | GUACCC | 3′ UTR of |
| | KKLSSLLEFAQ | GGAGGGGCCCCUGUG | CC | CCGUGG | SEQ ID |
| | YLLAHSMFSRL | CAAGAAGCUGAGCCU | | UCUUUG | NO: 117 |
| | SFCQELWKIQS | GUCCAAGGUGAUCG | | AAUAA | |
| | SLLLEAVWHLH | ACUGCGACAGCAGCG | | AGUCUG | |
| | VQGIVSLQELL | AGGCCUACGCCAACC | | AGUGG | |
| | ESHPDMHAVG | ACAGCUCCUCCUUCA | | GCGGC | |
| | SWLFRNLCCLC | UCGGCUCCGCCCUGC | | | |
| | EQMEASCQHA | AGGAUCAAGCUAGCC | | | |
| | DVARAMLSDF | GGCUGGGCGUUCCAG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCUCCCG | | | |
| | LAAGVQEESST | CCGAGACCAGCCACC | | | |
| | HKIVRCWFGVF | CCGUGCUGCUGACCG | | | |
| | SGHTLGSVISTD | UGGAGCAGCGCAAG | | | |
| | PLKRFFSHTLT | AAGCUGUCAAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCUCCCGC | | | |
| | YRRLFVMLSAE | CUGAGCUUCUGCCAG | | | |
| | ELVGHLQEVLE | GAGCUGUGGAAGAU | | | |
| | TQEVHWQRVL | CCAGUCCAGCCUGCU | | | |
| | SFVSALVVCFP | ACUGGAGGCCGUGU | | | |
| | EAQQLLEDWV | GGCACUUACACGUGC | | | |
| | ARLMAQAFESC | AGGGCAUCGUGAGCC | | | |
| | QLDSMVTAFLV | UGCAGGAGCUACUG | | | |
| | VRQAALEGPSA | GAGAGCCACCCCGAC | | | |
| | FLSYADWFKAS | AUGCACGCCGUGGGG | | | |
| | FGSTRGYHGCS | UCCUGGCUGUUCCGC | | | |
| | KKALVFLFTFL | AACCUGUGCUGCCUG | | | |
| | SELVPFESPRYL | UGCGAGCAGAUGGA | | | |
| | QVHILHPPLVP | AGCCUCCUGCCAACA | | | |
| | GKYRSLLTDYI | CGCCGACGUUGCACG | | | |
| | SLAKTRLADLK | GGCAAUGCUGAGCG | | | |
| | VSIENMGLYED | ACUUCGUGCAGAUG | | | |
| | LSSAGDITEPHS | UUCGUGCUGCGCGGG | | | |
| | QALQDVEKAI | UUCCAGAAGAACUCA | | | |
| | MVFEHTGNIPV | GACCUCCGGAGGACC | | | |
| | TVMEASIFRRP | GUGGAGCCUGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UUGCCCUGGACGCUC | | | |
| | QACSAAEEKPE | UCGCUGCCGGAGUGC | | | |
| | DAALGVRAEP | AGGAGGAGAGCUCC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCUCCGGCCAC | | | |
| | VAVISERLRAV | ACCCUGGGCUCCGUG | | | |
| | LGHNEDDSSVE | AUCUCCACCGAUCCC | | | |
| | ISKIQLSINTPRL | CUGAAGCGGUUCUUC | | | |
| | EPREHMAVDLL | UCCCACACCCUGACC | | | |
| | LTSFCQNLMAA | CAGAUCCUGACCCAC | | | |
| | SSVAPPERQGP | UCCCCUGUGCUGAAG | | | |
| | WAALFVRTMC | GCCAGCGACGCCGUG | | | |
| | GRVLPAVLTRL | CAGAUGCAGCGCGAG | | | |
| | CQLLRHQGPSL | UGGUCCUUCGCCCGC | | | |
| | SAPHVLGLAAL | ACCCAUCCCCUGCUG | | | |
| | AVHLGESRSAL | ACCAGCCUGUACCGG | | | |
| | PEVDVGPPAPG | CGGCUGUUCGUGAU | | | |
| | AGLPVPALFDS | GCUGUCCGCCGAGGA | | | |
| | LLTCRTRDSLFF | GCUGGUGGGCCACCU | | | |
| | CLKFCTAAISYS | GCAGGAGGUGCUGG | | | |
| | LCKFSSQSRDT | AGACCCAGGAGGUGC | | | |
| | LCSCLSPGLIKK | ACUGGCAGCGCGUGC | | | |
| | FQFLMFRLFSE | UGAGCUUCGUGAGC | | | |
| | ARQPLSEEDVA | GCCCUGGUGGUGUGC | | | |
| | SLSWRPLHLPS | UUCCCCGAGGCCCAG | | | |
| | ADWQRAALSL | CAGCUCCUGGAGGAC | | | |
| | WTHRTFREVLK | UGGGUGGCCCGGCUG | | | |
| | EEDVHLTYQD | AUGGCCCAGGCCUUC | | | |
| | WLHLELEIQPE | GAGAGCUGCCAGCUG | | | |
| | ADALSDTERQD | GACAGCAUGGUGACC | | | |
| | FHQWAIHEHFL | GCCUUCCUGGUGGUG | | | |
| | PESSASGGCDG | CGUCAAGCAGCCCUG | | | |
| | DLQAACTILVN | GAGGGCCCAAGCGCC | | | |
| | ALMDFHQSSRS | UUCCUGAGCUACGCC | | | |
| | YDHSENSDLVF | GACUGGUUCAAGGCC | | | |
| | GGRTGNEDIISR | AGCUUCGGGUCCACC | | | |
| | LQEMVADLEL | CGCGGGUACCACGGC | | | |
| | QQDLIVPLGHT | UGCUCCAAGAAGGCC | | | |
| | PSQEHFLFEIFR | CUGGUGUUCCUGUU | | | |
| | RRLQALTSGWS | UACCUUCCUGAGCGA | | | |
| | VAASLQRQREL | GCUGGUGCCCUUCGA | | | |
| | LMYKRILLRLP | GUCGCCCCGCUACCU | | | |
| | SSVLCGSSFQA | GCAGGUGCACAUCCU | | | |
| | EQPITARCEQFF | GCACCCACCCCUGGU | | | |
| | HLVNSEMRNFC | CCCUGGCAAGUACCG | | | |
| | SHGGALTQDIT | GAGCCUGCUGACCGA | | | |
| | AHFFRGLLNAC | CUACAUCUCCCUGGC | | | |
| | LRSRDPSLMVD | CAAGACCCGGCUGGC | | | |
| | FILAKCQTKCP | CGACCUGAAGGUGA | | | |
| | LILTSALVWWP | GCAUCGAGAACAUG | | | |
| | SLEPVLLCRWR | GGCCUGUACGAGGAC | | | |
| | RHCQSPLPREL | CUGUCCAGCGCCGGC | | | |
| | QKLQEGRQFAS | GACAUCACCGAGCCC | | | |
| | DFLSPEAASPAP | CACAGCCAGGCCCUG | | | |
| | NPDWLSAAAL | CAGGACGUGGAGAA | | | |
| | HFAIQQVREENI | GGCCAUCAUGGUGU | | | |
| | RKQLKKLDCER | UCGAGCACACCGGCA | | | |
| | EELLVFLFFFSL | ACAUCCCCGUGACCG | | | |
| | MGLLSSHLTSN | UGAUGGAGGCUAGU | | | |
| | STTDLPKAFHV | AUCUUCCGGCGGCCC | | | |
| | CAAILECLEKR | UACUACGUGUCCCAC | | | |
| | KISWLALFQLT | UUCCUGCCCGCCCUG | | | |
| | ESDLRLGRLLL | CUGACACCCCGGGUG | | | |
| | RVAPDQHTRLL | CUGCCCAAGGUGCCC | | | |
| | PFAFYSLLSYFH | GACAGUCGCGUCGCC | | | |
| | EDAAIREEAFL | UUCAUCGAGAGCCUG | | | |
| | HVAVDMYLKL | AAGCGGGCCGACAAG | | | |
| | VQLFVAGDTST | AUCCCGCCCUCCCUG | | | |
| | VSPPAGRSLEL | UACUCCACCUACUGC | | | |
| | KGQGNPVELIT | CAGGCUUGCUCCGCC | | | |
| | KARLFLLQLIPR | GCCGAGGAGAAGCCC | | | |
| | CPKKSFSHVAE | GAGGACGCAGCCUUA | | | |
| | LLADRGDCDPE | GGAGUACGCGCCGAG | | | |
| | VSAALQSRQQA | CCCAACAGCGCCGAG | | | |
| | APDADLSQEPH | GAGCCACUGGGCCAG | | | |
| | LF | UUGACCGCUGCCCUG | | | |
| | | GGAGAGCUGCGGGCC | | | |
| | | AGCAUGACCGACCCC | | | |
| | | AGCCAACGCGACGUG | | | |
| | | AUCAGCGCCCAGGUG | | | |
| | | GCCGUGAUCAGCGAG | | | |
| | | AGGCUGCGGGCAGU | | | |
| | | GCUGGGCCACAACGA | | | |
| | | GGACGACAGCAGCGU | | | |
| | | GGAGAUCAGCAAGA | | | |
| | | UCCAGCUGAGCAUCA | | | |
| | | ACACACCCAGGCUGG | | | |
| | | AGCCCAGAGAGCACA | | | |
| | | UGGCCGUGGACCUGC | | | |
| | | UGCUGACCUCCUUCU | | | |
| | | GCCAGAACCUGAUGG | | | |
| | | CCGCAAGCAGCGUGG | | | |
| | | CUCCUCCAGAACGCC | | | |
| | | AAGGCCCUUGGGCUG | | | |
| | | CCCUGUUCGUGCGCA | | | |
| | | CCAUGUGUGGCCGCG | | | |
| | | UCCUGCCAGCAGUGC | | | |
| | | UGACCCGGCUGUGCC | | | |
| | | AGCUUCUGCGGCACC | | | |
| | | AAGGGCCCUCCCUGA | | | |
| | | GCGCUCCCCACGUGC | | | |
| | | UUGGGUUGGCCGCCC | | | |
| | | UCGCUGUGCACCUGG | | | |
| | | GCGAGAGCAGGAGC | | | |
| | | GCCCUUCCCGAGGUG | | | |
| | | GACGUGGGUCCUCCA | | | |
| | | GCCCCAGGUGCCGGA | | | |
| | | CUCCCCGUGCCAGCC | | | |
| | | CUGUUCGACAGCCUG | | | |
| | | CUGACUUGUCGCACC | | | |
| | | CGGGACUCCCUGUUC | | | |
| | | UUCUGCCUGAAGUUC | | | |
| | | UGCACCGCCGCCAUC | | | |
| | | UCCUACUCCCUGUGC | | | |
| | | AAGUUCUCCAGCCAG | | | |
| | | UCCCGGGACACCCUG | | | |
| | | UGCUCCUGCCUGUCU | | | |
| | | CCCGGCCUGAUCAAG | | | |
| | | AAGUUCCAGUUCCUG | | | |
| | | AUGUUCCGCCUGUUC | | | |
| | | AGCGAAGCACGCCAG | | | |
| | | CCCCUGUCCGAGGAG | | | |
| | | GACGUGGCCUCCCUG | | | |
| | | UCCUGGCGACCCCUG | | | |
| | | CACCUGCCCUCCGCC | | | |
| | | GAUUGGCAGCGCGCC | | | |
| | | GCCCUUAGCCUGUGG | | | |
| | | ACCCACCGGACCUUC | | | |
| | | CGCGAGGUGCUGAA | | | |
| | | GGAGGAAGACGUGC | | | |
| | | ACCUGACCUACCAGG | | | |
| | | ACUGGCUGCACCUGG | | | |
| | | AGCUGGAGAUCCAGC | | | |
| | | CCGAAGCCGACGCCC | | | |
| | | UGAGCGACACCGAGC | | | |
| | | GCCAGGACUUUCACC | | | |
| | | AGUGGGCCAUCCACG | | | |
| | | AGCACUUUCUGCCCG | | | |
| | | AGAGCAGCGCCAGUG | | | |
| | | GAGGCUGCGACGGCG | | | |
| | | ACCUGCAAGCCGCCU | | | |
| | | GCACCAUCCUGGUGA | | | |
| | | ACGCCCUGAUGGACU | | | |
| | | UCCACCAGAGCUCCC | | | |
| | | GCAGCUACGACCACU | | | |
| | | CCGAGAACAGCGACC | | | |
| | | UGGUGUUUGGAGGC | | | |
| | | CGCACCGGGAACGAG | | | |
| | | GACAUCAUCAGCCGC | | | |
| | | CUGCAGGAGAUGGU | | | |
| | | GGCCGACCUGGAGCU | | | |
| | | CCAGCAGGACCUGAU | | | |
| | | CGUGCCACUGGGGCA | | | |
| | | CACCCCAAGCCAGGA | | | |
| | | GCACUUCCUGUUCGA | | | |
| | | GAUCUUCCGCAGACG | | | |
| | | CCUGCAGGCACUGAC | | | |
| | | CUCCGGCUGGAGUGU | | | |
| | | GGCCGCAAGCCUGCA | | | |
| | | GCGCCAACGCGAGCU | | | |
| | | GCUGAUGUACAAGC | | | |
| | | GGAUCCUGCUGCGCC | | | |
| | | UGCCCUCCUCCGUGC | | | |
| | | UGUGCGGCUCCUCCU | | | |
| | | UCCAGGCCGAGCAGC | | | |
| | | CCAUCACCGCCCGGU | | | |
| | | GCGAGCAGUUCUUCC | | | |
| | | ACCUGGUGAACAGCG | | | |
| | | AGAUGCGGAACUUC | | | |
| | | UGCUCUCACGGCGGC | | | |
| | | GCUCUGACCCAGGAC | | | |
| | | AUCACCGCCCACUUC | | | |
| | | UUCCGCGGCCUGCUG | | | |
| | | AACGCCUGCCUGCGG | | | |
| | | AGCAGGGACCCCAGC | | | |
| | | CUGAUGGUCGACUUC | | | |
| | | AUCCUGGCCAAGUGC | | | |
| | | CAGACCAAGUGUCCC | | | |
| | | CUGAUCCUGACCAGC | | | |
| | | GCCCUGGUUUGGUG | | | |
| | | GCCCUCACUGGAGCC | | | |
| | | CGUGCUCCUGUGUCG | | | |
| | | CUGGAGGCGGCACUG | | | |
| | | CCAGUCACCCCUGCC | | | |
| | | CCGAGAGCUGCAGAA | | | |
| | | GCUGCAGGAGGGCCG | | | |
| | | GCAGUUCGCCAGCGA | | | |
| | | CUUCCUGUCACCAGA | | | |
| | | GGCAGCCUCUCCCGC | | | |
| | | UCCUAACCCCGAUUG | | | |
| | | GCUAUCCGCGGCCGC | | | |
| | | UCUGCACUUCGCCAU | | | |
| | | CCAGCAGGUGCGGGA | | | |
| | | GGAGAACAUCCGGA | | | |
| | | AGCAGCUGAAGAAG | | | |
| | | CUGGACUGCGAGAG | | | |
| | | GGAGGAGCUGCUUG | | | |
| | | UGUUCCUGUUCUUCU | | | |
| | | UCAGCCUGAUGGGCC | | | |
| | | UGCUGUCCUCCCACC | | | |
| | | UGACCAGCAACUCCA | | | |
| | | CCACCGACCUGCCCA | | | |
| | | AGGCCUUCCACGUGU | | | |
| | | GCGCCGCCAUCCUGG | | | |
| | | AGUGCCUGGAGAAG | | | |
| | | CGGAAGAUCAGCUG | | | |
| | | GCUGGCCCUGUUCCA | | | |
| | | GCUGACCGAGAGCGA | | | |
| | | CCUGCGCCUGGGGAG | | | |
| | | ACUGCUGCUGCGCGU | | | |
| | | GGCUCCCGACCAGCA | | | |
| | | CACCAGGCUGCUGCC | | | |
| | | CUUCGCCUUCUACUC | | | |
| | | CCUGCUGUCCUACUU | | | |
| | | CCACGAGGACGCCGC | | | |
| | | CAUCCGGGAGGAGGC | | | |
| | | CUUCCUGCACGUGGC | | | |
| | | CGUGGACAUGUACCU | | | |
| | | GAAGCUGGUGCAGC | | | |
| | | UGUUCGUGGCCGGCG | | | |
| | | ACACCUCCACCGUGA | | | |
| | | GCCCUCCUGCUGGCC | | | |
| | | GAAGCCUGGAGCUG | | | |
| | | AAGGGCCAGGGCAAC | | | |
| | | CCCGUGGAGCUGAUC | | | |
| | | ACCAAGGCCCGCCUG | | | |
| | | UUCCUGCUGCAGCUG | | | |
| | | AUCCCUCGCUGCCCC | | | |
| | | AAGAAGAGCUUCAG | | | |
| | | CCACGUGGCCGAGCU | | | |
| | | GCUGGCUGACAGGG | | | |
| | | GAGACUGCGACCCCG | | | |
| | | AGGUGAGCGCUGCCC | | | |
| | | UGCAGAGCCGUCAAC | | | |
| | | AAGCGGCCCCUGACG | | | |
| | | CCGAUCUGAGCCAGG | | | |
| | | AGCCCCACCUGUUC | | | |
| | SEQ ID NO: | | | | |
| | 1 | 2 | 80 | 117 | 28 |
| FANCA_08 | MSDSWVPNSA | AUGUCCGACUCCUGG | GGACUC | UGAUA | SEQ ID |
| Chemistry: G5 | SGQDPGGRRRA | GUGCCCAACUCCGCU | ACUAUU | AUAGGC | NO: 28 |
| Cap: C1 | WAELLAGRVK | AGCGGACAAGAUCCA | UGUUU | UGGAGC | consists |
| Poly A tail: | REKYNPERAQK | GGUGGCCGCAGGAG | UCGCGC | CUCCAC | from 5′ to |
| 100 nt (SEQ ID | LKESAVRLLRS | AGCGUGGGCUGAAC | CCAGUU | CGCGUU | 3′ end: 5′ |
| NO: 121) | HQDLNALLLEV | UGCUUGCCGGGCGAG | GCAAAA | AUCCGU | UTR of |
| | EGPLCKKLSLS | UGAAGCGCGAGAAG | A | UCCUCG | SEQ ID |
| | KVIDCDSSEAY | UACAACCCCGAGCGC | | UAGGCU | NO: 80, |
| | ANHSSSFIGSAL | GCCCAGAAGCUGAAG | | GGUCCU | ORF |
| | QDQASRLGVPV | GAGAGCGCCGUACGG | | GGGGA | Sequence |
| | GILSAGMVASS | CUGCUGCGCAGCCAC | | ACGGGU | of SEQ ID |
| | VGQICTAPAET | CAGGACCUGAACGCC | | CGGCGG | NO: 2, and |
| | SHPVLLTVEQR | CUGCUGCUGGAGGU | | GUACCC | 3′ UTR of |
| | KKLSSLLEFAQ | GGAGGGGCCCCUGUG | | CCGUGG | SEQ ID |
| | YLLAHSMFSRL | CAAGAAGCUGAGCCU | | UCUUUG | NO: 117 |
| | SFCQELWKIQS | GUCCAAGGUGAUCG | | AAUAA | |
| | SLLLEAVWHLH | ACUGCGACAGCAGCG | | AGUCUG | |
| | VQGIVSLQELL | AGGCCUACGCCAACC | | AGUGG | |
| | ESHPDMHAVG | ACAGCUCCUCCUUCA | | GCGGC | |
| | SWLFRNLCCLC | UCGGCUCCGCCCUGC | | | |
| | EQMEASCQHA | AGGAUCAAGCUAGCC | | | |
| | DVARAMLSDF | GGCUGGGCGUUCCAG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCUCCCG | | | |
| | LAAGVQEESST | CCGAGACCAGCCACC | | | |
| | HKIVRCWFGVF | CCGUGCUGCUGACCG | | | |
| | SGHTLGSVISTD | UGGAGCAGCGCAAG | | | |
| | PLKRFFSHTLT | AAGCUGUCAAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCUCCCGC | | | |
| | YRRLFVMLSAE | CUGAGCUUCUGCCAG | | | |
| | ELVGHLQEVLE | GAGCUGUGGAAGAU | | | |
| | TQEVHWQRVL | CCAGUCCAGCCUGCU | | | |
| | SFVSALVVCFP | ACUGGAGGCCGUGU | | | |
| | EAQQLLEDWV | GGCACUUACACGUGC | | | |
| | ARLMAQAFESC | AGGGCAUCGUGAGCC | | | |
| | QLDSMVTAFLV | UGCAGGAGCUACUG | | | |
| | VRQAALEGPSA | GAGAGCCACCCCGAC | | | |
| | FLSYADWFKAS | AUGCACGCCGUGGGG | | | |
| | FGSTRGYHGCS | UCCUGGCUGUUCCGC | | | |
| | KKALVFLFTFL | AACCUGUGCUGCCUG | | | |
| | SELVPFESPRYL | UGCGAGCAGAUGGA | | | |
| | QVHILHPPLVP | AGCCUCCUGCCAACA | | | |
| | GKYRSLLTDYI | CGCCGACGUUGCACG | | | |
| | SLAKTRLADLK | GGCAAUGCUGAGCG | | | |
| | VSIENMGLYED | ACUUCGUGCAGAUG | | | |
| | LSSAGDITEPHS | UUCGUGCUGCGCGGG | | | |
| | QALQDVEKAI | UUCCAGAAGAACUCA | | | |
| | MVFEHTGNIPV | GACCUCCGGAGGACC | | | |
| | TVMEASIFRRP | GUGGAGCCUGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UUGCCCUGGACGCUC | | | |
| | QACSAAEEKPE | UCGCUGCCGGAGUGC | | | |
| | DAALGVRAEP | AGGAGGAGAGCUCC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCUCCGGCCAC | | | |
| | VAVISERLRAV | ACCCUGGGCUCCGUG | | | |
| | LGHNEDDSSVE | AUCUCCACCGAUCCC | | | |
| | ISKIQLSINTPRL | CUGAAGCGGUUCUUC | | | |
| | EPREHMAVDLL | UCCCACACCCUGACC | | | |
| | LTSFCQNLMAA | CAGAUCCUGACCCAC | | | |
| | SSVAPPERQGP | UCCCCUGUGCUGAAG | | | |
| | WAALFVRTMC | GCCAGCGACGCCGUG | | | |
| | GRVLPAVLTRL | CAGAUGCAGCGCGAG | | | |
| | CQLLRHQGPSL | UGGUCCUUCGCCCGC | | | |
| | SAPHVLGLAAL | ACCCAUCCCCUGCUG | | | |
| | AVHLGESRSAL | ACCAGCCUGUACCGG | | | |
| | PEVDVGPPAPG | CGGCUGUUCGUGAU | | | |
| | AGLPVPALFDS | GCUGUCCGCCGAGGA | | | |
| | LLTCRTRDSLFF | GCUGGUGGGCCACCU | | | |
| | CLKFCTAAISYS | GCAGGAGGUGCUGG | | | |
| | LCKFSSQSRDT | AGACCCAGGAGGUGC | | | |
| | LCSCLSPGLIKK | ACUGGCAGCGCGUGC | | | |
| | FQFLMFRLFSE | UGAGCUUCGUGAGC | | | |
| | ARQPLSEEDVA | GCCCUGGUGGUGUGC | | | |
| | SLSWRPLHLPS | UUCCCCGAGGCCCAG | | | |
| | ADWQRAALSL | CAGCUCCUGGAGGAC | | | |
| | WTHRTFREVLK | UGGGUGGCCCGGCUG | | | |
| | EEDVHLTYQD | AUGGCCCAGGCCUUC | | | |
| | WLHLELEIQPE | GAGAGCUGCCAGCUG | | | |
| | ADALSDTERQD | GACAGCAUGGUGACC | | | |
| | FHQWAIHEHFL | GCCUUCCUGGUGGUG | | | |
| | PESSASGGCDG | CGUCAAGCAGCCCUG | | | |
| | DLQAACTILVN | GAGGGCCCAAGCGCC | | | |
| | ALMDFHQSSRS | UUCCUGAGCUACGCC | | | |
| | YDHSENSDLVF | GACUGGUUCAAGGCC | | | |
| | GGRTGNEDIISR | AGCUUCGGGUCCACC | | | |
| | LQEMVADLEL | CGCGGGUACCACGGC | | | |
| | QQDLIVPLGHT | UGCUCCAAGAAGGCC | | | |
| | PSQEHFLFEIFR | CUGGUGUUCCUGUU | | | |
| | RRLQALTSGWS | UACCUUCCUGAGCGA | | | |
| | VAASLQRQREL | GCUGGUGCCCUUCGA | | | |
| | LMYKRILLRLP | GUCGCCCCGCUACCU | | | |
| | SSVLCGSSFQA | GCAGGUGCACAUCCU | | | |
| | EQPITARCEQFF | GCACCCACCCCUGGU | | | |
| | HLVNSEMRNFC | CCCUGGCAAGUACCG | | | |
| | SHGGALTQDIT | GAGCCUGCUGACCGA | | | |
| | AHFFRGLLNAC | CUACAUCUCCCUGGC | | | |
| | LRSRDPSLMVD | CAAGACCCGGCUGGC | | | |
| | FILAKCQTKCP | CGACCUGAAGGUGA | | | |
| | LILTSALVWWP | GCAUCGAGAACAUG | | | |
| | SLEPVLLCRWR | GGCCUGUACGAGGAC | | | |
| | RHCQSPLPREL | CUGUCCAGCGCCGGC | | | |
| | QKLQEGRQFAS | GACAUCACCGAGCCC | | | |
| | DFLSPEAASPAP | CACAGCCAGGCCCUG | | | |
| | NPDWLSAAAL | CAGGACGUGGAGAA | | | |
| | HFAIQQVREENI | GGCCAUCAUGGUGU | | | |
| | RKQLKKLDCER | UCGAGCACACCGGCA | | | |
| | EELLVFLFFFSL | ACAUCCCCGUGACCG | | | |
| | MGLLSSHLTSN | UGAUGGAGGCUAGU | | | |
| | STTDLPKAFHV | AUCUUCCGGCGGCCC | | | |
| | CAAILECLEKR | UACUACGUGUCCCAC | | | |
| | KISWLALFQLT | UUCCUGCCCGCCCUG | | | |
| | ESDLRLGRLLL | CUGACACCCCGGGUG | | | |
| | RVAPDQHTRLL | CUGCCCAAGGUGCCC | | | |
| | PFAFYSLLSYFH | GACAGUCGCGUCGCC | | | |
| | EDAAIREEAFL | UUCAUCGAGAGCCUG | | | |
| | HVAVDMYLKL | AAGCGGGCCGACAAG | | | |
| | VQLFVAGDTST | AUCCCGCCCUCCCUG | | | |
| | VSPPAGRSLEL | UACUCCACCUACUGC | | | |
| | KGQGNPVELIT | CAGGCUUGCUCCGCC | | | |
| | KARLFLLQLIPR | GCCGAGGAGAAGCCC | | | |
| | CPKKSFSHVAE | GAGGACGCAGCCUUA | | | |
| | LLADRGDCDPE | GGAGUACGCGCCGAG | | | |
| | VSAALQSRQQA | CCCAACAGCGCCGAG | | | |
| | APDADLSQEPH | GAGCCACUGGGCCAG | | | |
| | LF | UUGACCGCUGCCCUG | | | |
| | | GGAGAGCUGCGGGCC | | | |
| | | AGCAUGACCGACCCC | | | |
| | | AGCCAACGCGACGUG | | | |
| | | AUCAGCGCCCAGGUG | | | |
| | | GCCGUGAUCAGCGAG | | | |
| | | AGGCUGCGGGCAGU | | | |
| | | GCUGGGCCACAACGA | | | |
| | | GGACGACAGCAGCGU | | | |
| | | GGAGAUCAGCAAGA | | | |
| | | UCCAGCUGAGCAUCA | | | |
| | | ACACACCCAGGCUGG | | | |
| | | AGCCCAGAGAGCACA | | | |
| | | UGGCCGUGGACCUGC | | | |
| | | UGCUGACCUCCUUCU | | | |
| | | GCCAGAACCUGAUGG | | | |
| | | CCGCAAGCAGCGUGG | | | |
| | | CUCCUCCAGAACGCC | | | |
| | | AAGGCCCUUGGGCUG | | | |
| | | CCCUGUUCGUGCGCA | | | |
| | | CCAUGUGUGGCCGCG | | | |
| | | UCCUGCCAGCAGUGC | | | |
| | | UGACCCGGCUGUGCC | | | |
| | | AGCUUCUGCGGCACC | | | |
| | | AAGGGCCCUCCCUGA | | | |
| | | GCGCUCCCCACGUGC | | | |
| | | UUGGGUUGGCCGCCC | | | |
| | | UCGCUGUGCACCUGG | | | |
| | | GCGAGAGCAGGAGC | | | |
| | | GCCCUUCCCGAGGUG | | | |
| | | GACGUGGGUCCUCCA | | | |
| | | GCCCCAGGUGCCGGA | | | |
| | | CUCCCCGUGCCAGCC | | | |
| | | CUGUUCGACAGCCUG | | | |
| | | CUGACUUGUCGCACC | | | |
| | | CGGGACUCCCUGUUC | | | |
| | | UUCUGCCUGAAGUUC | | | |
| | | UGCACCGCCGCCAUC | | | |
| | | UCCUACUCCCUGUGC | | | |
| | | AAGUUCUCCAGCCAG | | | |
| | | UCCCGGGACACCCUG | | | |
| | | UGCUCCUGCCUGUCU | | | |
| | | CCCGGCCUGAUCAAG | | | |
| | | AAGUUCCAGUUCCUG | | | |
| | | AUGUUCCGCCUGUUC | | | |
| | | AGCGAAGCACGCCAG | | | |
| | | CCCCUGUCCGAGGAG | | | |
| | | GACGUGGCCUCCCUG | | | |
| | | UCCUGGCGACCCCUG | | | |
| | | CACCUGCCCUCCGCC | | | |
| | | GAUUGGCAGCGCGCC | | | |
| | | GCCCUUAGCCUGUGG | | | |
| | | ACCCACCGGACCUUC | | | |
| | | CGCGAGGUGCUGAA | | | |
| | | GGAGGAAGACGUGC | | | |
| | | ACCUGACCUACCAGG | | | |
| | | ACUGGCUGCACCUGG | | | |
| | | AGCUGGAGAUCCAGC | | | |
| | | CCGAAGCCGACGCCC | | | |
| | | UGAGCGACACCGAGC | | | |
| | | GCCAGGACUUUCACC | | | |
| | | AGUGGGCCAUCCACG | | | |
| | | AGCACUUUCUGCCCG | | | |
| | | AGAGCAGCGCCAGUG | | | |
| | | GAGGCUGCGACGGCG | | | |
| | | ACCUGCAAGCCGCCU | | | |
| | | GCACCAUCCUGGUGA | | | |
| | | ACGCCCUGAUGGACU | | | |
| | | UCCACCAGAGCUCCC | | | |
| | | GCAGCUACGACCACU | | | |
| | | CCGAGAACAGCGACC | | | |
| | | UGGUGUUUGGAGGC | | | |
| | | CGCACCGGGAACGAG | | | |
| | | GACAUCAUCAGCCGC | | | |
| | | CUGCAGGAGAUGGU | | | |
| | | GGCCGACCUGGAGCU | | | |
| | | CCAGCAGGACCUGAU | | | |
| | | CGUGCCACUGGGGCA | | | |
| | | CACCCCAAGCCAGGA | | | |
| | | GCACUUCCUGUUCGA | | | |
| | | GAUCUUCCGCAGACG | | | |
| | | CCUGCAGGCACUGAC | | | |
| | | CUCCGGCUGGAGUGU | | | |
| | | GGCCGCAAGCCUGCA | | | |
| | | GCGCCAACGCGAGCU | | | |
| | | GCUGAUGUACAAGC | | | |
| | | GGAUCCUGCUGCGCC | | | |
| | | UGCCCUCCUCCGUGC | | | |
| | | UGUGCGGCUCCUCCU | | | |
| | | UCCAGGCCGAGCAGC | | | |
| | | CCAUCACCGCCCGGU | | | |
| | | GCGAGCAGUUCUUCC | | | |
| | | ACCUGGUGAACAGCG | | | |
| | | AGAUGCGGAACUUC | | | |
| | | UGCUCUCACGGCGGC | | | |
| | | GCUCUGACCCAGGAC | | | |
| | | AUCACCGCCCACUUC | | | |
| | | UUCCGCGGCCUGCUG | | | |
| | | AACGCCUGCCUGCGG | | | |
| | | AGCAGGGACCCCAGC | | | |
| | | CUGAUGGUCGACUUC | | | |
| | | AUCCUGGCCAAGUGC | | | |
| | | CAGACCAAGUGUCCC | | | |
| | | CUGAUCCUGACCAGC | | | |
| | | GCCCUGGUUUGGUG | | | |
| | | GCCCUCACUGGAGCC | | | |
| | | CGUGCUCCUGUGUCG | | | |
| | | CUGGAGGCGGCACUG | | | |
| | | CCAGUCACCCCUGCC | | | |
| | | CCGAGAGCUGCAGAA | | | |
| | | GCUGCAGGAGGGCCG | | | |
| | | GCAGUUCGCCAGCGA | | | |
| | | CUUCCUGUCACCAGA | | | |
| | | GGCAGCCUCUCCCGC | | | |
| | | UCCUAACCCCGAUUG | | | |
| | | GCUAUCCGCGGCCGC | | | |
| | | UCUGCACUUCGCCAU | | | |
| | | CCAGCAGGUGCGGGA | | | |
| | | GGAGAACAUCCGGA | | | |
| | | AGCAGCUGAAGAAG | | | |
| | | CUGGACUGCGAGAG | | | |
| | | GGAGGAGCUGCUUG | | | |
| | | UGUUCCUGUUCUUCU | | | |
| | | UCAGCCUGAUGGGCC | | | |
| | | UGCUGUCCUCCCACC | | | |
| | | UGACCAGCAACUCCA | | | |
| | | CCACCGACCUGCCCA | | | |
| | | AGGCCUUCCACGUGU | | | |
| | | GCGCCGCCAUCCUGG | | | |
| | | AGUGCCUGGAGAAG | | | |
| | | CGGAAGAUCAGCUG | | | |
| | | GCUGGCCCUGUUCCA | | | |
| | | GCUGACCGAGAGCGA | | | |
| | | CCUGCGCCUGGGGAG | | | |
| | | ACUGCUGCUGCGCGU | | | |
| | | GGCUCCCGACCAGCA | | | |
| | | CACCAGGCUGCUGCC | | | |
| | | CUUCGCCUUCUACUC | | | |
| | | CCUGCUGUCCUACUU | | | |
| | | CCACGAGGACGCCGC | | | |
| | | CAUCCGGGAGGAGGC | | | |
| | | CUUCCUGCACGUGGC | | | |
| | | CGUGGACAUGUACCU | | | |
| | | GAAGCUGGUGCAGC | | | |
| | | UGUUCGUGGCCGGCG | | | |
| | | ACACCUCCACCGUGA | | | |
| | | GCCCUCCUGCUGGCC | | | |
| | | GAAGCCUGGAGCUG | | | |
| | | AAGGGCCAGGGCAAC | | | |
| | | CCCGUGGAGCUGAUC | | | |
| | | ACCAAGGCCCGCCUG | | | |
| | | UUCCUGCUGCAGCUG | | | |
| | | AUCCCUCGCUGCCCC | | | |
| | | AAGAAGAGCUUCAG | | | |
| | | CCACGUGGCCGAGCU | | | |
| | | GCUGGCUGACAGGG | | | |
| | | GAGACUGCGACCCCG | | | |
| | | AGGUGAGCGCUGCCC | | | |
| | | UGCAGAGCCGUCAAC | | | |
| | | AAGCGGCCCCUGACG | | | |
| | | CCGAUCUGAGCCAGG | | | |
| | | AGCCCCACCUGUUC | | | |
| | SEQ ID NO: | | | | |
| | 1 | 2 | 80 | 182 | 29 |
| FANCA_09 | MSDSWVPNSA | AUGUCCGACUCCUGG | GGACUC | UAAGCC | SEQ ID |
| Chemistry: G5 | SGQDPGGRRRA | GUGCCCAACUCCGCU | ACUAUU | CCUCCG | NO: 29 |
| Cap: C1 | WAELLAGRVK | AGCGGACAAGAUCCA | UGUUU | GGGGCC | consists |
| Poly A tail: | REKYNPERAQK | GGUGGCCGCAGGAG | UCGCGC | UCCACC | from 5′ to |
| 100 nt (SEQ ID | LKESAVRLLRS | AGCGUGGGCUGAAC | CCAGUU | GCGUUA | 3′ end: 5′ |
| NO: 121) | HQDLNALLLEV | UGCUUGCCGGGCGAG | GCAAAA | UCCGUU | UTR of |
| | EGPLCKKLSLS | UGAAGCGCGAGAAG | A | CCUCGU | SEQ ID |
| | KVIDCDSSEAY | UACAACCCCGAGCGC | | AGGCUG | NO: 80, |
| | ANHSSSFIGSAL | GCCCAGAAGCUGAAG | | GUCCUG | ORF |
| | QDQASRLGVPV | GAGAGCGCCGUACGG | | GGGAAC | Sequence |
| | GILSAGMVASS | CUGCUGCGCAGCCAC | | GGGUCG | of SEQ ID |
| | VGQICTAPAET | CAGGACCUGAACGCC | | GCGGGU | NO: 2, and |
| | SHPVLLTVEQR | CUGCUGCUGGAGGU | | ACCCCC | 3′ UTR of |
| | KKLSSLLEFAQ | GGAGGGGCCCCUGUG | | GUGGUC | SEQ ID |
| | YLLAHSMFSRL | CAAGAAGCUGAGCCU | | UUUGA | NO: 140 |
| | SFCQELWKIQS | GUCCAAGGUGAUCG | | AUAAA | |
| | SLLLEAVWHLH | ACUGCGACAGCAGCG | | GUCUGA | |
| | VQGIVSLQELL | AGGCCUACGCCAACC | | GUGGGC | |
| | ESHPDMHAVG | ACAGCUCCUCCUUCA | | GGC | |
| | SWLFRNLCCLC | UCGGCUCCGCCCUGC | | | |
| | EQMEASCQHA | AGGAUCAAGCUAGCC | | | |
| | DVARAMLSDF | GGCUGGGCGUUCCAG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCUCCCG | | | |
| | LAAGVQEESST | CCGAGACCAGCCACC | | | |
| | HKIVRCWFGVF | CCGUGCUGCUGACCG | | | |
| | SGHTLGSVISTD | UGGAGCAGCGCAAG | | | |
| | PLKRFFSHTLT | AAGCUGUCAAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCUCCCGC | | | |
| | YRRLFVMLSAE | CUGAGCUUCUGCCAG | | | |
| | ELVGHLQEVLE | GAGCUGUGGAAGAU | | | |
| | TQEVHWQRVL | CCAGUCCAGCCUGCU | | | |
| | SFVSALVVCFP | ACUGGAGGCCGUGU | | | |
| | EAQQLLEDWV | GGCACUUACACGUGC | | | |
| | ARLMAQAFESC | AGGGCAUCGUGAGCC | | | |
| | QLDSMVTAFLV | UGCAGGAGCUACUG | | | |
| | VRQAALEGPSA | GAGAGCCACCCCGAC | | | |
| | FLSYADWFKAS | AUGCACGCCGUGGGG | | | |
| | FGSTRGYHGCS | UCCUGGCUGUUCCGC | | | |
| | KKALVFLFTFL | AACCUGUGCUGCCUG | | | |
| | SELVPFESPRYL | UGCGAGCAGAUGGA | | | |
| | QVHILHPPLVP | AGCCUCCUGCCAACA | | | |
| | GKYRSLLTDYI | CGCCGACGUUGCACG | | | |
| | SLAKTRLADLK | GGCAAUGCUGAGCG | | | |
| | VSIENMGLYED | ACUUCGUGCAGAUG | | | |
| | LSSAGDITEPHS | UUCGUGCUGCGCGGG | | | |
| | QALQDVEKAI | UUCCAGAAGAACUCA | | | |
| | MVFEHTGNIPV | GACCUCCGGAGGACC | | | |
| | TVMEASIFRRP | GUGGAGCCUGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UUGCCCUGGACGCUC | | | |
| | QACSAAEEKPE | UCGCUGCCGGAGUGC | | | |
| | DAALGVRAEP | AGGAGGAGAGCUCC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCUCCGGCCAC | | | |
| | VAVISERLRAV | ACCCUGGGCUCCGUG | | | |
| | LGHNEDDSSVE | AUCUCCACCGAUCCC | | | |
| | ISKIQLSINTPRL | CUGAAGCGGUUCUUC | | | |
| | EPREHMAVDLL | UCCCACACCCUGACC | | | |
| | LTSFCQNLMAA | CAGAUCCUGACCCAC | | | |
| | SSVAPPERQGP | UCCCCUGUGCUGAAG | | | |
| | WAALFVRTMC | GCCAGCGACGCCGUG | | | |
| | GRVLPAVLTRL | CAGAUGCAGCGCGAG | | | |
| | CQLLRHQGPSL | UGGUCCUUCGCCCGC | | | |
| | SAPHVLGLAAL | ACCCAUCCCCUGCUG | | | |
| | AVHLGESRSAL | ACCAGCCUGUACCGG | | | |
| | PEVDVGPPAPG | CGGCUGUUCGUGAU | | | |
| | AGLPVPALFDS | GCUGUCCGCCGAGGA | | | |
| | LLTCRTRDSLFF | GCUGGUGGGCCACCU | | | |
| | CLKFCTAAISYS | GCAGGAGGUGCUGG | | | |
| | LCKFSSQSRDT | AGACCCAGGAGGUGC | | | |
| | LCSCLSPGLIKK | ACUGGCAGCGCGUGC | | | |
| | FQFLMFRLFSE | UGAGCUUCGUGAGC | | | |
| | ARQPLSEEDVA | GCCCUGGUGGUGUGC | | | |
| | SLSWRPLHLPS | UUCCCCGAGGCCCAG | | | |
| | ADWQRAALSL | CAGCUCCUGGAGGAC | | | |
| | WTHRTFREVLK | UGGGUGGCCCGGCUG | | | |
| | EEDVHLTYQD | AUGGCCCAGGCCUUC | | | |
| | WLHLELEIQPE | GAGAGCUGCCAGCUG | | | |
| | ADALSDTERQD | GACAGCAUGGUGACC | | | |
| | FHQWAIHEHFL | GCCUUCCUGGUGGUG | | | |
| | PESSASGGCDG | CGUCAAGCAGCCCUG | | | |
| | DLQAACTILVN | GAGGGCCCAAGCGCC | | | |
| | ALMDFHQSSRS | UUCCUGAGCUACGCC | | | |
| | YDHSENSDLVF | GACUGGUUCAAGGCC | | | |
| | GGRTGNEDIISR | AGCUUCGGGUCCACC | | | |
| | LQEMVADLEL | CGCGGGUACCACGGC | | | |
| | QQDLIVPLGHT | UGCUCCAAGAAGGCC | | | |
| | PSQEHFLFEIFR | CUGGUGUUCCUGUU | | | |
| | RRLQALTSGWS | UACCUUCCUGAGCGA | | | |
| | VAASLQRQREL | GCUGGUGCCCUUCGA | | | |
| | LMYKRILLRLP | GUCGCCCCGCUACCU | | | |
| | SSVLCGSSFQA | GCAGGUGCACAUCCU | | | |
| | EQPITARCEQFF | GCACCCACCCCUGGU | | | |
| | HLVNSEMRNFC | CCCUGGCAAGUACCG | | | |
| | SHGGALTQDIT | GAGCCUGCUGACCGA | | | |
| | AHFFRGLLNAC | CUACAUCUCCCUGGC | | | |
| | LRSRDPSLMVD | CAAGACCCGGCUGGC | | | |
| | FILAKCQTKCP | CGACCUGAAGGUGA | | | |
| | LILTSALVWWP | GCAUCGAGAACAUG | | | |
| | SLEPVLLCRWR | GGCCUGUACGAGGAC | | | |
| | RHCQSPLPREL | CUGUCCAGCGCCGGC | | | |
| | QKLQEGRQFAS | GACAUCACCGAGCCC | | | |
| | DFLSPEAASPAP | CACAGCCAGGCCCUG | | | |
| | NPDWLSAAAL | CAGGACGUGGAGAA | | | |
| | HFAIQQVREENI | GGCCAUCAUGGUGU | | | |
| | RKQLKKLDCER | UCGAGCACACCGGCA | | | |
| | EELLVFLFFFSL | ACAUCCCCGUGACCG | | | |
| | MGLLSSHLTSN | UGAUGGAGGCUAGU | | | |
| | STTDLPKAFHV | AUCUUCCGGCGGCCC | | | |
| | CAAILECLEKR | UACUACGUGUCCCAC | | | |
| | KISWLALFQLT | UUCCUGCCCGCCCUG | | | |
| | ESDLRLGRLLL | CUGACACCCCGGGUG | | | |
| | RVAPDQHTRLL | CUGCCCAAGGUGCCC | | | |
| | PFAFYSLLSYFH | GACAGUCGCGUCGCC | | | |
| | EDAAIREEAFL | UUCAUCGAGAGCCUG | | | |
| | HVAVDMYLKL | AAGCGGGCCGACAAG | | | |
| | VQLFVAGDTST | AUCCCGCCCUCCCUG | | | |
| | VSPPAGRSLEL | UACUCCACCUACUGC | | | |
| | KGQGNPVELIT | CAGGCUUGCUCCGCC | | | |
| | KARLFLLQLIPR | GCCGAGGAGAAGCCC | | | |
| | CPKKSFSHVAE | GAGGACGCAGCCUUA | | | |
| | LLADRGDCDPE | GGAGUACGCGCCGAG | | | |
| | VSAALQSRQQA | CCCAACAGCGCCGAG | | | |
| | APDADLSQEPH | GAGCCACUGGGCCAG | | | |
| | LF | UUGACCGCUGCCCUG | | | |
| | | GGAGAGCUGCGGGCC | | | |
| | | AGCAUGACCGACCCC | | | |
| | | AGCCAACGCGACGUG | | | |
| | | AUCAGCGCCCAGGUG | | | |
| | | GCCGUGAUCAGCGAG | | | |
| | | AGGCUGCGGGCAGU | | | |
| | | GCUGGGCCACAACGA | | | |
| | | GGACGACAGCAGCGU | | | |
| | | GGAGAUCAGCAAGA | | | |
| | | UCCAGCUGAGCAUCA | | | |
| | | ACACACCCAGGCUGG | | | |
| | | AGCCCAGAGAGCACA | | | |
| | | UGGCCGUGGACCUGC | | | |
| | | UGCUGACCUCCUUCU | | | |
| | | GCCAGAACCUGAUGG | | | |
| | | CCGCAAGCAGCGUGG | | | |
| | | CUCCUCCAGAACGCC | | | |
| | | AAGGCCCUUGGGCUG | | | |
| | | CCCUGUUCGUGCGCA | | | |
| | | CCAUGUGUGGCCGCG | | | |
| | | UCCUGCCAGCAGUGC | | | |
| | | UGACCCGGCUGUGCC | | | |
| | | AGCUUCUGCGGCACC | | | |
| | | AAGGGCCCUCCCUGA | | | |
| | | GCGCUCCCCACGUGC | | | |
| | | UUGGGUUGGCCGCCC | | | |
| | | UCGCUGUGCACCUGG | | | |
| | | GCGAGAGCAGGAGC | | | |
| | | GCCCUUCCCGAGGUG | | | |
| | | GACGUGGGUCCUCCA | | | |
| | | GCCCCAGGUGCCGGA | | | |
| | | CUCCCCGUGCCAGCC | | | |
| | | CUGUUCGACAGCCUG | | | |
| | | CUGACUUGUCGCACC | | | |
| | | CGGGACUCCCUGUUC | | | |
| | | UUCUGCCUGAAGUUC | | | |
| | | UGCACCGCCGCCAUC | | | |
| | | UCCUACUCCCUGUGC | | | |
| | | AAGUUCUCCAGCCAG | | | |
| | | UCCCGGGACACCCUG | | | |
| | | UGCUCCUGCCUGUCU | | | |
| | | CCCGGCCUGAUCAAG | | | |
| | | AAGUUCCAGUUCCUG | | | |
| | | AUGUUCCGCCUGUUC | | | |
| | | AGCGAAGCACGCCAG | | | |
| | | CCCCUGUCCGAGGAG | | | |
| | | GACGUGGCCUCCCUG | | | |
| | | UCCUGGCGACCCCUG | | | |
| | | CACCUGCCCUCCGCC | | | |
| | | GAUUGGCAGCGCGCC | | | |
| | | GCCCUUAGCCUGUGG | | | |
| | | ACCCACCGGACCUUC | | | |
| | | CGCGAGGUGCUGAA | | | |
| | | GGAGGAAGACGUGC | | | |
| | | ACCUGACCUACCAGG | | | |
| | | ACUGGCUGCACCUGG | | | |
| | | AGCUGGAGAUCCAGC | | | |
| | | CCGAAGCCGACGCCC | | | |
| | | UGAGCGACACCGAGC | | | |
| | | GCCAGGACUUUCACC | | | |
| | | AGUGGGCCAUCCACG | | | |
| | | AGCACUUUCUGCCCG | | | |
| | | AGAGCAGCGCCAGUG | | | |
| | | GAGGCUGCGACGGCG | | | |
| | | ACCUGCAAGCCGCCU | | | |
| | | GCACCAUCCUGGUGA | | | |
| | | ACGCCCUGAUGGACU | | | |
| | | UCCACCAGAGCUCCC | | | |
| | | GCAGCUACGACCACU | | | |
| | | CCGAGAACAGCGACC | | | |
| | | UGGUGUUUGGAGGC | | | |
| | | CGCACCGGGAACGAG | | | |
| | | GACAUCAUCAGCCGC | | | |
| | | CUGCAGGAGAUGGU | | | |
| | | GGCCGACCUGGAGCU | | | |
| | | CCAGCAGGACCUGAU | | | |
| | | CGUGCCACUGGGGCA | | | |
| | | CACCCCAAGCCAGGA | | | |
| | | GCACUUCCUGUUCGA | | | |
| | | GAUCUUCCGCAGACG | | | |
| | | CCUGCAGGCACUGAC | | | |
| | | CUCCGGCUGGAGUGU | | | |
| | | GGCCGCAAGCCUGCA | | | |
| | | GCGCCAACGCGAGCU | | | |
| | | GCUGAUGUACAAGC | | | |
| | | GGAUCCUGCUGCGCC | | | |
| | | UGCCCUCCUCCGUGC | | | |
| | | UGUGCGGCUCCUCCU | | | |
| | | UCCAGGCCGAGCAGC | | | |
| | | CCAUCACCGCCCGGU | | | |
| | | GCGAGCAGUUCUUCC | | | |
| | | ACCUGGUGAACAGCG | | | |
| | | AGAUGCGGAACUUC | | | |
| | | UGCUCUCACGGCGGC | | | |
| | | GCUCUGACCCAGGAC | | | |
| | | AUCACCGCCCACUUC | | | |
| | | UUCCGCGGCCUGCUG | | | |
| | | AACGCCUGCCUGCGG | | | |
| | | AGCAGGGACCCCAGC | | | |
| | | CUGAUGGUCGACUUC | | | |
| | | AUCCUGGCCAAGUGC | | | |
| | | CAGACCAAGUGUCCC | | | |
| | | CUGAUCCUGACCAGC | | | |
| | | GCCCUGGUUUGGUG | | | |
| | | GCCCUCACUGGAGCC | | | |
| | | CGUGCUCCUGUGUCG | | | |
| | | CUGGAGGCGGCACUG | | | |
| | | CCAGUCACCCCUGCC | | | |
| | | CCGAGAGCUGCAGAA | | | |
| | | GCUGCAGGAGGGCCG | | | |
| | | GCAGUUCGCCAGCGA | | | |
| | | CUUCCUGUCACCAGA | | | |
| | | GGCAGCCUCUCCCGC | | | |
| | | UCCUAACCCCGAUUG | | | |
| | | GCUAUCCGCGGCCGC | | | |
| | | UCUGCACUUCGCCAU | | | |
| | | CCAGCAGGUGCGGGA | | | |
| | | GGAGAACAUCCGGA | | | |
| | | AGCAGCUGAAGAAG | | | |
| | | CUGGACUGCGAGAG | | | |
| | | GGAGGAGCUGCUUG | | | |
| | | UGUUCCUGUUCUUCU | | | |
| | | UCAGCCUGAUGGGCC | | | |
| | | UGCUGUCCUCCCACC | | | |
| | | UGACCAGCAACUCCA | | | |
| | | CCACCGACCUGCCCA | | | |
| | | AGGCCUUCCACGUGU | | | |
| | | GCGCCGCCAUCCUGG | | | |
| | | AGUGCCUGGAGAAG | | | |
| | | CGGAAGAUCAGCUG | | | |
| | | GCUGGCCCUGUUCCA | | | |
| | | GCUGACCGAGAGCGA | | | |
| | | CCUGCGCCUGGGGAG | | | |
| | | ACUGCUGCUGCGCGU | | | |
| | | GGCUCCCGACCAGCA | | | |
| | | CACCAGGCUGCUGCC | | | |
| | | CUUCGCCUUCUACUC | | | |
| | | CCUGCUGUCCUACUU | | | |
| | | CCACGAGGACGCCGC | | | |
| | | CAUCCGGGAGGAGGC | | | |
| | | CUUCCUGCACGUGGC | | | |
| | | CGUGGACAUGUACCU | | | |
| | | GAAGCUGGUGCAGC | | | |
| | | UGUUCGUGGCCGGCG | | | |
| | | ACACCUCCACCGUGA | | | |
| | | GCCCUCCUGCUGGCC | | | |
| | | GAAGCCUGGAGCUG | | | |
| | | AAGGGCCAGGGCAAC | | | |
| | | CCCGUGGAGCUGAUC | | | |
| | | ACCAAGGCCCGCCUG | | | |
| | | UUCCUGCUGCAGCUG | | | |
| | | AUCCCUCGCUGCCCC | | | |
| | | AAGAAGAGCUUCAG | | | |
| | | CCACGUGGCCGAGCU | | | |
| | | GCUGGCUGACAGGG | | | |
| | | GAGACUGCGACCCCG | | | |
| | | AGGUGAGCGCUGCCC | | | |
| | | UGCAGAGCCGUCAAC | | | |
| | | AAGCGGCCCCUGACG | | | |
| | | CCGAUCUGAGCCAGG | | | |
| | | AGCCCCACCUGUUC | | | |
| | SEQ ID NO: | | | | |
| | 1 | 14 | 50 | 117 | 30 |
| FANCA_010 | MSDSWVPNSA | AUGAGCGACAGCUG | GGAAA | UGAUA | SEQ ID |
| Chemistry: G5 | SGQDPGGRRRA | GGUGCCCAACAGUGC | UCGCAA | AUAGGC | NO: 30 |
| Cap: C1 | WAELLAGRVK | UUCCGGGCAGGAUCC | AAUUU | UGGAGC | consists |
| PolyA tail: | REKYNPERAQK | CGGCGGUAGAAGAC | GCUCUU | CUCCAC | from 5′ to |
| 100 nt (SEQ ID | LKESAVRLLRS | GGGCUUGGGCUGAG | CGCGUU | CGCGUU | 3′ end: 5′ |
| NO: 121) | HQDLNALLLEV | CUGCUGGCCGGCAGG | AGAUU | AUCCGU | UTR of |
| | EGPLCKKLSLS | GUGAAGCGGGAGAA | UCUUUU | UCCUCG | SEQ ID |
| | KVIDCDSSEAY | GUACAACCCCGAGCG | AGUUU | UAGGCU | NO: 50, |
| | ANHSSSFIGSAL | GGCCCAGAAGCUGAA | UCUCGC | GGUCCU | ORF |
| | QDQASRLGVPV | GGAGAGUGCCGUGC | AACUAG | GGGGA | Sequence |
| | GILSAGMVASS | GGCUGCUGCGGAGCC | CAAGCU | ACGGGU | of SEQ ID |
| | VGQICTAPAET | ACCAGGACCUGAACG | UUUUG | CGGCGG | NO: 14, |
| | SHPVLLTVEQR | CCCUCCUGCUGGAGG | UUCUCG | GUACCC | and 3′ |
| | KKLSSLLEFAQ | UCGAGGGCCCACUGU | CC | CCGUGG | UTR of |
| | YLLAHSMFSRL | GCAAGAAGCUCAGCC | | UCUUUG | SEQ ID |
| | SFCQELWKIQS | UGAGCAAGGUGAUC | | AAUAA | NO: 117 |
| | SLLLEAVWHLH | GACUGUGACAGCAGC | | AGUCUG | |
| | VQGIVSLQELL | GAGGCCUACGCCAAC | | AGUGG | |
| | ESHPDMHAVG | CACAGCUCCAGCUUC | | GCGGC | |
| | SWLFRNLCCLC | AUCGGCAGCGCCCUG | | | |
| | EQMEASCQHA | CAGGACCAGGCCAGC | | | |
| | DVARAMLSDF | AGACUGGGCGUGCCC | | | |
| | VQMFVLRGFQ | GUGGGCAUCCUGAGC | | | |
| | KNSDLRRTVEP | GCCGGCAUGGUGGCC | | | |
| | EKMPQVTVDV | AGCUCGGUGGGCCAG | | | |
| | LQRMLIFALDA | AUCUGCACCGCUCCC | | | |
| | LAAGVQEESST | GCCGAGACCAGCCAC | | | |
| | HKIVRCWFGVF | CCCGUGCUGCUCACC | | | |
| | SGHTLGSVISTD | GUGGAGCAGCGGAA | | | |
| | PLKRFFSHTLT | GAAGCUGAGCAGCCU | | | |
| | QILTHSPVLKAS | GCUGGAGUUCGCCCA | | | |
| | DAVQMQREWS | GUACCUGCUGGCCCA | | | |
| | FARTHPLLTSL | CAGCAUGUUCUCCCG | | | |
| | YRRLFVMLSAE | GCUGAGCUUUUGCCA | | | |
| | ELVGHLQEVLE | AGAGCUGUGGAAGA | | | |
| | TQEVHWQRVL | UCCAGAGCUCCCUGC | | | |
| | SFVSALVVCFP | UGCUGGAGGCCGUG | | | |
| | EAQQLLEDWV | UGGCACCUUCACGUG | | | |
| | ARLMAQAFESC | CAGGGCAUCGUGAGC | | | |
| | QLDSMVTAFLV | CUGCAGGAACUGCUG | | | |
| | VRQAALEGPSA | GAGAGCCACCCCGAC | | | |
| | FLSYADWFKAS | AUGCACGCCGUGGGC | | | |
| | FGSTRGYHGCS | AGCUGGCUGUUCCGG | | | |
| | KKALVFLFTFL | AACCUGUGCUGCCUG | | | |
| | SELVPFESPRYL | UGCGAGCAGAUGGA | | | |
| | QVHILHPPLVP | GGCUAGCUGCCAGCA | | | |
| | GKYRSLLTDYI | CGCCGACGUGGCACG | | | |
| | SLAKTRLADLK | GGCCAUGCUGAGCGA | | | |
| | VSIENMGLYED | CUUCGUGCAGAUGU | | | |
| | LSSAGDITEPHS | UCGUGCUGCGGGGCU | | | |
| | QALQDVEKAI | UCCAGAAGAAUAGC | | | |
| | MVFEHTGNIPV | GACCUACGGCGGACC | | | |
| | TVMEASIFRRP | GUGGAGCCCGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UCGCCCUGGACGCUC | | | |
| | QACSAAEEKPE | UGGCCGCUGGCGUGC | | | |
| | DAALGVRAEP | AGGAGGAGAGCAGC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCAGCGGCCAC | | | |
| | VAVISERLRAV | ACCCUGGGCAGCGUG | | | |
| | LGHNEDDSSVE | AUCAGCACCGACCCA | | | |
| | ISKIQLSINTPRL | CUGAAGCGGUUCUUC | | | |
| | EPREHMAVDLL | AGCCACACCCUCACC | | | |
| | LTSFCQNLMAA | CAGAUCCUGACCCAC | | | |
| | SSVAPPERQGP | AGCCCCGUGCUGAAG | | | |
| | WAALFVRTMC | GCCAGCGACGCCGUG | | | |
| | GRVLPAVLTRL | CAGAUGCAGCGGGA | | | |
| | CQLLRHQGPSL | GUGGAGCUUCGCCCG | | | |
| | SAPHVLGLAAL | GACCCACCCACUGCU | | | |
| | AVHLGESRSAL | GACUAGCCUGUACCG | | | |
| | PEVDVGPPAPG | GCGGCUGUUCGUGA | | | |
| | AGLPVPALFDS | UGCUGAGCGCAGAG | | | |
| | LLTCRTRDSLFF | GAGCUGGUGGGCCAC | | | |
| | CLKFCTAAISYS | CUGCAGGAGGUGCU | | | |
| | LCKFSSQSRDT | GGAGACCCAGGAGG | | | |
| | LCSCLSPGLIKK | UGCACUGGCAGCGGG | | | |
| | FQFLMFRLFSE | UGCUGAGCUUCGUG | | | |
| | ARQPLSEEDVA | AGCGCCCUGGUGGUG | | | |
| | SLSWRPLHLPS | UGCUUCCCCGAGGCC | | | |
| | ADWQRAALSL | CAGCAGCUCCUGGAG | | | |
| | WTHRTFREVLK | GACUGGGUGGCCCGG | | | |
| | EEDVHLTYQD | CUGAUGGCCCAGGCC | | | |
| | WLHLELEIQPE | UUCGAGAGCUGCCAG | | | |
| | ADALSDTERQD | CUGGACAGCAUGGU | | | |
| | FHQWAIHEHFL | GACCGCCUUCCUGGU | | | |
| | PESSASGGCDG | GGUGCGGCAGGCUGC | | | |
| | DLQAACTILVN | CCUGGAAGGCCCCAG | | | |
| | ALMDFHQSSRS | CGCUUUCCUGAGCUA | | | |
| | YDHSENSDLVF | CGCCGACUGGUUCAA | | | |
| | GGRTGNEDIISR | GGCCAGCUUCGGCUC | | | |
| | LQEMVADLEL | CACCCGGGGCUACCA | | | |
| | QQDLIVPLGHT | CGGCUGCAGCAAGAA | | | |
| | PSQEHFLFEIFR | GGCCCUGGUGUUCCU | | | |
| | RRLQALTSGWS | GUUCACCUUCCUGAG | | | |
| | VAASLQRQREL | CGAGCUGGUGCCCUU | | | |
| | LMYKRILLRLP | CGAGUCACCCCGGUA | | | |
| | SSVLCGSSFQA | CCUGCAGGUGCACAU | | | |
| | EQPITARCEQFF | CCUGCAUCCUCCGCU | | | |
| | HLVNSEMRNFC | GGUGCCCGGCAAGUA | | | |
| | SHGGALTQDIT | CCGGAGCCUGCUGAC | | | |
| | AHFFRGLLNAC | CGACUACAUCAGCCU | | | |
| | LRSRDPSLMVD | GGCCAAGACCCGGCU | | | |
| | FILAKCQTKCP | GGCCGACCUGAAGGU | | | |
| | LILTSALVWWP | GAGCAUCGAGAACA | | | |
| | SLEPVLLCRWR | UGGGCCUGUACGAG | | | |
| | RHCQSPLPREL | GACCUGAGCAGCGCC | | | |
| | QKLQEGRQFAS | GGCGACAUCACCGAG | | | |
| | DFLSPEAASPAP | CCCCACAGCCAGGCC | | | |
| | NPDWLSAAAL | CUGCAAGACGUGGA | | | |
| | HFAIQQVREENI | GAAGGCCAUCAUGG | | | |
| | RKQLKKLDCER | UGUUCGAGCACACCG | | | |
| | EELLVFLFFFSL | GCAACAUCCCCGUGA | | | |
| | MGLLSSHLTSN | CCGUCAUGGAGGCCA | | | |
| | STTDLPKAFHV | GCAUCUUCCGGCGGC | | | |
| | CAAILECLEKR | CCUACUACGUGAGCC | | | |
| | KISWLALFQLT | ACUUCCUGCCCGCCC | | | |
| | ESDLRLGRLLL | UGCUGACCCCACGGG | | | |
| | RVAPDQHTRLL | UGCUGCCCAAGGUGC | | | |
| | PFAFYSLLSYFH | CCGACAGCCGGGUGG | | | |
| | EDAAIREEAFL | CCUUCAUCGAGAGCC | | | |
| | HVAVDMYLKL | UGAAGCGGGCCGACA | | | |
| | VQLFVAGDTST | AGAUCCCACCCAGCC | | | |
| | VSPPAGRSLEL | UGUACAGCACCUACU | | | |
| | KGQGNPVELIT | GCCAGGCCUGCUCUG | | | |
| | KARLFLLQLIPR | CCGCCGAGGAGAAGC | | | |
| | CPKKSFSHVAE | CCGAGGACGCCGCAU | | | |
| | LLADRGDCDPE | UAGGCGUGCGGGCCG | | | |
| | VSAALQSRQQA | AGCCCAACAGCGCCG | | | |
| | APDADLSQEPH | AGGAACCCCUGGGGC | | | |
| | LF | AGCUGACCGCAGCCC | | | |
| | | UGGGAGAGCUGCGG | | | |
| | | GCCAGCAUGACCGAC | | | |
| | | CCCAGCCAGCGGGAC | | | |
| | | GUGAUCAGCGCCCAG | | | |
| | | GUGGCCGUCAUCAGC | | | |
| | | GAGCGGCUGCGGGCA | | | |
| | | GUGCUGGGCCACAAC | | | |
| | | GAGGACGACAGCAGC | | | |
| | | GUGGAGAUCAGCAA | | | |
| | | GAUCCAGCUGAGCAU | | | |
| | | CAACACCCCACGGCU | | | |
| | | GGAACCUCGGGAGCA | | | |
| | | CAUGGCCGUGGACCU | | | |
| | | GCUGCUGACCAGCUU | | | |
| | | CUGCCAGAACCUGAU | | | |
| | | GGCCGCCUCUAGCGU | | | |
| | | GGCCCCACCAGAACG | | | |
| | | GCAGGGUCCCUGGGC | | | |
| | | UGCCCUGUUCGUGCG | | | |
| | | GACCAUGUGCGGCCG | | | |
| | | GGUUCUGCCCGCUGU | | | |
| | | GCUGACCCGGCUGUG | | | |
| | | CCAGCUGCUGCGGCA | | | |
| | | CCAGGGCCCUAGCCU | | | |
| | | GAGCGCUCCCCACGU | | | |
| | | GCUGGGCCUAGCUGC | | | |
| | | CCUGGCCGUGCACCU | | | |
| | | UGGGGAGUCCCGAA | | | |
| | | GCGCCCUGCCCGAGG | | | |
| | | UGGACGUGGGCCCUC | | | |
| | | CAGCCCCAGGUGCUG | | | |
| | | GCCUUCCCGUGCCCG | | | |
| | | CUCUGUUCGACAGUC | | | |
| | | UGCUGACCUGCCGGA | | | |
| | | CCCGGGACAGCCUGU | | | |
| | | UCUUCUGCCUGAAGU | | | |
| | | UCUGCACCGCCGCCA | | | |
| | | UCAGCUACAGCCUGU | | | |
| | | GCAAGUUCAGCAGCC | | | |
| | | AGAGCCGGGACACCC | | | |
| | | UGUGCAGCUGCCUGA | | | |
| | | GCCCCGGCCUGAUCA | | | |
| | | AGAAGUUCCAGUUCC | | | |
| | | UGAUGUUCCGGCUG | | | |
| | | UUCAGCGAAGCCCGG | | | |
| | | CAGCCCCUGAGCGAG | | | |
| | | GAGGACGUCGCCAGC | | | |
| | | CUGUCCUGGAGACCC | | | |
| | | CUGCACCUGCCCAGC | | | |
| | | GCCGACUGGCAGAGA | | | |
| | | GCCGCCCUGAGCCUG | | | |
| | | UGGACCCACCGGACC | | | |
| | | UUCCGGGAGGUGCU | | | |
| | | GAAGGAAGAGGACG | | | |
| | | UGCACCUGACCUACC | | | |
| | | AGGACUGGCUGCACC | | | |
| | | UGGAGCUGGAGAUU | | | |
| | | CAGCCCGAGGCCGAC | | | |
| | | GCCCUGAGCGACACC | | | |
| | | GAGCGGCAGGAUUU | | | |
| | | CCACCAGUGGGCCAU | | | |
| | | CCACGAGCAUUUCCU | | | |
| | | GCCCGAGAGCAGCGC | | | |
| | | CAGCGGCGGCUGUGA | | | |
| | | CGGCGACCUGCAGGC | | | |
| | | CGCCUGCACCAUCCU | | | |
| | | GGUGAACGCCCUGAU | | | |
| | | GGACUUCCACCAGAG | | | |
| | | CAGCCGGAGCUACGA | | | |
| | | CCACAGCGAGAACAG | | | |
| | | CGACCUGGUGUUCGG | | | |
| | | CGGACGGACCGGCAA | | | |
| | | CGAGGACAUCAUCAG | | | |
| | | CCGGCUGCAGGAGAU | | | |
| | | GGUCGCCGACCUGGA | | | |
| | | GUUGCAGCAGGACCU | | | |
| | | GAUCGUGCCCCUGGG | | | |
| | | CCACACUCCCAGCCA | | | |
| | | GGAGCACUUCCUGUU | | | |
| | | CGAGAUCUUCCGCCG | | | |
| | | GAGACUGCAGGCCCU | | | |
| | | GACCUCAGGCUGGAG | | | |
| | | CGUGGCCGCCAGCCU | | | |
| | | GCAACGGCAGCGGGA | | | |
| | | GCUGCUGAUGUACA | | | |
| | | AGCGGAUCCUGCUGC | | | |
| | | GGCUGCCCAGCAGCG | | | |
| | | UGCUGUGCGGCAGCA | | | |
| | | GCUUCCAGGCCGAGC | | | |
| | | AGCCCAUCACGGCCC | | | |
| | | GGUGCGAGCAGUUC | | | |
| | | UUCCACCUGGUGAAC | | | |
| | | AGCGAGAUGCGGAA | | | |
| | | CUUCUGCAGCCACGG | | | |
| | | AGGCGCCCUGACCCA | | | |
| | | GGACAUCACCGCCCA | | | |
| | | CUUCUUCCGGGGCCU | | | |
| | | GCUGAACGCCUGCCU | | | |
| | | GCGGAGCAGAGACCC | | | |
| | | CAGCCUGAUGGUGG | | | |
| | | ACUUCAUCCUGGCCA | | | |
| | | AGUGCCAGACCAAGU | | | |
| | | GUCCCCUGAUCCUGA | | | |
| | | CCUCCGCCCUGGUGU | | | |
| | | GGUGGCCCAGCCUGG | | | |
| | | AGCCCGUGCUUCUGU | | | |
| | | GUCGGUGGCGGCGGC | | | |
| | | ACUGCCAGAGCCCUC | | | |
| | | UGCCCCGGGAGCUGC | | | |
| | | AGAAGCUGCAGGAG | | | |
| | | GGCCGGCAGUUCGCC | | | |
| | | AGCGACUUCCUGUCU | | | |
| | | CCCGAGGCCGCUAGC | | | |
| | | CCAGCCCCAAACCCC | | | |
| | | GACUGGCUGAGCGCU | | | |
| | | GCCGCCCUGCACUUC | | | |
| | | GCCAUCCAGCAGGUG | | | |
| | | CGGGAGGAGAACAU | | | |
| | | CCGGAAGCAGCUGAA | | | |
| | | GAAGCUGGACUGCG | | | |
| | | AGCGGGAGGAGCUG | | | |
| | | CUCGUGUUCCUCUUC | | | |
| | | UUCUUCAGCCUCAUG | | | |
| | | GGCCUUCUGAGCAGC | | | |
| | | CACCUGACCAGCAAC | | | |
| | | AGCACCACCGACCUG | | | |
| | | CCCAAGGCCUUCCAC | | | |
| | | GUGUGCGCCGCCAUC | | | |
| | | CUGGAGUGCCUGGA | | | |
| | | GAAGCGGAAGAUCA | | | |
| | | GCUGGCUGGCCCUGU | | | |
| | | UCCAGCUGACCGAGA | | | |
| | | GCGACCUGCGGCUGG | | | |
| | | GCCGACUGCUGCUGC | | | |
| | | GGGUGGCCCCAGACC | | | |
| | | AGCACACCCGGCUGC | | | |
| | | UGCCCUUCGCCUUCU | | | |
| | | ACAGCCUGCUGAGCU | | | |
| | | ACUUCCACGAGGACG | | | |
| | | CUGCCAUCCGGGAGG | | | |
| | | AGGCCUUUCUGCACG | | | |
| | | UGGCAGUGGACAUG | | | |
| | | UACCUGAAGCUGGU | | | |
| | | GCAGCUGUUCGUGGC | | | |
| | | CGGCGACACCAGCAC | | | |
| | | CGUGUCACCACCAGC | | | |
| | | CGGCCGCUCCCUGGA | | | |
| | | GCUGAAGGGCCAGG | | | |
| | | GCAACCCCGUGGAGC | | | |
| | | UGAUCACCAAGGCCC | | | |
| | | GGCUGUUCCUGCUGC | | | |
| | | AGCUGAUCCCACGGU | | | |
| | | GCCCCAAGAAGAGCU | | | |
| | | UCAGCCACGUGGCCG | | | |
| | | AGCUGCUUGCCGACC | | | |
| | | GGGGAGACUGCGACC | | | |
| | | CCGAGGUGAGCGCCG | | | |
| | | CCUUGCAGAGCCGGC | | | |
| | | AGCAGGCCGCACCUG | | | |
| | | ACGCCGACCUGAGCC | | | |
| | | AGGAGCCCCACCUGU | | | |
| | | UC | | | |
| | SEQ ID NO: | | | | |
| | 1 | 2 | 61 | 117 | 31 |
| FANCA_011 | MSDSWVPNSA | AUGUCCGACUCCUGG | GGAAAC | UGAUA | SEQ ID |
| Chemistry: G5 | SGQDPGGRRRA | GUGCCCAACUCCGCU | UUUAU | AUAGGC | NO: 31 |
| Cap: C1 | WAELLAGRVK | AGCGGACAAGAUCCA | UUAGU | UGGAGC | consists |
| PolyA tail: | REKYNPERAQK | GGUGGCCGCAGGAG | GUUACU | CUCCAC | from 5′ to |
| 100 nt (SEQ ID | LKESAVRLLRS | AGCGUGGGCUGAAC | UUAUU | CGCGUU | 3′ end: 5′ |
| NO: 121) | HQDLNALLLEV | UGCUUGCCGGGCGAG | UUCUGU | AUCCGU | UTR of |
| | EGPLCKKLSLS | UGAAGCGCGAGAAG | UUAUU | UCCUCG | SEQ ID |
| | KVIDCDSSEAY | UACAACCCCGAGCGC | UGUGU | UAGGCU | NO: 61, |
| | ANHSSSFIGSAL | GCCCAGAAGCUGAAG | UUCUUC | GGUCCU | ORF |
| | QDQASRLGVPV | GAGAGCGCCGUACGG | AGUGG | GGGGA | Sequence |
| | GILSAGMVASS | CUGCUGCGCAGCCAC | GUUUG | ACGGGU | of SEQ ID |
| | VGQICTAPAET | CAGGACCUGAACGCC | UUCUAA | CGGCGG | NO: 2, and |
| | SHPVLLTVEQR | CUGCUGCUGGAGGU | UUUCCU | GUACCC | 3′ UTR of |
| | KKLSSLLEFAQ | GGAGGGGCCCCUGUG | UGGCCG | CCGUGG | SEQ ID |
| | YLLAHSMFSRL | CAAGAAGCUGAGCCU | CC | UCUUUG | NO: 117 |
| | SFCQELWKIQS | GUCCAAGGUGAUCG | | AAUAA | |
| | SLLLEAVWHLH | ACUGCGACAGCAGCG | | AGUCUG | |
| | VQGIVSLQELL | AGGCCUACGCCAACC | | AGUGG | |
| | ESHPDMHAVG | ACAGCUCCUCCUUCA | | GCGGC | |
| | SWLFRNLCCLC | UCGGCUCCGCCCUGC | | | |
| | EQMEASCQHA | AGGAUCAAGCUAGCC | | | |
| | DVARAMLSDF | GGCUGGGCGUUCCAG | | | |
| | VQMFVLRGFQ | UGGGCAUCCUGAGCG | | | |
| | KNSDLRRTVEP | CCGGCAUGGUGGCCA | | | |
| | EKMPQVTVDV | GCAGCGUGGGCCAGA | | | |
| | LQRMLIFALDA | UCUGCACCGCUCCCG | | | |
| | LAAGVQEESST | CCGAGACCAGCCACC | | | |
| | HKIVRCWFGVF | CCGUGCUGCUGACCG | | | |
| | SGHTLGSVISTD | UGGAGCAGCGCAAG | | | |
| | PLKRFFSHTLT | AAGCUGUCAAGCCUG | | | |
| | QILTHSPVLKAS | CUGGAGUUCGCCCAG | | | |
| | DAVQMQREWS | UACCUGCUGGCCCAC | | | |
| | FARTHPLLTSL | AGCAUGUUCUCCCGC | | | |
| | YRRLFVMLSAE | CUGAGCUUCUGCCAG | | | |
| | ELVGHLQEVLE | GAGCUGUGGAAGAU | | | |
| | TQEVHWQRVL | CCAGUCCAGCCUGCU | | | |
| | SFVSALVVCFP | ACUGGAGGCCGUGU | | | |
| | EAQQLLEDWV | GGCACUUACACGUGC | | | |
| | ARLMAQAFESC | AGGGCAUCGUGAGCC | | | |
| | QLDSMVTAFLV | UGCAGGAGCUACUG | | | |
| | VRQAALEGPSA | GAGAGCCACCCCGAC | | | |
| | FLSYADWFKAS | AUGCACGCCGUGGGG | | | |
| | FGSTRGYHGCS | UCCUGGCUGUUCCGC | | | |
| | KKALVFLFTFL | AACCUGUGCUGCCUG | | | |
| | SELVPFESPRYL | UGCGAGCAGAUGGA | | | |
| | QVHILHPPLVP | AGCCUCCUGCCAACA | | | |
| | GKYRSLLTDYI | CGCCGACGUUGCACG | | | |
| | SLAKTRLADLK | GGCAAUGCUGAGCG | | | |
| | VSIENMGLYED | ACUUCGUGCAGAUG | | | |
| | LSSAGDITEPHS | UUCGUGCUGCGCGGG | | | |
| | QALQDVEKAI | UUCCAGAAGAACUCA | | | |
| | MVFEHTGNIPV | GACCUCCGGAGGACC | | | |
| | TVMEASIFRRP | GUGGAGCCUGAGAA | | | |
| | YYVSHFLPALL | GAUGCCCCAGGUGAC | | | |
| | TPRVLPKVPDS | CGUGGACGUGCUGCA | | | |
| | RVAFIESLKRA | GCGGAUGCUGAUCU | | | |
| | DKIPPSLYSTYC | UUGCCCUGGACGCUC | | | |
| | QACSAAEEKPE | UCGCUGCCGGAGUGC | | | |
| | DAALGVRAEP | AGGAGGAGAGCUCC | | | |
| | NSAEEPLGQLT | ACCCACAAGAUCGUG | | | |
| | AALGELRASMT | CGGUGCUGGUUCGGC | | | |
| | DPSQRDVISAQ | GUGUUCUCCGGCCAC | | | |
| | VAVISERLRAV | ACCCUGGGCUCCGUG | | | |
| | LGHNEDDSSVE | AUCUCCACCGAUCCC | | | |
| | ISKIQLSINTPRL | CUGAAGCGGUUCUUC | | | |
| | EPREHMAVDLL | UCCCACACCCUGACC | | | |
| | LTSFCQNLMAA | CAGAUCCUGACCCAC | | | |
| | SSVAPPERQGP | UCCCCUGUGCUGAAG | | | |
| | WAALFVRTMC | GCCAGCGACGCCGUG | | | |
| | GRVLPAVLTRL | CAGAUGCAGCGCGAG | | | |
| | CQLLRHQGPSL | UGGUCCUUCGCCCGC | | | |
| | SAPHVLGLAAL | ACCCAUCCCCUGCUG | | | |
| | AVHLGESRSAL | ACCAGCCUGUACCGG | | | |
| | PEVDVGPPAPG | CGGCUGUUCGUGAU | | | |
| | AGLPVPALFDS | GCUGUCCGCCGAGGA | | | |
| | LLTCRTRDSLFF | GCUGGUGGGCCACCU | | | |
| | CLKFCTAAISYS | GCAGGAGGUGCUGG | | | |
| | LCKFSSQSRDT | AGACCCAGGAGGUGC | | | |
| | LCSCLSPGLIKK | ACUGGCAGCGCGUGC | | | |
| | FQFLMFRLFSE | UGAGCUUCGUGAGC | | | |
| | ARQPLSEEDVA | GCCCUGGUGGUGUGC | | | |
| | SLSWRPLHLPS | UUCCCCGAGGCCCAG | | | |
| | ADWQRAALSL | CAGCUCCUGGAGGAC | | | |
| | WTHRTFREVLK | UGGGUGGCCCGGCUG | | | |
| | EEDVHLTYQD | AUGGCCCAGGCCUUC | | | |
| | WLHLELEIQPE | GAGAGCUGCCAGCUG | | | |
| | ADALSDTERQD | GACAGCAUGGUGACC | | | |
| | FHQWAIHEHFL | GCCUUCCUGGUGGUG | | | |
| | PESSASGGCDG | CGUCAAGCAGCCCUG | | | |
| | DLQAACTILVN | GAGGGCCCAAGCGCC | | | |
| | ALMDFHQSSRS | UUCCUGAGCUACGCC | | | |
| | YDHSENSDLVF | GACUGGUUCAAGGCC | | | |
| | GGRTGNEDIISR | AGCUUCGGGUCCACC | | | |
| | LQEMVADLEL | CGCGGGUACCACGGC | | | |
| | QQDLIVPLGHT | UGCUCCAAGAAGGCC | | | |
| | PSQEHFLFEIFR | CUGGUGUUCCUGUU | | | |
| | RRLQALTSGWS | UACCUUCCUGAGCGA | | | |
| | VAASLQRQREL | GCUGGUGCCCUUCGA | | | |
| | LMYKRILLRLP | GUCGCCCCGCUACCU | | | |
| | SSVLCGSSFQA | GCAGGUGCACAUCCU | | | |
| | EQPITARCEQFF | GCACCCACCCCUGGU | | | |
| | HLVNSEMRNFC | CCCUGGCAAGUACCG | | | |
| | SHGGALTQDIT | GAGCCUGCUGACCGA | | | |
| | AHFFRGLLNAC | CUACAUCUCCCUGGC | | | |
| | LRSRDPSLMVD | CAAGACCCGGCUGGC | | | |
| | FILAKCQTKCP | CGACCUGAAGGUGA | | | |
| | LILTSALVWWP | GCAUCGAGAACAUG | | | |
| | SLEPVLLCRWR | GGCCUGUACGAGGAC | | | |
| | RHCQSPLPREL | CUGUCCAGCGCCGGC | | | |
| | QKLQEGRQFAS | GACAUCACCGAGCCC | | | |
| | DFLSPEAASPAP | CACAGCCAGGCCCUG | | | |
| | NPDWLSAAAL | CAGGACGUGGAGAA | | | |
| | HFAIQQVREENI | GGCCAUCAUGGUGU | | | |
| | RKQLKKLDCER | UCGAGCACACCGGCA | | | |
| | EELLVFLFFFSL | ACAUCCCCGUGACCG | | | |
| | MGLLSSHLTSN | UGAUGGAGGCUAGU | | | |
| | STTDLPKAFHV | AUCUUCCGGCGGCCC | | | |
| | CAAILECLEKR | UACUACGUGUCCCAC | | | |
| | KISWLALFQLT | UUCCUGCCCGCCCUG | | | |
| | ESDLRLGRLLL | CUGACACCCCGGGUG | | | |
| | RVAPDQHTRLL | CUGCCCAAGGUGCCC | | | |
| | PFAFYSLLSYFH | GACAGUCGCGUCGCC | | | |
| | EDAAIREEAFL | UUCAUCGAGAGCCUG | | | |
| | HVAVDMYLKL | AAGCGGGCCGACAAG | | | |
| | VQLFVAGDTST | AUCCCGCCCUCCCUG | | | |
| | VSPPAGRSLEL | UACUCCACCUACUGC | | | |
| | KGQGNPVELIT | CAGGCUUGCUCCGCC | | | |
| | KARLFLLQLIPR | GCCGAGGAGAAGCCC | | | |
| | CPKKSFSHVAE | GAGGACGCAGCCUUA | | | |
| | LLADRGDCDPE | GGAGUACGCGCCGAG | | | |
| | VSAALQSRQQA | CCCAACAGCGCCGAG | | | |
| | APDADLSQEPH | GAGCCACUGGGCCAG | | | |
| | LF | UUGACCGCUGCCCUG | | | |
| | | GGAGAGCUGCGGGCC | | | |
| | | AGCAUGACCGACCCC | | | |
| | | AGCCAACGCGACGUG | | | |
| | | AUCAGCGCCCAGGUG | | | |
| | | GCCGUGAUCAGCGAG | | | |
| | | AGGCUGCGGGCAGU | | | |
| | | GCUGGGCCACAACGA | | | |
| | | GGACGACAGCAGCGU | | | |
| | | GGAGAUCAGCAAGA | | | |
| | | UCCAGCUGAGCAUCA | | | |
| | | ACACACCCAGGCUGG | | | |
| | | AGCCCAGAGAGCACA | | | |
| | | UGGCCGUGGACCUGC | | | |
| | | UGCUGACCUCCUUCU | | | |
| | | GCCAGAACCUGAUGG | | | |
| | | CCGCAAGCAGCGUGG | | | |
| | | CUCCUCCAGAACGCC | | | |
| | | AAGGCCCUUGGGCUG | | | |
| | | CCCUGUUCGUGCGCA | | | |
| | | CCAUGUGUGGCCGCG | | | |
| | | UCCUGCCAGCAGUGC | | | |
| | | UGACCCGGCUGUGCC | | | |
| | | AGCUUCUGCGGCACC | | | |
| | | AAGGGCCCUCCCUGA | | | |
| | | GCGCUCCCCACGUGC | | | |
| | | UUGGGUUGGCCGCCC | | | |
| | | UCGCUGUGCACCUGG | | | |
| | | GCGAGAGCAGGAGC | | | |
| | | GCCCUUCCCGAGGUG | | | |
| | | GACGUGGGUCCUCCA | | | |
| | | GCCCCAGGUGCCGGA | | | |
| | | CUCCCCGUGCCAGCC | | | |
| | | CUGUUCGACAGCCUG | | | |
| | | CUGACUUGUCGCACC | | | |
| | | CGGGACUCCCUGUUC | | | |
| | | UUCUGCCUGAAGUUC | | | |
| | | UGCACCGCCGCCAUC | | | |
| | | UCCUACUCCCUGUGC | | | |
| | | AAGUUCUCCAGCCAG | | | |
| | | UCCCGGGACACCCUG | | | |
| | | UGCUCCUGCCUGUCU | | | |
| | | CCCGGCCUGAUCAAG | | | |
| | | AAGUUCCAGUUCCUG | | | |
| | | AUGUUCCGCCUGUUC | | | |
| | | AGCGAAGCACGCCAG | | | |
| | | CCCCUGUCCGAGGAG | | | |
| | | GACGUGGCCUCCCUG | | | |
| | | UCCUGGCGACCCCUG | | | |
| | | CACCUGCCCUCCGCC | | | |
| | | GAUUGGCAGCGCGCC | | | |
| | | GCCCUUAGCCUGUGG | | | |
| | | ACCCACCGGACCUUC | | | |
| | | CGCGAGGUGCUGAA | | | |
| | | GGAGGAAGACGUGC | | | |
| | | ACCUGACCUACCAGG | | | |
| | | ACUGGCUGCACCUGG | | | |
| | | AGCUGGAGAUCCAGC | | | |
| | | CCGAAGCCGACGCCC | | | |
| | | UGAGCGACACCGAGC | | | |
| | | GCCAGGACUUUCACC | | | |
| | | AGUGGGCCAUCCACG | | | |
| | | AGCACUUUCUGCCCG | | | |
| | | AGAGCAGCGCCAGUG | | | |
| | | GAGGCUGCGACGGCG | | | |
| | | ACCUGCAAGCCGCCU | | | |
| | | GCACCAUCCUGGUGA | | | |
| | | ACGCCCUGAUGGACU | | | |
| | | UCCACCAGAGCUCCC | | | |
| | | GCAGCUACGACCACU | | | |
| | | CCGAGAACAGCGACC | | | |
| | | UGGUGUUUGGAGGC | | | |
| | | CGCACCGGGAACGAG | | | |
| | | GACAUCAUCAGCCGC | | | |
| | | CUGCAGGAGAUGGU | | | |
| | | GGCCGACCUGGAGCU | | | |
| | | CCAGCAGGACCUGAU | | | |
| | | CGUGCCACUGGGGCA | | | |
| | | CACCCCAAGCCAGGA | | | |
| | | GCACUUCCUGUUCGA | | | |
| | | GAUCUUCCGCAGACG | | | |
| | | CCUGCAGGCACUGAC | | | |
| | | CUCCGGCUGGAGUGU | | | |
| | | GGCCGCAAGCCUGCA | | | |
| | | GCGCCAACGCGAGCU | | | |
| | | GCUGAUGUACAAGC | | | |
| | | GGAUCCUGCUGCGCC | | | |
| | | UGCCCUCCUCCGUGC | | | |
| | | UGUGCGGCUCCUCCU | | | |
| | | UCCAGGCCGAGCAGC | | | |
| | | CCAUCACCGCCCGGU | | | |
| | | GCGAGCAGUUCUUCC | | | |
| | | ACCUGGUGAACAGCG | | | |
| | | AGAUGCGGAACUUC | | | |
| | | UGCUCUCACGGCGGC | | | |
| | | GCUCUGACCCAGGAC | | | |
| | | AUCACCGCCCACUUC | | | |
| | | UUCCGCGGCCUGCUG | | | |
| | | AACGCCUGCCUGCGG | | | |
| | | AGCAGGGACCCCAGC | | | |
| | | CUGAUGGUCGACUUC | | | |
| | | AUCCUGGCCAAGUGC | | | |
| | | CAGACCAAGUGUCCC | | | |
| | | CUGAUCCUGACCAGC | | | |
| | | GCCCUGGUUUGGUG | | | |
| | | GCCCUCACUGGAGCC | | | |
| | | CGUGCUCCUGUGUCG | | | |
| | | CUGGAGGCGGCACUG | | | |
| | | CCAGUCACCCCUGCC | | | |
| | | CCGAGAGCUGCAGAA | | | |
| | | GCUGCAGGAGGGCCG | | | |
| | | GCAGUUCGCCAGCGA | | | |
| | | CUUCCUGUCACCAGA | | | |
| | | GGCAGCCUCUCCCGC | | | |
| | | UCCUAACCCCGAUUG | | | |
| | | GCUAUCCGCGGCCGC | | | |
| | | UCUGCACUUCGCCAU | | | |
| | | CCAGCAGGUGCGGGA | | | |
| | | GGAGAACAUCCGGA | | | |
| | | AGCAGCUGAAGAAG | | | |
| | | CUGGACUGCGAGAG | | | |
| | | GGAGGAGCUGCUUG | | | |
| | | UGUUCCUGUUCUUCU | | | |
| | | UCAGCCUGAUGGGCC | | | |
| | | UGCUGUCCUCCCACC | | | |
| | | UGACCAGCAACUCCA | | | |
| | | CCACCGACCUGCCCA | | | |
| | | AGGCCUUCCACGUGU | | | |
| | | GCGCCGCCAUCCUGG | | | |
| | | AGUGCCUGGAGAAG | | | |
| | | CGGAAGAUCAGCUG | | | |
| | | GCUGGCCCUGUUCCA | | | |
| | | GCUGACCGAGAGCGA | | | |
| | | CCUGCGCCUGGGGAG | | | |
| | | ACUGCUGCUGCGCGU | | | |
| | | GGCUCCCGACCAGCA | | | |
| | | CACCAGGCUGCUGCC | | | |
| | | CUUCGCCUUCUACUC | | | |
| | | CCUGCUGUCCUACUU | | | |
| | | CCACGAGGACGCCGC | | | |
| | | CAUCCGGGAGGAGGC | | | |
| | | CUUCCUGCACGUGGC | | | |
| | | CGUGGACAUGUACCU | | | |
| | | GAAGCUGGUGCAGC | | | |
| | | UGUUCGUGGCCGGCG | | | |
| | | ACACCUCCACCGUGA | | | |
| | | GCCCUCCUGCUGGCC | | | |
| | | GAAGCCUGGAGCUG | | | |
| | | AAGGGCCAGGGCAAC | | | |
| | | CCCGUGGAGCUGAUC | | | |
| | | ACCAAGGCCCGCCUG | | | |
| | | UUCCUGCUGCAGCUG | | | |
| | | AUCCCUCGCUGCCCC | | | |
| | | AAGAAGAGCUUCAG | | | |
| | | CCACGUGGCCGAGCU | | | |
| | | GCUGGCUGACAGGG | | | |
| | | GAGACUGCGACCCCG | | | |
| | | AGGUGAGCGCUGCCC | | | |
| | | UGCAGAGCCGUCAAC | | | |
| | | AAGCGGCCCCUGACG | | | |
| | | CCGAUCUGAGCCAGG | | | |
| | | AGCCCCACCUGUUC | | | |

### Example 4. Expression of FANCA mRNA

mRNAs FANCA_01 (SEQ ID NO:4) and FANCA_02 (SEQ ID NO:5) were each generated with a codon-optimized ORF encoding FANCA (SEQ ID NO:1) and mRNA control elements, including a 5′ UTR and a 3′ UTR.

FaDu cells were acquired from the Fanconi Anemia Cancer Cell Line Resource and derived from a sporadic head and neck squamous cell carcinoma (ATCC #HTB43). A FANCA-knockout (KO) cell like was clonally derived from the FaDu cell line after CAS9-mediated deletion targeting of the N-terminus of the FANCA gene, resulting in loss of FANCA protein expression and function.

FaDu, Tg (transgene complemented from clonally derived FANCA−/− subline transfected with GFP mRNA), and FaDu-KO cells were seeded in the afternoon one day before transfection in 6 well plates (250,000-300,000 cells/well). The morning after, cells were transfected with the indicated quantities of FANCA_01-FANCA_11 mRNA or with GFP mRNA (as a control) using Lipofectamine MessengerMAX (Invitrogen, ref #LMRNA003) at a ratio of 1.5:1 μl/ug ratio of lipofectamine:mRNA according to the manufacturer's instructions. Media was changed 6 hours post-transfection and replaced with the appropriate indicated treatment. Cells were lysed either 6, 24, 48, or 72 hours after transfection in 1XNuPAGE LDS sample buffer. FANCA protein expression was determined by western blot using Cell Signaling 14657 antibody. Protein loading was normalized using Cell Signaling 87792 antibody for Nucleolin. The fold change expression of FANCA/nucleolin relative to the average expression in mock-treated FaDu cells.

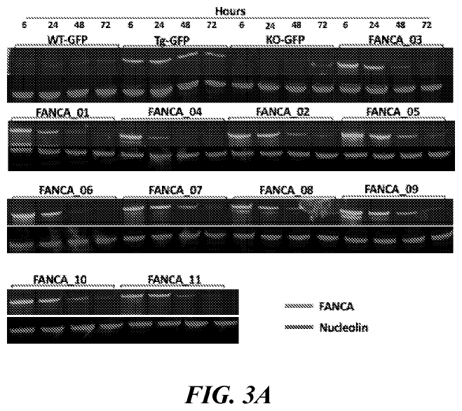

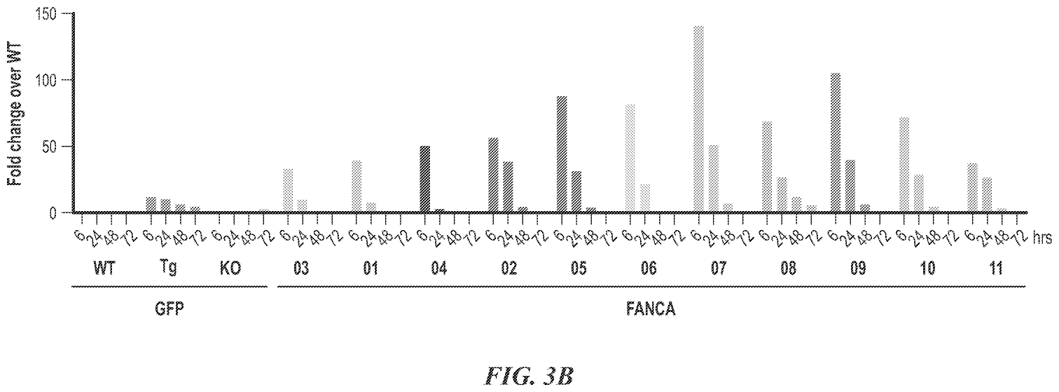

FIG. 3A is an image of a western blot showing the protein levels for FANCA and Nucleolin in the FaDu trio cell line at the indicated time points. Fold change FANCA expression was calculated normalized to loading control and wild type (WT) average. FANCA mRNA expressed protein in vitro up to three days.

FIG. 3B is a graph showing the fold change expression of FANCA/nucleolin relative to the average expression in WT. All FANCA constructs maintained protein expression levels comparable to or higher than WT up to 72 hours post-transfection.

### Example 5. FANCA mRNA Rescues Cell Survival After Treatment with MMC

FA patients commonly have increased sensitivity to interstrand DNA cross-linking agents (such as DEB and MMC). To determine if expression of FANCA mRNA can rescue FANCA-deficient cells from cell cycle arrest in the G2_M phase, FaDu, Tg, and FaDu-KO cells were transfected with 1 μg of GFP mRNA or FANCA mRNA, 24 hours after they were treated PBS (mock), DEB or MMC. In case of MMC, it was removed after 1 hr of treatment. Forty-eight hours later, the cells were collected, fixed and stained with DAPI in the presence of RNAse A for cell cycle analysis. To determine if the expression of FANCA mRNA can rescue FANCA-deficient cells from sensitivity to MMC, FaDu, Tg, and FaDu-KO cells were transfected with 1 μg of GFP mRNA or FANCA mRNA, 6 hours after they were treated PBS (mock) or different concentrations of MMC. Five days after culture in the presence of MMC, the treated cells were subsequently lysed using Cell Titer Glo reagent (Promega #G7570) to release total ATP and luminescence was read on a Bio-Tek Synergy HI. Five replicates per data point were normalized to untreated or transfection controls.

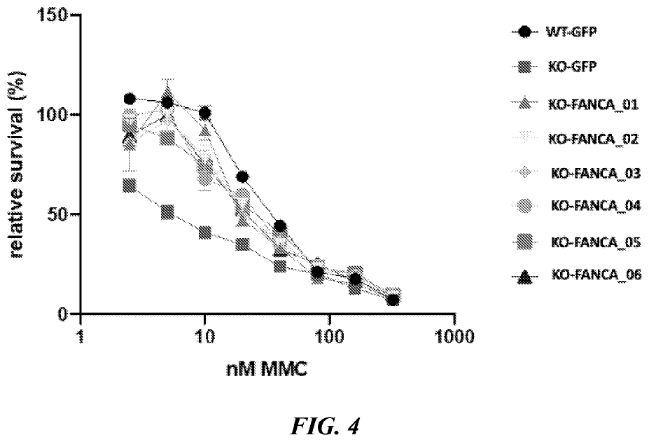

FIG. 4 is a graph showing percent survival of FaDu-WT, FaDu-KO transfected with lug GFP mRNA construct or FaDu-KO transfected with lug FANCA construct and treated with MMC at the indicated concentrations 24 hours post transfection. Survival was assessed 5 days post treatment using the cell titer Glo.

Re-expression of FANCA in FANCA-knockout cells rescues survival to levels similar to wild type.

### Example 6. FANCA mRNA Partially Rescues Cell Cycle

To determine if expression of FANCA mRNA can rescue FANCA-deficient cells from cell cycle arrest in the G2_M phase after treatment with DEB or MMC, GFP or FANCA (lug) mRNA transfected FaDu, Tg, and FaDu-KO cells were mock treated with PBS or treated with 0.05 ug/ml MMC (Sigma, ref #M0503) for 1 hour or with 0.1 ug/ml DEB (Sigma, ref #90474) for 48 hours. Cells were mock treated or treated with MMC or DEB 6 hours (FIG. 3A-FIG. 3B) or 72 hours (FIG. 4A-FIG. 4B) after transfection with the mRNA constructs. Cells were harvested using 0.25% trypsin and washed with PBS 48 hours post the addition of treatment.

Dead cells were labeled using LIVE/DEAD fixable far red (Invitrogen, ref #L10120) stain before being fixed using BD Cytofix/Cytoperm (BD, ref #554714) according to manufacturer's instructions. After fixation, cells were reconstituted in PBS containing 1 μg/ml 4′,6-diamidino-2-phenylindole (DAPI) (Invitrogen, ref #D21490) and 100ug/ml RNaseA (Thermo Scientific, ref #EN0531) for 15 minutes before being analyzed using the SONY-SH800Z.

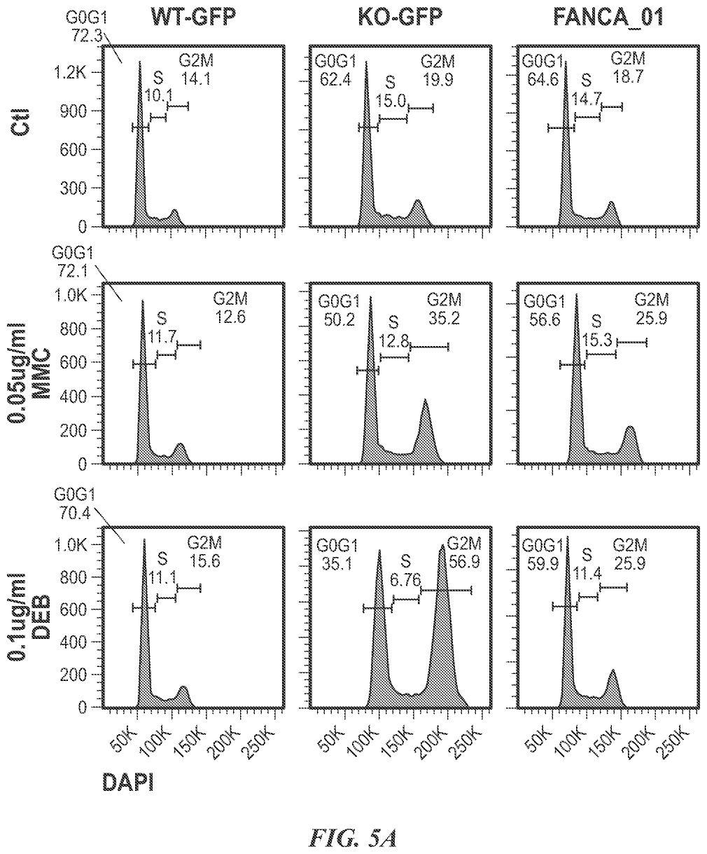

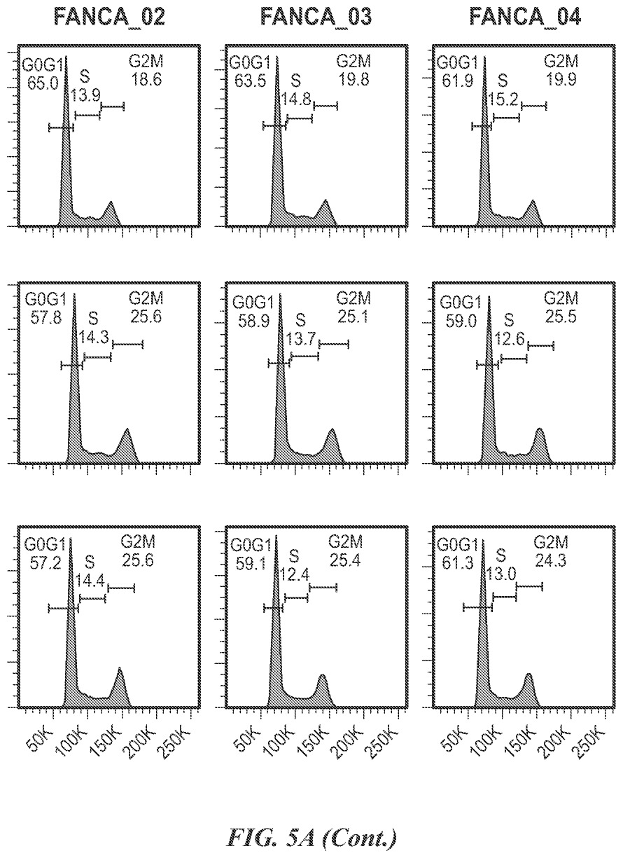

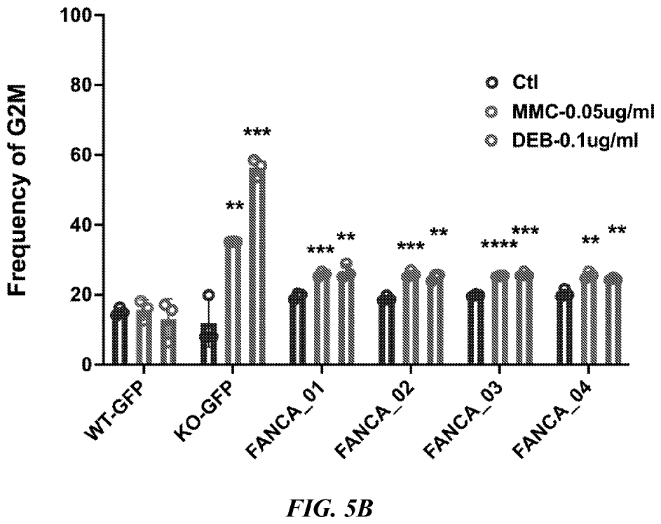

FIG. 5A is a graph showing accumulation of cells in G2_M phase of the cell cycle after transfection with 1 μg of FANCA mRNA or GFP mRNA followed by treatment with control (PBS), DEB, or MMC 6 hours later. In comparison to the GFP transfected FaDu-WT and the FaDu KO cells, cell cycle analysis demonstrated that transfection of 1 μg of FANCA mRNA constructs resulted in partial rescue of the accumulation of FaDu KO cells in the G2_M phase of the cell cycle 48 hours after treatment with DEB and MMC.

FIG. 5B is a graph showing frequency of G2M for the data of FIG. 5A, presented as mean±SD.

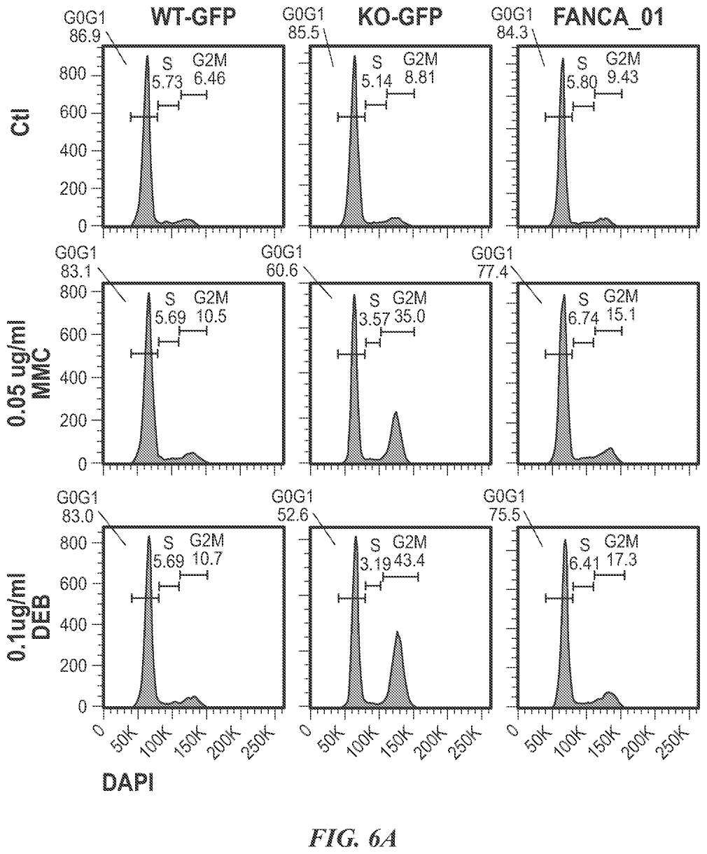

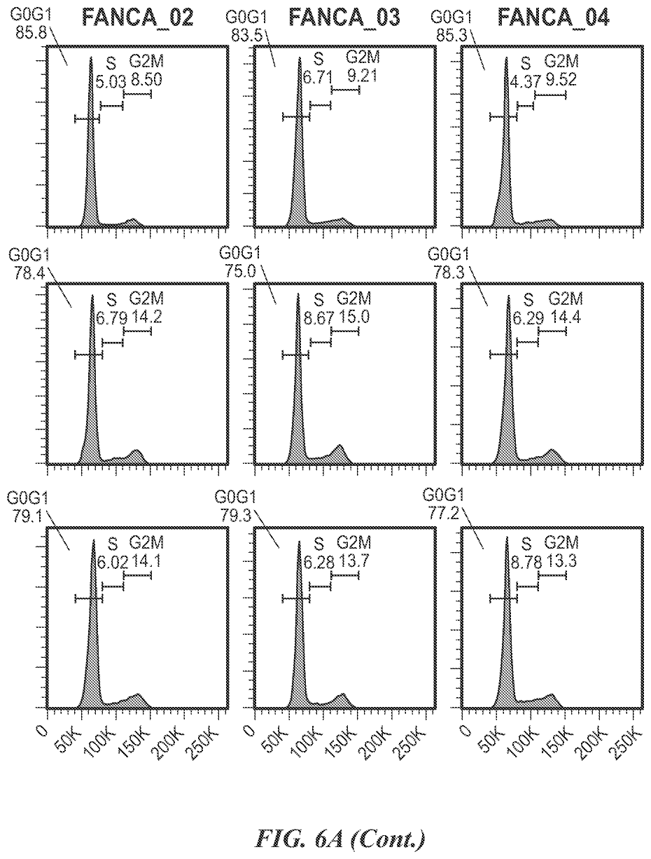

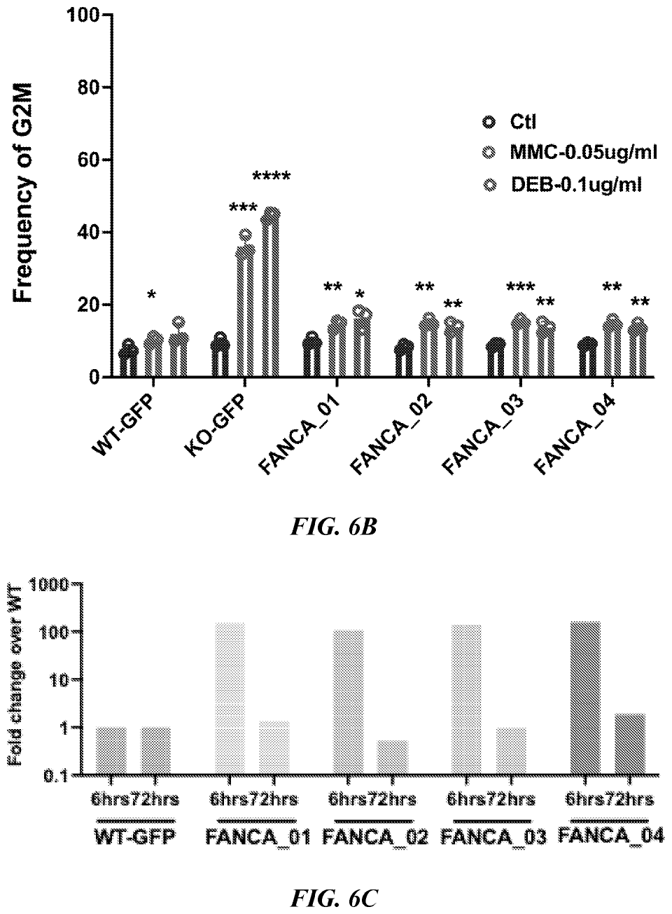

FIG. 6A is a graph showing accumulation of cells in G2_M phase of the cell cycle after transfection with 1 μg of FANCA mRNA or GFP mRNA followed by treatment with control (PBS), DEB, or MMC 72 hours later. In comparison to the GFP transfected FaDu-WT and the FaDu KO cells, cell cycle analysis demonstrated that transfection of 1 μg of FANCA mRNA constructs resulted in partial rescue of the accumulation of FaDu KO cells in the G2_M phase of the cell cycle 48 hours after treatment with DEB and MMC.

FIG. 6B is a graph showing frequency of G2M for the data of FIG. 6A, presented as mean±SD.

FIG. 6C is a graph showing expression levels of FANCA normalized to nucleolin and expressed as fold change over the of FANCA in the WT FaDu cell line.

### Example 7. In vivo Effect of mRNA Having the v2.0 5′ UTR in Combination with kappa 3′UTR

This Example describes in vivo assessments of vaccine induced immune responses.

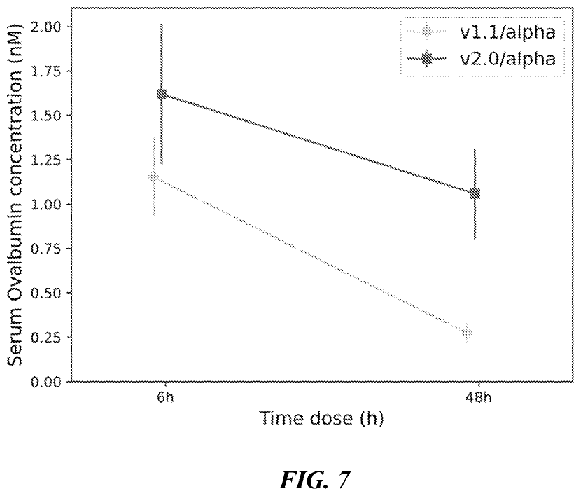

For evaluating the effect that the kappa 3′ UTR (SEQ ID NO: 139) has when combined with either vl.1 5′ UTR (SEQ ID NO: 56) or v2.0 5′ UTR (SEQ ID NO: 50) in vivo in mice, CL57BL/6 mice (N=8) were injected intramuscularly with 2 μg of mRNA formulated in heptadecan-9-yl 8-((2-hydroxyethyl)(6-oxo-6-(undecyloxy)hexyl)amino)octanoate. The mRNA encoded the secreted protein 15 Ovalbumin. At 6 h and 48h post-dose, 50 μl of blood was taken and serum generated. Ovalbumin concentration in the sera were assessed by ELISA (FIG. 7).

Two doses were administered 28 days apart. Antibody levels in serum were evaluated at two time points: day 28 (i.e. 4 weeks post-prime) and day 43 (i.e. 2 weeks post-boost). Antibody levels are expressed as endpoint titers, which is the greatest dilution of serum where antibody binding to a plate coated with Ovalbumin antigen can be detected.

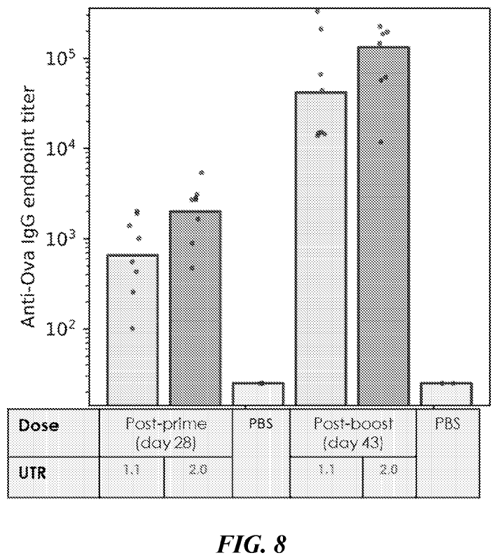

Results show that an mRNA comprising v2.0 5′ UTR (SEQ ID NO: 50) and kappa 3′UTR (SEQ ID NO: 139) increased antibody responses by ˜25× post-prime and ˜5× post-boost (FIG. 8).

### Example 8. In vivo effect of having a FUT8 sequence in the 3′ UTR

For evaluating the effect that a FUT8 sequence in the 3′ UTR would have on mRNAs, mRNAs encoding a protein of interest were formulated as an LNP (Compound II+GL67), then layered onto a primary human bronchial epithelial cell model. Several sequence variants were assessed:
1) A historical reference sequence (v1.0 UTRs)
2) An mRNA with v2.0 5′UTR (SEQ ID: NO: 50) and a re-optimized coding sequence
3) An mRNA with the elements from (2) plus a 3′UTR that bears the ‘delta’ stop cassette and ‘FUT8’ sequence (the 3′ UTR corresponds to SEQ ID NO:140).

Additionally, two positive controls that can rescue the activity of the mutant gene were assessed.

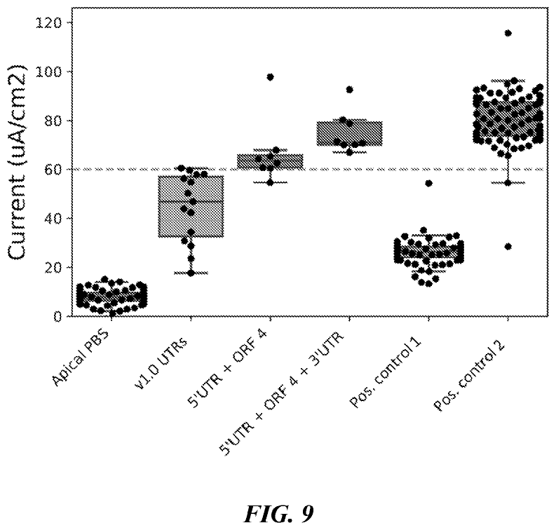

FIG. 9 shows the effect of a FUT8 sequence in the 3′UTR. Results show that having FUT8 increases target protein activity.

### Example 9. v2.0 5′ UTR enhances immunogenicity of an mRNA encoding hemagglutinin in mice

BALB c mice were injected with a co-formulation of four mRNAs encoding four hemagglutinin (HA) proteins (HA1, HA2, HA3, and HA4) having the v2.0 5′ UTR and the vl.1 3′ UTR with iota stop cassette (SEQ ID NOs: 50 and 180, respectively).

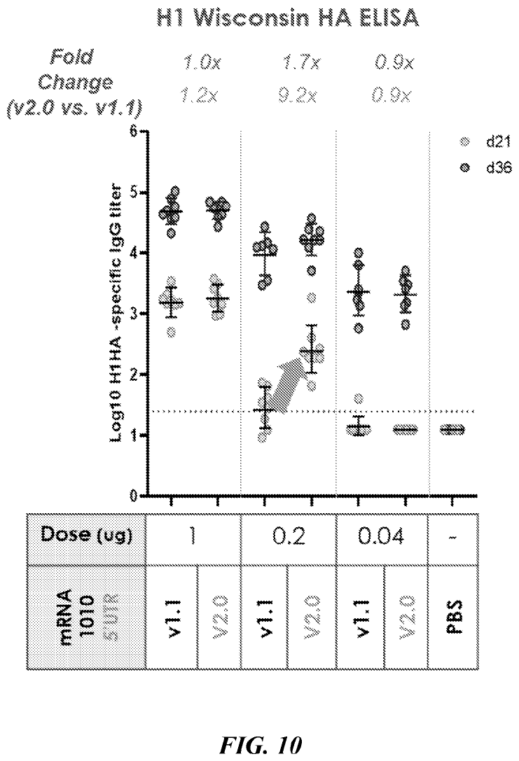

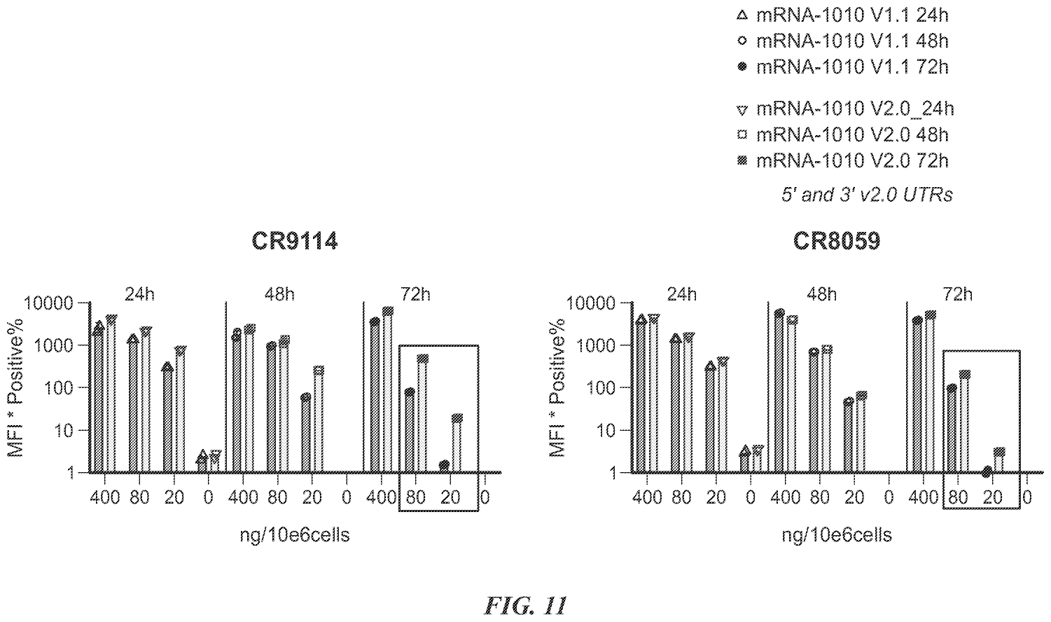

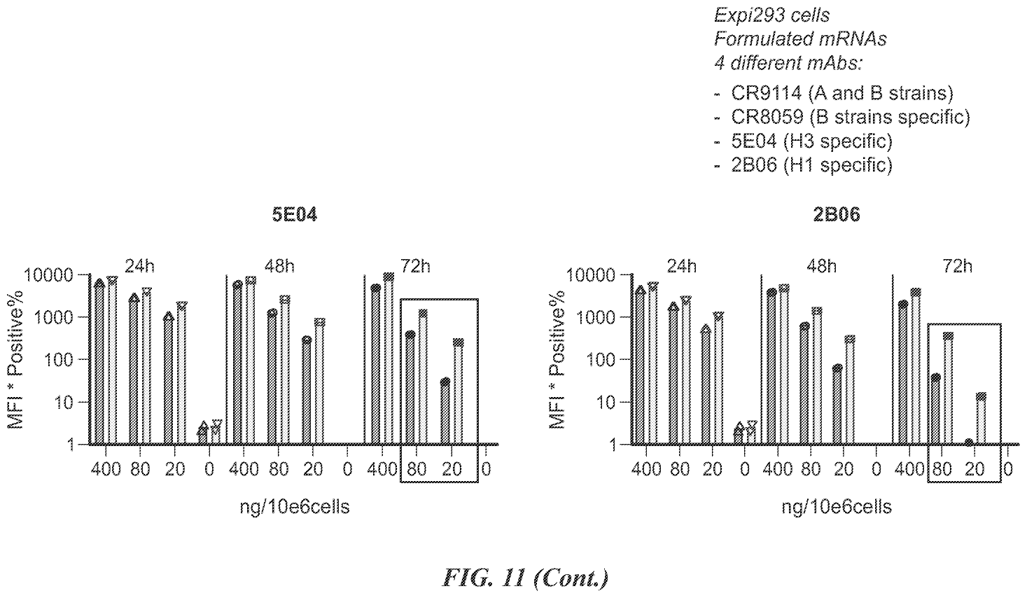

Specifically, HA mRNAs bearing the UTRs were co-formulated in SM-102 LNPs. LNPs were dose intramuscularly with a 3-week prime-boost interval. Antibody levels were assessed in serum at the days indicated, representing post-dose 1 and post-dose 2 (d21 and d36 in the Figure). The following doses were assessed: 1 μg, 0.2 pg, and 0.04 pg, along with PBS alone as a control. An increased antibody titer was mostly observed post-dose 1 for all 4 hemagglutinins (HAs). No impact on CD4 T cell responses to H1 and H3 HAs were observed in the spleen post-dose 2. Higher CD8 CD107a T cell responses against H1 HA in the spleen post-dose 2 was observed (see, e.g., FIG. 10). Increased IP-10 levels 6 hrs post-dose I and post-dose 2 was also observed. In general, the presence of the v2.0 5′ UTR enhanced immunogenicity after the first dose and at the medium dose level.

### Example 10. Combination of v2.0 5′ UTR and kappa 3′ UTR enhance in vitro expression of an mRNA encoding hemagglutinin

Similar to Example 9, cells were transfected with a co-formulation of four mRNAs encoding four hemagglutinin (HA) proteins (HA1, HA2, HA3, and HA4) having either the vl.1 5′ UTR and the vl.1 3′ UTR (SEQ ID NOs: 56 and 180, respectively) or the v2.0 5′ UTR and the kappa 3′ UTR (SEQ ID NO: 50 and SEQ ID NO: 139, respectively). Once again, HA mRNAs bearing either set of UTRs were co-formulated in SM-102 LNPs. LNPs were dose intramuscularly with a 3-week prime-boost interval.

Antibody levels were assessed in serum at the days indicated, representing post-dose 1 and post-dose 2. The cells were also treated with four different known flu vaccines (CR9114, CR8059, 5e04, and 2B06). The number of MFI positive cells were monitored at 24 h, 48h, and 72h, post dosing with the mRNA. As shown in FIG. 11, an increase in the expression of the four HA proteins was seen primarily at 72 hours with low dose of the co-formulated mRNA.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A messenger RNA (mRNA) comprising a 5′ UTR, an open reading frame encoding a polypeptide, and a 3′ UTR, wherein the 3′ UTR comprises:
(i) a nucleotide sequence at least 98% identical to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147; or
(ii) a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147, or a deletional variant thereof wherein 1 to 75 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147, wherein the nucleic acid sequence or the deletional variant thereof is modified to include:
a) one or more miRNA binding sites inserted within the nucleic acid sequence or the deletional variant thereof, and/or
b) a TENT recruiting sequence, a FUT8 recruiting sequence, one or more Identification and Ratio Determination (IDR) sequences, one or more ribosome engagement detection assay (REDA) sequences, or a combination of one or more IDR sequences and one or more REDA sequences inserted within the nucleic acid sequence or the deletional variant thereof.

2. The mRNA of claim 1, wherein the 3′ UTR comprises: a nucleotide sequence at least 99% identical to the nucleic acid sequence of SEQ ID NO:139, a nucleotide sequence at least 99% identical to the nucleic acid sequence of SEQ ID NO:140, a nucleotide sequence at least 99% identical to the nucleic acid sequence of SEQ ID NO:141, a nucleotide sequence at least 99% identical to the nucleic acid sequence of SEQ ID NO: 142, a nucleotide sequence at least 99% identical to the nucleic acid sequence of SEQ ID NO:143, a nucleotide sequence at least 99% identical to the nucleic acid sequence of SEO ID NO:144, a nucleotide sequence at least 99% identical to the nucleic acid sequence of SEQ ID NO:145, a nucleotide sequence at least 99% identical to the nucleic acid sequence of SEQ ID NO:146, or a nucleotide sequence at least 99% identical to the nucleic acid sequence of SEQ ID NO:147.

3-19. (canceled)

20. The mRNA of claim 1, wherein the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include one or more miRNA binding sites inserted within the nucleic acid sequence.

21. The mRNA of claim 20, wherein the one or more miRNA binding sites are selected from SEQ ID NOs:148-157.

22. The mRNA of claim 20, wherein the one or more miRNA binding sites comprise:
i) at least one copy of SEQ ID NO:149 and at least one copy of SEQ ID NO:150,
(ii) at least three copies of SEQ ID NO:150,
(iii) at least two copies of SEQ ID NO:149,
(iv) at least two copies of SEQ ID NO:149 and at least one copy of SEQ ID NO:150, or
(v) at least three copies of SEQ ID NO:148.

23-26. (canceled)

27. The mRNA of claim 1, wherein the 3′ UTR comprises a nucleotide sequence corresponding to the nucleic acid sequence of SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO: 147, wherein the nucleic acid sequence is modified to include: a TENT recruiting sequence inserted within the nucleic acid sequence, a FUT8 recruiting sequence inserted within the nucleic acid sequence, one or more IDR sequences inserted within the nucleic acid sequence, or one or more REDA sequences inserted within the nucleic acid sequence.

28-30. (canceled)

31. The mRNA of claim 1, wherein in the deletional variant 1 to 60, 1 to 50, 1 to 40, 1 to 30, 1 to 20, 1 to 10, or less than 10 consecutive nucleotides are deleted from SEQ ID NO:139, SEQ ID NO:140, SEQ ID NO:141, SEQ ID NO:142, SEQ ID NO:143, SEQ ID NO:144, SEQ ID NO:145, SEQ ID NO:146, or SEQ ID NO:147.

32-37. (canceled)

38. The mRNA of any one of claim 1, wherein the 5′ UTR comprises a nucleotide sequence at least 80%, 85%, 90%, 95%, 96%, 97%, 98%, 99% or 100% identical to SEQ ID NO:50.

39. (canceled)

40. The mRNA of claim 1,
a)wherein the 3′ UTR comprises the nucleic acid sequence set forth in SEQ ID NO:139, and wherein the 5′ UTR comprises the nucleic acid sequence set forth in SEQ ID NO:50, (b) wherein the 3′ UTR comprises the nucleic acid sequence set forth in SEQ ID NO:140, and wherein the 5′ UTR comprises the nucleic acid sequence set forth in SEQ ID NO:50, (c) wherein the 3′ UTR comprises the nucleic acid sequence set forth in SEQ ID NO:141, and wherein the 5′ UTR comprises the nucleic acid sequence set forth in SEQ ID NO:50, (d) wherein the 3′ UTR comprises the nucleic acid sequence set forth in SEQ ID NO:142, and wherein the 5′ UTR comprises the nucleic acid sequence set forth in SEO ID NO:50,
(e) wherein the 3′ UTR comprises the nucleic acid sequence set forth in SEQ ID NO:143, and wherein the 5′ UTR comprises the nucleic acid sequence set forth in SEQ ID NO:50,
(f) wherein the 3′ UTR comprises the nucleic acid sequence set forth in SEQ ID NO:144, and wherein the 5′ UTR comprises the nucleic acid sequence set forth in SEQ ID NO:50,
(g) wherein the 3′ UTR comprises the nucleic acid sequence set forth in SEQ ID NO:145, and wherein the 5′ UTR comprises the nucleic acid sequence set forth in SEQ ID NO:50,
(h) wherein the 3′ UTR comprises the nucleic acid sequence set forth in SEQ ID NO:146, and wherein the 5′ UTR comprises the nucleic acid sequence set forth in SEQ ID NO:50, or
(i) wherein the 3′ UTR comprises the nucleic acid sequence set forth in SEQ ID NO:147, and wherein the 5′ UTR comprises the nucleic acid sequence set forth in SEQ ID NO:50.

41-48. (canceled)

49. The mRNA of any one of claim 1, wherein the mRNA comprises a stop cassette.

50-51. (canceled)

52. The mRNA of claim 1, wherein the mRNA comprises a 5′ terminal cap.

53. (canceled)

54. The mRNA of claim 1, wherein the mRNA comprises a poly-A region.

55-56. (canceled)

57. The mRNA of claim 1, wherein the poly-A region comprises A100-UCUAG-A20-inverted deoxy-thymidine (SEQ ID NO:183).

58. The mRNA of claim 1, wherein the mRNA comprises at least one chemically modified nucleobase, sugar, backbone, or any combination thereof.

59. The mRNA of claim 58, wherein the at least one chemically modified nucleobase is selected from the group consisting of pseudouracil (ψ), N₁-methylpseudouracil (m1ψ), 1-ethylpseudouracil, 2-thiouracil (s2U), 4′-thiouracil, 5-methylcytosine, 5-methyluracil, 5-methoxyuracil, and any combination thereof.

60. The mRNA of claim 1, wherein the polypeptide comprises a secreted protein, a membrane-bound protein, or an intercellular protein.

61. The mRNA of claim 60, wherein the polypeptide is a cytokine, an antibody, a vaccine, a receptor, an enzyme, a hormone, a transcription factor, a ligand, a membrane transporter, a structural protein, a nuclease, or a component, variant or fragment thereof.

62. A pharmaceutical composition comprising the mRNA of claim 1 and a pharmaceutically acceptable carrier.

63. A lipid nanoparticle comprising the mRNA of claim 1.

64. The lipid nanoparticle of claim 63, wherein the lipid nanoparticle comprises:
(i) an ionizable lipid,
(ii) a phospholipid,
(iii) a structural lipid, and
(iv) a PEG-lipid.

65. The lipid nanoparticle of claim 63, wherein the lipid nanoparticle comprises a compound of Formula (I): 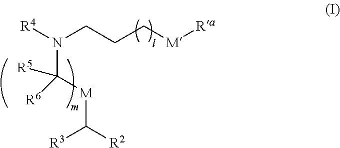
or its N-oxide, or a salt or isomer thereof,
wherein R^{′ a} is R^{′branched}; wherein
R^{′branched} is: 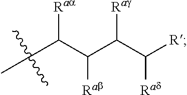
wherein 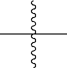
denotes a point of attachment;
wherein R^{aα}, Raβ, Raγ, and Raδ are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH2)nOH, wherein n is selected from the group consisting of 1, 2, 3, 4, and 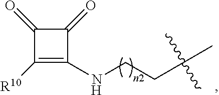
wherein 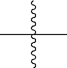
denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

66-70. (canceled)

71. The lipid nanoparticle of claim 63, wherein the lipid nanoparticle is formulated for intravenous, subcutaneous, intramuscular, intranasal, intraocular, rectal, pulmonary or oral delivery.

72. A pharmaceutical composition comprising the lipid nanoparticle of claim 63.

73. A cell comprising the lipid nanoparticle of claim 63.

74. A method of increasing expression of a polypeptide, comprising administering to a cell the lipid nanoparticle of claim 63.

75. A method of delivering the lipid nanoparticle of claim 63 to a cell, comprising contacting the cell in vitro, in vivo or ex vivo with the lipid nanoparticle.

76. A method of delivering the lipid nanoparticle of claim 63 to a human subject having a disease or disorder, comprising administering to the human subject in need thereof an effective amount of the lipid nanoparticle.

77. A method of treating, preventing, or preventing a symptom of, a disease or disorder in a human subject in need thereof, comprising administering to the human subject an effective amount of the lipid nanoparticle of claim 63.
